(19) United States
(12) Patent Application Publication
(10) Pub. No.: US 2011/0217740 A1
(43) Pub. Date: Sep. 8, 2011

(54) METHODS, MICROORGANISMS, AND COMPOSITIONS FOR PLANT BIOMASS PROCESSING

(75) Inventors: Michael W.W. Adams, Athens, GA (US); Janet Westpheling, Bogart, GA (US); Scott Hamilton-Brehm, Oak Ridge, TN (US); Irina Kataeva, Athens, GA (US); Sung-Jae Yang, Athens, GA (US); Farris Poole, Bogart, GA (US)

(73) Assignee: University of Georgia Research, Athens, GA (US)

(21) Appl. No.: 13/061,278

(22) PCT Filed: Aug. 26, 2009
(86) PCT No.: PCT/US09/55049

§ 371 Date: May 19, 2011

Related U.S. Application Data
(60) Provisional application No. 61/190,181, filed on Aug. 26, 2008.

(51) Int. Cl. C12P 1/04; C12P 7/04; C12P 7/06; C12P 7/16; C12P 7/64; C12P 3/00; C12P 7/46; C12P 7/48; C12P 7/50; C12P 7/44; C12P 7/40; C12P 19/12; C12P 19/02; C12N 15/63; C12N 15/81; C12N 1/00; C12N 1/21; C12N 9/14

(52) U.S. Cl. 435/100; 435/170; 435/157; 435/161; 435/160; 435/134; 435/168; 435/145; 435/144; 435/143; 435/142; 435/136; 435/105; 435/471; 435/243; 435/252.3; 435/195

(57) ABSTRACT

Disclosed herein are methods of degrading plant biomass, and microorganisms and polypeptides used in such methods, hi certain embodiments, the methods include growing Anaerocellum thermophilum on a substrate that comprises plant biomass under conditions effective for the A. thermophilum to convert at least a portion of the plant biomass to a water soluble product or a water insoluble product, hi some cases, the method can further include one or more steps to further process the water soluble product or a water insoluble product to produce, for example, a biofuel or commodity chemical. In another aspect, microorganisms that include at least one A. thermophilum plant biomass utilization polynucleotide are disclosed. Also disclosed are methods of transferring one or more A. thermophilum plant biomass utilization polynucleotides to a recipient microorganism. A. thermophilum plant biomass utilization polynucleotides and polypeptides encoded by such polynucleotides are also disclosed. Also disclosed are methods of degrading plant biomass by providing an isolated A. thermophilum polypeptide capable of degrading unprocessed plant biomass, and contacting the A. thermophilum polypeptide with plant biomass under conditions effective for the A. thermophilum polypeptide to at least partially degrade the plant biomass.

### CROSS REFERENCE TO RELATED APPLICATIONS

This application claims priority to U.S. Provisional Patent Application Ser. No. 61/190,181, filed Aug. 26, 2008.

### STATEMENT OF GOVERNMENT RIGHTS

This invention was made with government support under a grant from the Department of Energy, Grant No. DE-PS02-06ER64304. The U.S. Government has certain rights in this invention.

### BACKGROUND

Biofuel can be broadly defined as solid, liquid, or gas fuel derived from recently dead biological material. The derivation of biofuel from recently dead biological material distinguishes it from fossil fuels, which are derived from long dead biological material. Biofuel can be theoretically produced from any biological carbon source, but a common source of biofuel is photosynthetic plants. Many different plants and plant-derived materials may be used for biofuel manufacture.

One strategy for producing biofuel involves growing crops high in either sugar (e.g., sugar cane, sugar beet, and sweet sorghum) or starch (e.g., corn/maize), and then using yeast fermentation to produce ethyl alcohol (ethanol). One challenge associated with this strategy is that competition between food markets and energy markets for the crops can increase food costs.

Thus, a second strategy involves converting biological material such as, for example, wood and its byproducts into biofuels such as, for example, woodgas, methanol, or ethanol fuel. It is also possible to make cellulosic biofuel—e.g., cellulosic ethanol—from non-edible plant parts. Cellulosic biofuel production can use non-food crops or inedible waste products. Thus, producing cellulosic biofuel need not divert food crops away from the animal or human food chain. Moreover, in some cases, biofuel can be produced from material that would otherwise present a disposal problem.

Producing biofuel from cellulose can be economically challenging, however. It often involves multiple processing steps to break down the cellulose and convert the biological material into material that is, or can be readily converted to, biofuel. Each processing step can make the overall process more costly and, therefore, decrease the economic feasibility of producing biofuel from cellulosic biological material. Thus, there is a need to develop methods that reduce the number of processing steps needed to convert cellulosic biological material to biofuel and other commercially desirable materials.

Anaerocellum thermophilum was first described in 1990. A. thermophilum DSM 6725 is a strict anaerobic microorganism with a temperature optimum at 72-75° C. It is freely available from a public culture collection at DSM-Deutsche Sammlung von Mikroorganismen and Zellkulturen GmbH, Mascheroder Weg 1b, D-3300 Braunschweig, Germany, under the accession number DSM 6725.

### SUMMARY OF THE INVENTION

The present invention relates to methods, microorganisms, and compositions useful for processing plant biomass. The application of this technology has the potential to render production of biofuels more economically feasible and to allow any microorganism to utilize recalcitrant biomass. The use of cellulosic materials as sources of bioenergy is currently limited by typically requiring pretreatment of the cellulosic material. Such pretreatments can be expensive. Thus, methods that reduce dependence of existing pretreatments of cellulosic materials may have a dramatic impact on the economics of the use of recalcitrant biomass for biofuels production.

In one aspect, the methods described herein involve processing plant biomass. Generally, the methods include growing Anaerocellum thermophilum on a substrate that comprises plant biomass under conditions effective for the A. thermophilum to convert at least a portion of the plant biomass to a product that may be water soluble or water insoluble. In some cases, methods described herein can yield both soluble and insoluble products that are more readily converted to biofuel, a polymer, or commodity chemicals than unprocessed plant biomass. In other cases, the methods themselves can include converting the plant biomass to biofuel, a polymer, and/or a commodity chemical.

In another aspect, methods described herein include transferring one or more polynucleotides that include at least one A. thermophilum coding region to a recipient microorganism. In some embodiments, the method involves direct or indirect cloning of an A. thermophilum polynucleotide, then introducing the A. thermophilum polynucleotide into a recipient microorganism. In other embodiments, A. thermophilum is co-cultivated with a recipient microorganism, wherein the A. thermophilum comprises a conjugative polynucleotide, and wherein the co-cultivation is under conditions suitable for conjugative transfer of at least a portion of the conjugative polynucleotide from the A. thermophilum to the recipient microorganism; and identifying a recipient microorganism exconjugant.

In another aspect, the present invention provides a genetically-modified microorganism comprising one or more A. thermophilum plant biomass utilization (PBU) coding regions. In some cases, the PBU coding region comprises a polysaccharide hydrolases and related enzymes (PHR) coding rgion.

In another aspect, the methods described herein involve using a microorganism for processing plant biomass. Generally, the methods include growing microorganisms comprising one or more A. thermophilum plant biomass utilization (PBU) coding regions on a substrate that comprises unprocessed or spent plant biomass under conditions effective for the microorganism to convert at least a portion of the plant biomass to a soluble product.

In another aspect, the present invention provides an isolated polypeptide, and compositions comprising the isolated polypeptide, in which the isolated polypeptide includes an amino acid sequence that is at least 80% identical to the amino acid sequence of a PBU polypeptide. In some embodiments, the PBU polypeptide comprises a PHR polypeptide.

In another aspect, the invention provides a method of making an isolated A. thermophilum polypeptide. Generally, the method includes growing a microorganism comprising at least one coding region encoding an A. thermophilum polypeptide under conditions effective for the microorganism to produce the A. thermophilum polypeptide, and isolating the A. thermophilum polypeptide.

In yet another aspect, the present invention provides a method of processing plant biomass using an isolated A. thermophilum polypeptide. Generally, the method includes providing an isolated A. thermophilum polypeptide; and contacting the A. thermophilum polypeptide with plant biomass under conditions effective for the A. thermophilum polypeptide to at least partially degrade the plant biomass.

The above summary of the present invention is not intended to describe each disclosed embodiment or every implementation of the present invention. The description that follows more particularly exemplifies illustrative embodiments. However, embodiments other than those expressly described are possible and may be made, used, and/or practiced under circumstances and/or conditions that are the same or different from the circumstances and/or conditions described in connection with the illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Growth of A. thermophilum on unprocessed wood and grass biomass.

FIG. 2. Growth of A. thermophilum on defined substrates: cellobiose, crystalline cellulose (Avicel), and xylan (oat spelt).

FIG. 3. End products of growth of A. thermophilum on defined substrates: cellobiose, crystalline cellulose (Avicel) and xylan (oat spelt).

FIG. 4. Growth of A. thermophilum on unprocessed switchgrass and poplar.

FIG. 5. End products of growth of A. thermophilum on unprocessed switchgrass or poplar.

FIG. 6. Growth of A. thermophilum in flushed cultures on defined and undefined substrates (poplar, xylan and cellobiose).

FIG. 7. End products of growth of A. thermophilum in flushed cultures on defined and undefined substrates (poplar, xylan and cellobiose).

FIG. 8. Growth of A. thermophilum on ‘spent’ poplar and switchgrass.

FIG. 9. End products of growth of A. thermophilum on ‘spent’ poplar and switchgrass.

FIG. 10. Growth of A. thermophilum on ‘spent’ crystalline cellulose (Avicel).

FIG. 11. End products of growth of A. thermophilum on ‘spent’ crystalline cellulose (Avicel).

FIG. 12. Growth of A. thermophilum on a defined medium (on cellobiose) and on untreated switchgrass and poplar in the absence of yeast extract.

FIG. 13. Growth of A. thermophilum and C. saccharolyticus on soluble and insoluble heat-treated (98° C./2 min) extracts of switchgrass.

FIG. 14. Growth of A. thermophilum and C. saccharolyticus on soluble and insoluble heat-treated extracts of poplar.

FIG. 15. Growth of A. thermophilum and C. saccharolyticus on soluble and insoluble heat-treated extracts of pine.

FIG. 16. CelA fragment encoding GH9-CBM (GH9 is catalytic domain, CBM is carbohydrate-binding domain).

FIG. 17. Signal sequence of P. furiosus amylase coding region.

FIG. 18. Plasmid pS2-SP used to generate the recombinant P. furiosus strain containing A. thermophilum CelA.

FIG. 19. Plasmid pS2-GH9 used to generate the recombinant P. furiosus strain containing A thermophilum CelA.

FIG. 20. PCR using primers GDHcasUP-HMGcasDOWN will amplify a 1500 bp fragment diagnostic of PF GDH-HMG cassette.

FIG. 21. Confirmation of GH9(CelA) and GH9sp(CelA+signal peptide) exconjugants.

FIG. 22. Confirmation of GH9(CelA) and GH9sp(CelA+signal peptide) exconjugants.

FIG. 23. Nucleotide and amino acid sequences of selected A. thermophilum plant biomass utilization (PBU) coding regions.

FIG. 23-01: Nucleotide sequence (SEQ ID NO:18) and amino acid sequence (SEQ ID NO:19) of Athe_{—}0010.

FIG. 23-02: Nucleotide sequence (SEQ ID NO:20) and amino acid sequence (SEQ ID NO:21) of Athe_{—}0011.

FIG. 23-03: Nucleotide sequence (SEQ ID NO:22) and amino acid sequence (SEQ ID NO:23) of Athe_{—}0012.

FIG. 23-04: Nucleotide sequence (SEQ ID NO:24) and amino acid sequence (SEQ ID NO:25) of Athe_{—}0013.

FIG. 23-05: Nucleotide sequence (SEQ ID NO:26) and amino acid sequence (SEQ ID NO:27) of Athe_{—}0014.

FIG. 23-06: Nucleotide sequence (SEQ ID NO:28) and amino acid sequence (SEQ ID NO:29) of Athe_{—}0015.

FIG. 23-07: Nucleotide sequence (SEQ ID NO:30) and amino acid sequence (SEQ ID NO:31) of Athe_{—}0016.

FIG. 23-08: Nucleotide sequence (SEQ ID NO:32) and amino acid sequence (SEQ ID NO:33) of Athe_{—}0017.

FIG. 23-09: Nucleotide sequence (SEQ ID NO:34) and amino acid sequence (SEQ ID NO:35) of Athe_{—}0052.

FIG. 23-10: Nucleotide sequence (SEQ ID NO:36) and amino acid sequence (SEQ ID NO:37) of Athe_{—}0053.

FIG. 23-11: Nucleotide sequence (SEQ ID NO:38) and amino acid sequence (SEQ ID NO:39) of Athe_{—}0054.

FIG. 23-12: Nucleotide sequence (SEQ ID NO:40) and amino acid sequence (SEQ ID NO:41) of Athe_{—}0055.

FIG. 23-13: Nucleotide sequence (SEQ ID NO:42) and amino acid sequence (SEQ ID NO:43) of Athe_{—}0056.

FIG. 23-14: Nucleotide sequence (SEQ ID NO:44) and amino acid sequence (SEQ ID NO:45) of Athe_{—}0057.

FIG. 23-15: Nucleotide sequence (SEQ ID NO:46) and amino acid sequence (SEQ ID NO:47) of Athe_{—}0058.

FIG. 23-16: Nucleotide sequence (SEQ ID NO:48) and amino acid sequence (SEQ ID NO:49) of Athe_{—}0059.

FIG. 23-17: Nucleotide sequence (SEQ ID NO:50) and amino acid sequence (SEQ ID NO:51) of Athe_{—}0060.

FIG. 23-18: Nucleotide sequence (SEQ ID NO:52) and amino acid sequence (SEQ ID NO:53) of Athe_{—}0061.

FIG. 23-19: Nucleotide sequence (SEQ ID NO:54) and amino acid sequence (SEQ ID NO:55) of Athe_{—}0077.

FIG. 23-20: Nucleotide sequence (SEQ ID NO:56) and amino acid sequence (SEQ ID NO:57) of Athe_{—}0088.

FIG. 23-21: Nucleotide sequence (SEQ ID NO:58) and amino acid sequence (SEQ ID NO:59) of Athe_{—}0089.

FIG. 23-22: Nucleotide sequence (SEQ ID NO:60) and amino acid sequence (SEQ ID NO:61) of Athe_{—}0090.

FIG. 23-23: Nucleotide sequence (SEQ ID NO:62) and amino acid sequence (SEQ ID NO:63) of Athe_{—}0153.

FIG. 23-24: Nucleotide sequence (SEQ ID NO:64) and amino acid sequence (SEQ ID NO:65) of Athe_{—}0154.

FIG. 23-25: Nucleotide sequence (SEQ ID NO:66) and amino acid sequence (SEQ ID NO:67) of Athe_{—}0155.

FIG. 23-26: Nucleotide sequence (SEQ ID NO:68) and amino acid sequence (SEQ ID NO:69) of Athe_{—}0156.

FIG. 23-27: Nucleotide sequence (SEQ ID NO:70) and amino acid sequence (SEQ ID NO:71) of Athe_{—}0157.

FIG. 23-28: Nucleotide sequence (SEQ ID NO:72) and amino acid sequence (SEQ ID NO:73) of Athe_{—}0158.

FIG. 23-29: Nucleotide sequence (SEQ ID NO:74) and amino acid sequence (SEQ ID NO:75) of Athe_{—}0159.

FIG. 23-30: Nucleotide sequence (SEQ ID NO:76) and amino acid sequence (SEQ ID NO:77) of Athe_{—}0160.

FIG. 23-31: Nucleotide sequence (SEQ ID NO:78) and amino acid sequence (SEQ ID NO:79) of Athe_{—}0450.

FIG. 23-32: Nucleotide sequence (SEQ ID NO:80) and amino acid sequence (SEQ ID NO:81) of Athe_{—}0451.

FIG. 23-33: Nucleotide sequence (SEQ ID NO:82) and amino acid sequence (SEQ ID NO:83) of Athe_{—}0452.

FIG. 23-34: Nucleotide sequence (SEQ ID NO:84) and amino acid sequence (SEQ ID NO:85) of Athe_{—}0607.

FIG. 23-35: Nucleotide sequence (SEQ ID NO:86) and amino acid sequence (SEQ ID NO:87) of Athe_{—}0608.

FIG. 23-36: Nucleotide sequence (SEQ ID NO:88) and amino acid sequence (SEQ ID NO:89) of Athe_{—}1853.

FIG. 23-37: Nucleotide sequence (SEQ ID NO:90) and amino acid sequence (SEQ ID NO:91) of Athe_{—}1854.

FIG. 23-38: Nucleotide sequence (SEQ ID NO:92) and amino acid sequence (SEQ ID NO:93) of Athe_{—}1855.

FIG. 23-39: Nucleotide sequence (SEQ ID NO:94) and amino acid sequence (SEQ ID NO:95) of Athe_{—}1856.

FIG. 23-40: Nucleotide sequence (SEQ ID NO:96) and amino acid sequence (SEQ ID NO:97) of Athe_{—}1989.

FIG. 23-41: Nucleotide sequence (SEQ ID NO:98) and amino acid sequence (SEQ ID NO:99) of Athe_{—}1990.

FIG. 23-42: Nucleotide sequence (SEQ ID NO:100) and amino acid sequence (SEQ ID NO:101) of Athe_{—}1991.

FIG. 23-43: Nucleotide sequence (SEQ ID NO:102) and amino acid sequence (SEQ ID NO:103) of Athe_{—}1992.

FIG. 23-44: Nucleotide sequence (SEQ ID NO:104) and amino acid sequence (SEQ ID NO:105) of Athe_{—}1993.

FIG. 23-45: Nucleotide sequence (SEQ ID NO:106) and amino acid sequence (SEQ ID NO:107) of Athe_{—}1994.

FIG. 23-46: Nucleotide sequence (SEQ ID NO:108) and amino acid sequence (SEQ ID NO:109) of Athe_{—}2076.

FIG. 23-47: Nucleotide sequence (SEQ ID NO:110) and amino acid sequence (SEQ ID NO:111) of Athe_{—}2077.

FIG. 23-48: Nucleotide sequence (SEQ ID NO:112) and amino acid sequence (SEQ ID NO:113) of Athe_{—}2078.

FIG. 23-49: Nucleotide sequence (SEQ ID NO:114) and amino acid sequence (SEQ ID NO:115) of Athe_{—}2079.

FIG. 23-50: Nucleotide sequence (SEQ ID NO:116) and amino acid sequence (SEQ ID NO:117) of Athe_{—}2080.

FIG. 23-51: Nucleotide sequence (SEQ ID NO:118) and amino acid sequence (SEQ ID NO:119) of Athe_{—}2081.

FIG. 23-52: Nucleotide sequence (SEQ ID NO:120) and amino acid sequence (SEQ ID NO:121) of Athe_{—}2082.

FIG. 23-53: Nucleotide sequence (SEQ ID NO:122) and amino acid sequence (SEQ ID NO:123) of Athe_{—}2083.

FIG. 23-54: Nucleotide sequence (SEQ ID NO:124) and amino acid sequence (SEQ ID NO:125) of Athe_{—}2084.

FIG. 23-55: Nucleotide sequence (SEQ ID NO:126) and amino acid sequence (SEQ ID NO:127) of Athe_{—}2085.

FIG. 23-56: Nucleotide sequence (SEQ ID NO:128) and amino acid sequence (SEQ ID NO:129) of Athe_{—}2086.

FIG. 23-57: Nucleotide sequence (SEQ ID NO:130) and amino acid sequence (SEQ ID NO:131) of Athe_{—}2087.

FIG. 23-58: Nucleotide sequence (SEQ ID NO:132) and amino acid sequence (SEQ ID NO:133) of Athe_{—}2088.

FIG. 23-59: Nucleotide sequence (SEQ ID NO:134) and amino acid sequence (SEQ ID NO:135) of Athe_{—}2089.

FIG. 23-60: Nucleotide sequence (SEQ ID NO:136) and amino acid sequence (SEQ ID NO:137) of Athe_{—}2090.

FIG. 23-61: Nucleotide sequence (SEQ ID NO:138) and amino acid sequence (SEQ ID NO:139) of Athe_{—}2091.

FIG. 23-62: Nucleotide sequence (SEQ ID NO:140) and amino acid sequence (SEQ ID NO:141) of Athe_{—}2092.

FIG. 23-63: Nucleotide sequence (SEQ ID NO:142) and amino acid sequence (SEQ ID NO:143) of Athe_{—}2093.

FIG. 23-64: Nucleotide sequence (SEQ ID NO:144) and amino acid sequence (SEQ ID NO:145) of Athe_{—}2094.

FIG. 23-65: Nucleotide sequence (SEQ ID NO:146) and amino acid sequence (SEQ ID NO:147) of Athe_{—}2371.

FIG. 23-66: Nucleotide sequence (SEQ ID NO:148) and amino acid sequence (SEQ ID NO:149) of Athe_{—}2372.

FIG. 23-67: Nucleotide sequence (SEQ ID NO:150) and amino acid sequence (SEQ ID NO:151) of Athe_{—}2373.

FIG. 23-68: Nucleotide sequence (SEQ ID NO:152) and amino acid sequence (SEQ ID NO:153) of Athe_{—}2374.

FIG. 23-69: Nucleotide sequence (SEQ ID NO:154) and amino acid sequence (SEQ ID NO:155) of Athe_{—}2375.

FIG. 23-70: Nucleotide sequence (SEQ ID NO:156) and amino acid sequence (SEQ ID NO:157) of Athe_{—}2376.

FIG. 23-71: Nucleotide sequence (SEQ ID NO:158) and amino acid sequence (SEQ ID NO:159) of Athe_{—}0423.

FIG. 23-72: Nucleotide sequence (SEQ ID NO:160) and amino acid sequence (SEQ ID NO:161) of Athe_{—}0603.

FIG. 23-73: Nucleotide sequence (SEQ ID NO:162) and amino acid sequence (SEQ ID NO:163) of Athe_{—}0610.

FIG. 24. Growth of A. thermophilum on washed and unwashed peanut shells.

FIG. 25. Gene clusters encoding multi-domain carbohydrate active enzymes from A. thermophilum and C. saccharolyticus.

FIG. 26. Construction of Shuttle Vector pDCW 31.

FIG. 27. Peptide domains common to A. thermophilum DSM6725 and C. saccharolyticus DSM8903.

FIG. 28. Peptide domains unique to A. thermophilum DSM 6725.

FIG. 29. Peptide domain re-arrangements in A. thermophilum compared to C. saccharolyticus.

FIG. 30. Peptide domains enriched in A. thermophilum DSM6725 and C. saccharolyticus DSM8903.

FIG. 31. Differential expression of extracellular proteins during growth of A. thermophilum DSM 6725 on crystalline cellulose.

FIG. 32. Non-catalytic extracellular (ExtP) or membrane-associated (Memb) proteins in A. thermophilum DSM 6750.

FIG. 33. Exemplary proteins produced by A. thermophilum during growth on cellulose, xylan, poplar and/or switchgrass that are not encoded in the C. saccharolyticus genome.

### DETAILED DESCRIPTION OF ILLUSTRATIVE EMBODIMENTS

The present invention relates to methods, microorganisms, and compositions useful for processing plant biomass. The invention relates, in certain aspects, to a group of coding regions, the expression of which can enable a microorganism to convert plant biomass such as, for example, poplar wood chips, to soluble products that can be used by the same or by another microorganism to produce an economically desirable product such as, for example, a biofuel (e.g., an alcohol and/or hydrogen gas (H₂)), polymer, or commodity chemical.

The application of this technology has the potential to render production of biofuels more economically feasible and to allow a broader range of microorganisms to utilize recalcitrant biomass. The use of cellulosic materials as sources of bioenergy is currently limited by typically requiring preprocessing of the cellulosic material. Such preprocessing methods can be expensive. Thus, methods that reduce dependence on preprocessing of cellulosic materials may have a dramatic impact on the economics of the use of recalcitrant biomass for biofuels production.

One challenge in converting biomass into liquid (e.g., ethanol, biodiesel) and gaseous (e.g., H₂) fuels is the recalcitrance and heterogeneity of the biological material. Consequently, effective and efficient conversion of the biological material cannot be achieved by a single naturally-occurring microorganism, a mixture of naturally-occurring microorganisms, or a mixture of enzymes. In certain aspects, the present invention involves exploiting a specific group of coding regions, the so-called plant biomass utilization (PBU) gene set of Anaerocellum thermophilum. Expression of one or more of these coding regions can enable processed, unprocessed, and/or spent samples of plant biomass to be utilized directly for biomass conversion. These coding regions can be expressed by various microorganisms by the appropriate genetic manipulations. The microorganisms may be thermophilic microorganisms such as, for example, A. thermophilum or may be mesophilic microorganisms. Moreover, the products of biomass conversion are not limited to biofuels, but extend to any polymer or commodity chemical derived from plant cell biomass.

In the description that follows, the following terms shall have the meanings set forth below.

“Biofuel” refers to a combustible material that can be produced through chemical, enzymatic, or microbiotic fermentation or processing of plant biomass (e.g., processed biomass, unprocessed biomass, spent biomass, etc.) and that can be used, alone or in combination with other materials, for the generation of energy.

“Commodity chemical” refers to any product (e.g., oxalic acid, succinic acid, lactic acid, pyruvic acid, salts thereof, amino acids, etc.) from the fermentation of plant biomass (e.g., processed biomass, unprocessed biomass, spent biomass, etc.) that can be the starting material for the production of other chemicals and/or materials.

“Extremophilic” refers to a microorganism that can thrive in, and may require, specific conditions that are unfavorable to other microorganisms.

“Exconjugant” refers to a cell that, after conjugation, has received DNA from a conjugation partner cell.

“Mesophilic” refers to a microorganism that has a temperature optimum for growth of from 20-37° C.

“Processed plant biomass” refers to plant biomass that has been subjected to chemical, physical, microbial, or enzymatic processing under conditions such that at least some of the complex organic polymers originally present in the plant biomass are degraded to smaller chemical subunits.

“Spent biomass” refers to water insoluble material that remains after a microbial culture is permitted to grow on plant biomass to late stationary phase. As one example, spent biomass can refer to water insoluble material remaining after a culture of A. thermophilum is permitted to grow to approximately 10⁸ cells/mL on plant biomass.

“Thermophilic” refers to a microorganism that has a temperature optimum for growth of from 50° C.-100° C. “Extremely thermophilic” refers to a microorganism that has a temperature optimum for growth of from 70° C.-100° C.

“Untreated plant biomass” refers to plant biomass that contains complex organic polymer such as, for example, lignin or a complex polysaccharide or heteropolysaccharide (e.g., cellulose, a hemicellulose such as xylan, pectin, etc.) that has not been subjected to chemical, physical, microbial, or enzymatic processing to degrade the biomass—i.e., degrade the complex organic polymer to smaller chemical subunits.

The term “and/or” means one or all of the listed elements or a combination of any two or more of the listed elements.

The terms “comprises” and variations thereof do not have a limiting meaning where these terms appear in the description and claims.

Unless otherwise specified, “a,” “an,” “the,” “one or more,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.). Unless otherwise indicated, all numbers expressing quantities of components, molecular weights, and so forth used in the specification and claims may be modified in each instance by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

For any method disclosed herein that includes discrete steps, the steps may be conducted in any feasible order. And, as appropriate, any combination of two or more steps may be conducted simultaneously.

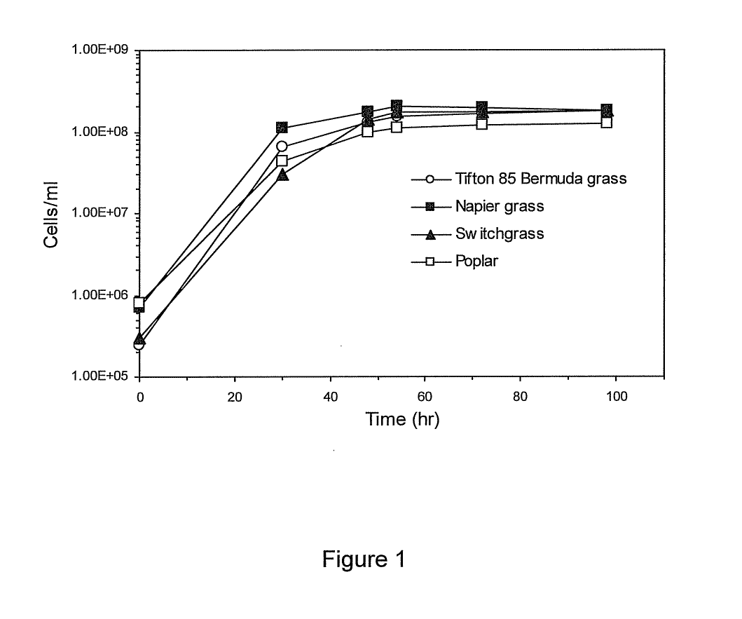

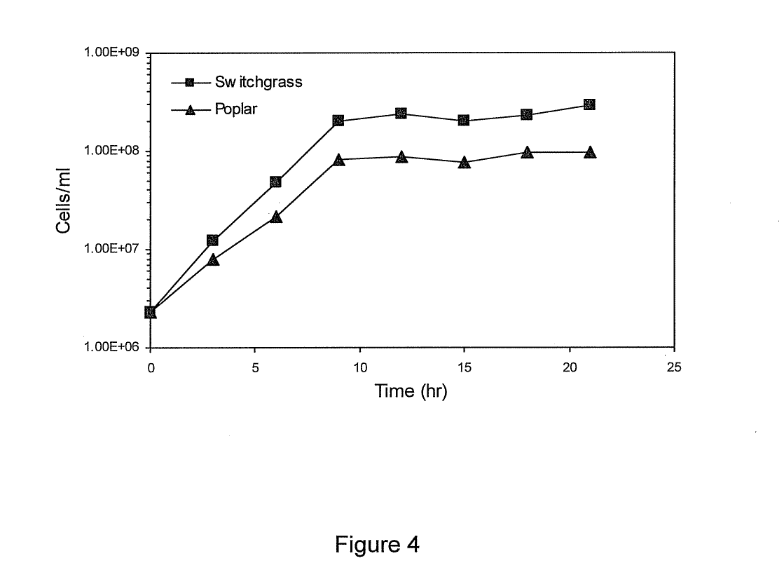

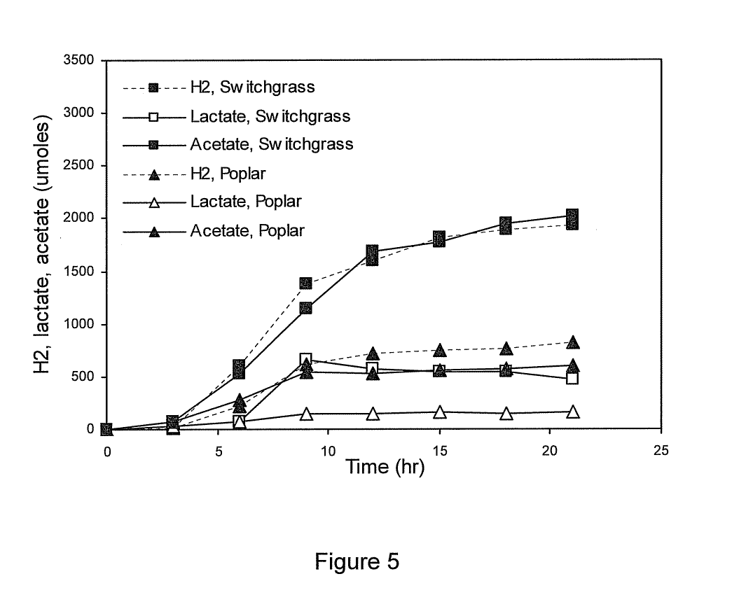

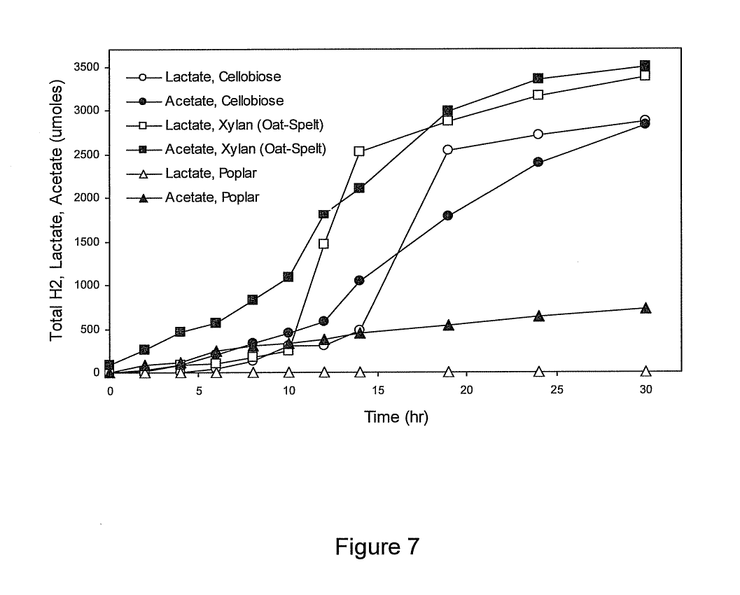

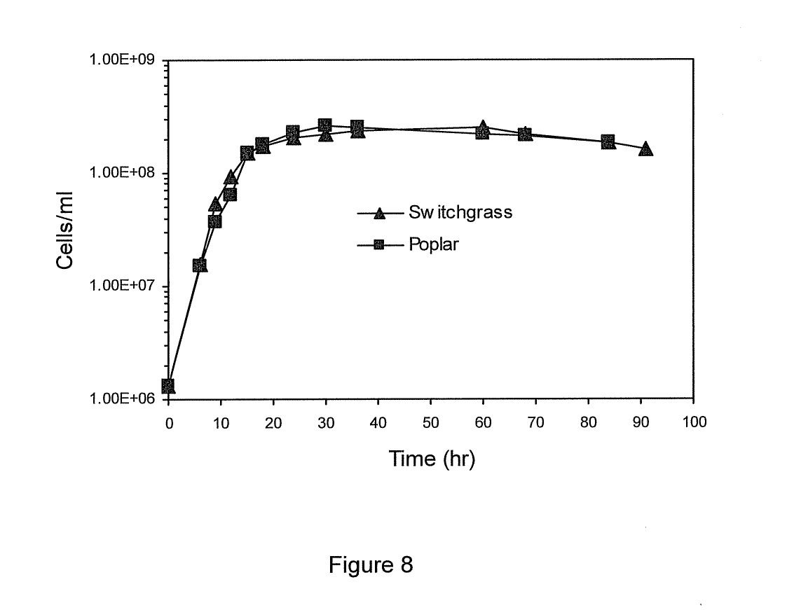

It has been found that A. thermophilum can grow efficiently on various types of untreated biomass (e.g., poplar woodchips, various types of grasses, and on the insoluble extracts of such biomass) (FIGS. 1-7). As used herein “efficient” growth refers to growth in which cells may be cultivated to a specified density within a specified time. For example, A. thermophilum can grow to a density of at least 5×10⁷ cells/milliliter (mL) such as, for example, a density of 10⁸ cells/mL. Methods for determining cell density of a culture are routine and known to those skilled in the art. Efficient growth of A. thermophilum on a substrate can be determined by measuring the cell density of the culture at a time no greater than 60 hours after the culture medium is inoculated. For example, efficient growth of A. thermophilum can be determined by measuring the cell density of the culture no greater than 30 hours, no greater than 24 hours, no greater than 16 hours, no greater than 12 hours, or no greater than 8 hours after inoculation of the culture.

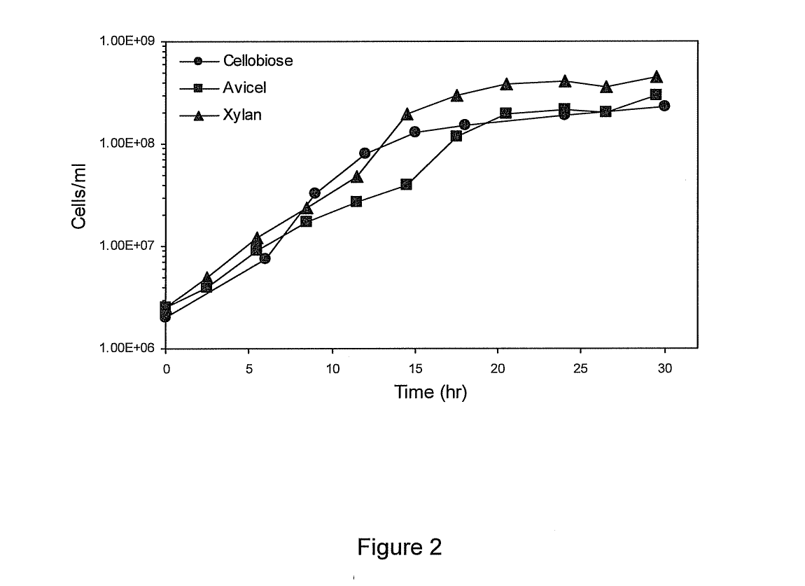

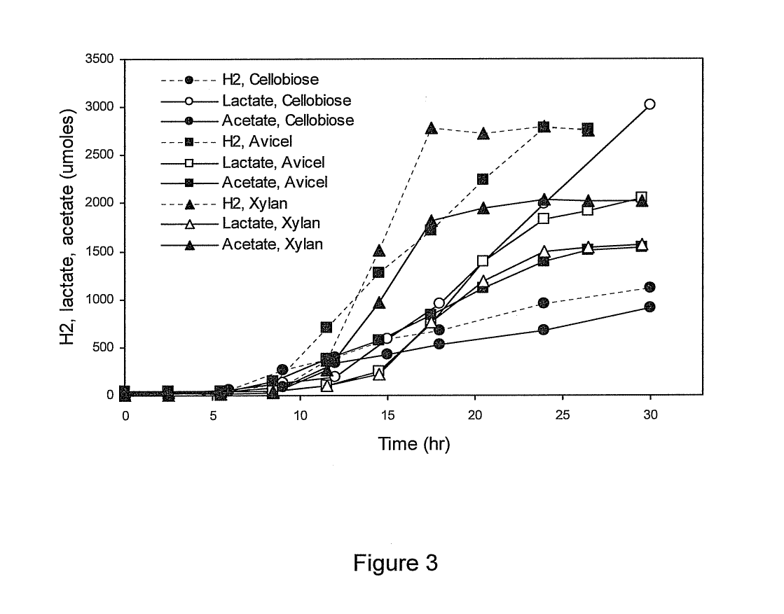

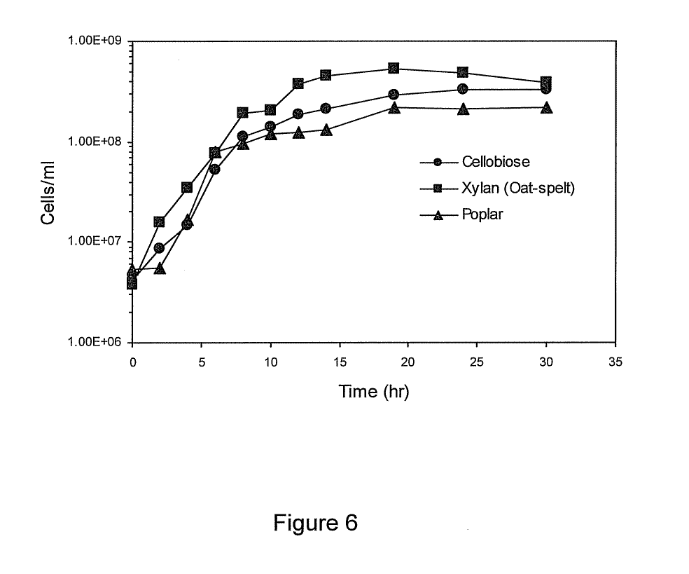

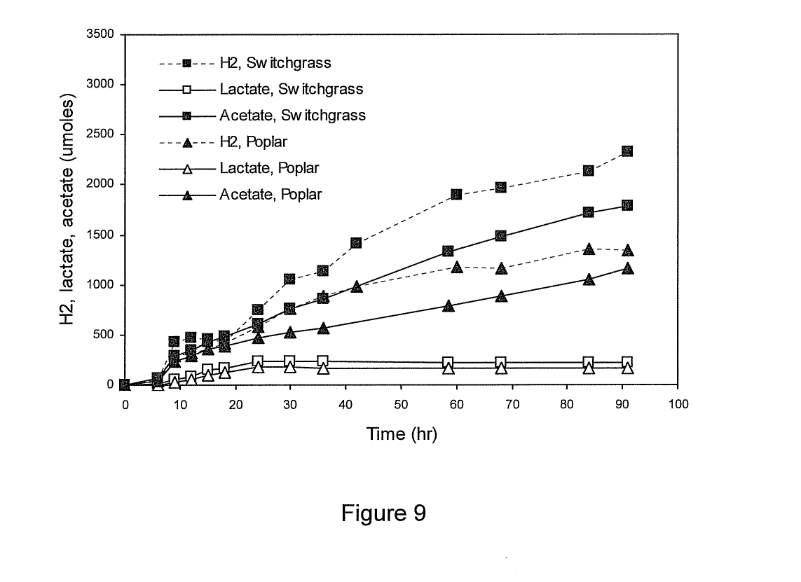

A. thermophilum can grow efficiently on crystalline cellulose and, in contrast to original reports (Svetlichnyi, V. A., T. P. Svetlichnaya, N. A. Chernykh, and G. A. Zavarzin. 1990. Anaerocellum thermophilum gen. nov., sp. nov., an extremely thermophilic cellulolytic eubacterium isolated from hot-springs in the valley of Geysers. Microbiology 59:598-604), can grow efficiently on xylan (oat spelt) (e.g., FIGS. 2 and 6). The main products when grown on untreated biomass substrates were lactate, acetate, and hydrogen gas (FIGS. 3 and 6). Moreover, the primary product is influenced at least somewhat by the biomass substrate. For example, FIG. 3 shows that when A. thermophilum is grown on a substrate of cellobiose, lactate is favored as a product over acetate and H₂. In contrast, FIG. 9 shows that when A. thermophilum is grown on a substrate of switchgrass, acetate and H₂ are favored products over lactate.

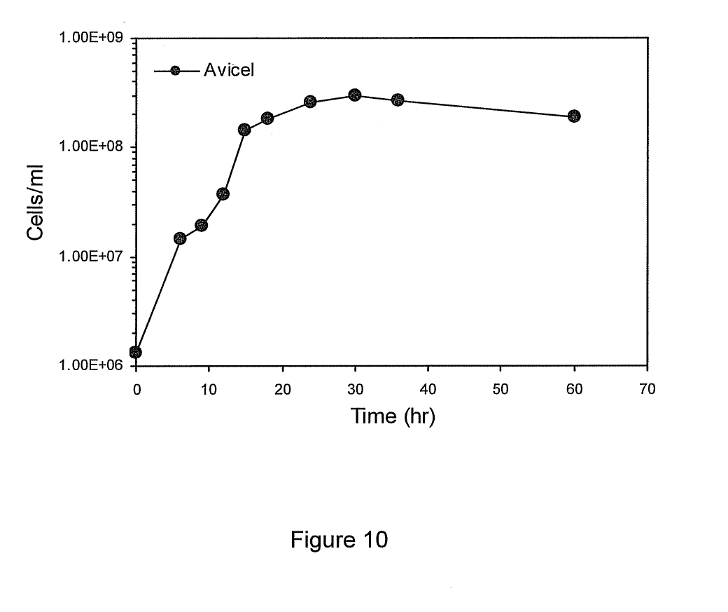

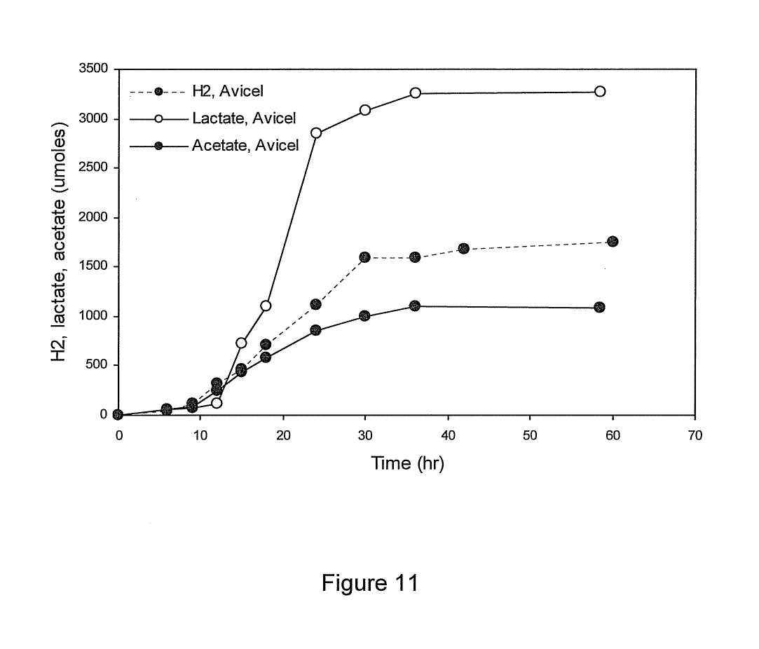

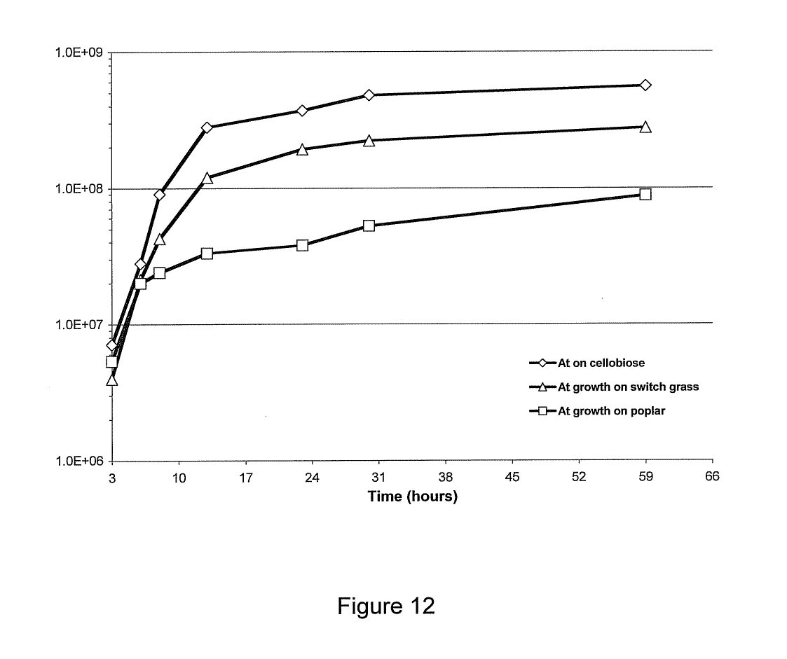

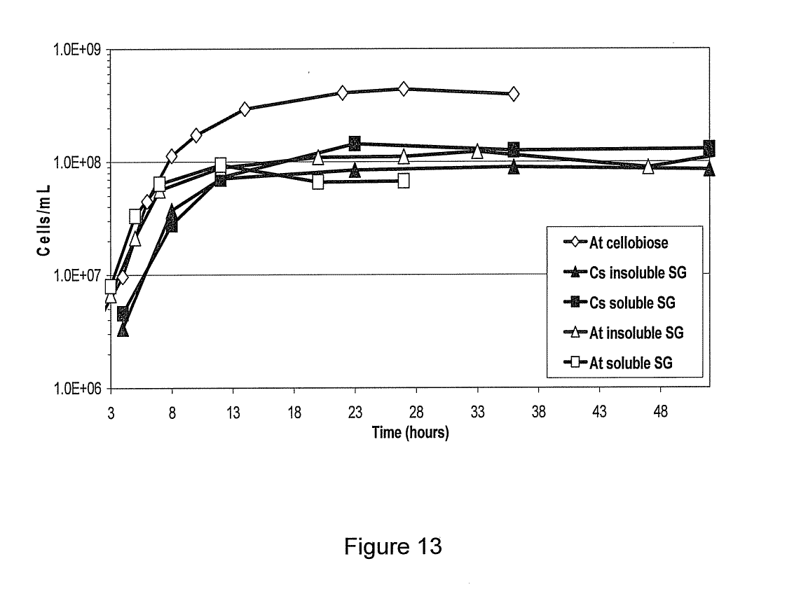

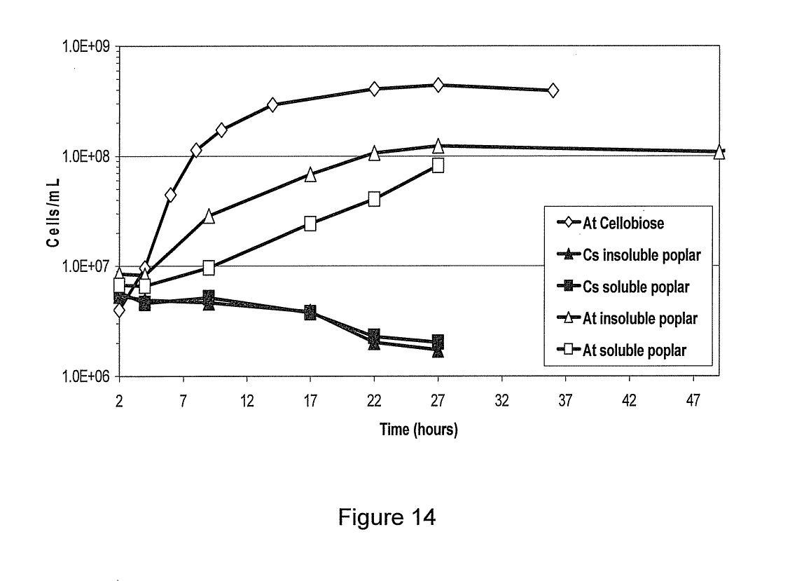

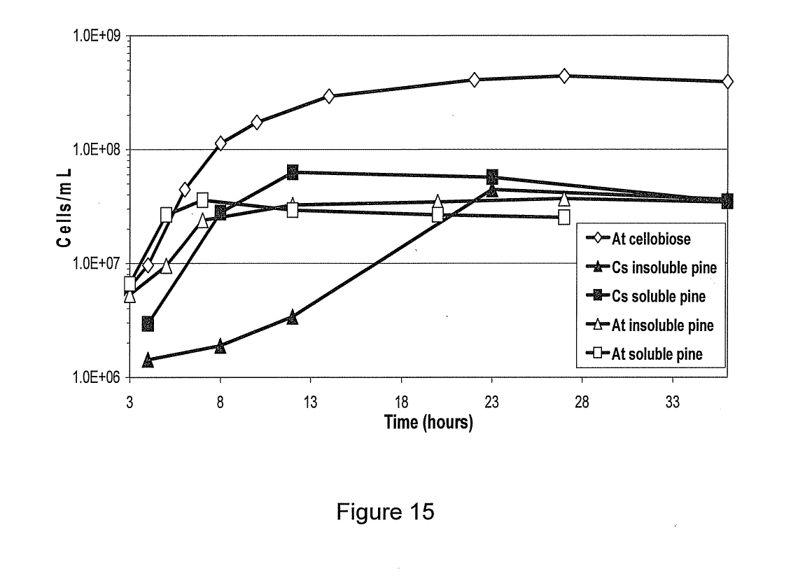

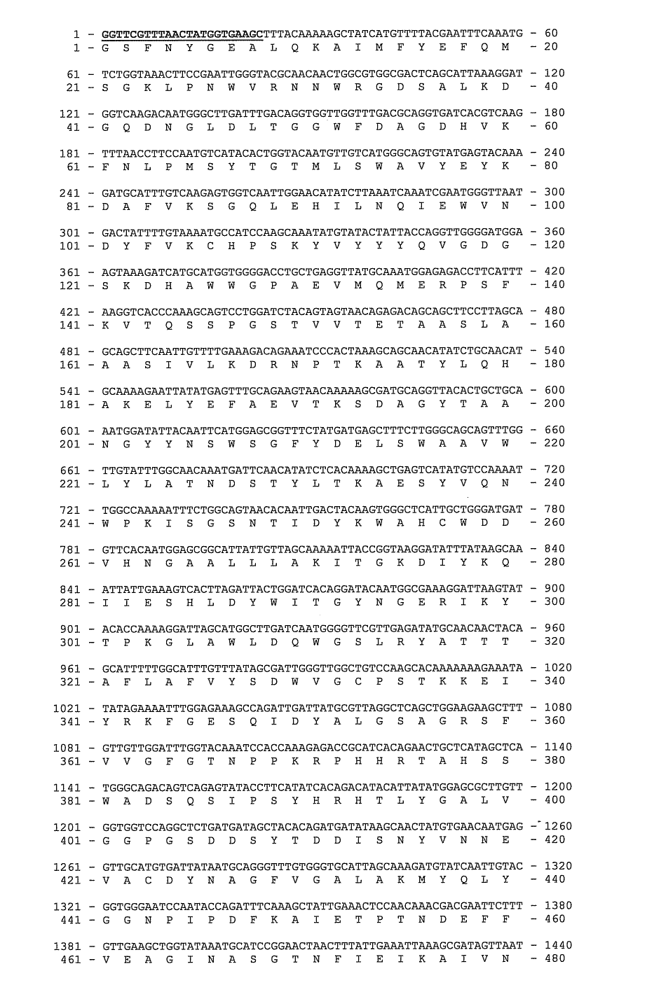

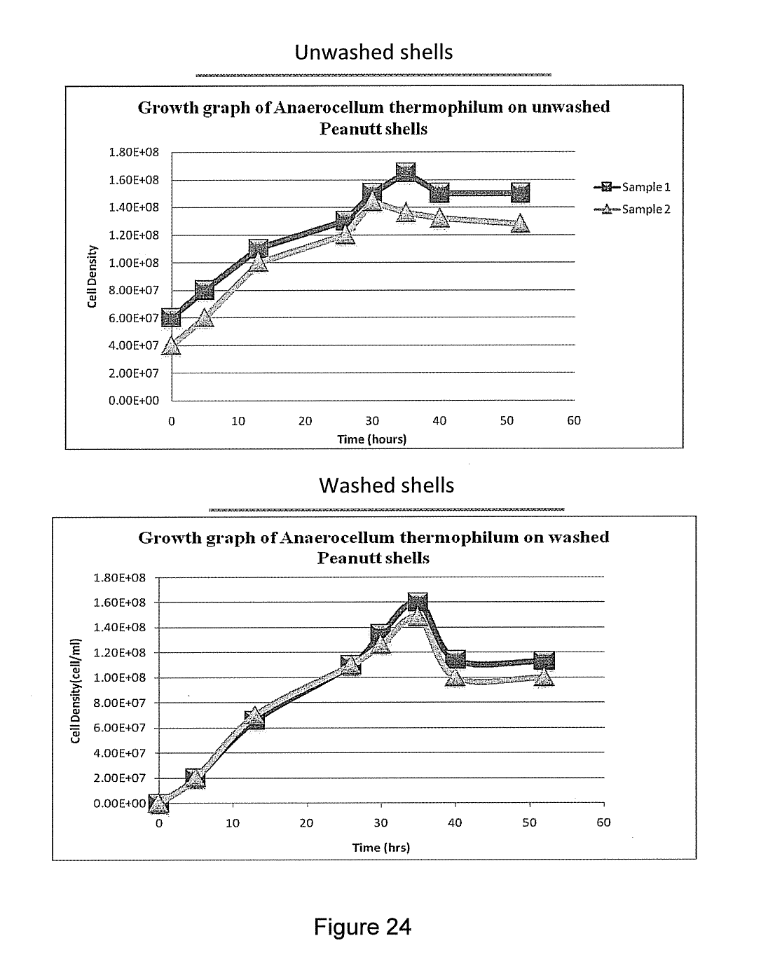

A. thermophilum also can grow efficiently on spent biomass—insoluble material that remains after a culture has grown to late stationary phase (e.g., greater than 10⁸ cells/mL) on untreated biomass (FIGS. 8 and 10). A. thermophilum also grew efficiently on cellobiose, untreated switchgrass, and untreated poplar (FIG. 12). A. thermophilum also grew on switchgrass and poplar that had been heated at 98° C. for two minutes. As shown in FIG. 13 and FIG. 14, A. thermophilum grew efficiently (greater than 10⁸ cells/ml) on both the soluble and insoluble materials obtained after heat treating the biomass. The microorganism also grew efficiently on the insoluble material obtained from pine wood after a similar heat treatment (FIG. 15). A. thermophilum also grew efficiently on peanut shells regardless of whether the peanut shells were first washed for 18 hours at 75° C. (FIG. 24).

Thus, in one aspect, the present invention provides methods of processing biomass—particularly but not exclusively water insoluble untreated plant biomass and/or water insoluble spent biomass. Generally, the methods include growing A. thermophilum on a substrate that includes plant biomass under conditions effective for the A. thermophilum to convert at least a portion of the plant biomass to a less complex water soluble product such as, for example, organic compounds (e.g., organic acids and/or simple carbohydrates such as, for example, monosaccharides and disaccharides) that are readily metabolizable by A. thermophilum and/or another microorganism. In some embodiments, the method can further include converting at least a portion of the water soluble product to a biofuel, a polymer, or a commodity chemical. In other cases, the water soluble product may itself be a biofuel, a polymer, and/or a commodity chemical. In other cases, the product of processing the biomass may be a water insoluble product that may itself be a biofuel. In particular embodiments, the methods include growing A. thermophilum on a substrate that includes plant biomass under conditions effective for the A. thermophilum to degrade cellulose present in the plant biomass.

The plant biomass can be any plant biomass that is degradable by A. thermophilum—i.e., any plant biomass in which A. thermophilum is capable of breaking down a complex organic polymer (e.g., lignin or a complex polysaccharide or heteropolysaccharide) component of the biomass to smaller, constituent subunits. In some embodiments, the plant biomass can include plant biomass not utilizable by Caldicellulosiruptor saccharolyticus such as, for example, C. saccharolyticus (DSM 8903). As used herein, plant biomass that is not utilizable by C. saccharolyticus refers to biomass on which C. saccharolyticus does not grow efficiently (e.g., soluble and/or insoluble heat-treated poplar, FIG. 14).

The plant biomass can include lignocellulosic material. Lignocellulosic material may be found, for example, in the stems, leaves, hulls, husks, and/or cobs of plants or leaves, branches, and wood of trees. Lignocellulosic material can also be, for example, herbaceous material, agricultural residues, forestry residues, municipal solid wastes, waste paper, and pulp and paper mill residues. In some cases, lignocellulosic material may be in the form of plant cell wall material containing lignin, cellulose, and hemicellulose in a mixed matrix. In some aspects the lignocellulosic material may include grass such as switchgrass, Bermudagrass, napiergrass; paper and/or pulp processing waste; corn waste such as corn stover and/or corn fiber; hardwood such as poplar and/or birch; softwood such as Douglas fir, pine (e.g., Pinus taeda) and/or spruce; cereal straw such as wheat straw and/or rice straw; municipal solid waste; industrial organic waste; sugarcane and/or bagasse; sugarbeets and/or pulp; sweet potatoes; food processing wastes; or any mixtures thereof.

Thus, in some embodiments, the plant biomass can include woody plant biomass such as, for example, treated and/or untreated wood, woodchips, sawdust, etc. The woody plant biomass may be, or be derived from, any species of woody plant. In some embodiments, the woody plant biomass may be derived from poplar (i.e., Populus spp.) or pine (i.e., Pinus spp.), but the methods may be practiced using woody plant biomass derived from other species of woody plants.

In other embodiments, the plant biomass may be, or be derived from, treated or untreated sources such as, for example, grasses, peanut shells (washed or unwashed), crystalline cellulose, cellobiose, or xylan.

In some embodiments, the plant biomass may include spent biomass. Thus, the methods offer the possibility of extracting compounds and/or energy from plant biomass that is commonly left unexploited.

In some embodiments, the plant biomass can include a combination of plant biomass from various sources (e.g., hardwood, softwood, grass, straw, pulp, etc.). Thus, a combination of plant biomass can include, for example, poplar and pine woodchips. Alternatively, in some embodiments, a combination of plant biomass can include, for example, plant biomass that excludes, for example, softwood sawdust (e.g., pine sawdust). As one example, such a combination of plant biomass can include grass (e.g., switchgrass, Bermudagrass, and/or napiergrass), straw (e.g., wheat straw and/or rice straw), and/or corn stover.

Also, the plant biomass can include a combination of treated, untreated, and spent biomass, with the nature (i.e., treated, untreated, or spent) of biomass from each source being independent of the nature of biomass from other sources in the combination.

The methods of processing biomass can include growing A. thermophilum on a substrate that includes plant biomass under conditions effective for the A. thermophilum to convert at least a portion of the plant biomass to a less complex—e.g., water soluble—product. Such conditions include conditions under which A. thermophilum may be grown in culture. Because A. thermophilum is a thermophilic microbe, in some embodiments, the conditions include a temperature of at least 70° C. such as, for example, at least 75° C., at least 80° C., at least 85° C., or at least 90° C. However, the methods described herein may be practiced at lower temperatures including, for example, a temperature of at least 37° C. or at least 30° C. Also, the growing conditions may be anaerobic. As used herein, “anaerobic” conditions refer to conditions in which the partial pressure of O₂ in the gas phase is less than 10 ppm, such as, for example, 1 ppm.

In another aspect, the invention provides a method of pretreating plant biomass. Generally, the method includes growing Anaerocellum thermophilum on a substrate that comprises plant biomass under conditions effective for the A. thermophilum to degrade cellulose of the plant biomass, thereby preparing the plant biomass for further processing by another biomass processing method. Pretreating plant biomass using A. thermophilum can reduce the need for chemical and/or heat pretreatments in order to make most efficient use of the plant biomass. Thus, in this aspect, the method can reduce, for example, the time, cost, and environmental impact of processing plant biomass and can increase, for example, the efficiency at which the plant biomass is processed.

In some aspects, described in more detail below, the invention can involve one or more coding regions that can encode polypeptides involved in the degradation of plant biomass and/or the synthesis of certain metabolic products (e.g., biofuels, commodity chemicals, and/or intermediates for the production of either biofuels or commodity chemicals). As used herein, “coding region” refers to a nucleotide sequence that encodes a polypeptide and, when placed under the control of appropriate regulatory sequences expresses the encoded polypeptide. The boundaries of a coding region are generally determined by a translation start codon at its 5′ end and a translation stop codon at its 3′ end. A “regulatory sequence” is a nucleotide sequence that regulates expression of a coding sequence to which it is operably linked. Regulatory sequences include, for example, promoters, enhancers, transcription initiation sites, translation start sites, translation stop sites, and transcription terminators. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

In some embodiments, the coding region can include a nucleotide sequence having at least 80% identity to a reference nucleotide sequence such as, for example, an A. thermophilum PBU coding region, an A. thermophilum PHR coding region, or any other identified coding region (each of which is described herein below). Nucleotide sequences of A. thermophilum coding regions such as, for example, PBU coding regions and PHR coding regions, are accessible via GenBank Accession No. CP001395 (version 1, created Feb. 5, 2009). In certain embodiments, a coding region can have at least 85% identity to the nucleotide sequence of a reference coding region such as for example, at least 86%, at least 87%, at least 88%, at least 89%, at least 90%, at least 91%, at least 92%, at least 93%, at least 94%, at least 95%, at least 96%, at least 97%, at least 98% or at least 99% identity to the nucleotide sequence of a reference coding region. Such nucleotide sequences may include one or more modifications relative to the nucleotide sequence of the reference coding region. As used herein, two nucleotide sequences may be compared and the nucleotide identity is resulting from that comparison may be referred to as “identities.” Two nucleotide sequences may be compared using the Blastn program of the BLAST 2 search algorithm, as described by Tatusova, et al. (FEMS Microbiol Lett, 174, 247250 (1999)), and available through the World Wide Web, for instance at the internet site maintained by the National Center for Biotechnology Information, National Institutes of Health. Preferably, the default values for all BLAST 2 search parameters are used, including reward for match=1, penalty for mismatch=−2, open gap penalty=5, extension gap penalty=2, gap x dropoff=50, expect=10, wordsize=11, and optionally, filter on.

In other aspects, the invention can involve the expression of an A. thermophilum polypeptide or a biologically active analog, subunit, or derivative thereof. An A. thermophilum polypeptide or a biologically active analog, subunit, or derivative thereof encoded by a PBU coding region may be referred to as a PBU polypeptide. Similarly, an A. thermophilum polypeptide or a biologically active analog, subunit, or derivative thereof encoded by a PHR coding region may be referred to as a PHR polypeptide.

In some embodiments, the A. thermophilum polypeptide may be isolated. As used herein, an “isolated” polypeptide is one that is separated from its natural environment to any degree. An isolated polypeptide may be, for example, at least 60% free, at least 75% free, at least 90% free, at least 91% free, at least 92% free, at least 93% free, at least 94% free, at least 95% free, at least 96%, at least 97% free, at least 98% free, or at least 99% free from other components with which it is naturally associated. Polypeptides that are produced outside the microorganism in which they naturally occur, e.g., through chemical or recombinant means, are considered to be isolated and purified by definition, since they were never present in a natural environment.

A “biologically active” analog, subunit, or derivative of an A. thermophilum polypeptide is a polypeptide that exhibits the ability to degrade water insoluble plant biomass material. A biologically active “analog” of an A. thermophilum polypeptide includes, for example, an A. thermophilum polypeptide that has been modified by the addition, substitution, or deletion of one or more contiguous or noncontiguous amino acids, or that has been chemically or enzymatically modified, e.g., by attachment of a reporter group, by an N-terminal, C-terminal or other functional group modification or derivatization, or by cyclization, as long as the analog retains biological activity. An analog can thus include additional amino acids at one or both of the termini of a polypeptide.

Substitutes for an amino acid in an A. thermophilum polypeptide are preferably conservative substitutions, which are selected from other members of the class to which the amino acid belongs. For example, it is well-known in the art of protein biochemistry that an amino acid belonging to a grouping of amino acids having a particular size or characteristic (such as charge, hydrophobicity and hydrophilicity) can generally be substituted for another amino acid without substantially altering the structure of a polypeptide. For the purposes of this invention, conservative amino acid substitutions are defined to result from exchange of amino acids residues from within one of the following classes of residues: Class I: Ala, Gly, Ser, Thr, and Pro (representing small aliphatic side chains and hydroxyl group side chains); Class H: Cys, Ser, Thr and Tyr (representing side chains including an —OH or —SH group); Class III: Glu, Asp, Asn and Gln (carboxyl group containing side chains): Class IV: His, Arg and Lys (representing basic side chains); Class V: Ile, Val, Leu, Phe and Met (representing hydrophobic side chains); and Class VI: Phe, Trp, Tyr and His (representing aromatic side chains). The classes also include related amino acids such as 3Hyp and 4Hyp in Class I; homocysteine in Class II; 2-aminoadipic acid, 2-aminopimelic acid, γ-carboxyglutamic acid, β-carboxyaspartic acid, and the corresponding amino acid amides in Class III; ornithine, homoarginine, N-methyl lysine, dimethyl lysine, trimethyl lysine, 2,3-diaminopropionic acid, 2,4-diaminobutyric acid, homoarginine, sarcosine and hydroxylysine in Class IV; substituted phenylalanines, norleucine, norvaline, 2-aminooctanoic acid, 2-aminoheptanoic acid, statine and β-valine in Class V; and naphthylalanines, substituted phenylalanines, tetrahydroisoquinoline-3-carboxylic acid, and halogenated tyrosines in Class VI.

The amino acid sequences of exemplary A. thermophilum polypeptides are accessible via GenBank Accession No. CP001395 (version 1, created Feb. 5, 2009). Certain biologically active analogs, subunits, or derivatives of a reference A. thermophilum polypeptide can include those analogs, subunits, or derivatives that have at least 80% identity to the reference A. thermophilum polypeptide. In some embodiments, the biologically active analog, subunit, or derivative can have at least 85% identity to a reference A. thermophilum polypeptide such as, for example, at least 86%, at least 87%, at least 88%, at least 89%, at least 90%, at least 91%, at least 92%, at least 93%, at least 94%, at least 95%, at least 96%, at least 97%, at least 98%, or at least 99% identity to a reference A. thermophilum polypeptide. Such analogs, subunits, or derivatives can contain one or more amino acid deletions, insertions, and/or substitutions relative to the reference A. thermophilum polypeptide, and may further include chemical and/or enzymatic modifications and/or derivatizations, as described above.

The degree of identity between two amino acid sequences can be determined using commercially available algorithms. Preferably, two amino acid sequences are compared using the BLASTP program of the BLAST 2 search algorithm, as described by Tatusova, et al., (FEMS Microbiol Lett 1999, 174:247-250), and available through the World Wide Web, for instance at the internet site maintained by the National Center for Biotechnology Information, National Institutes of Health. Preferably, the default values for all BLAST 2 search parameters are used, including matrix=BLOSUM62; open gap penalty=11, extension gap penalty=1, gap x_dropoff=50, expect=10, wordsize=3, and optionally, filter on.

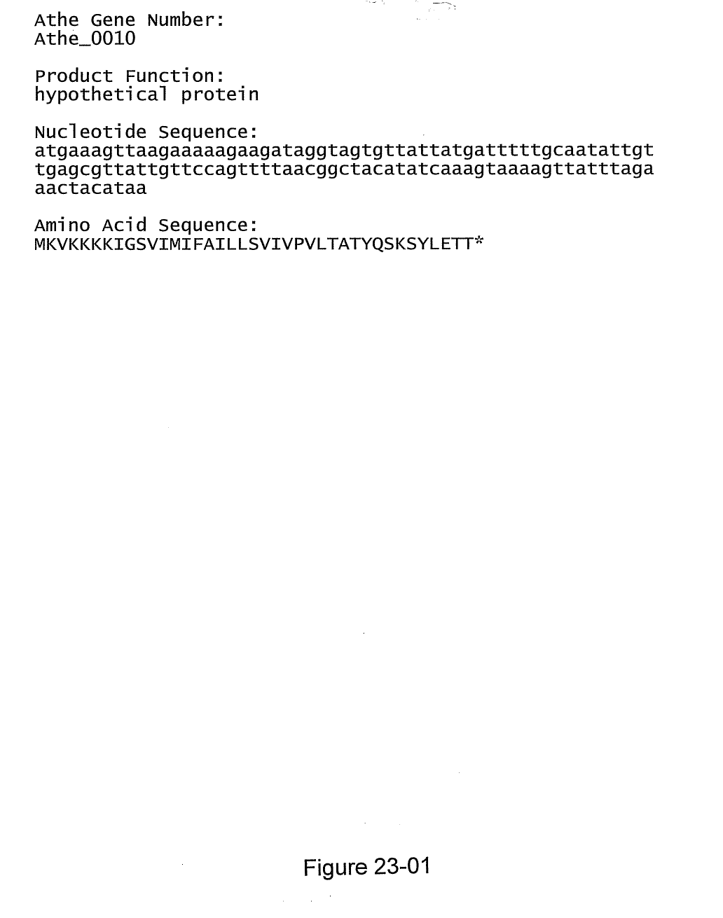

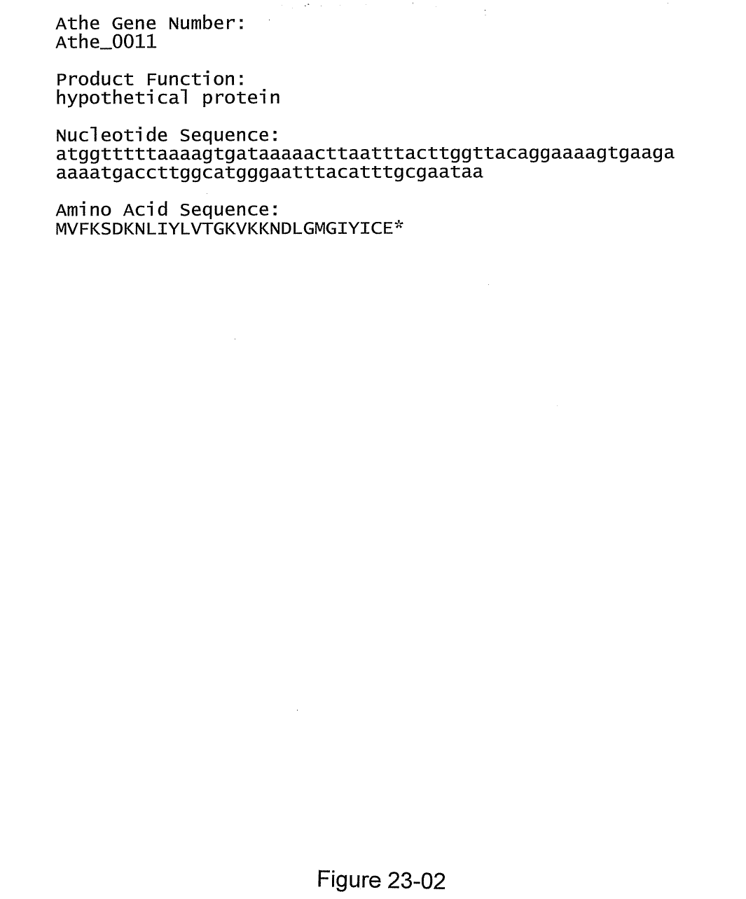

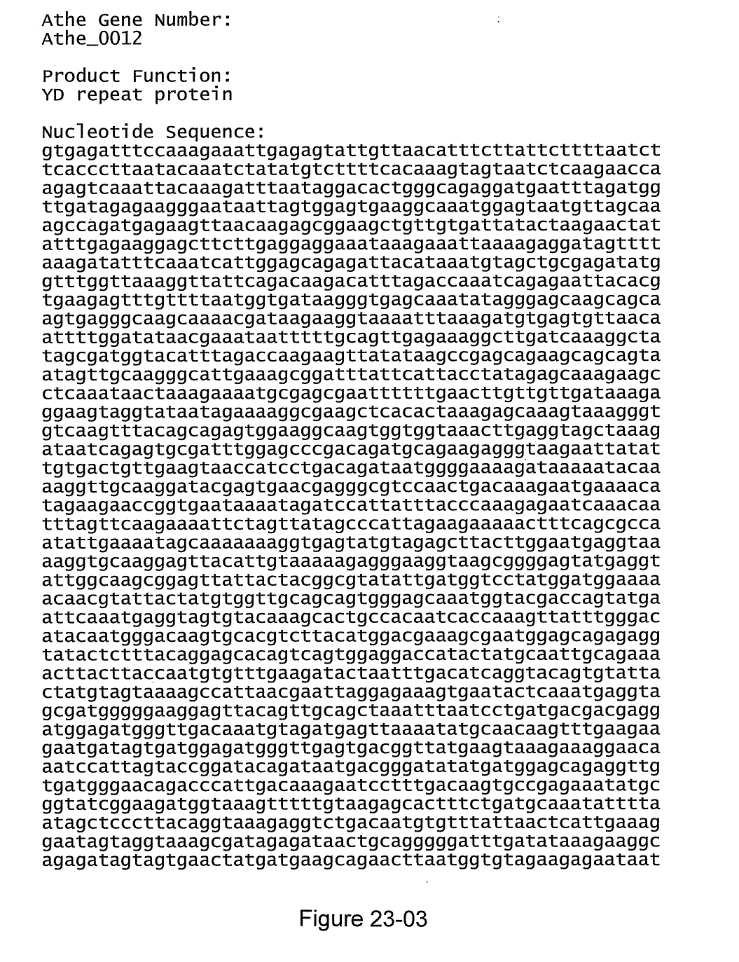

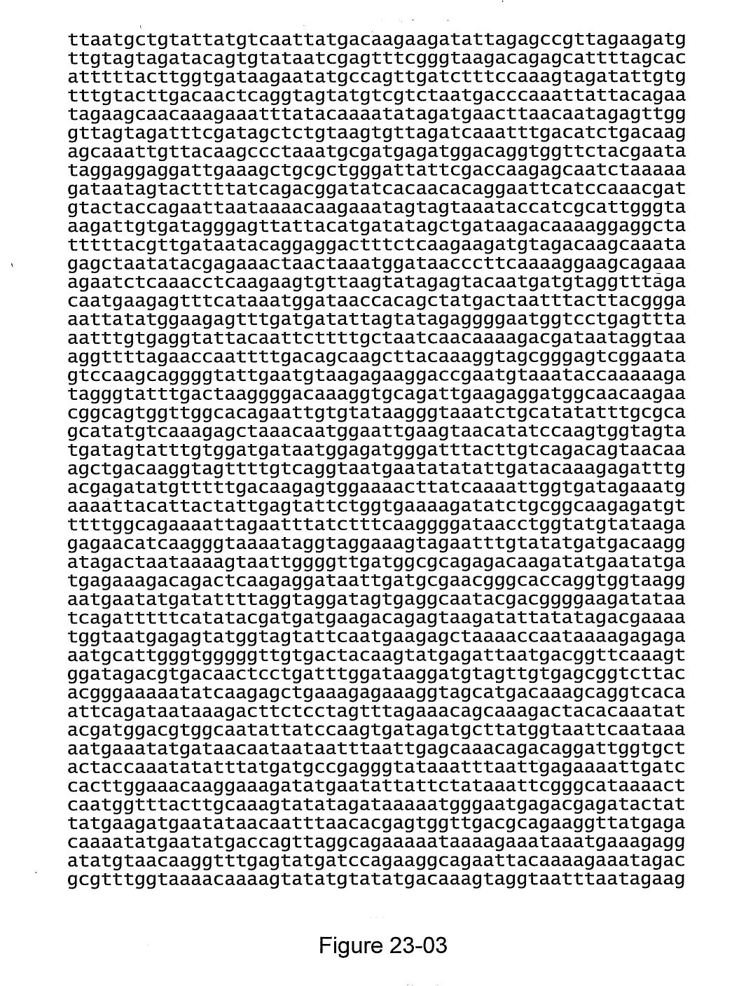

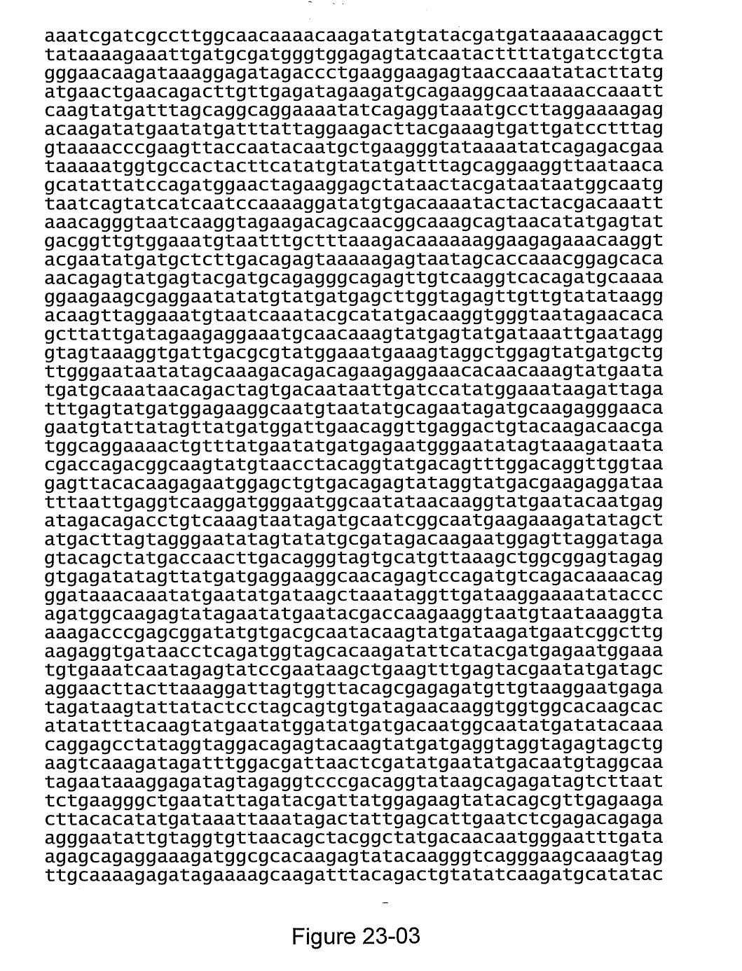

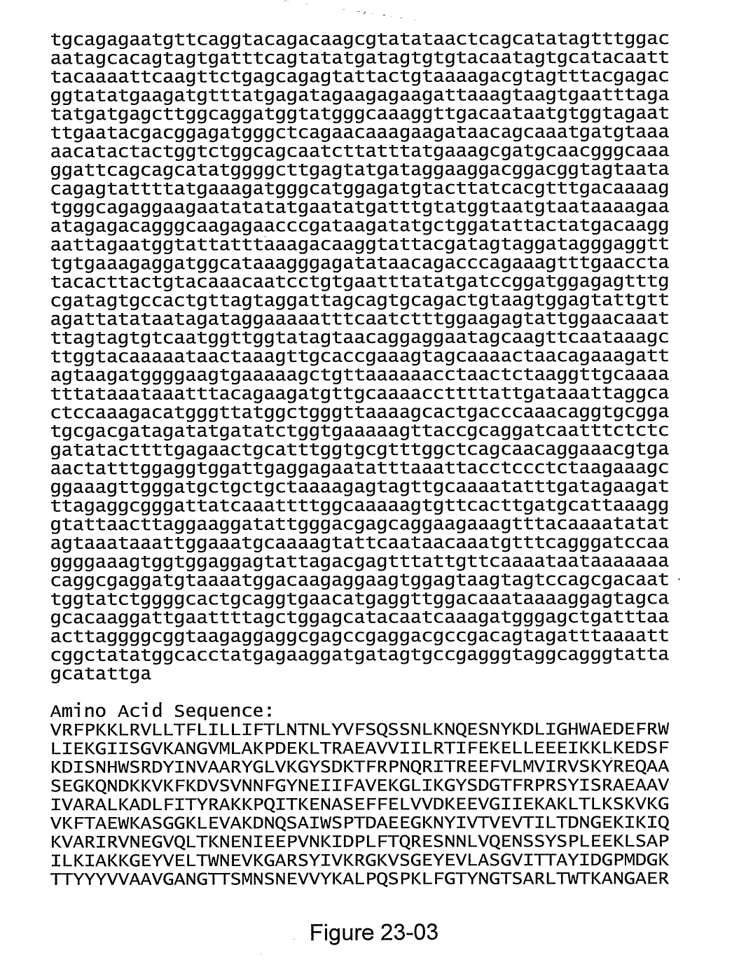

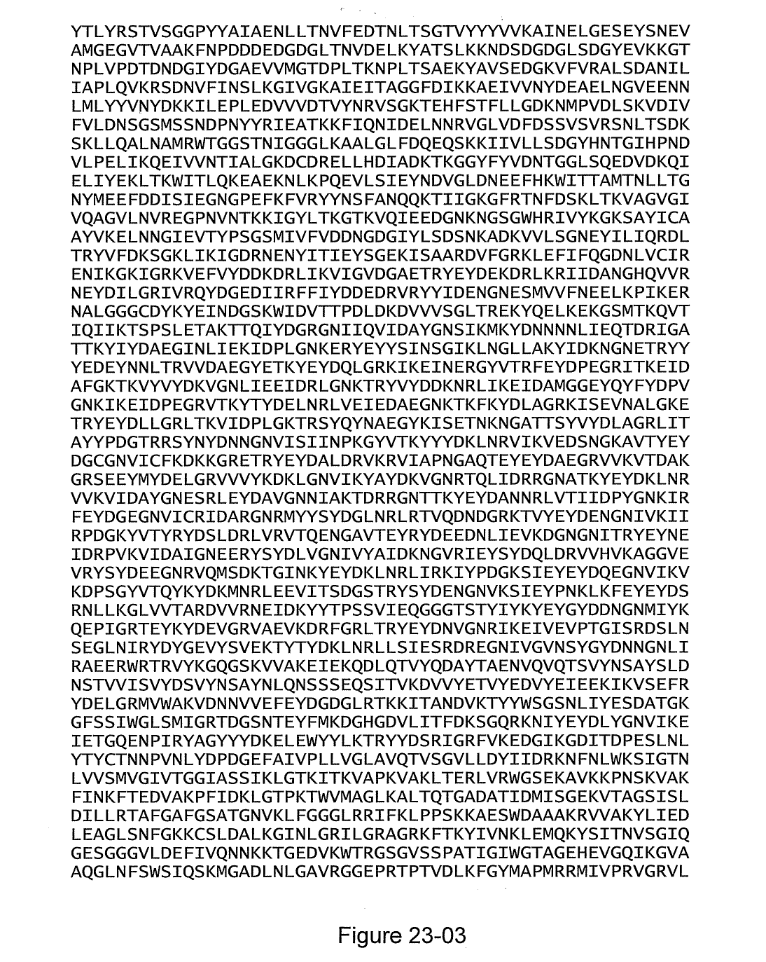

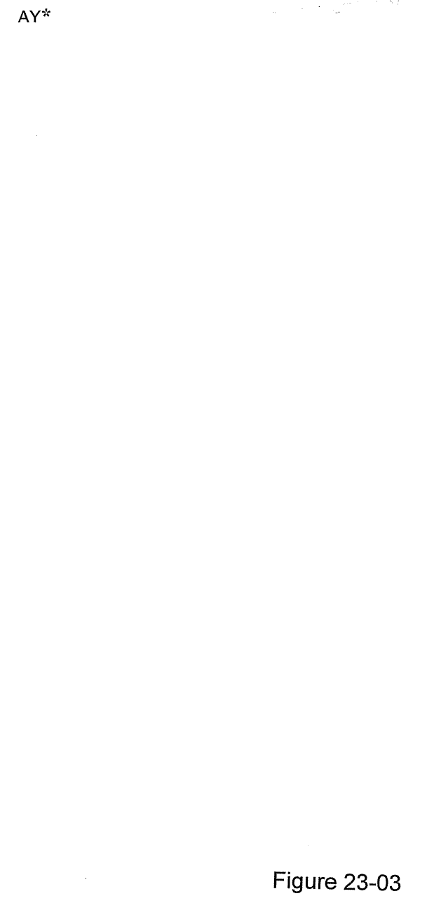

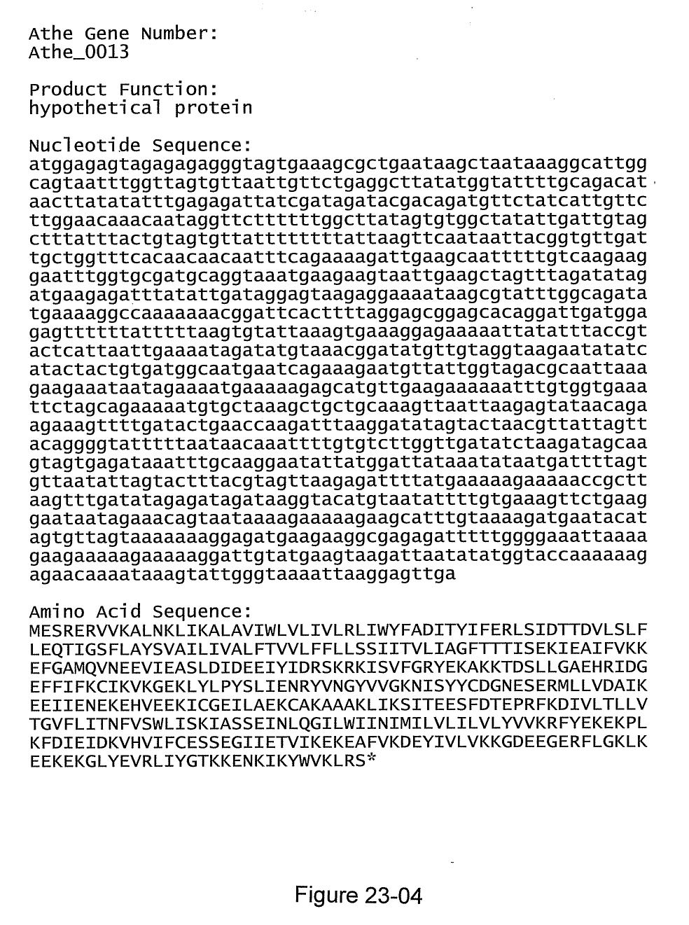

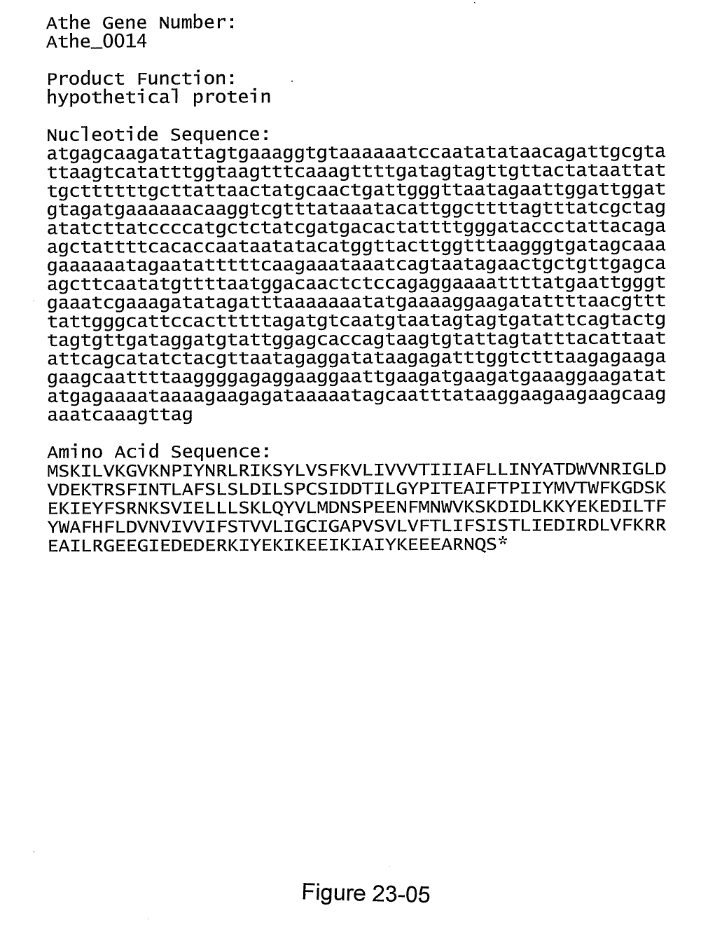

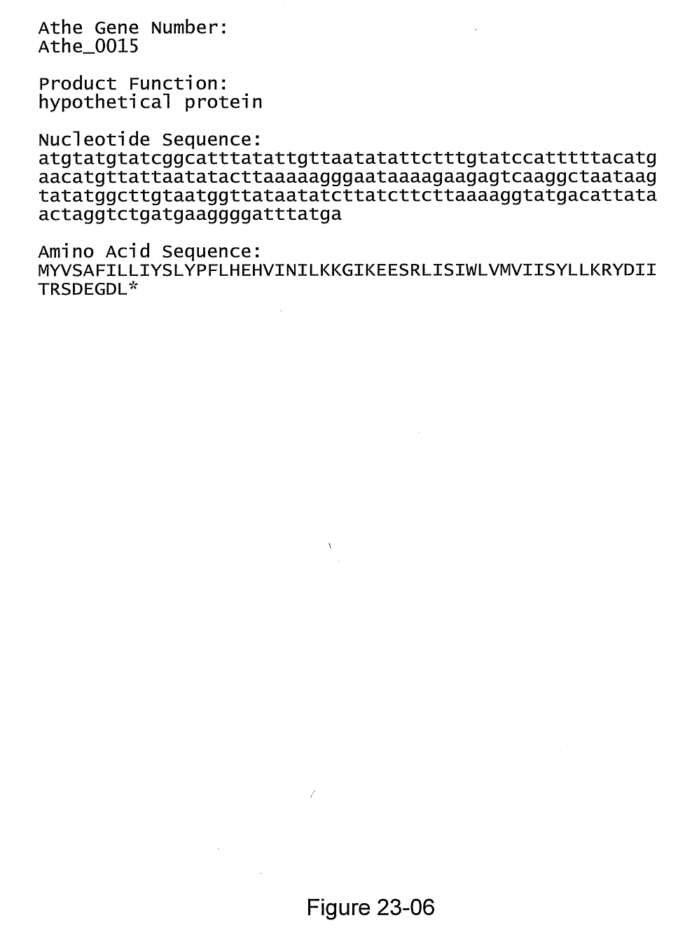

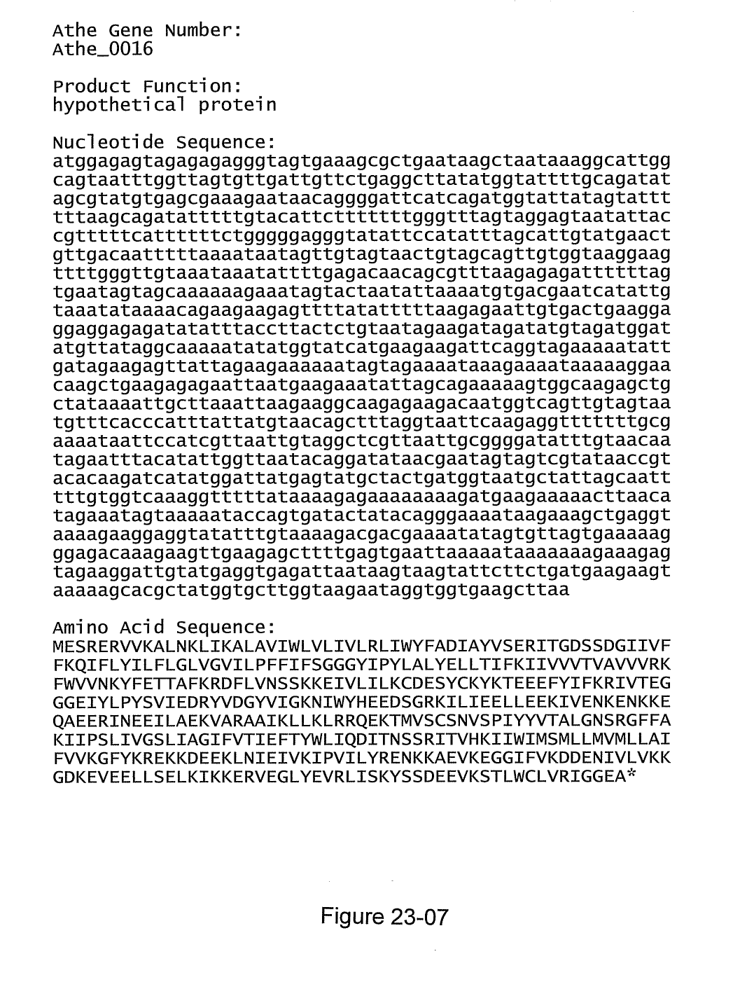

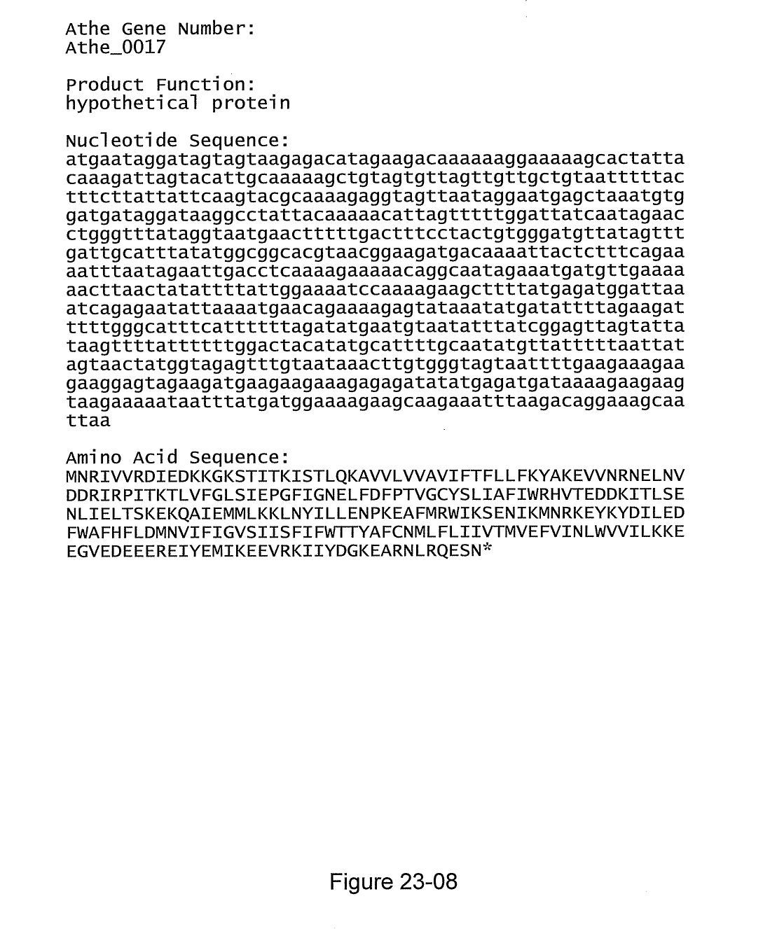

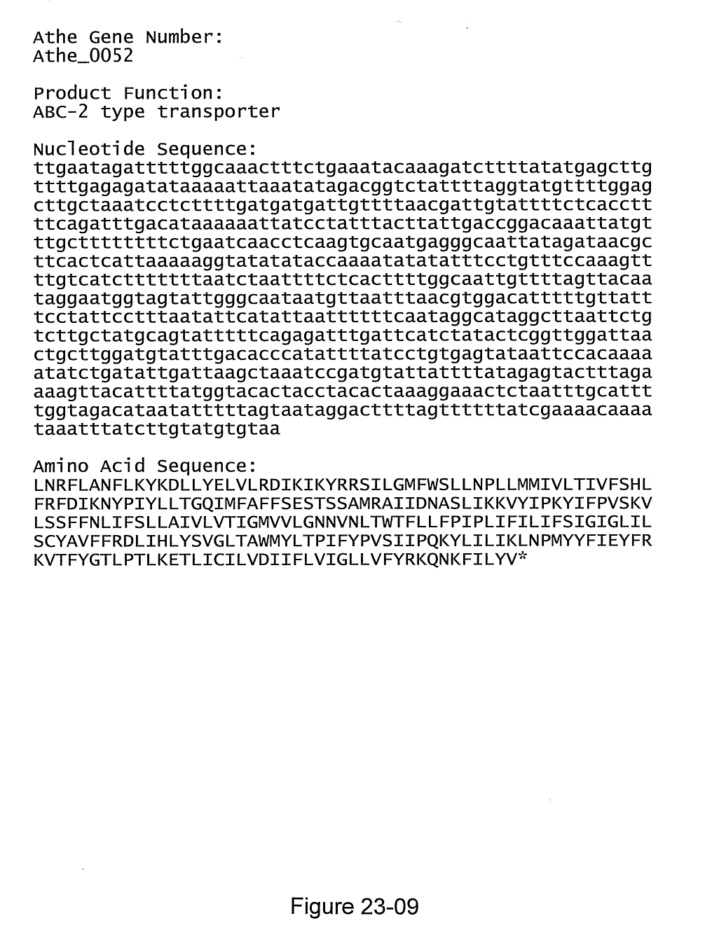

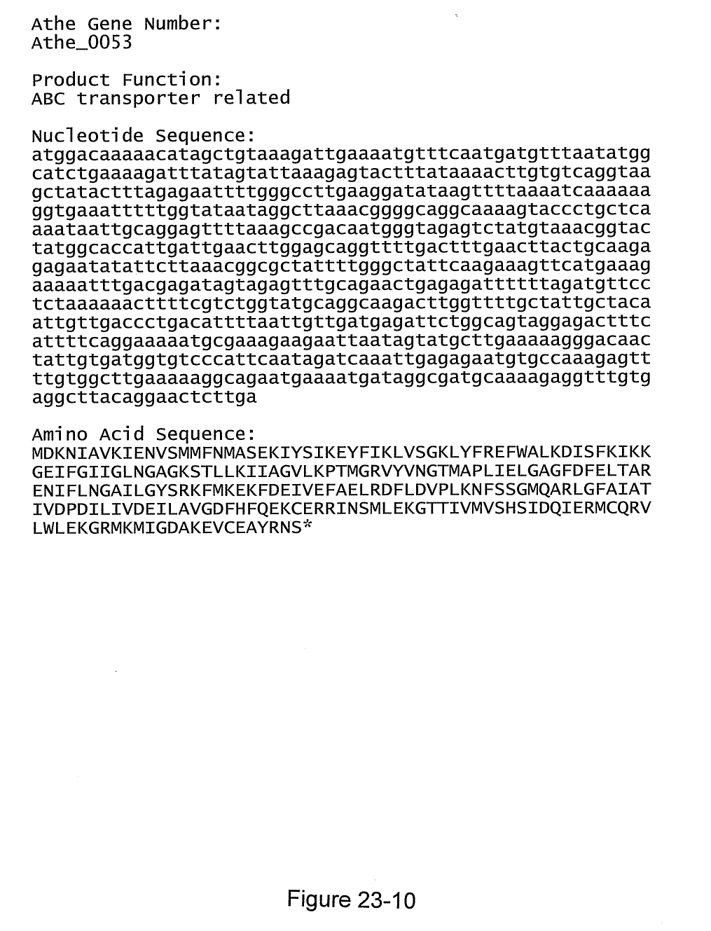

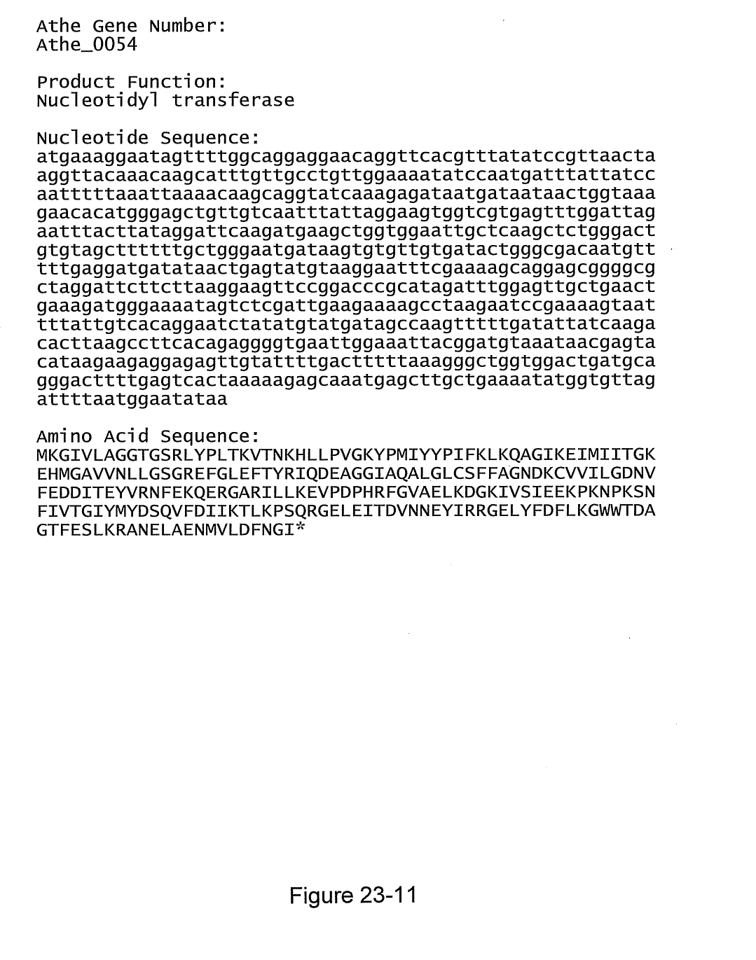

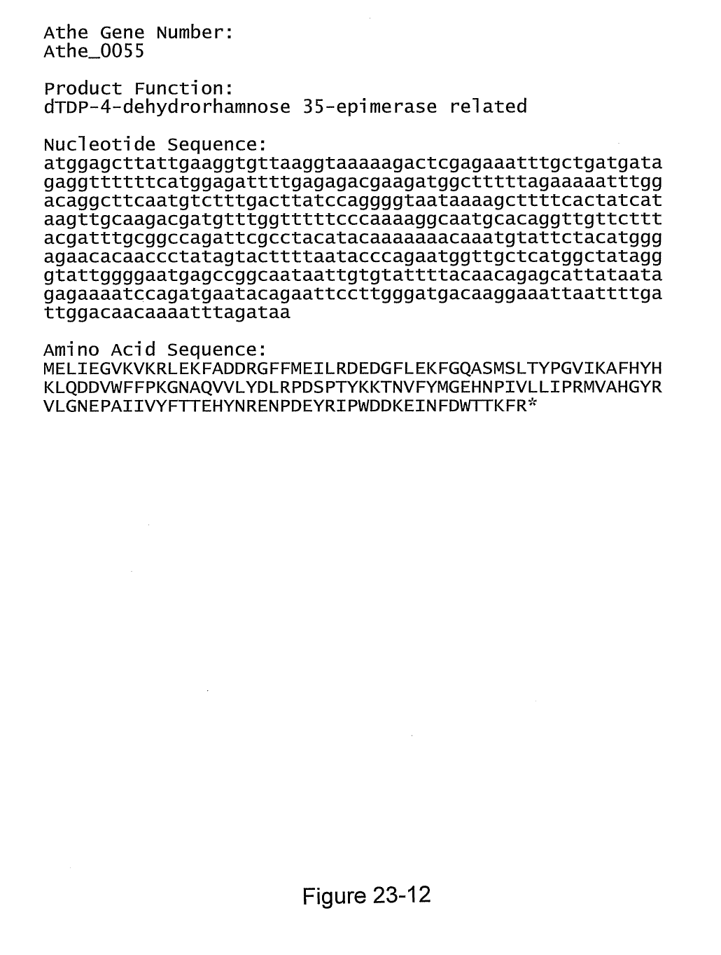

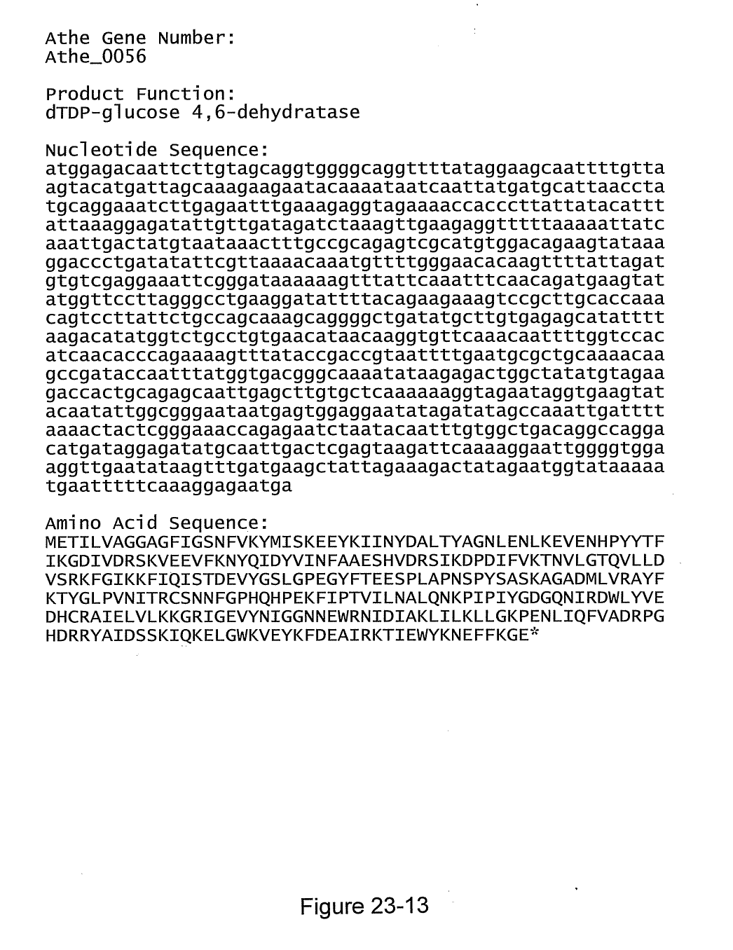

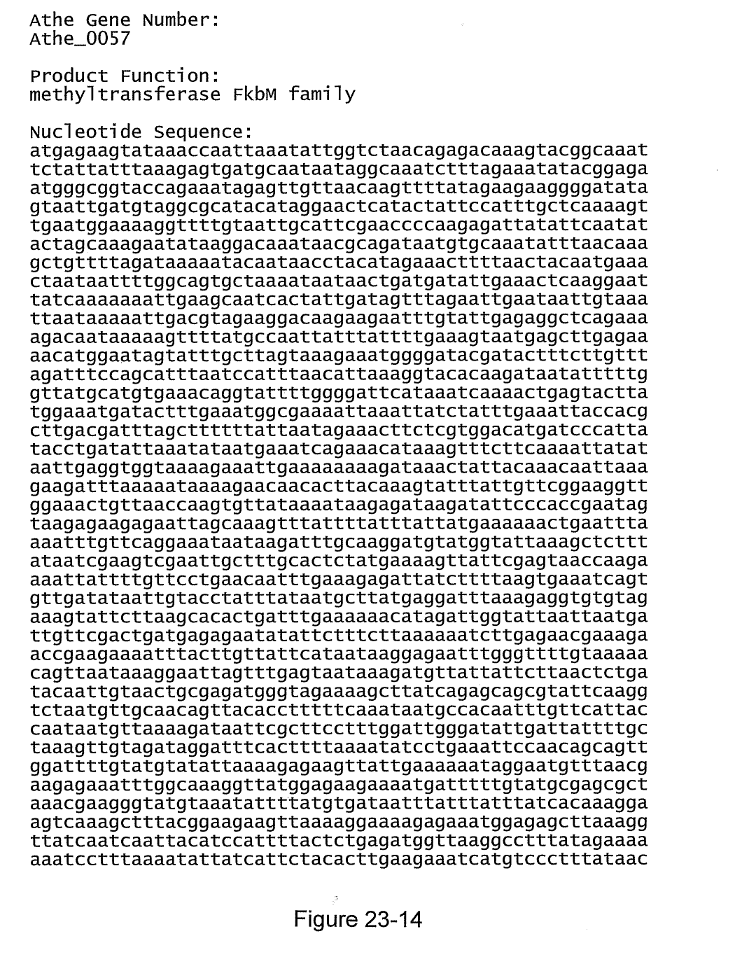

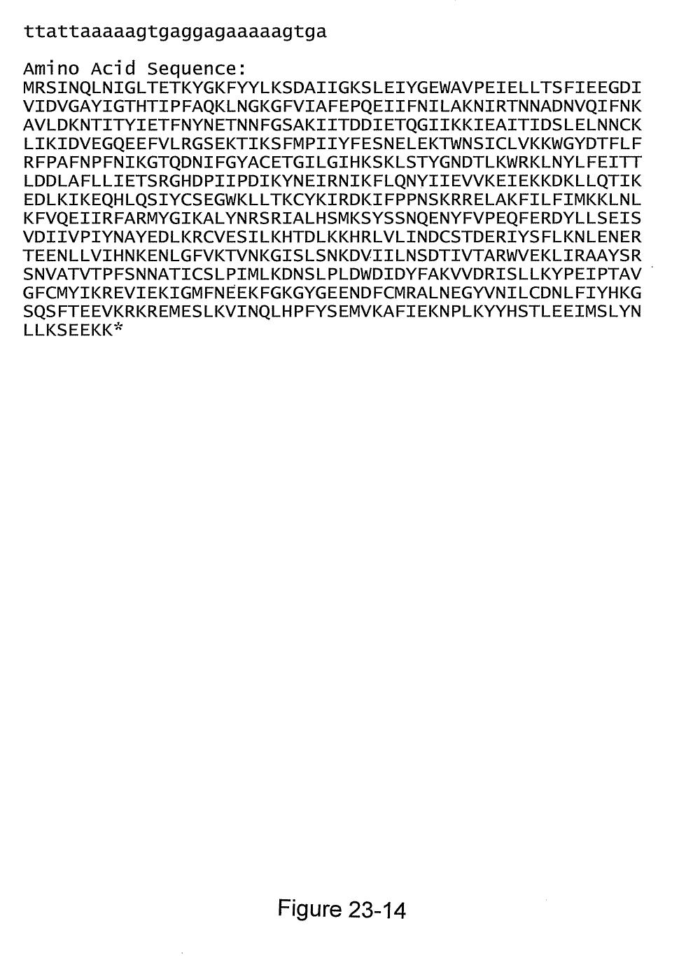

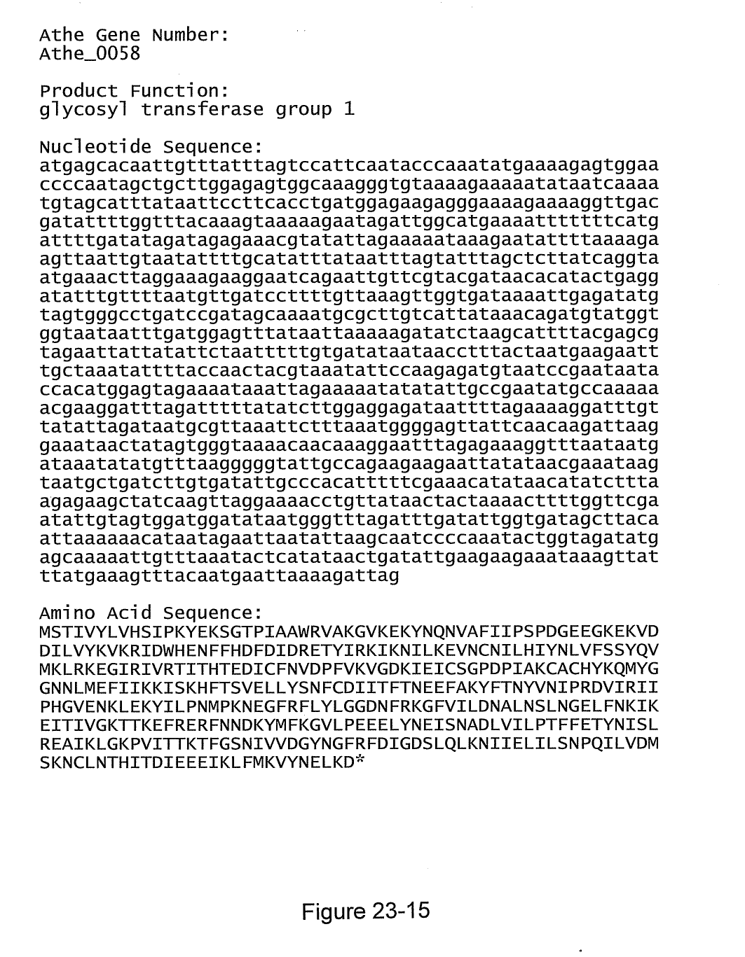

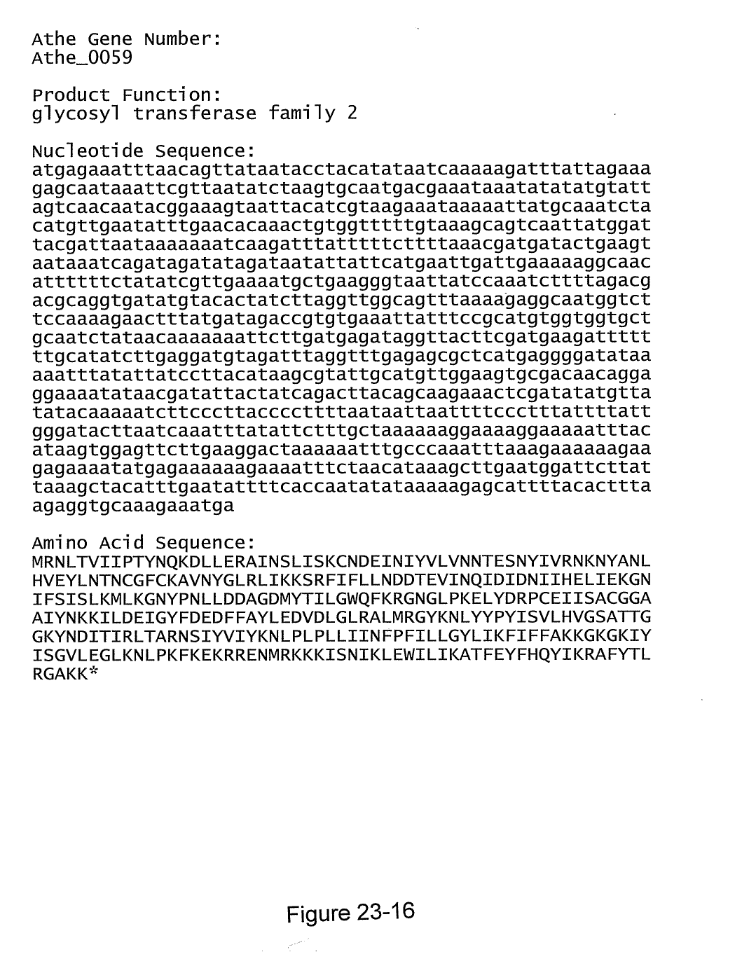

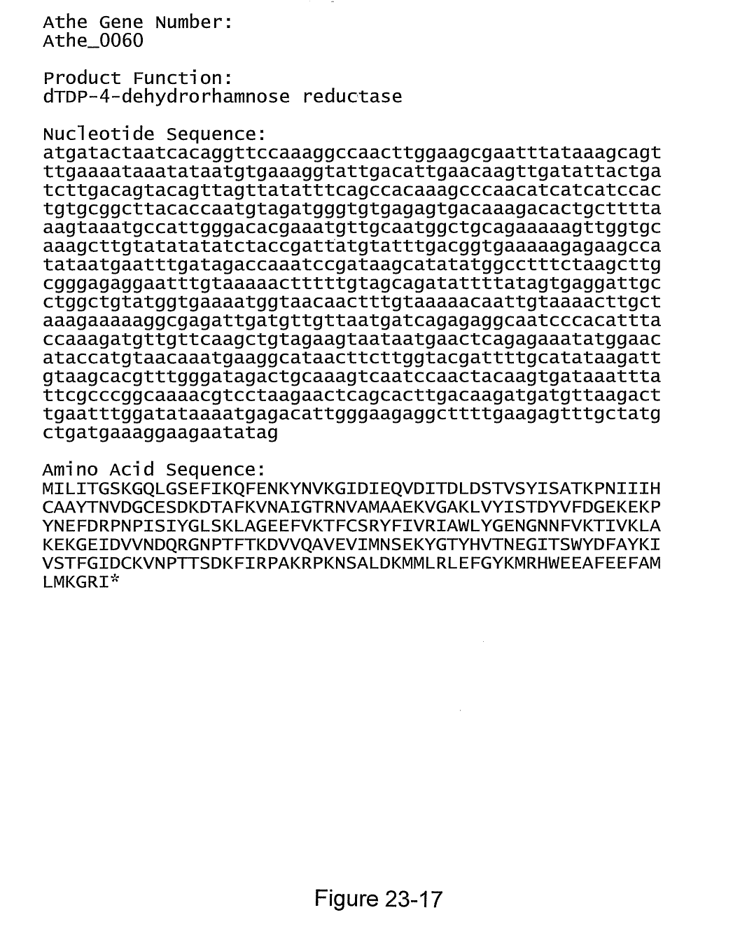

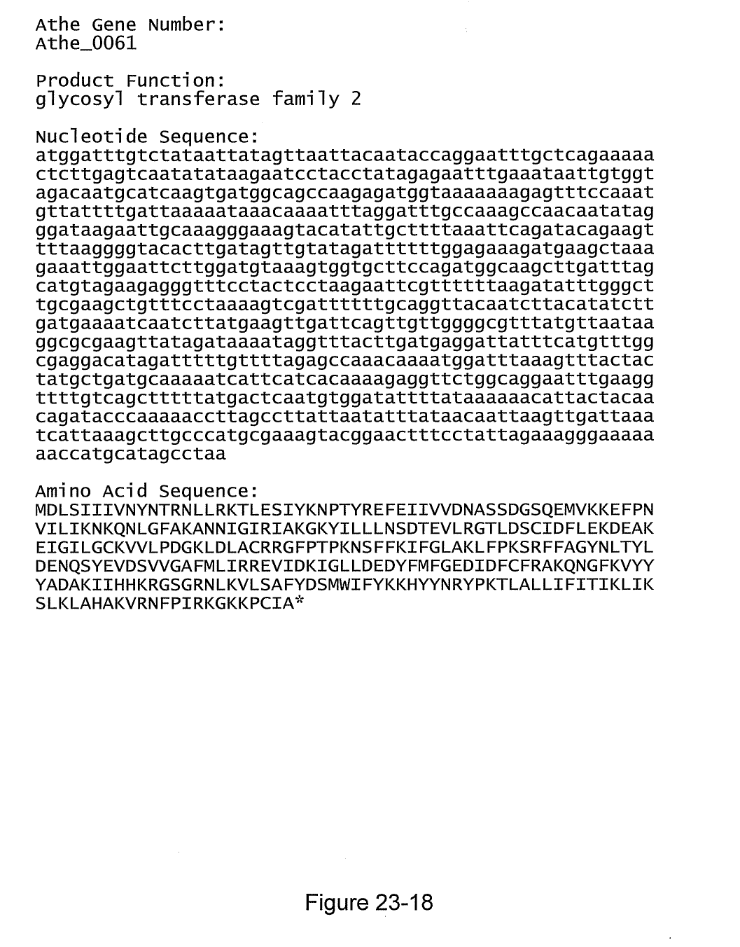

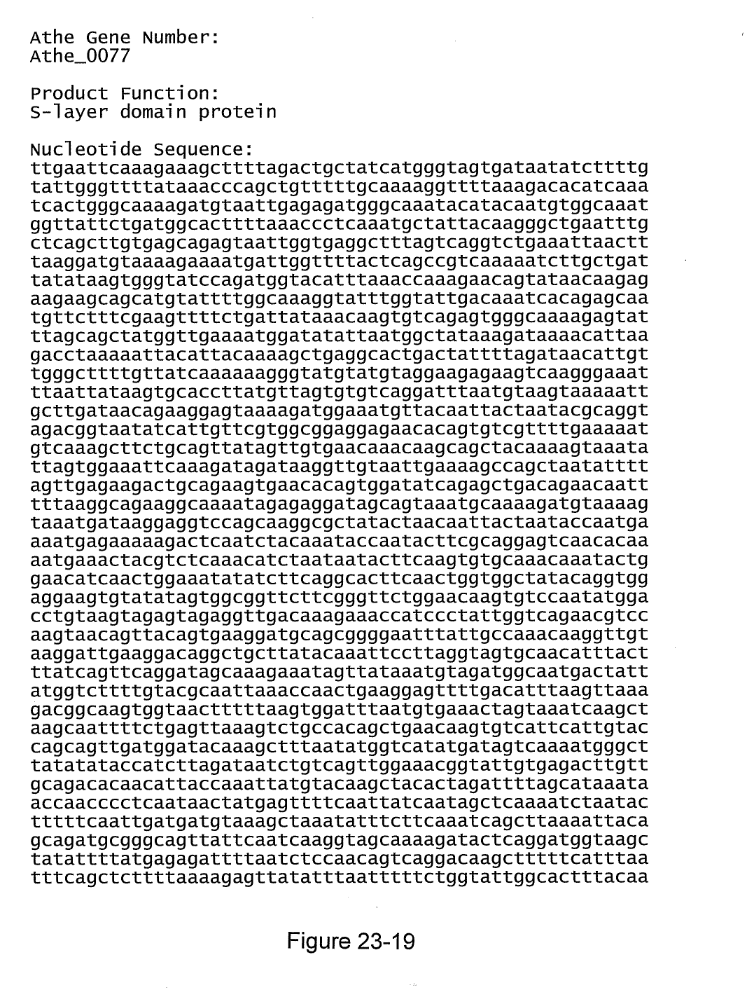

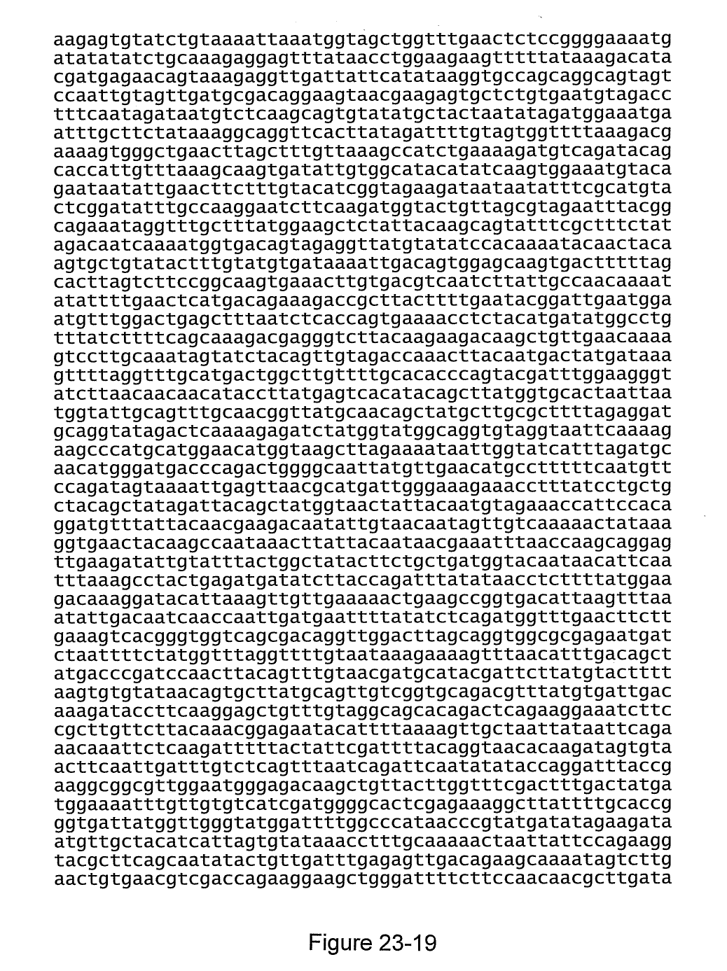

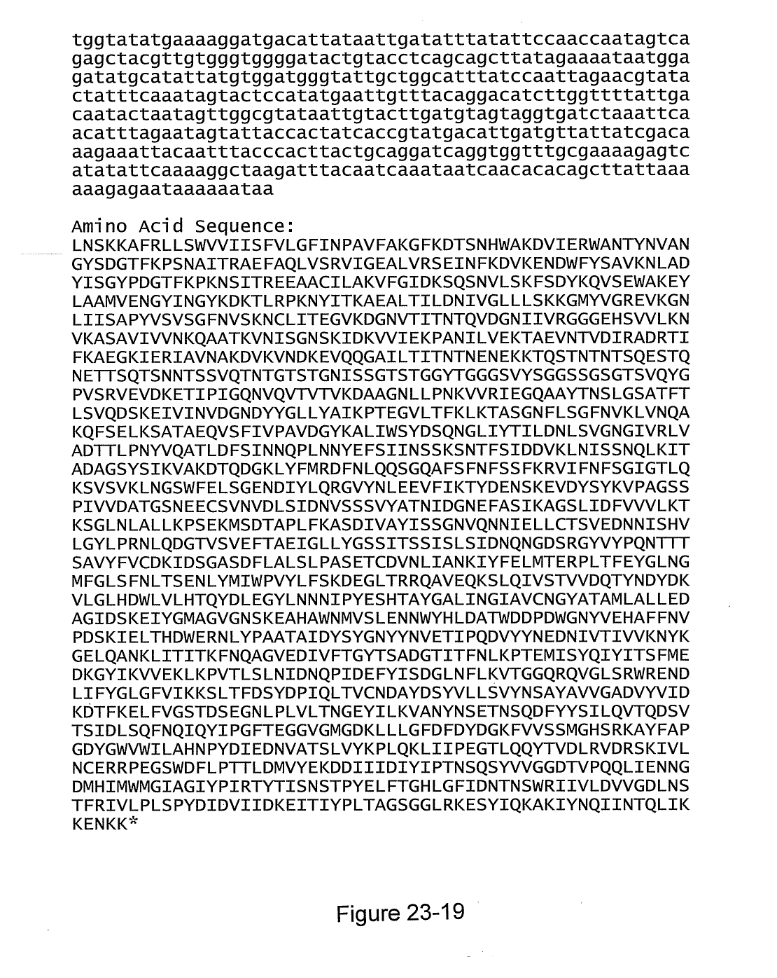

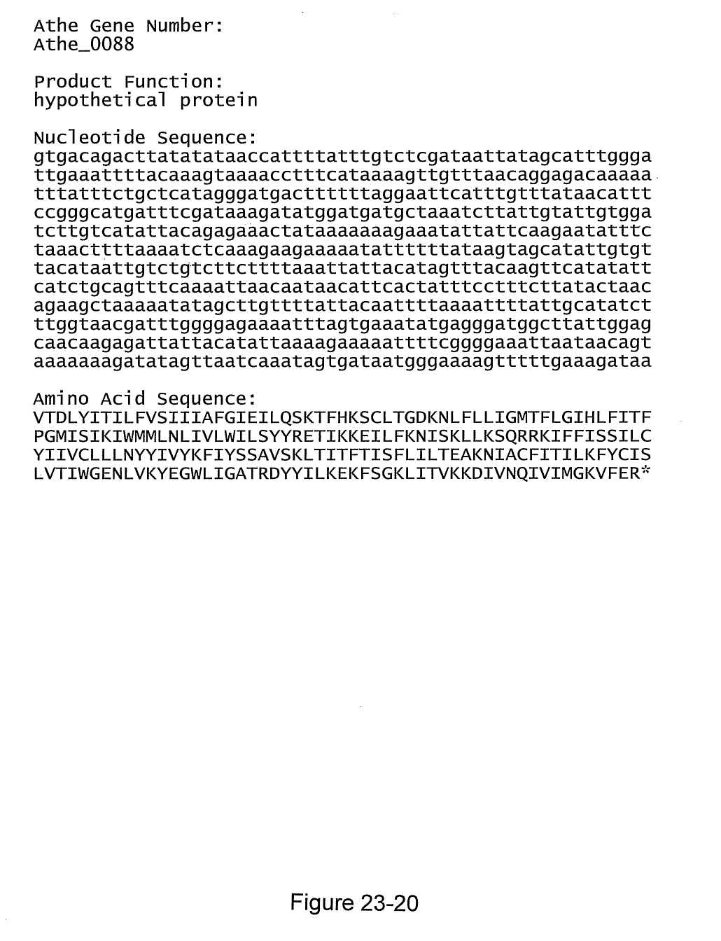

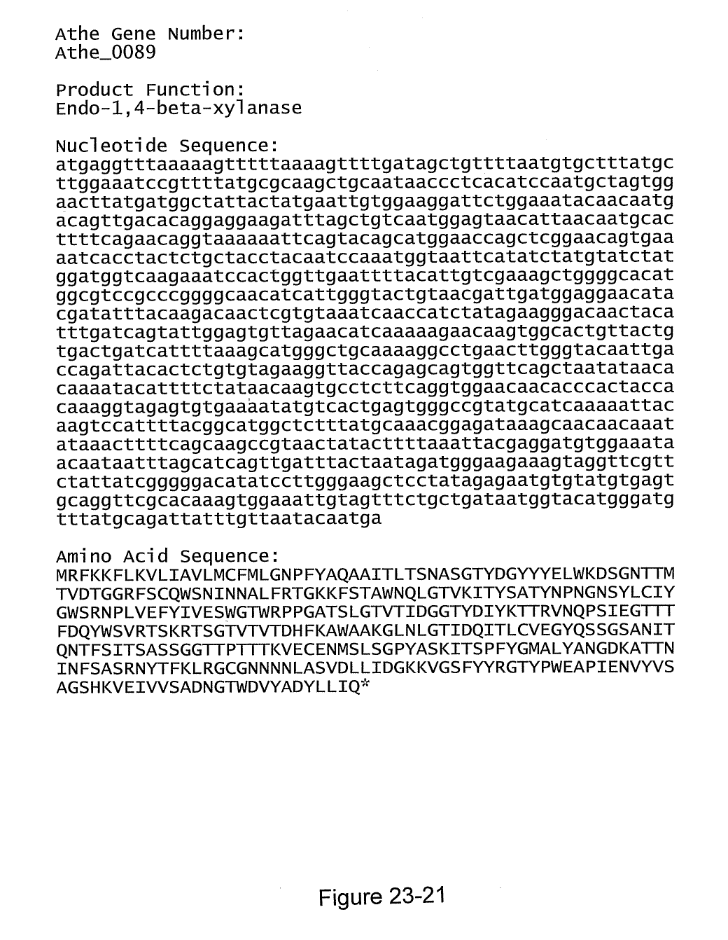

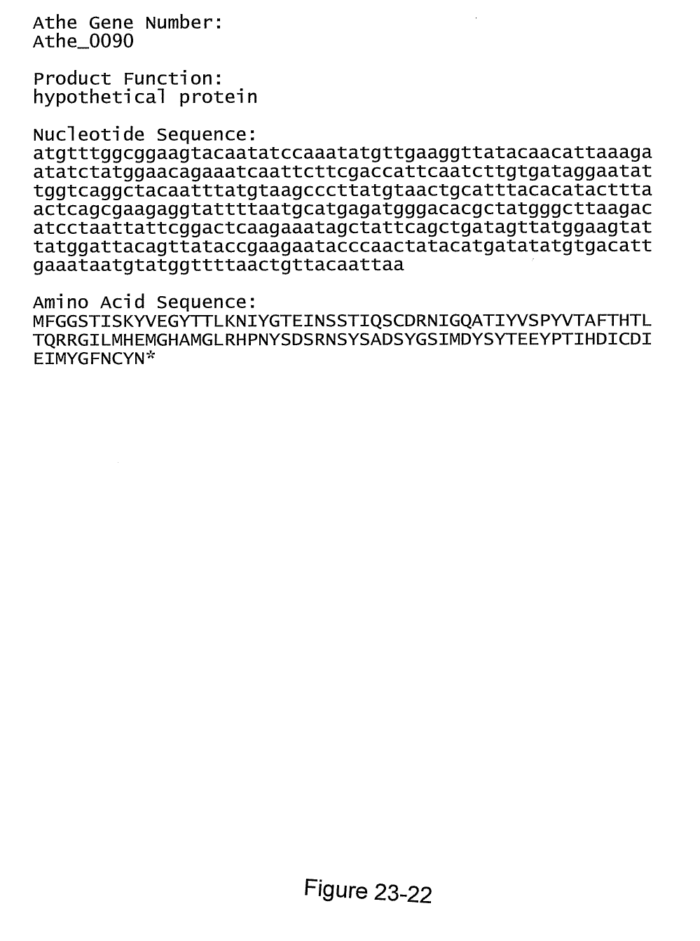

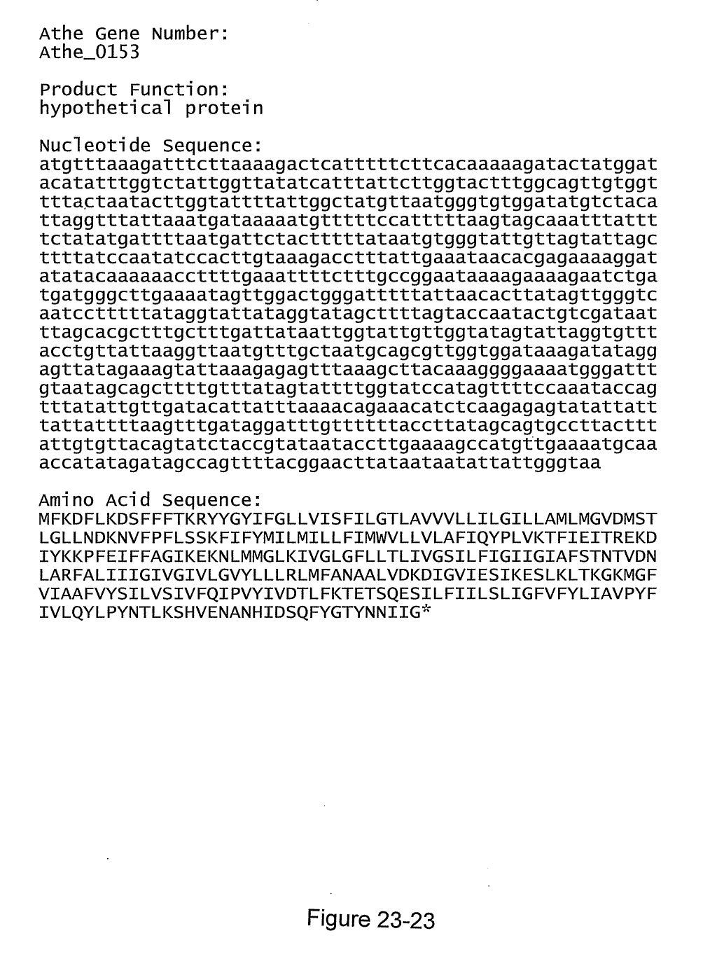

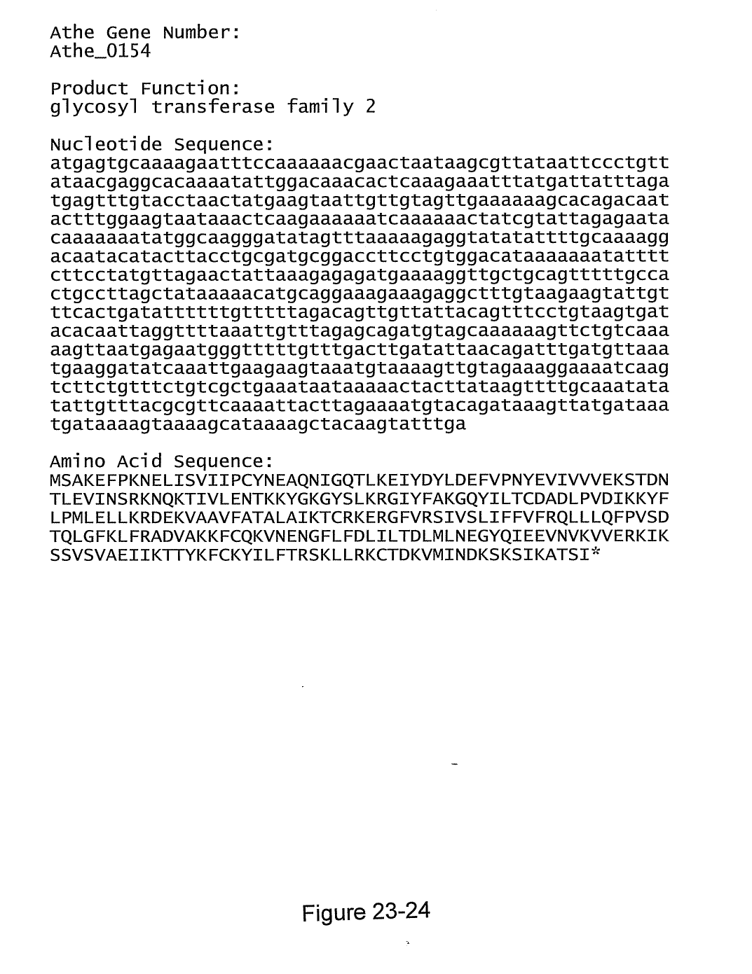

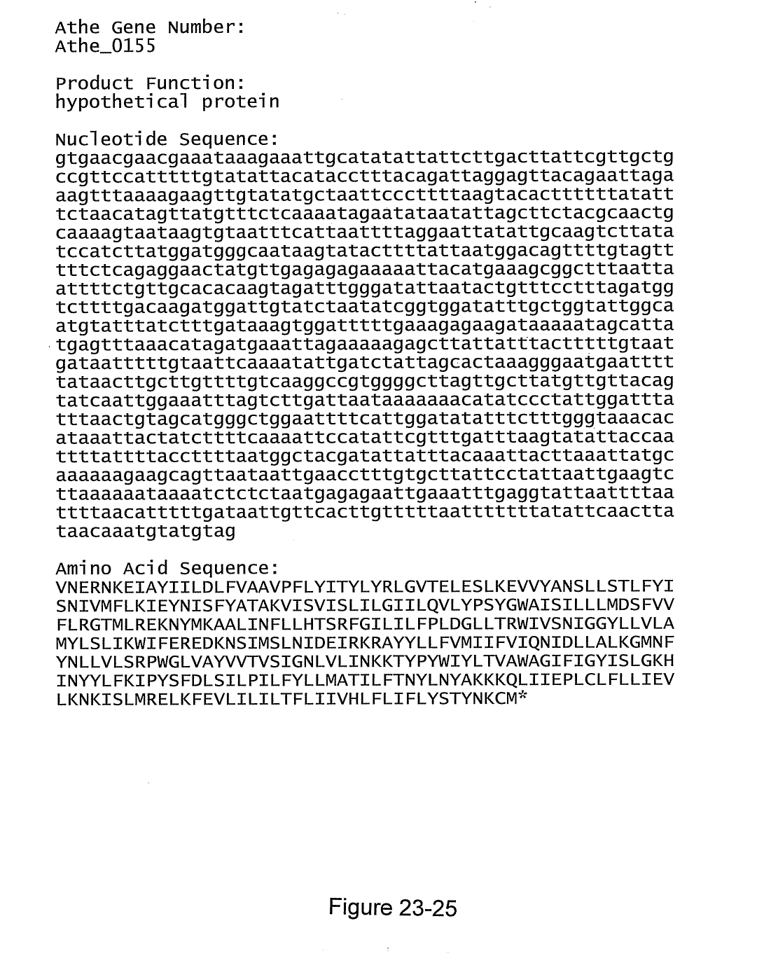

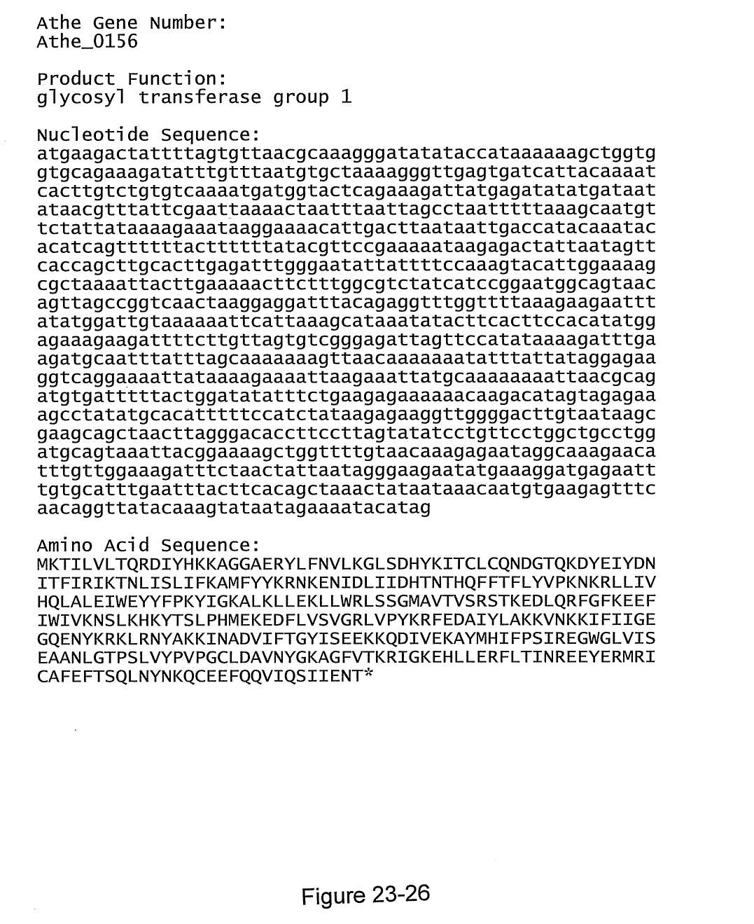

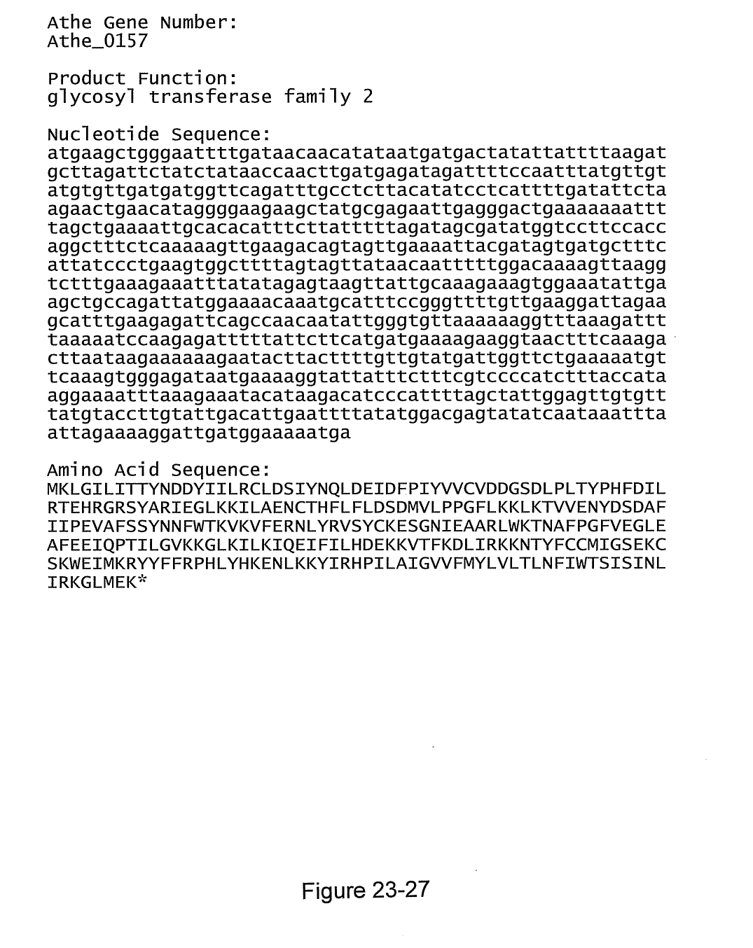

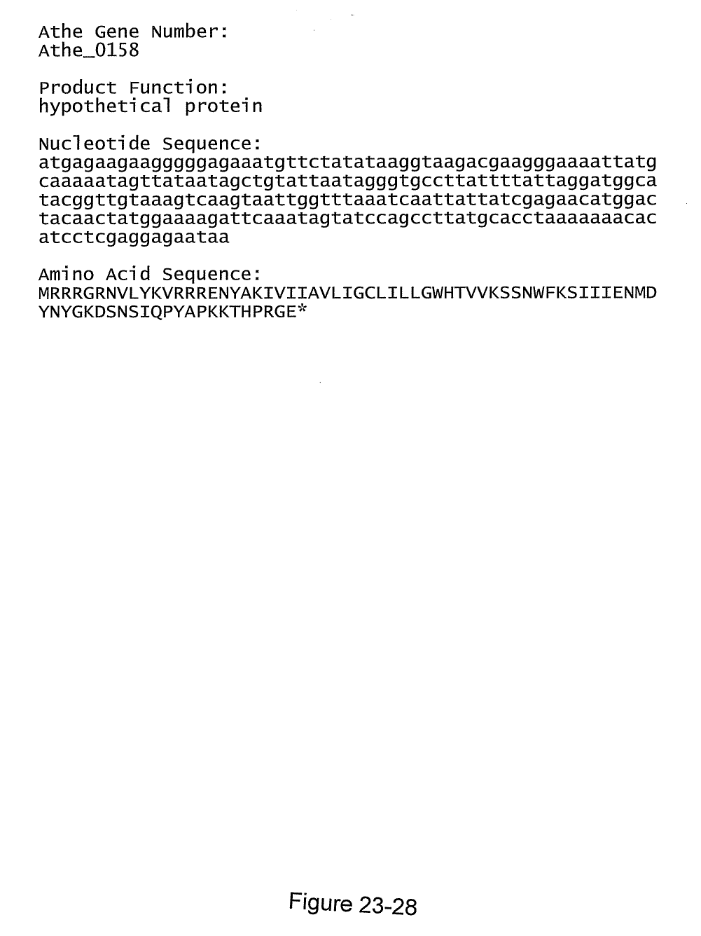

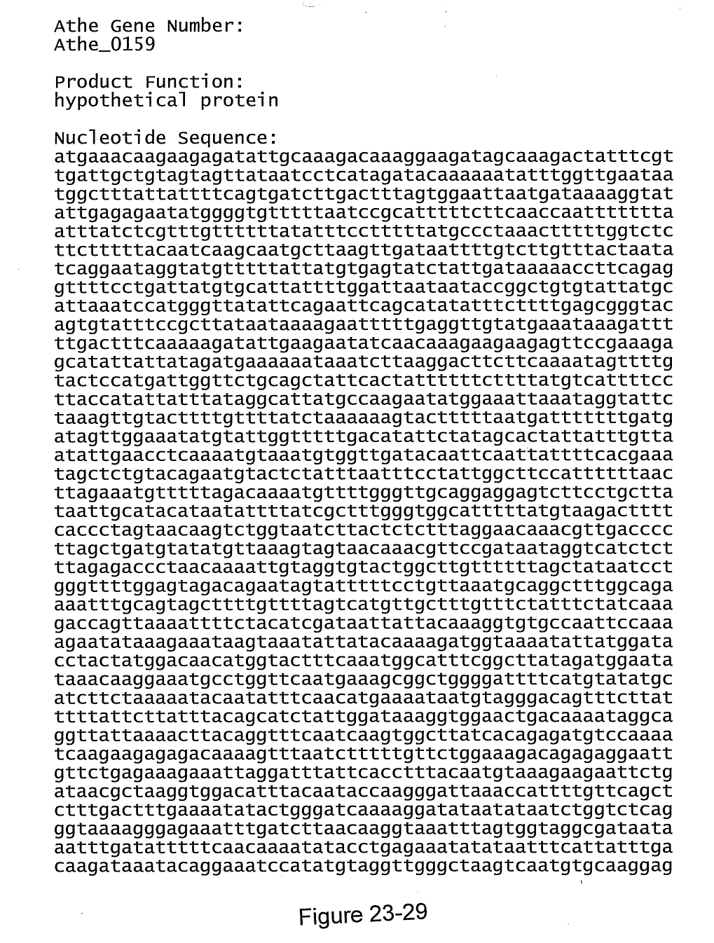

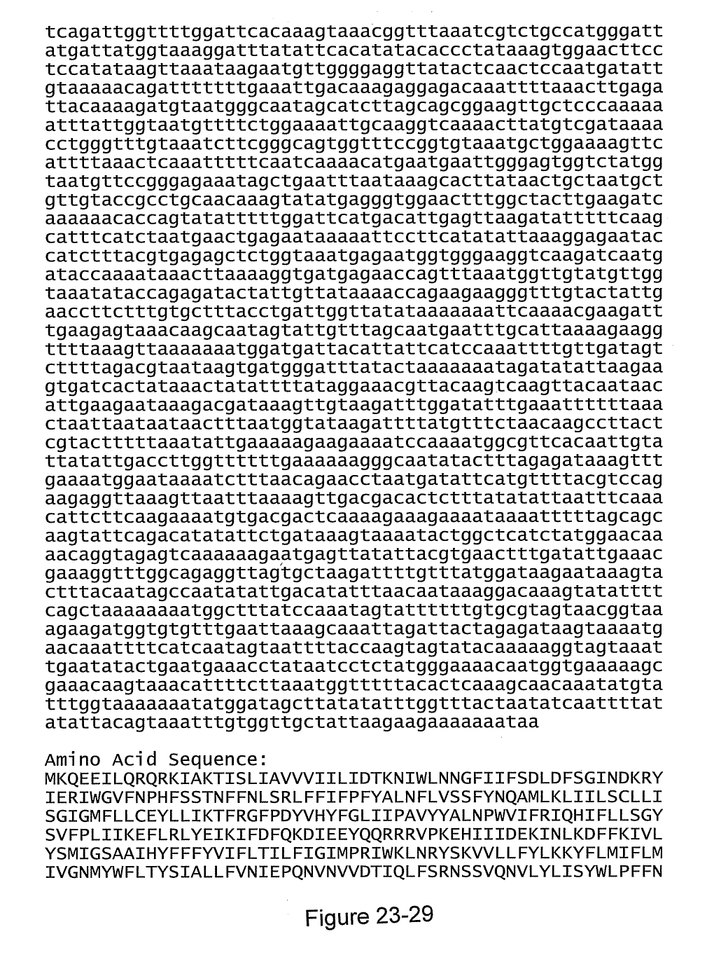

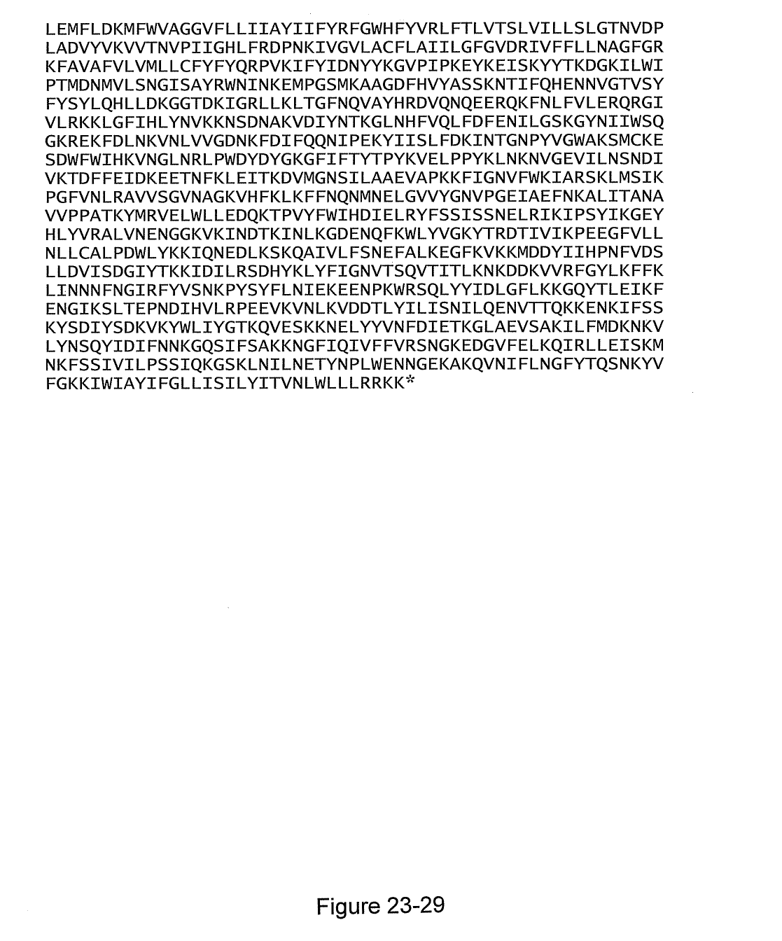

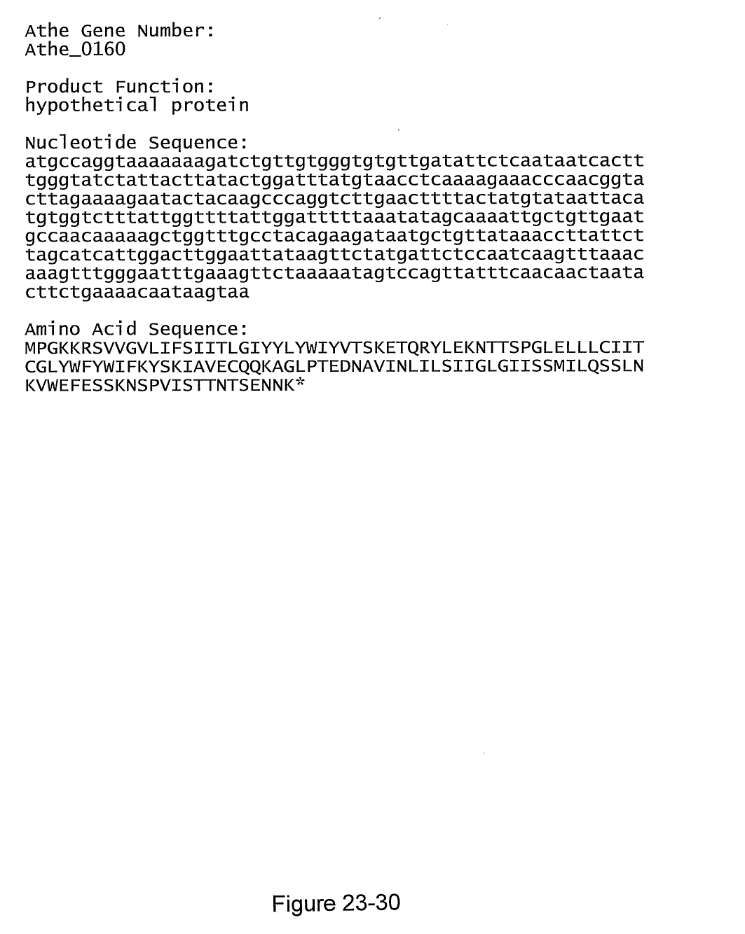

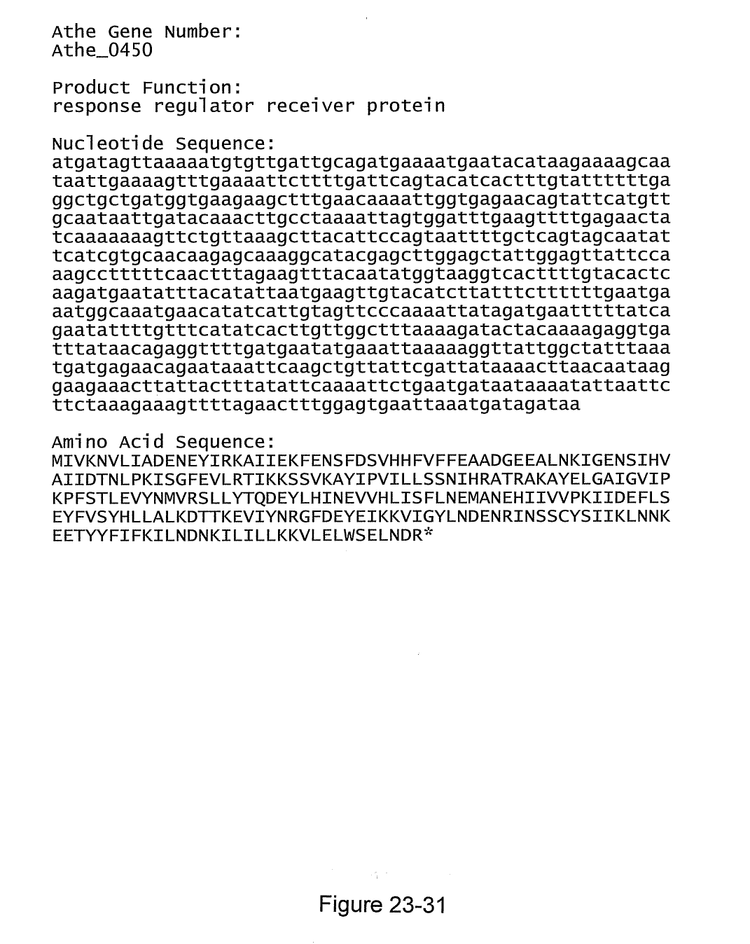

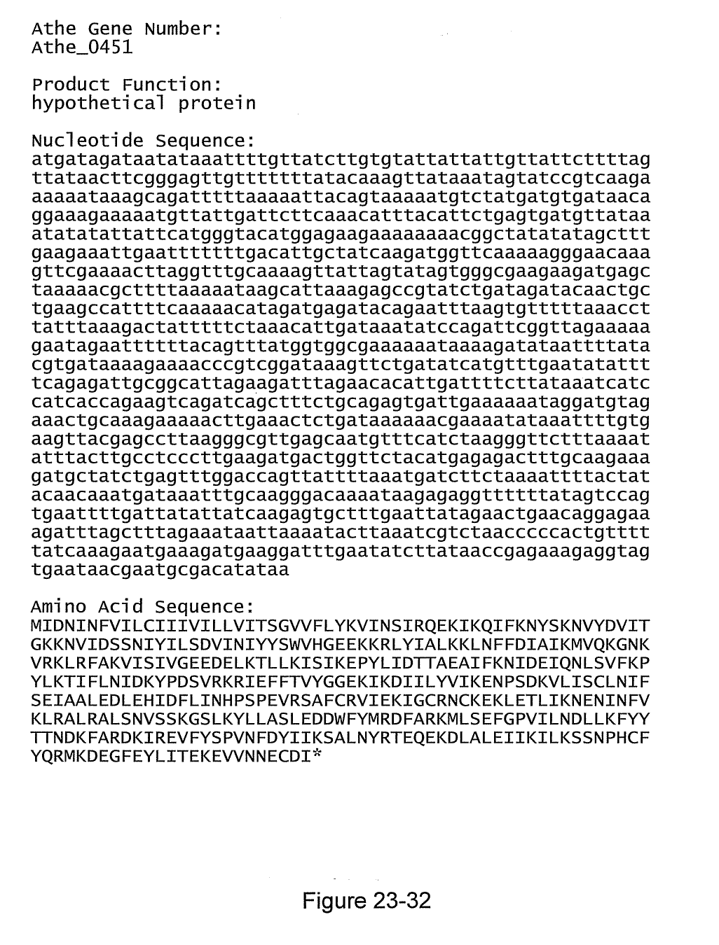

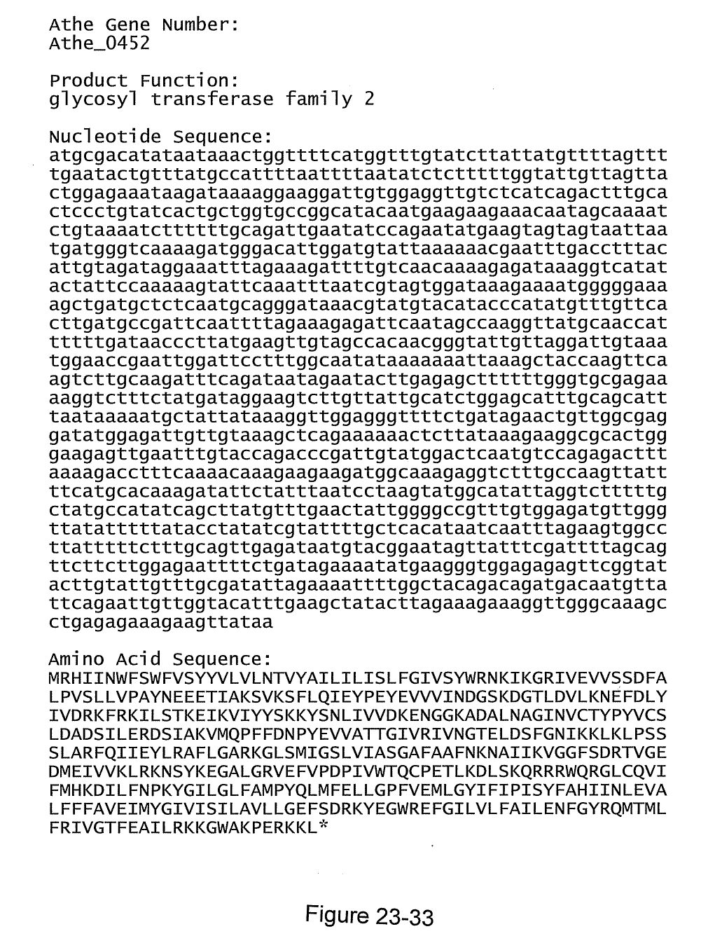

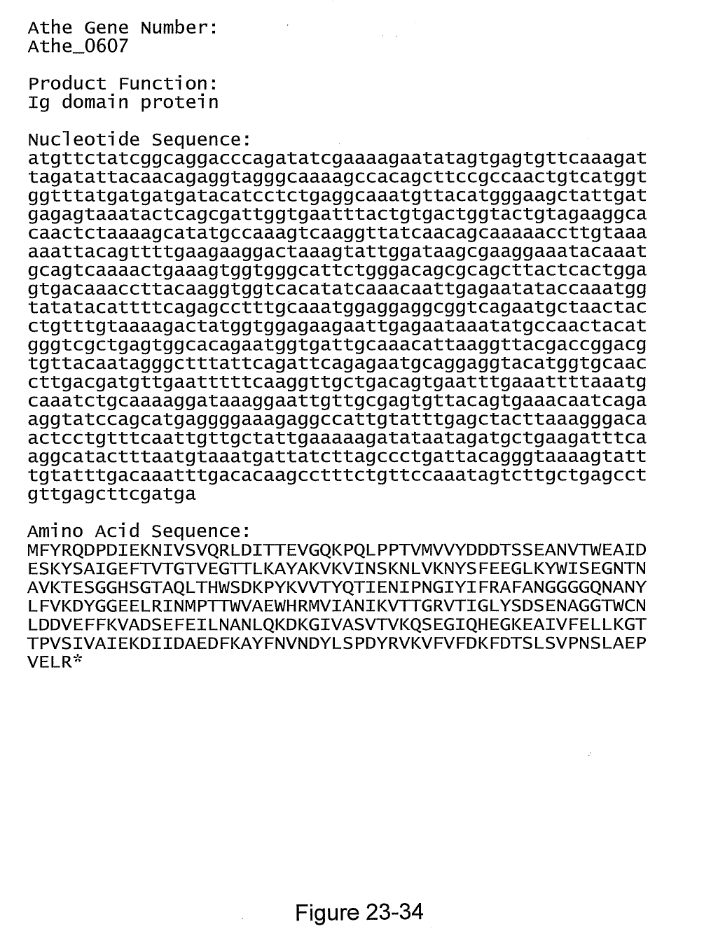

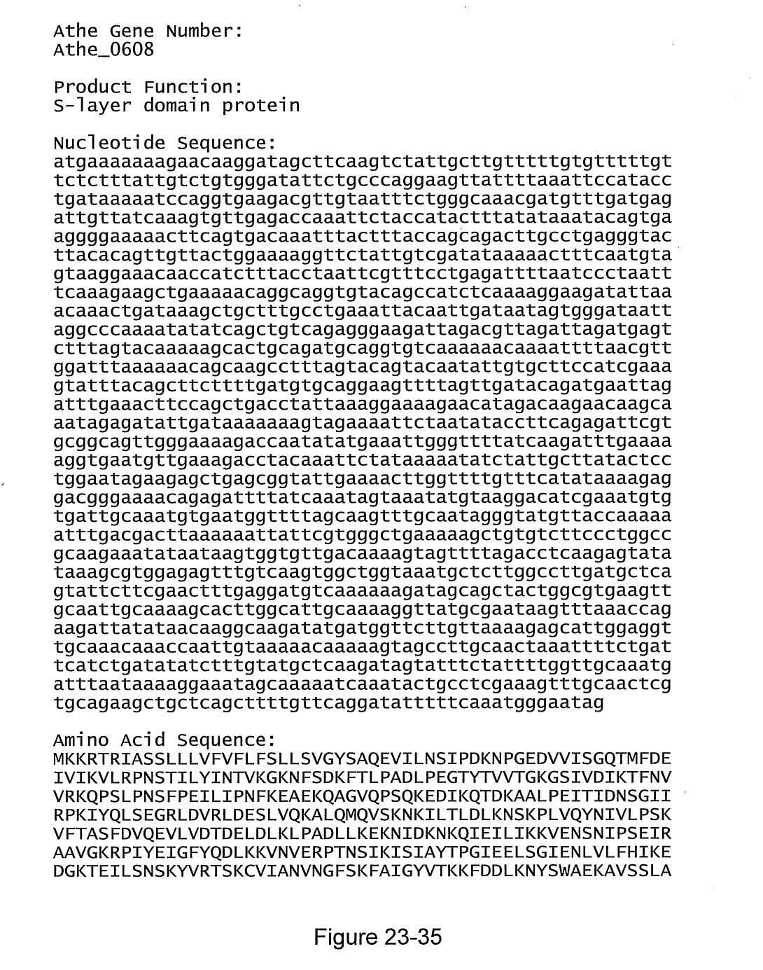

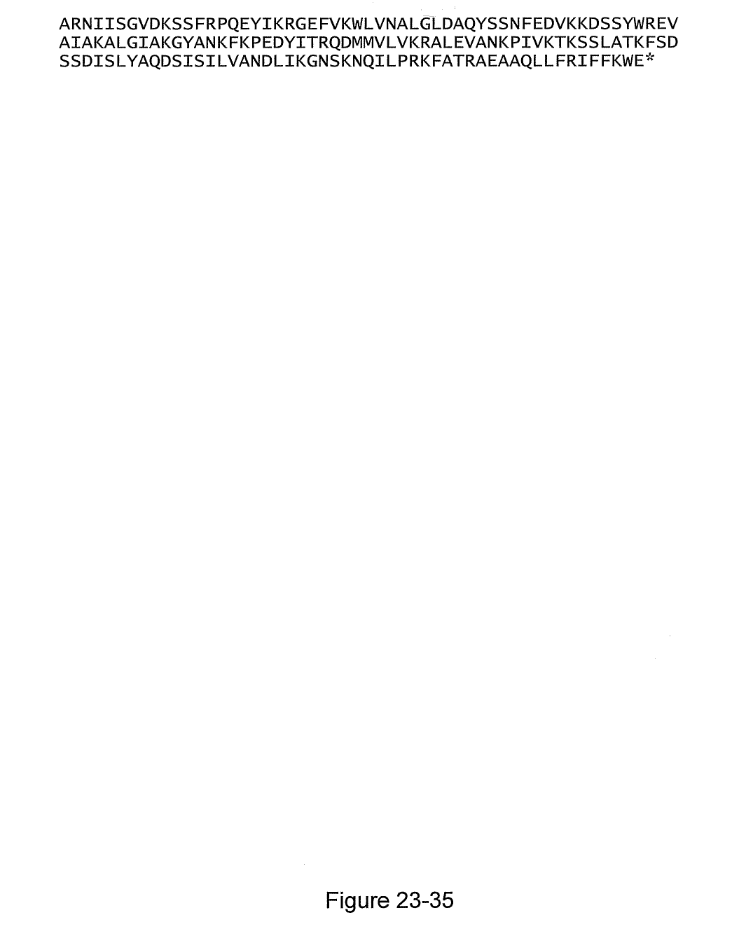

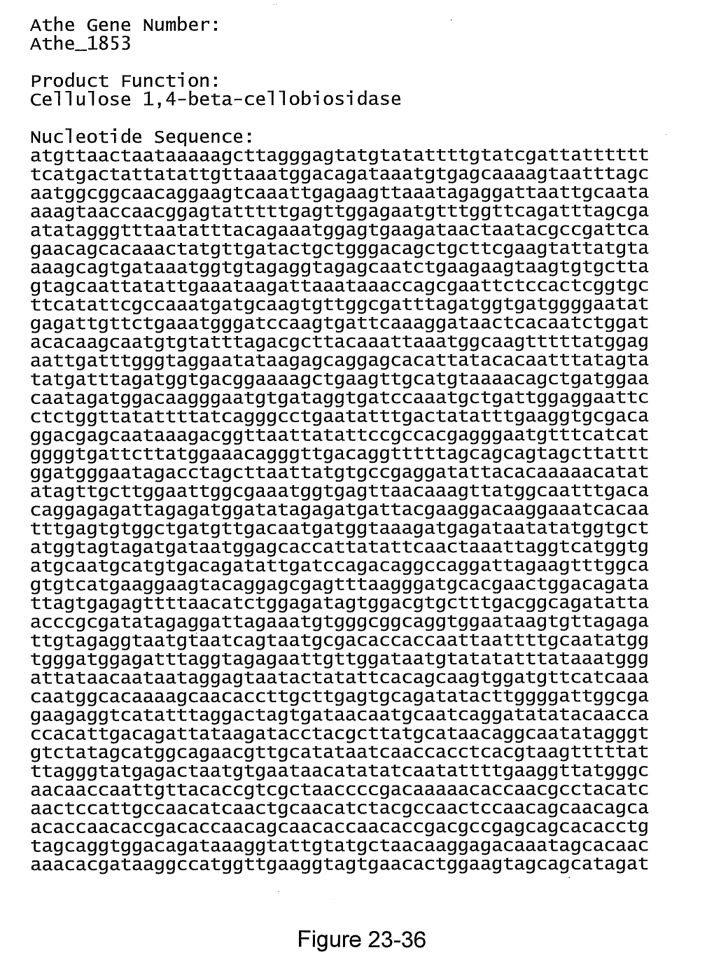

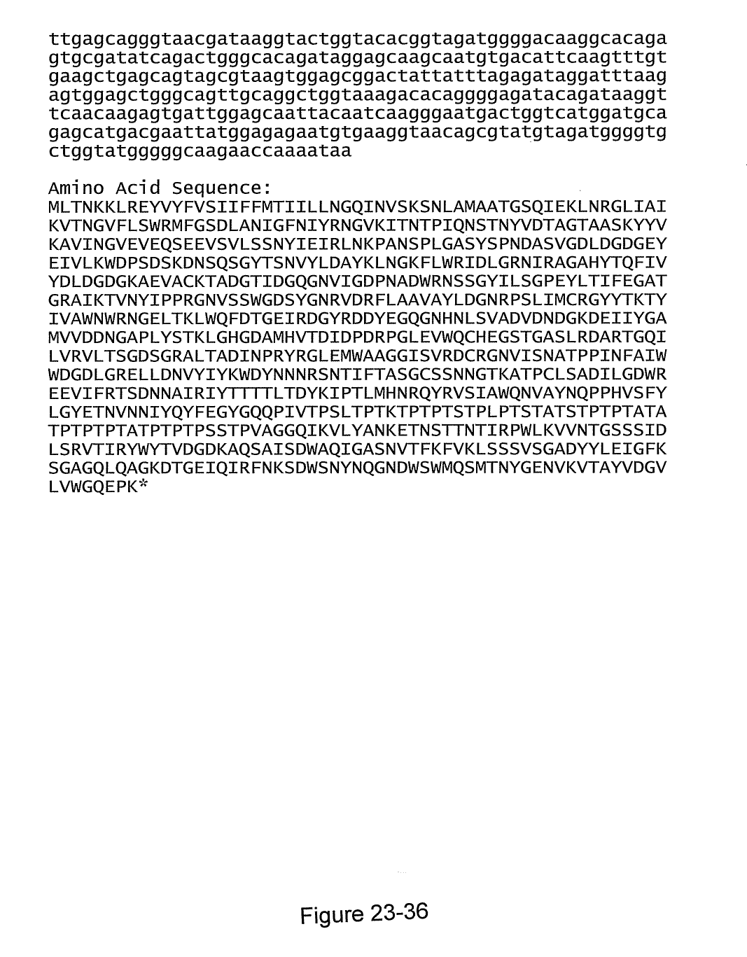

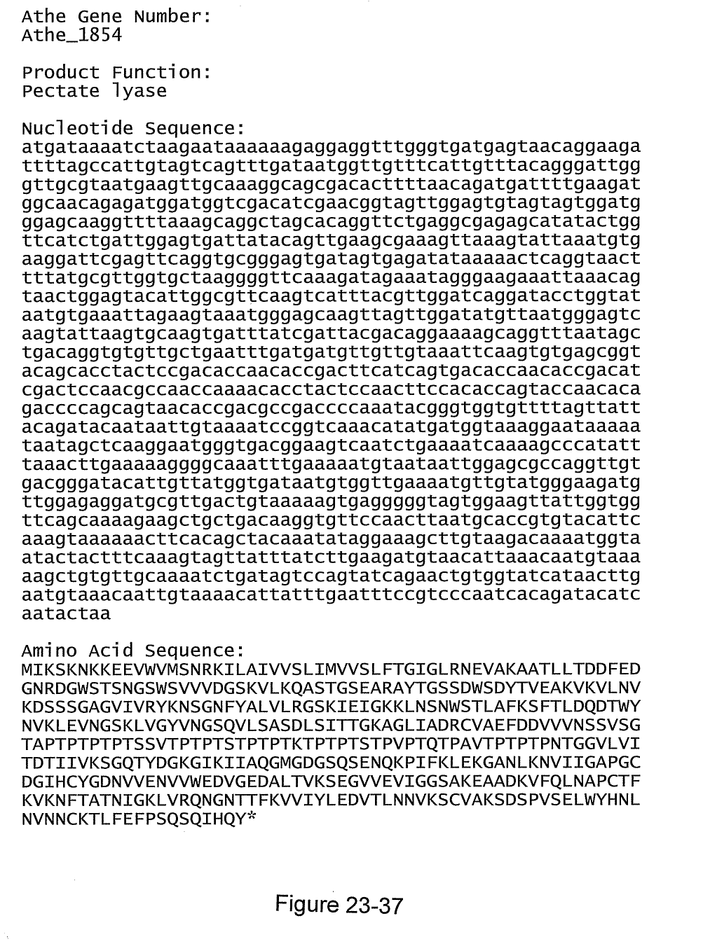

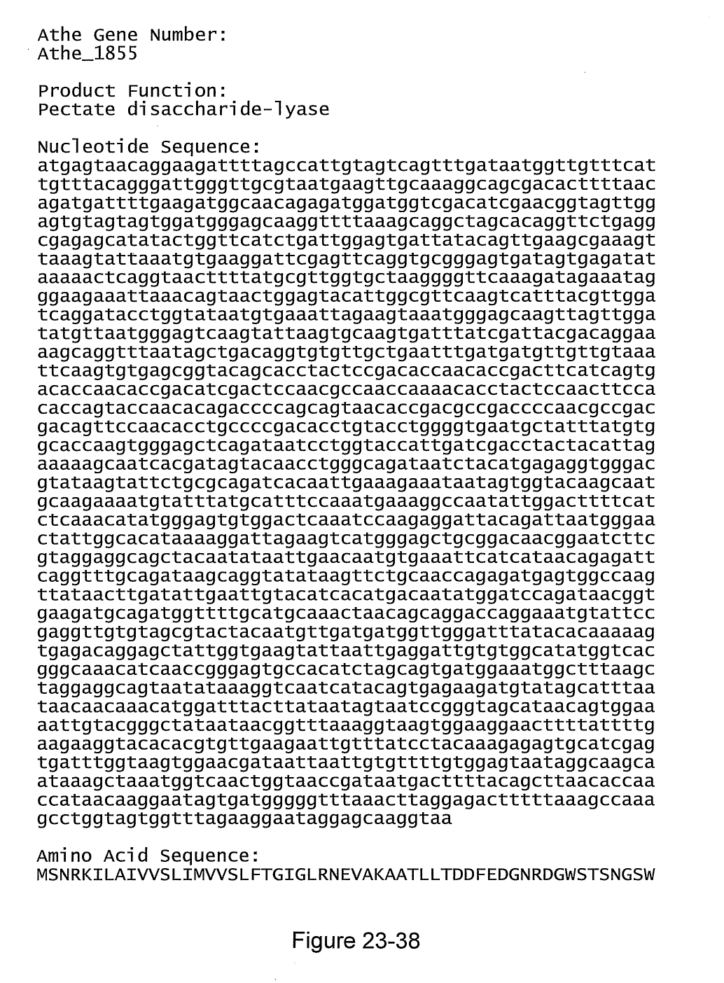

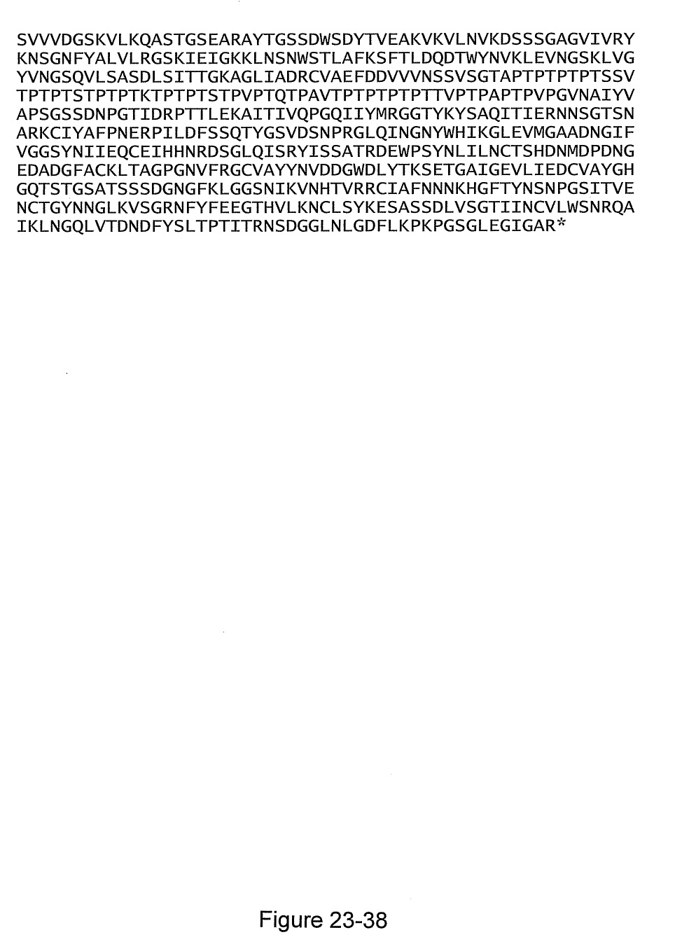

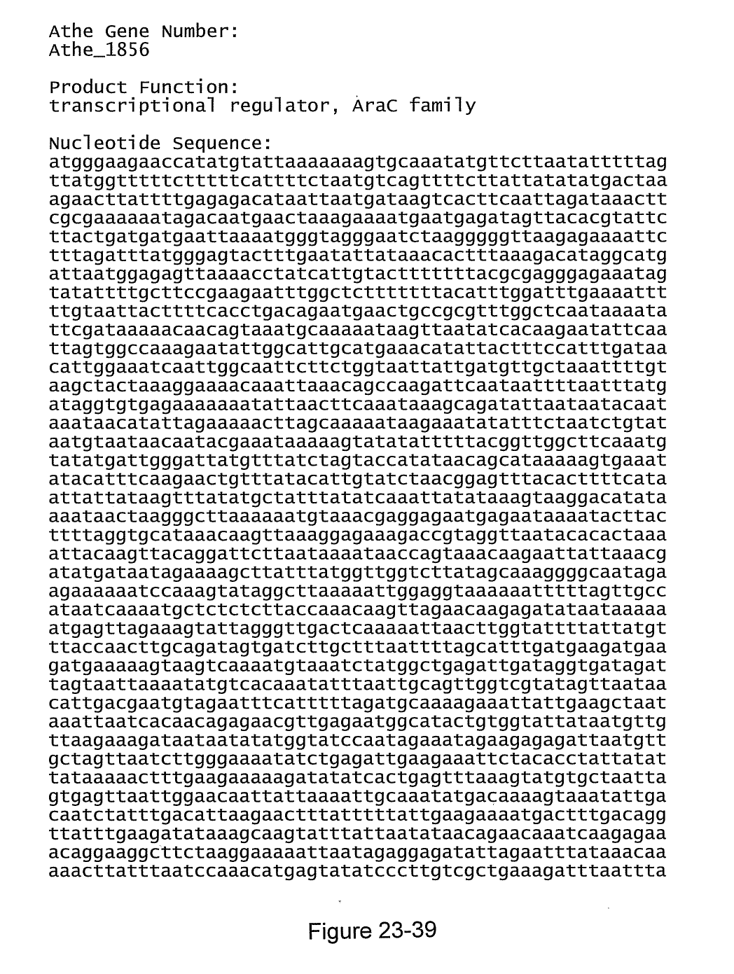

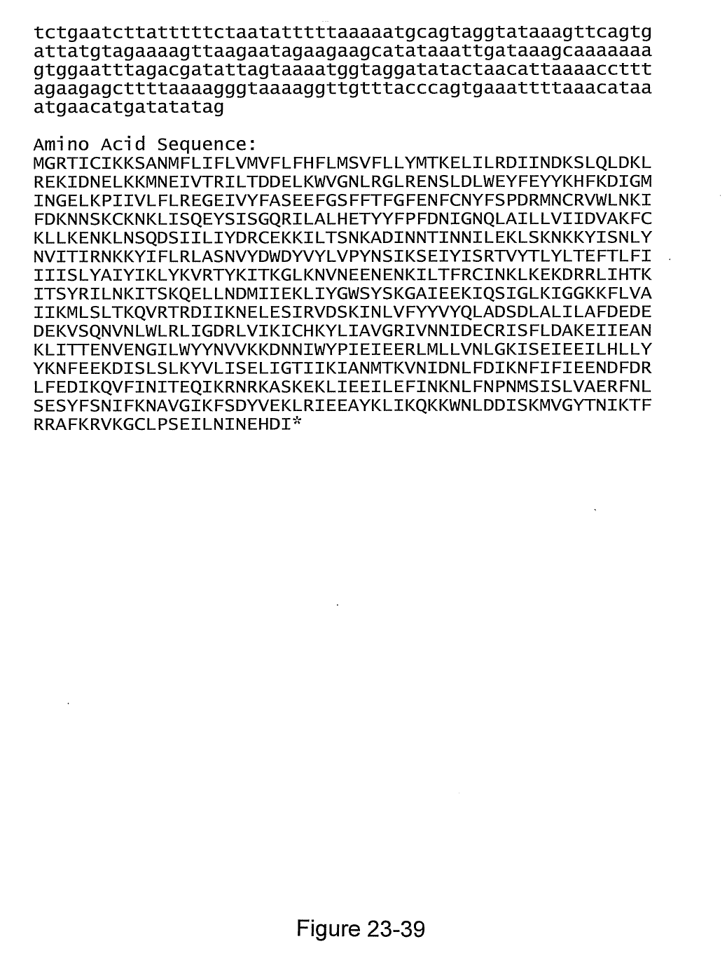

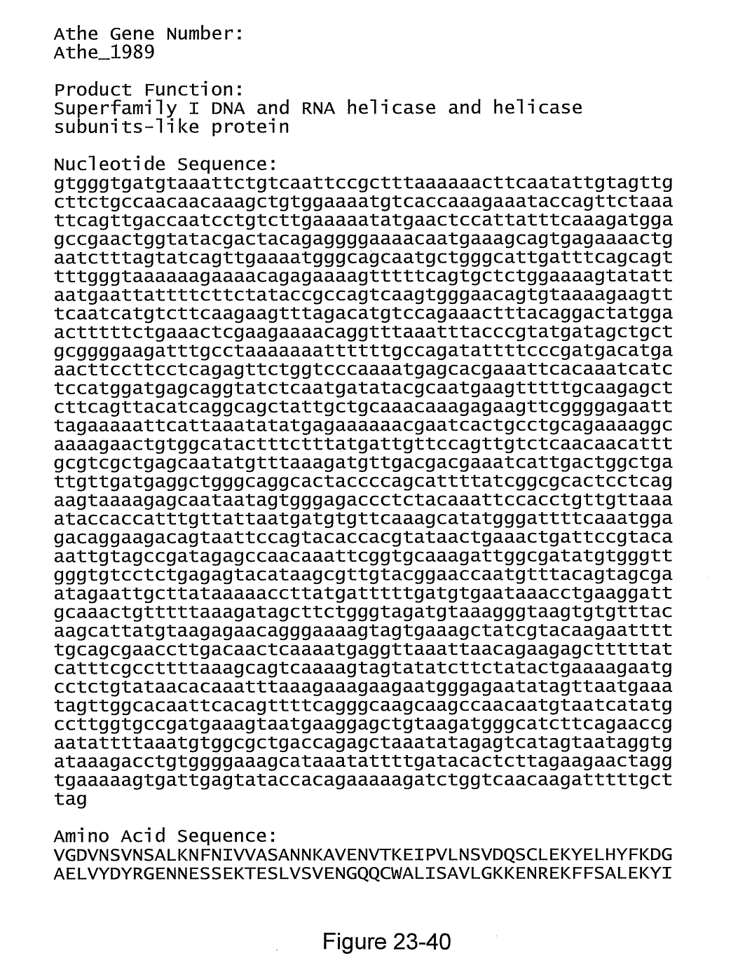

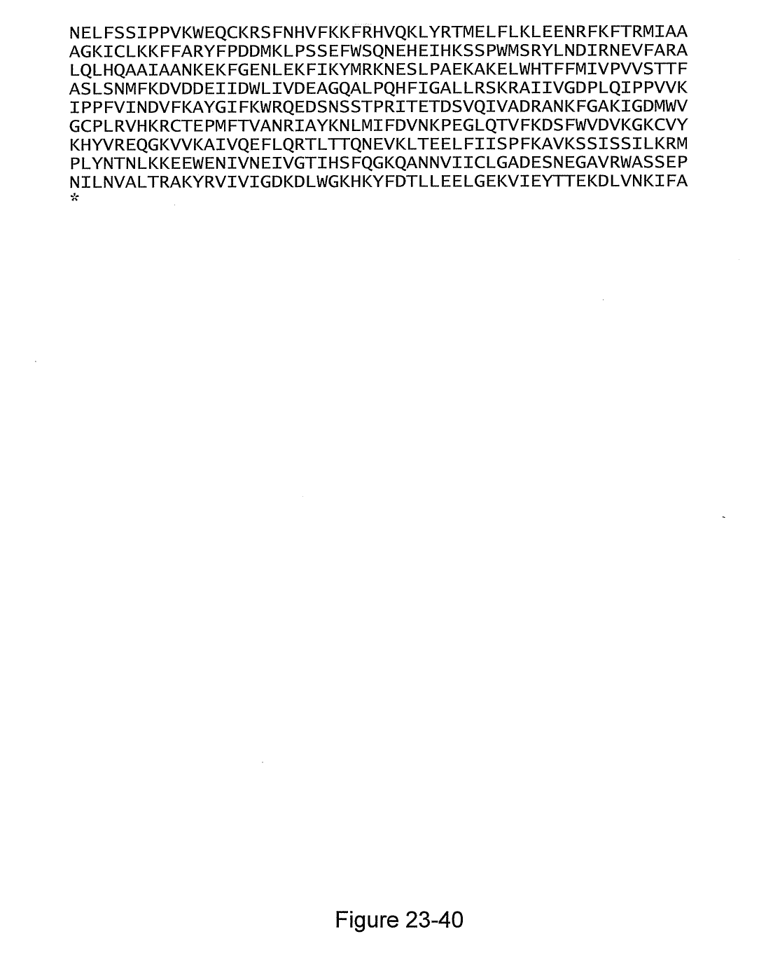

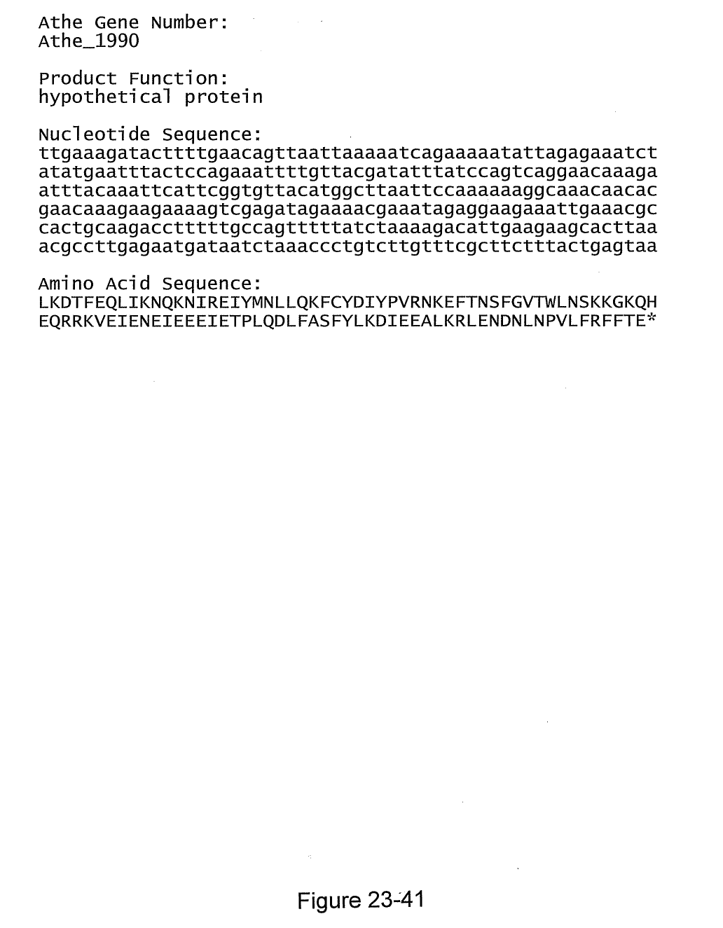

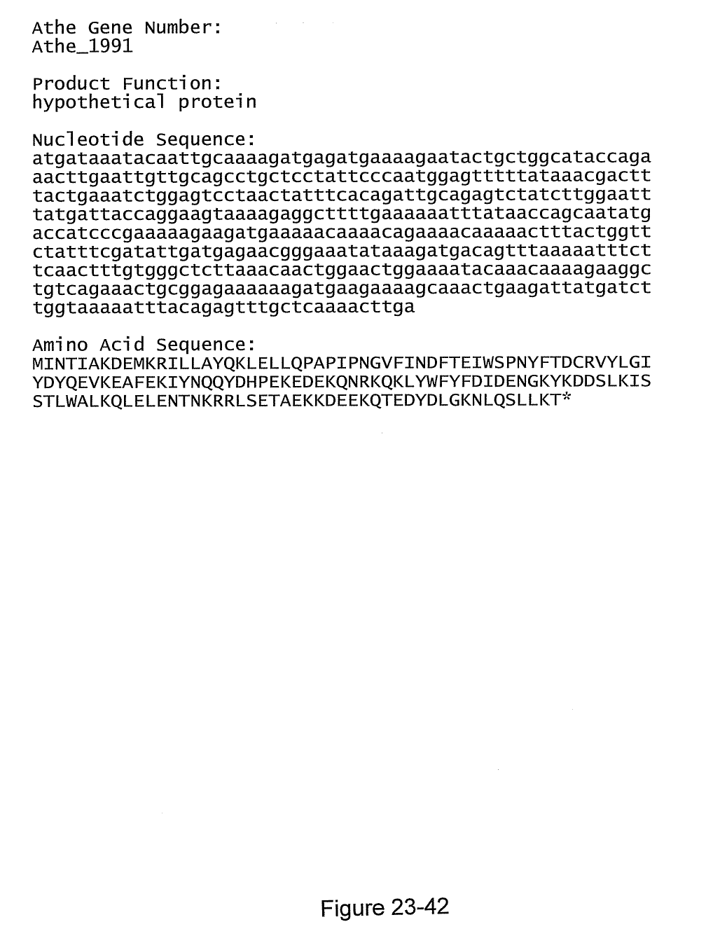

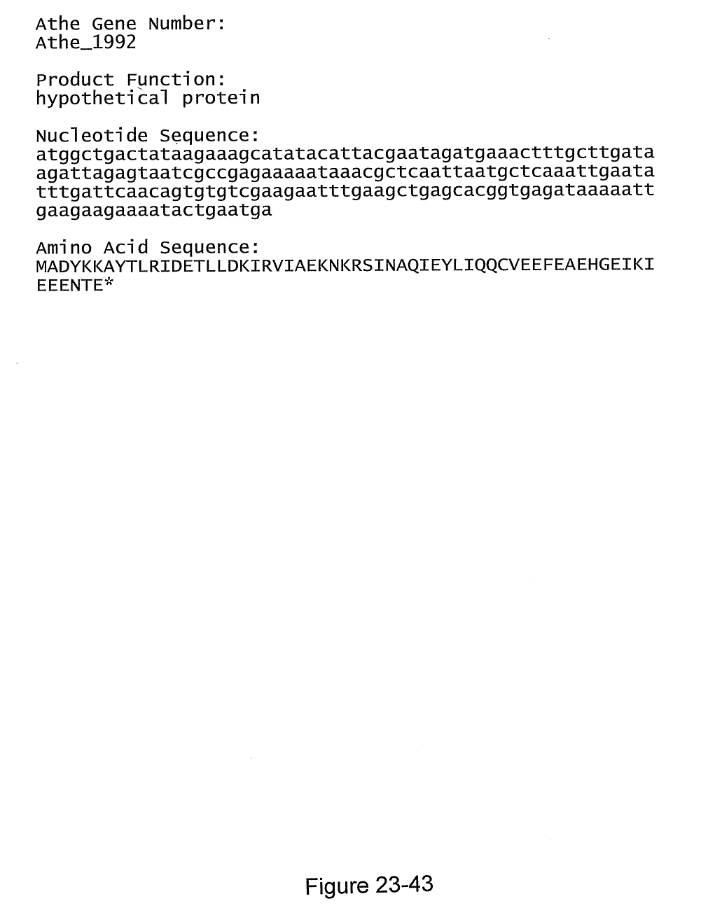

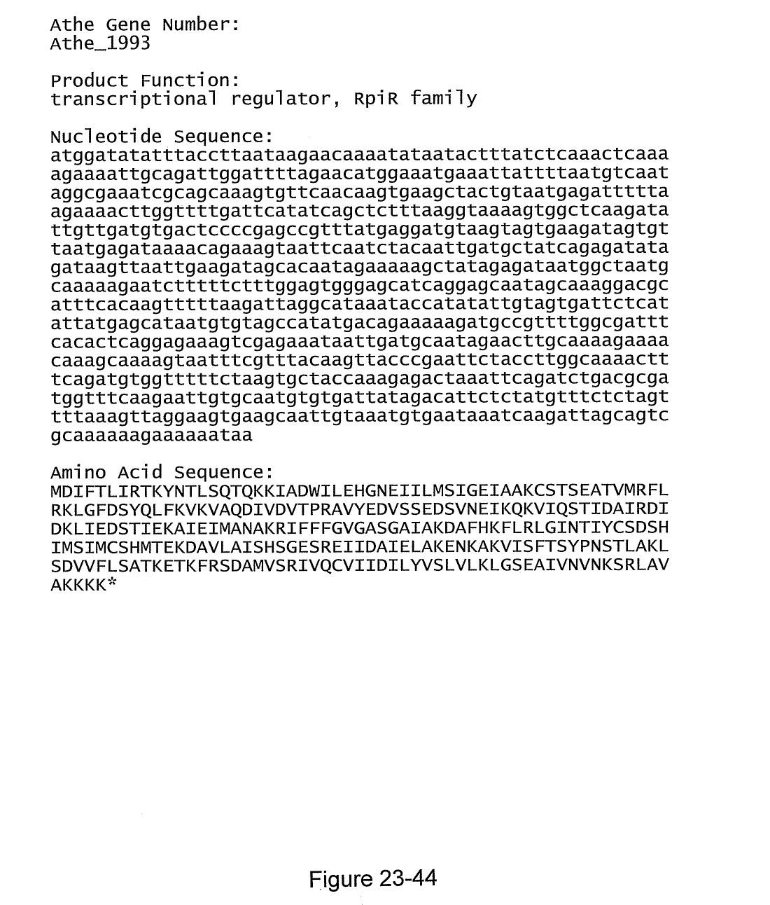

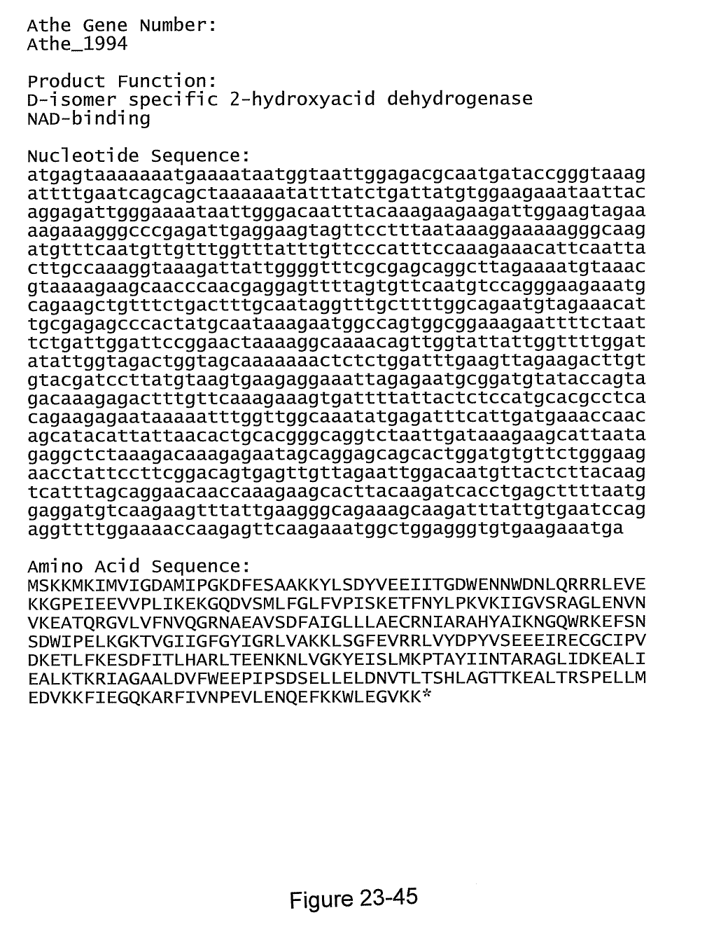

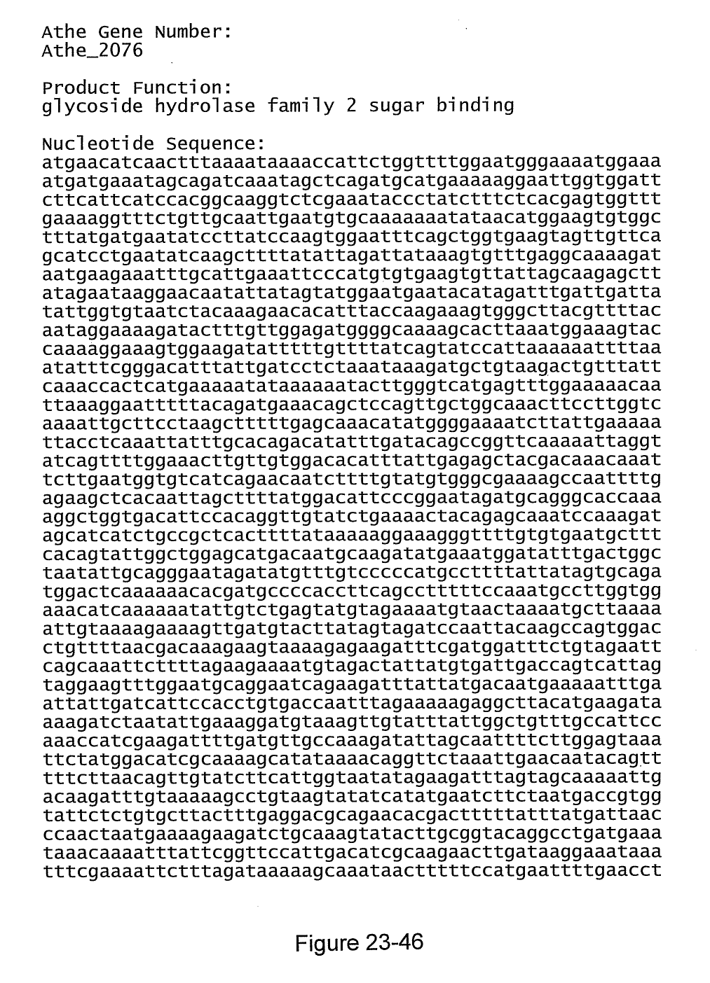

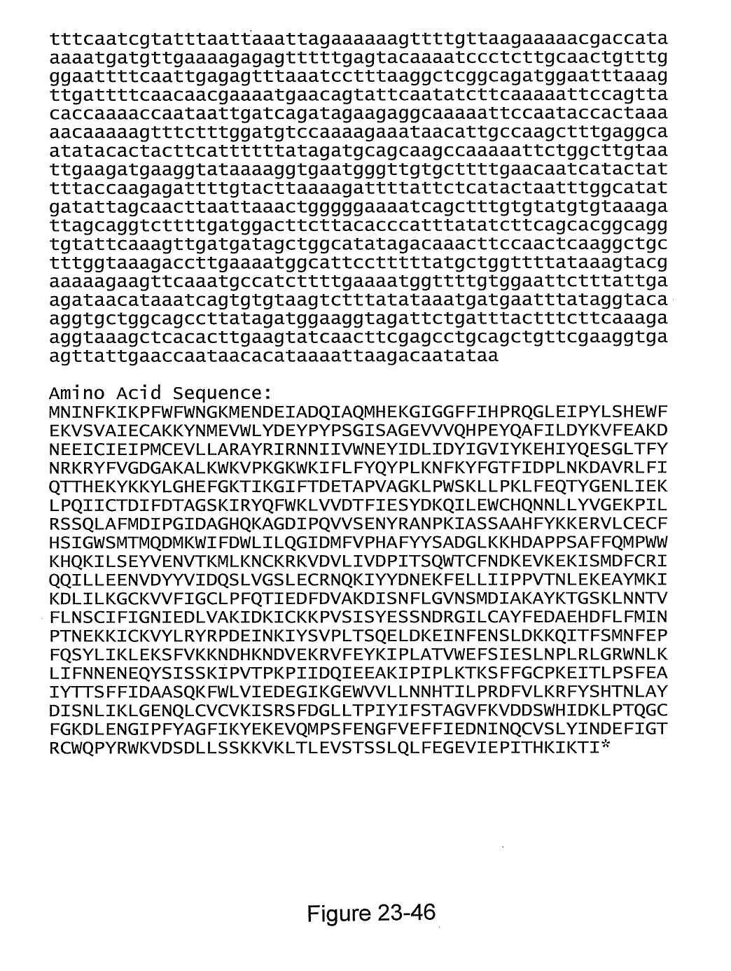

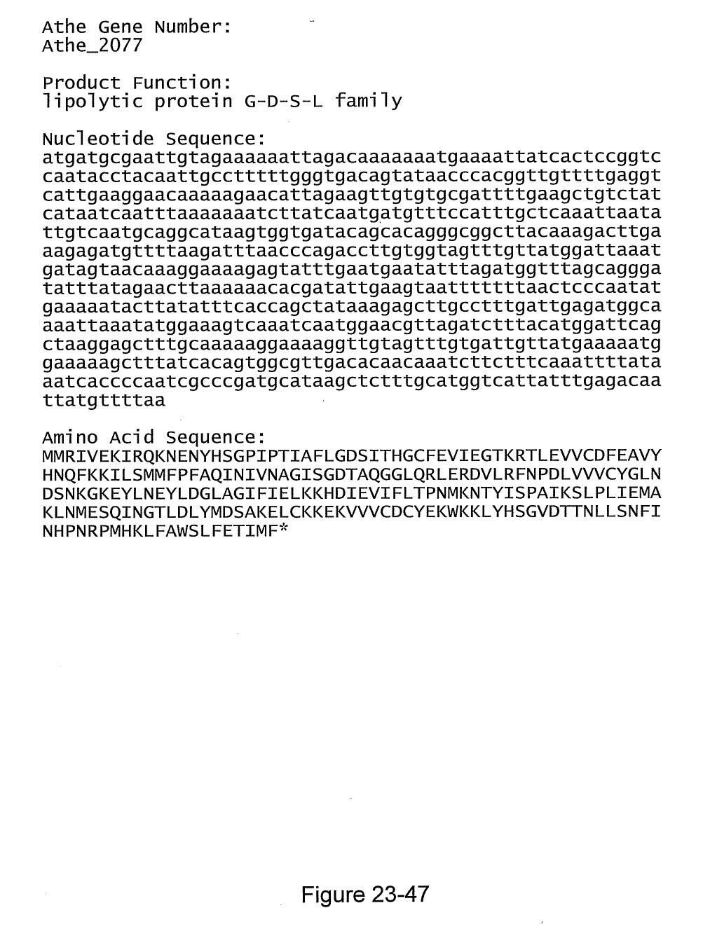

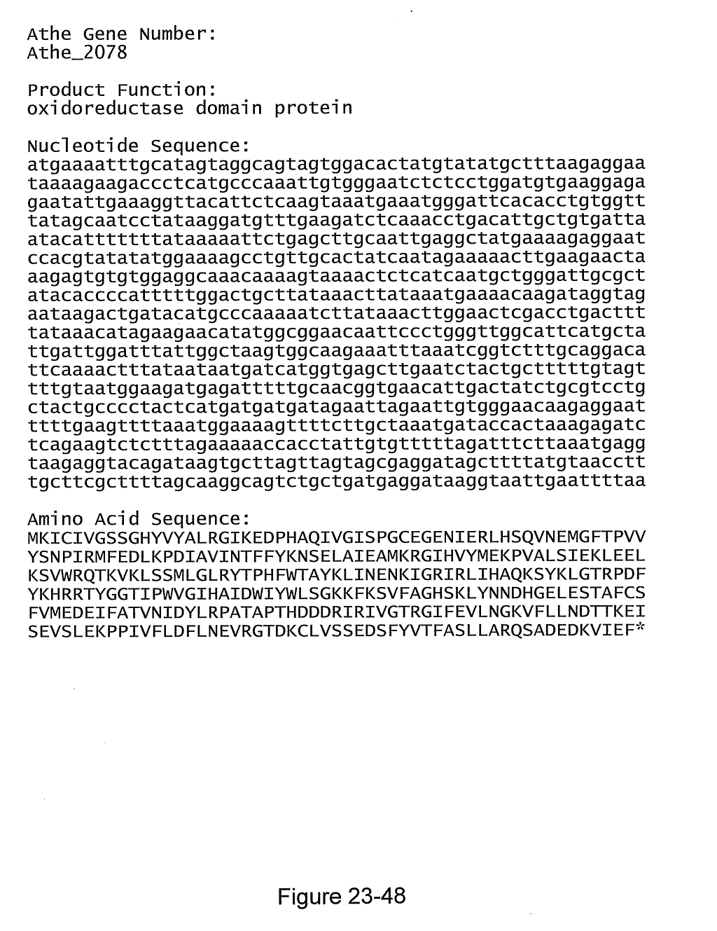

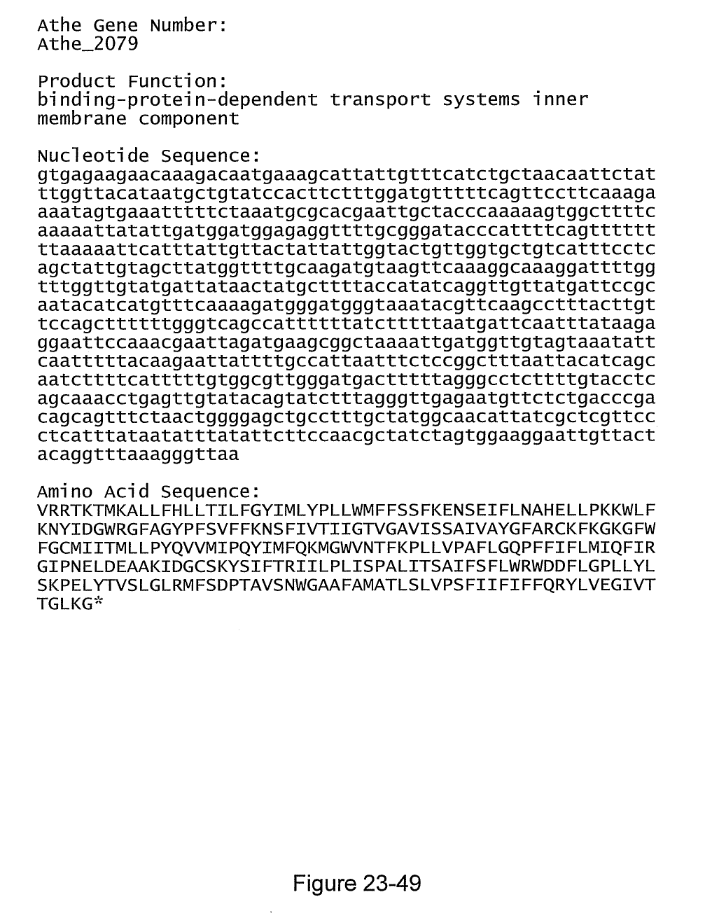

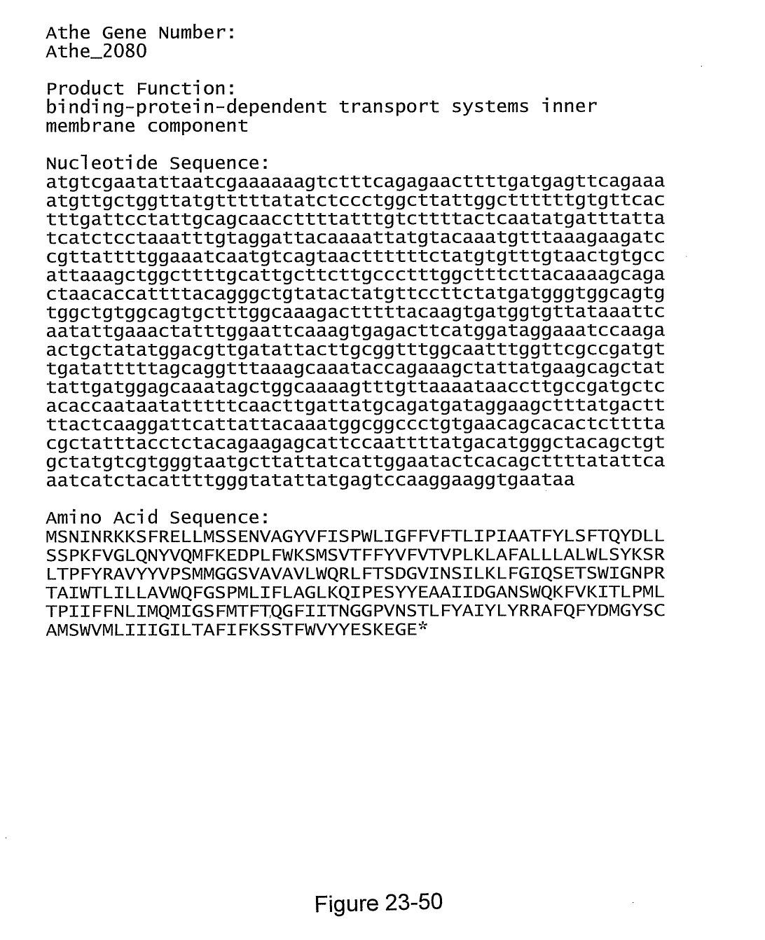

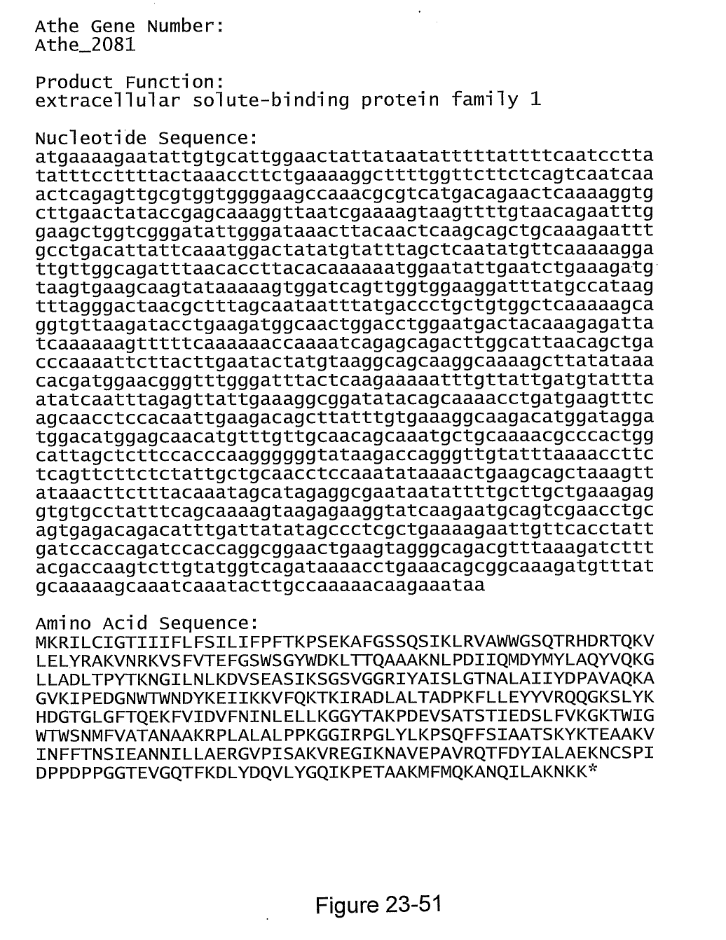

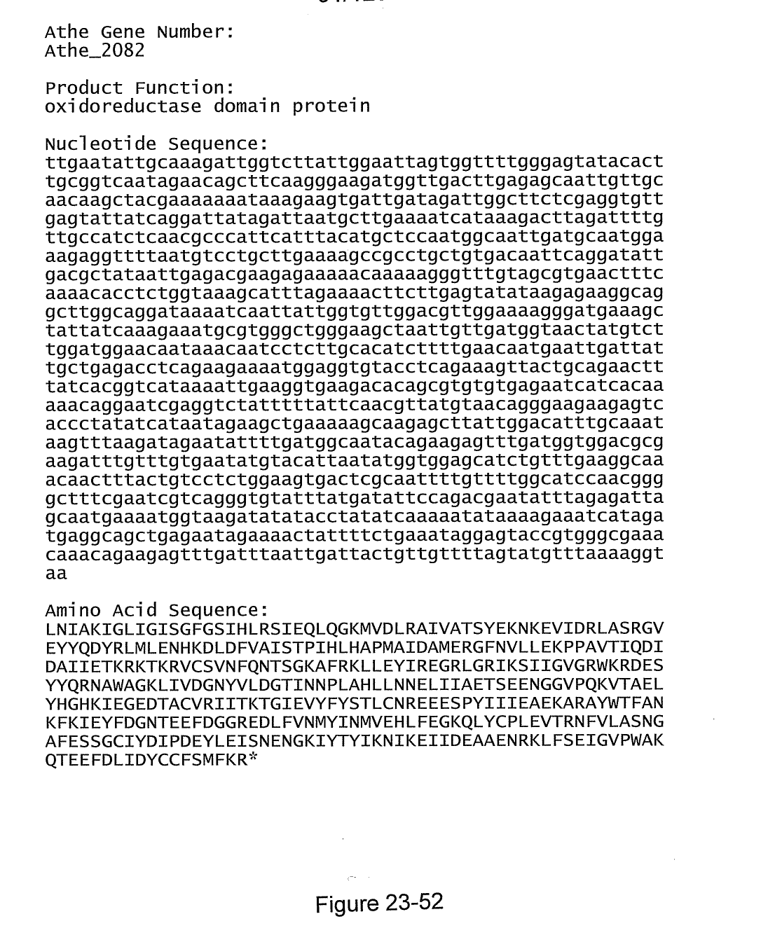

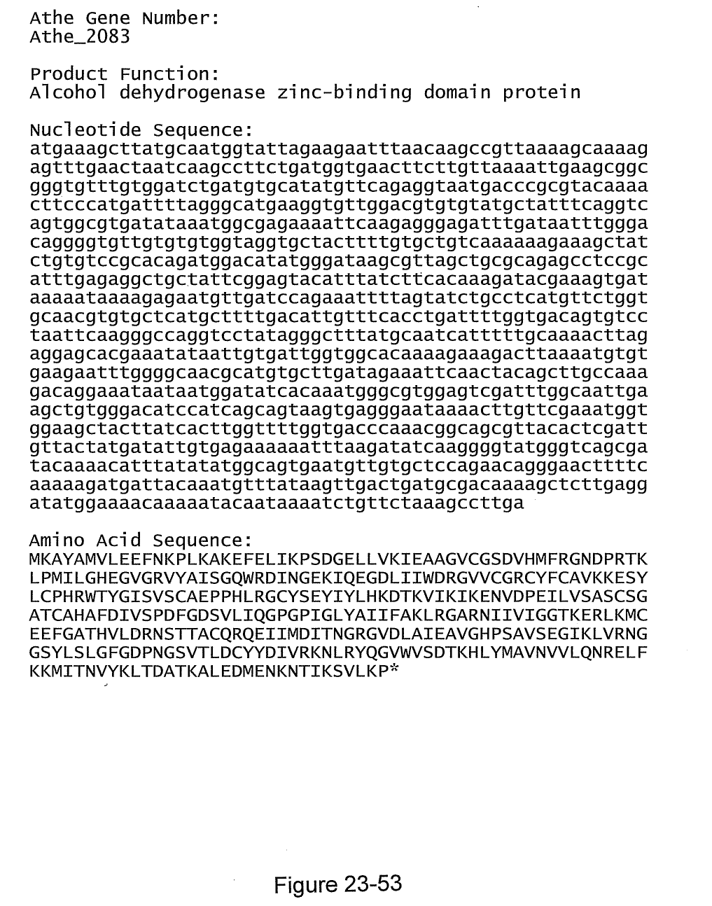

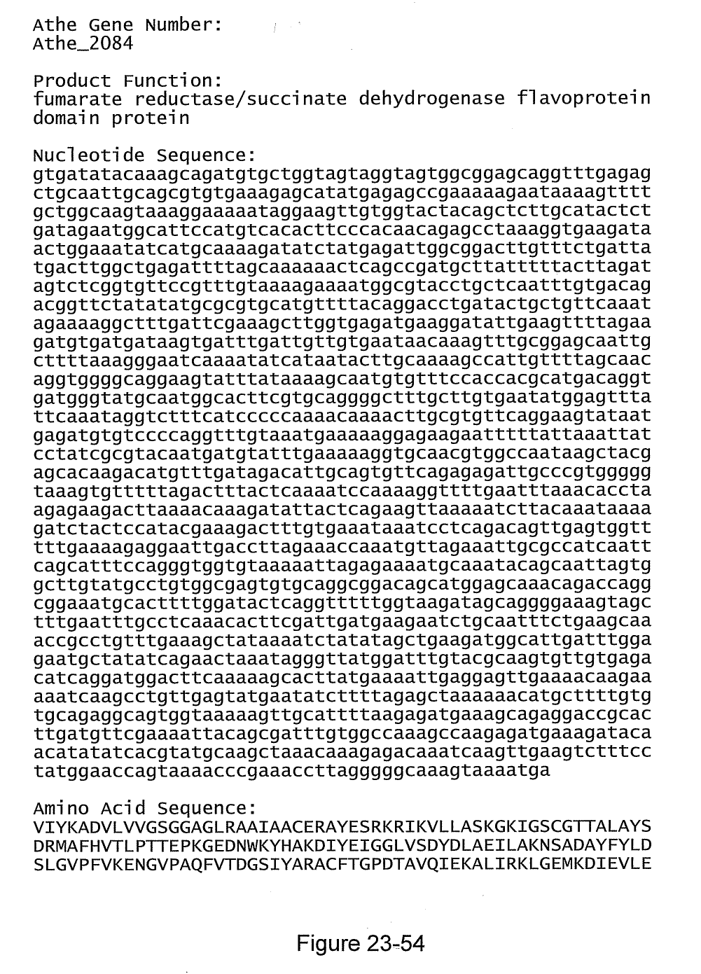

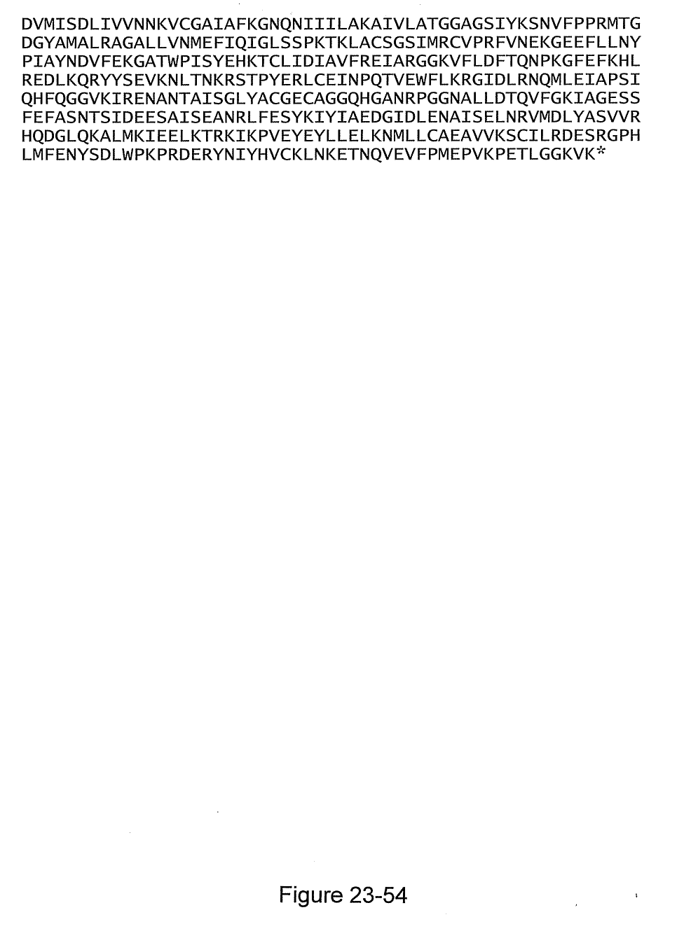

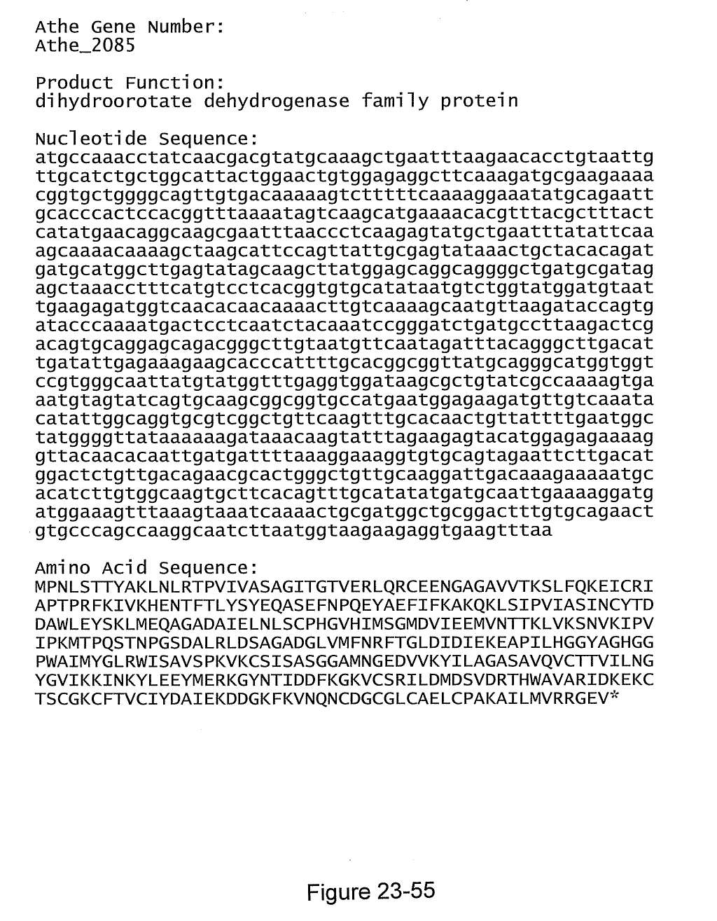

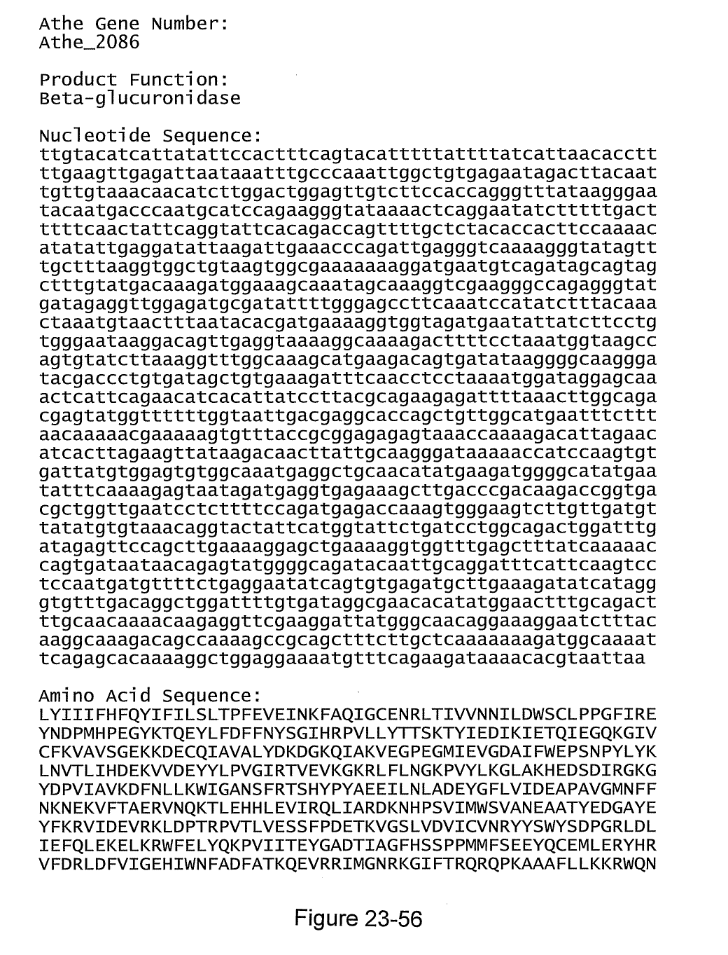

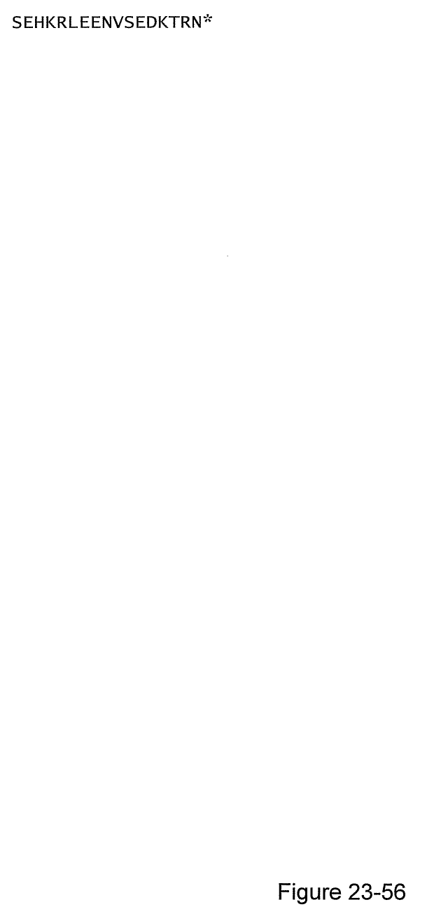

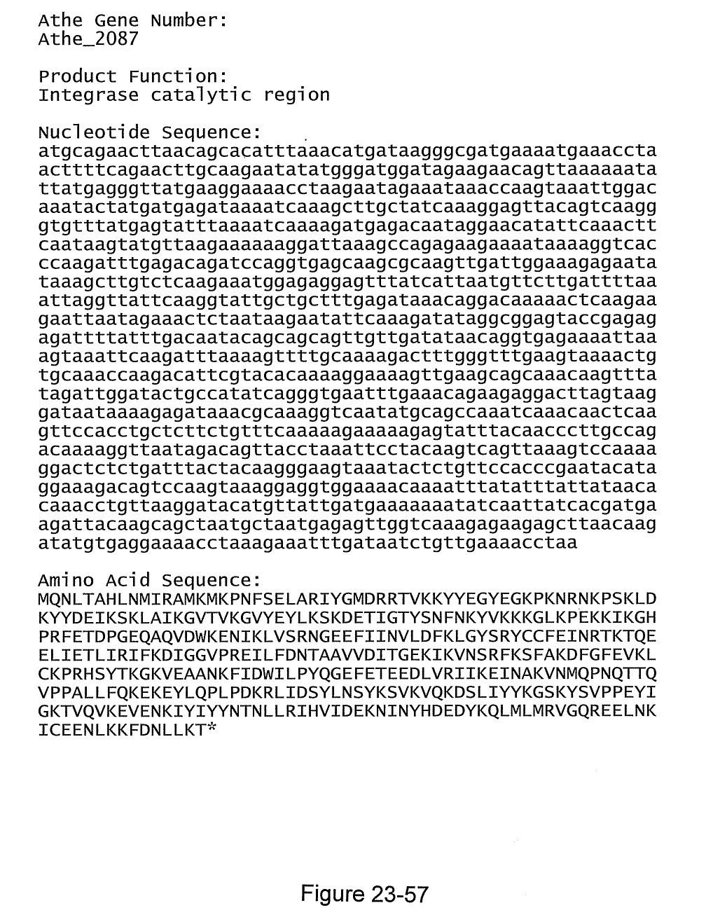

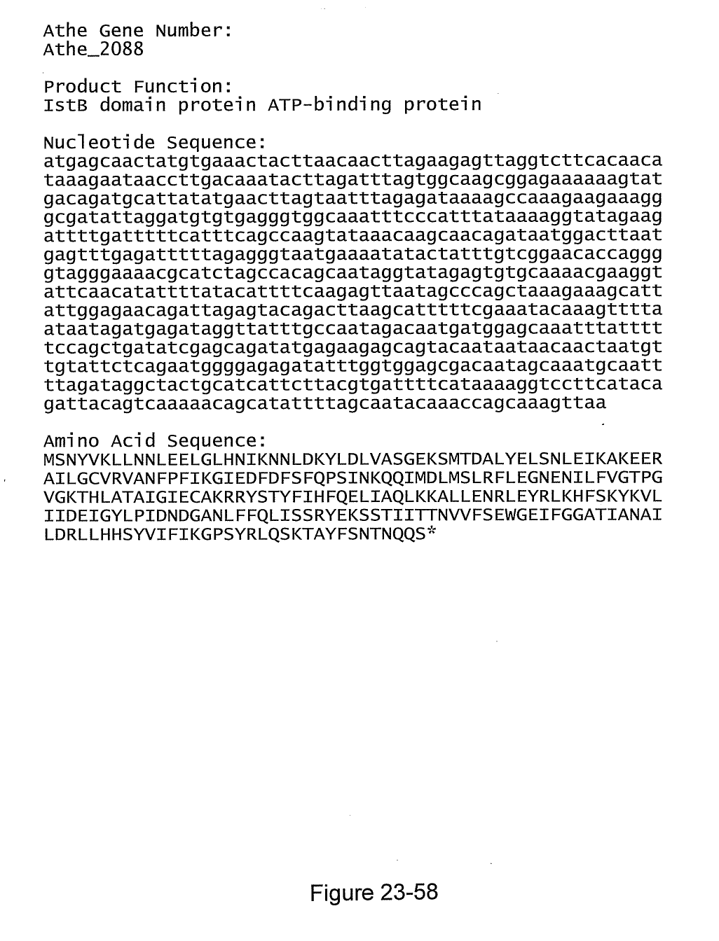

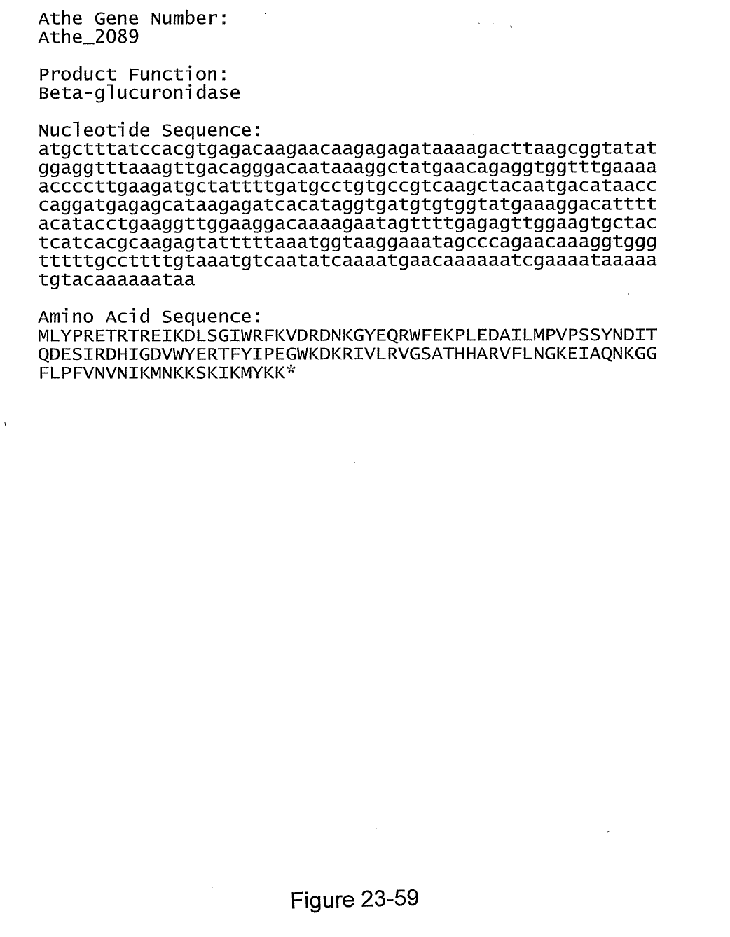

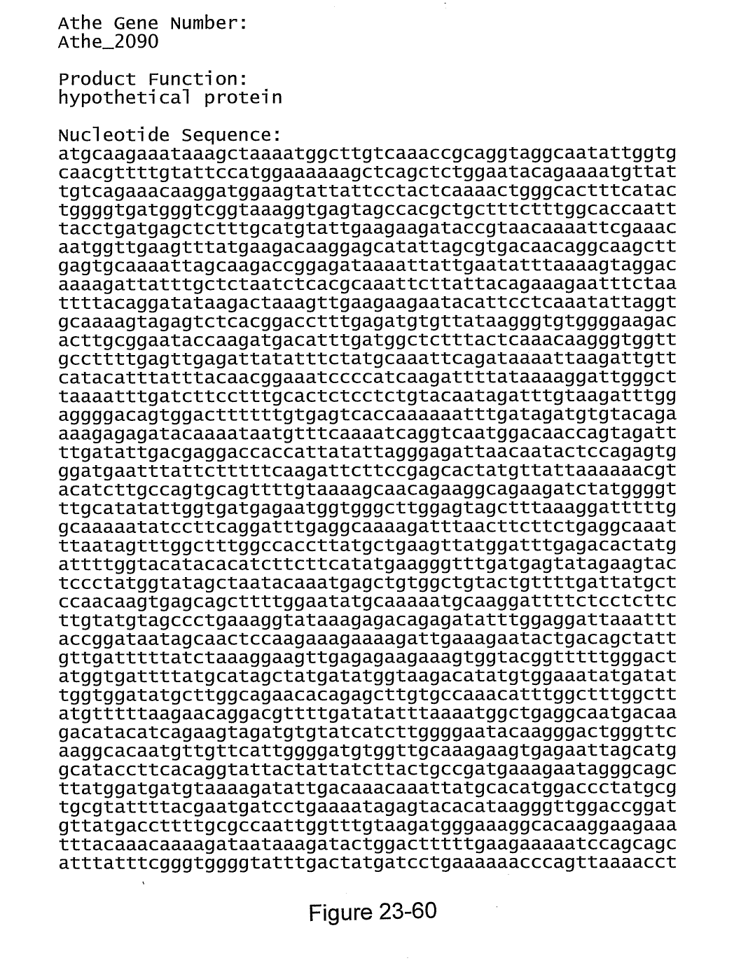

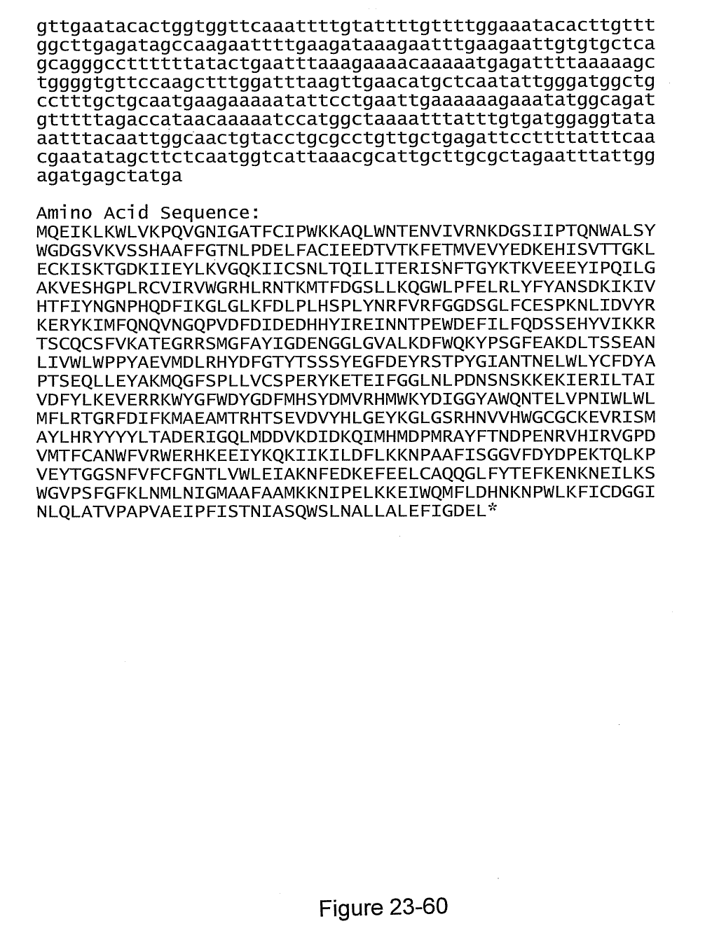

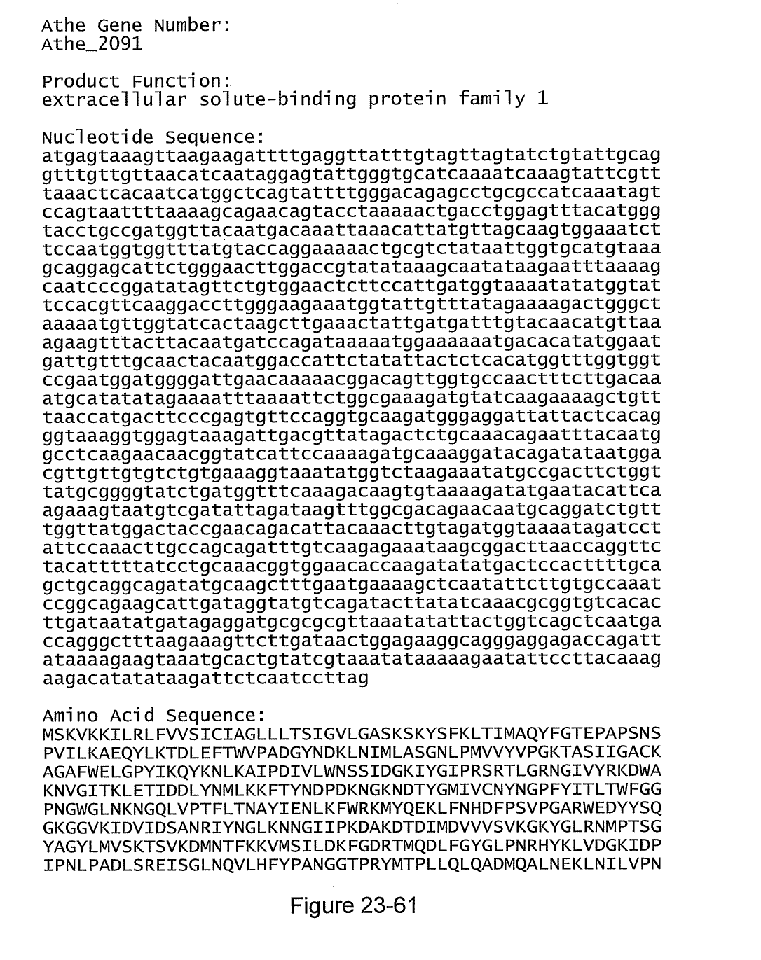

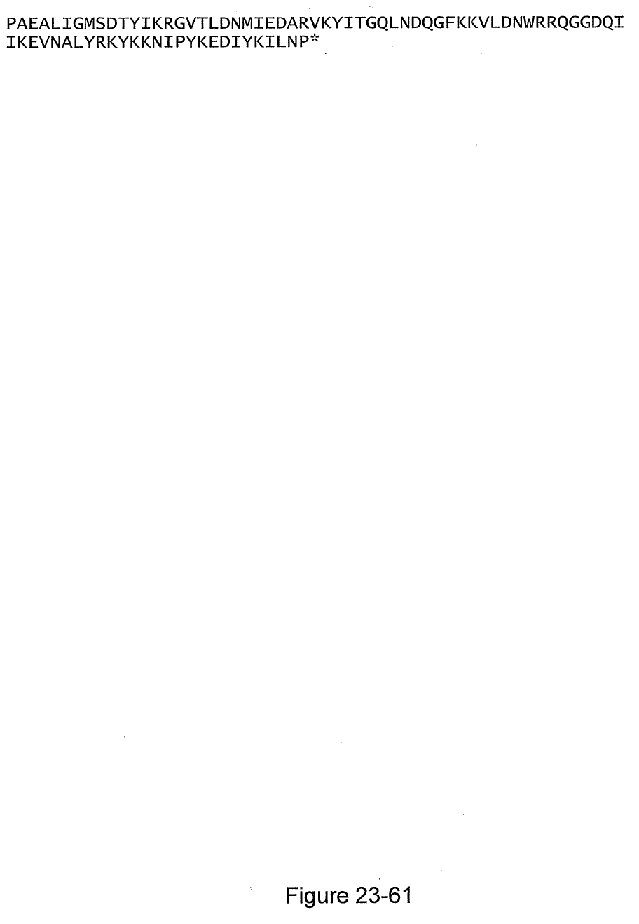

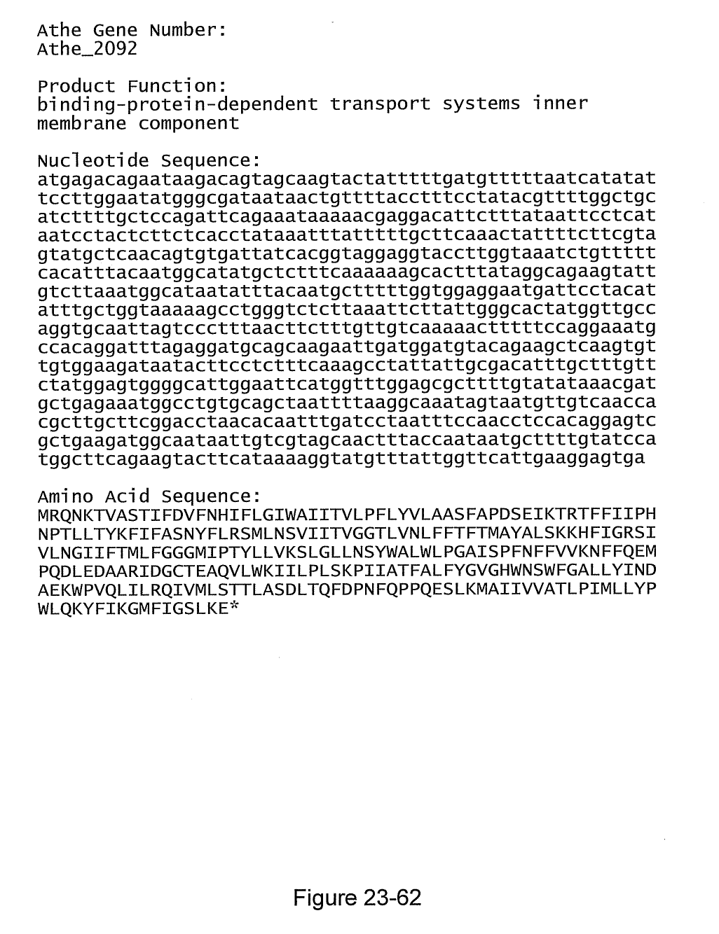

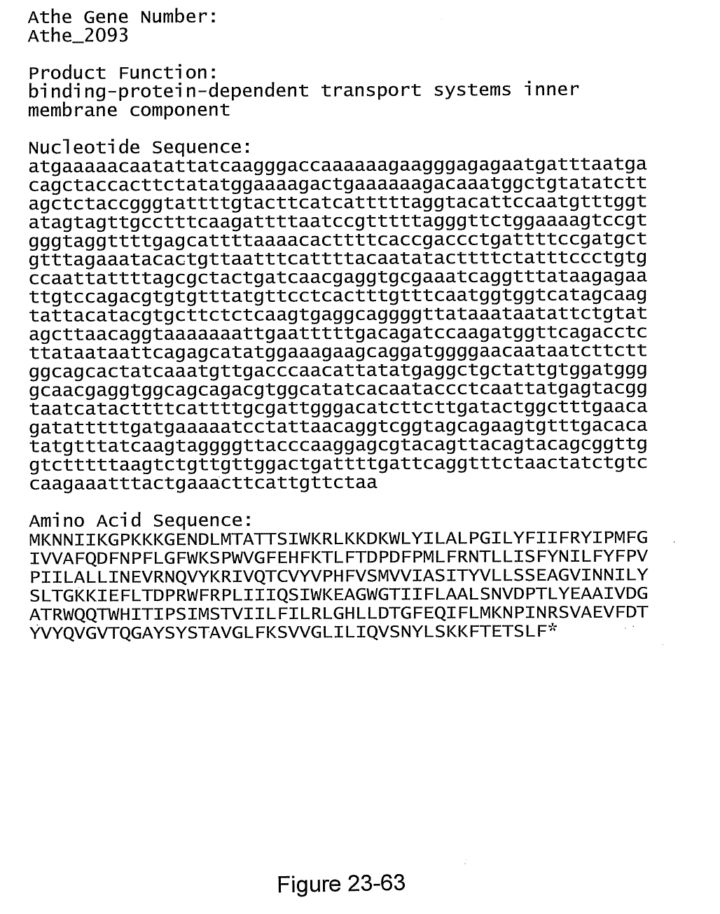

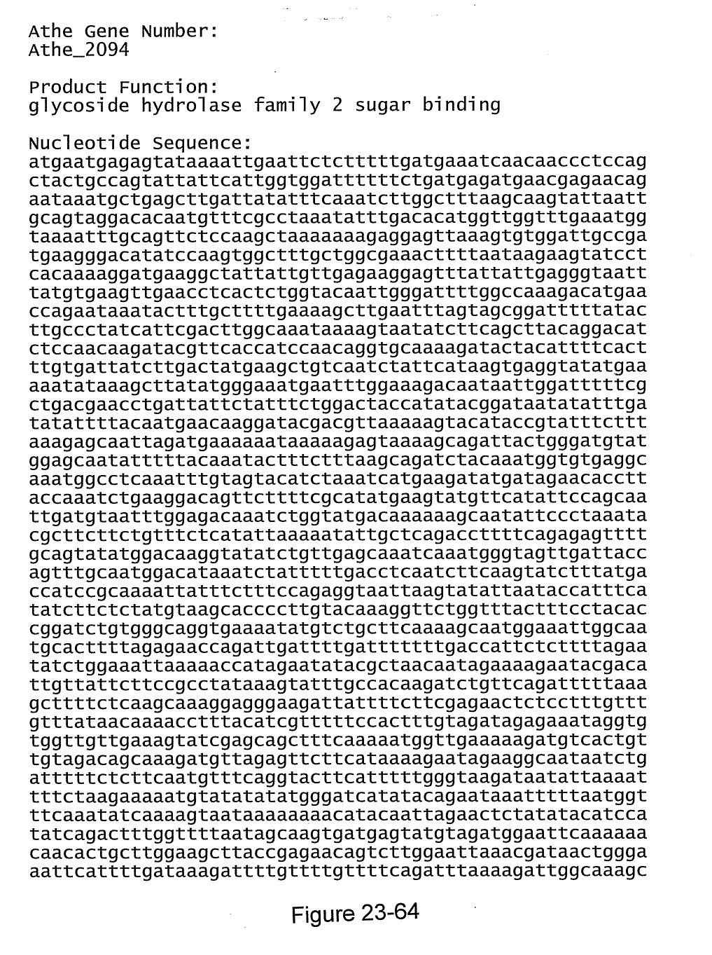

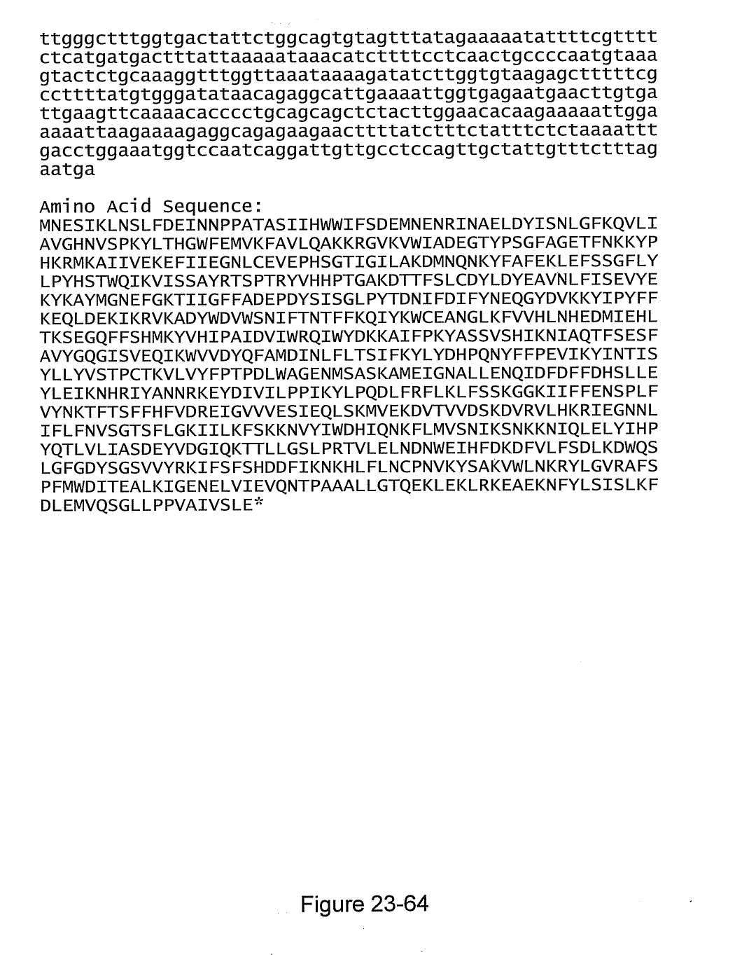

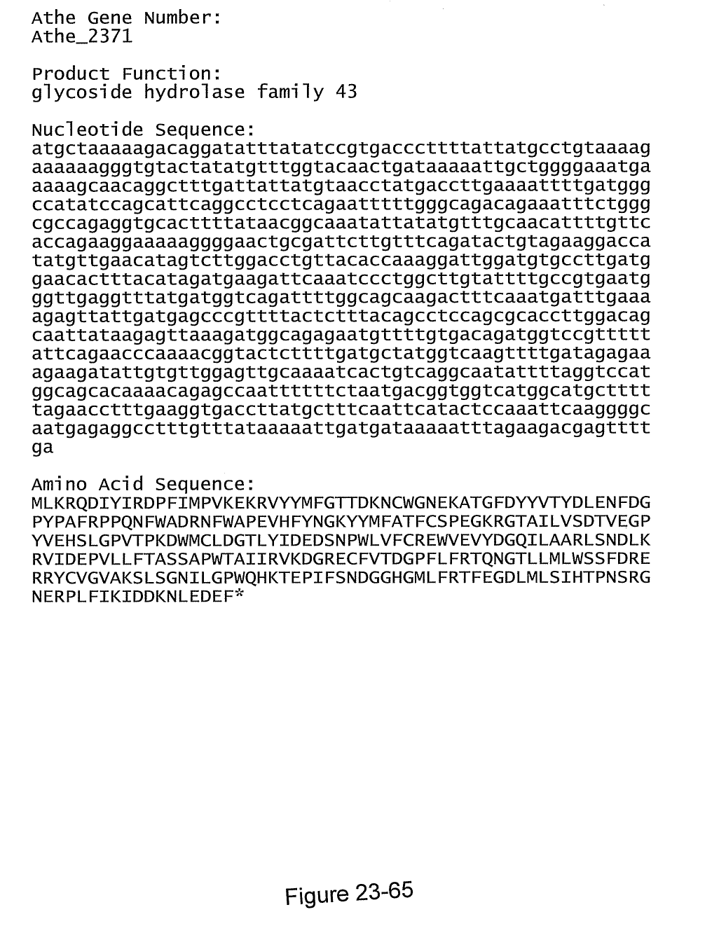

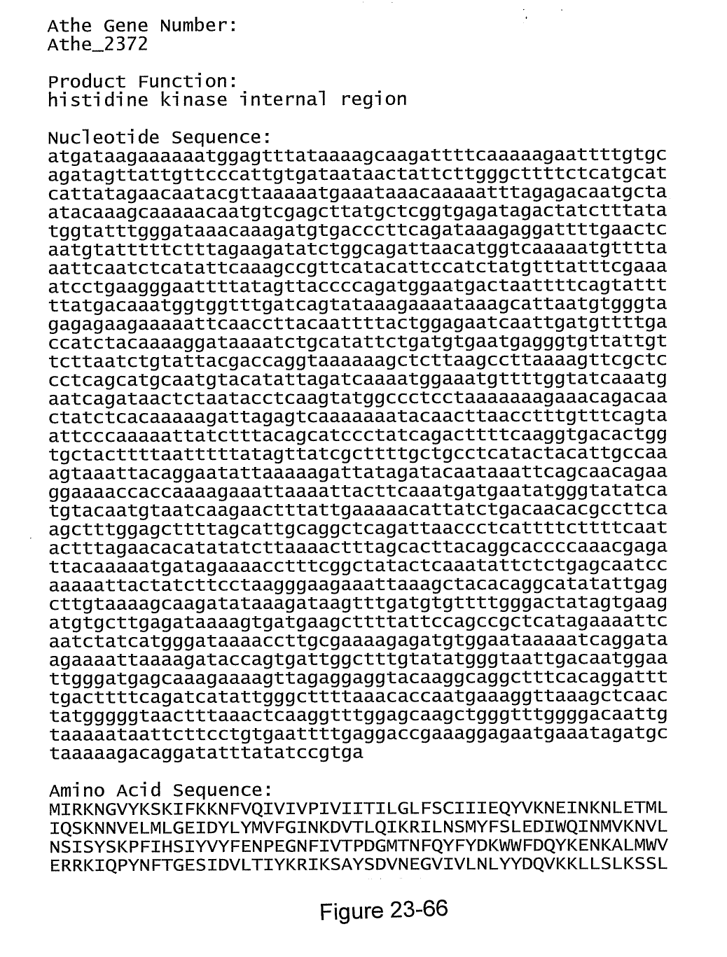

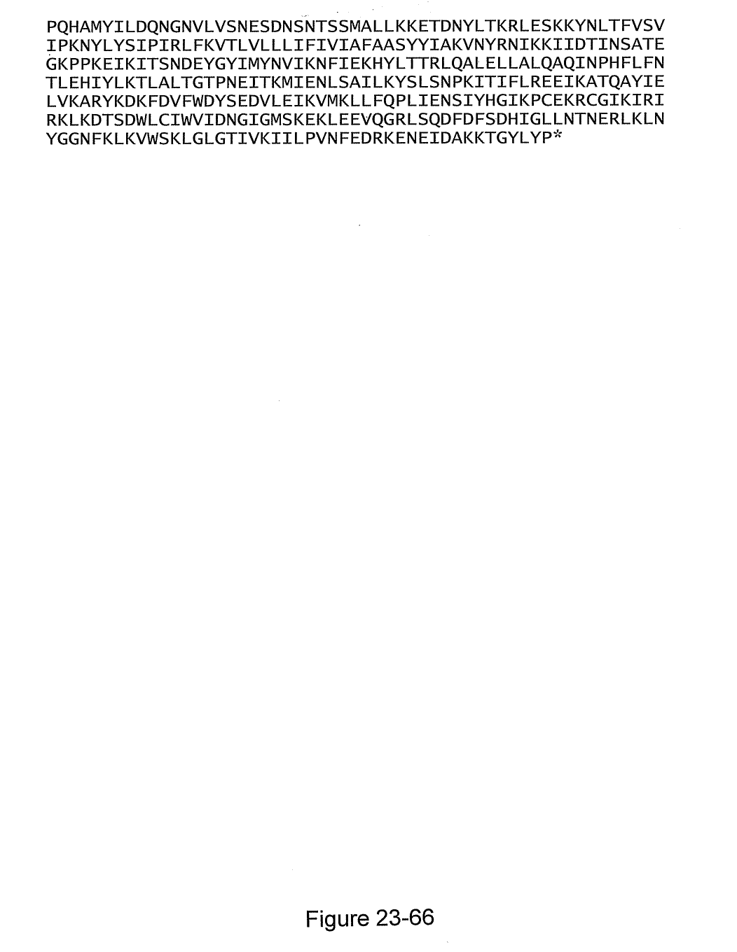

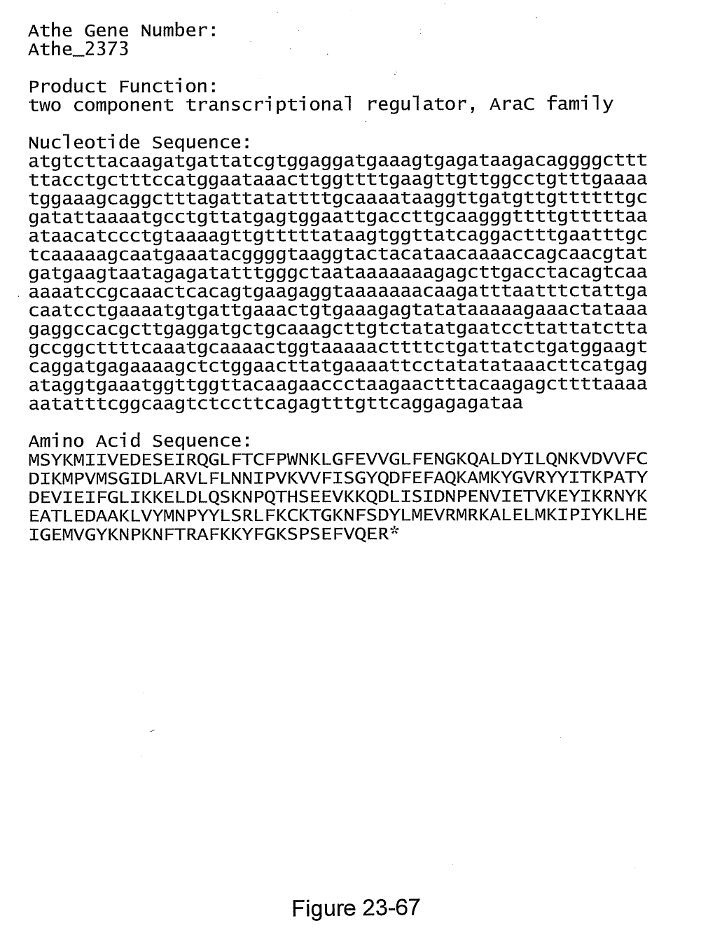

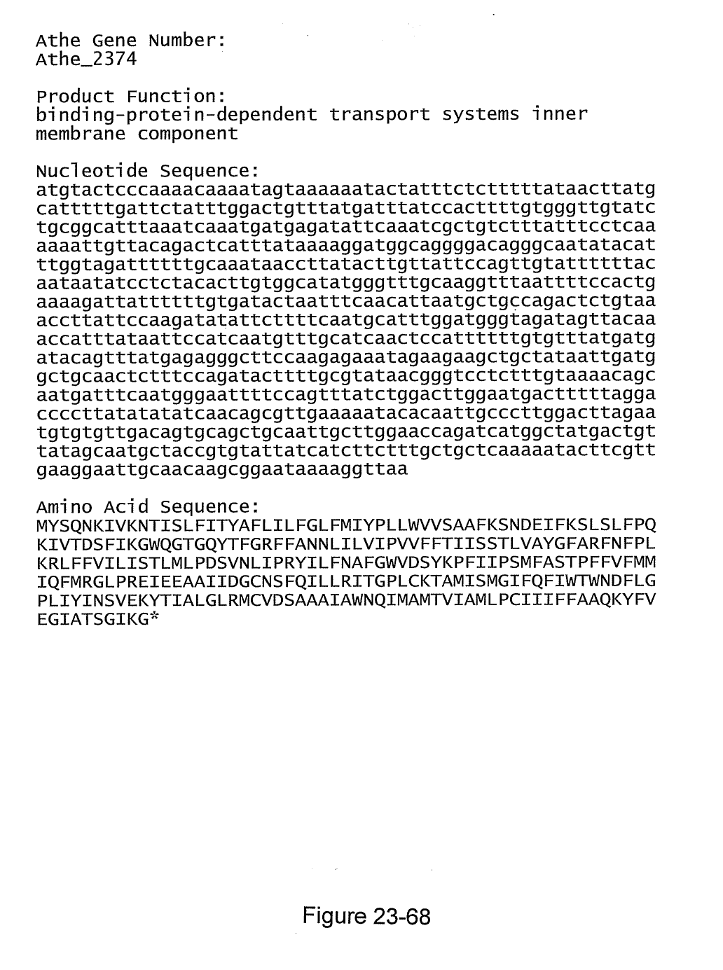

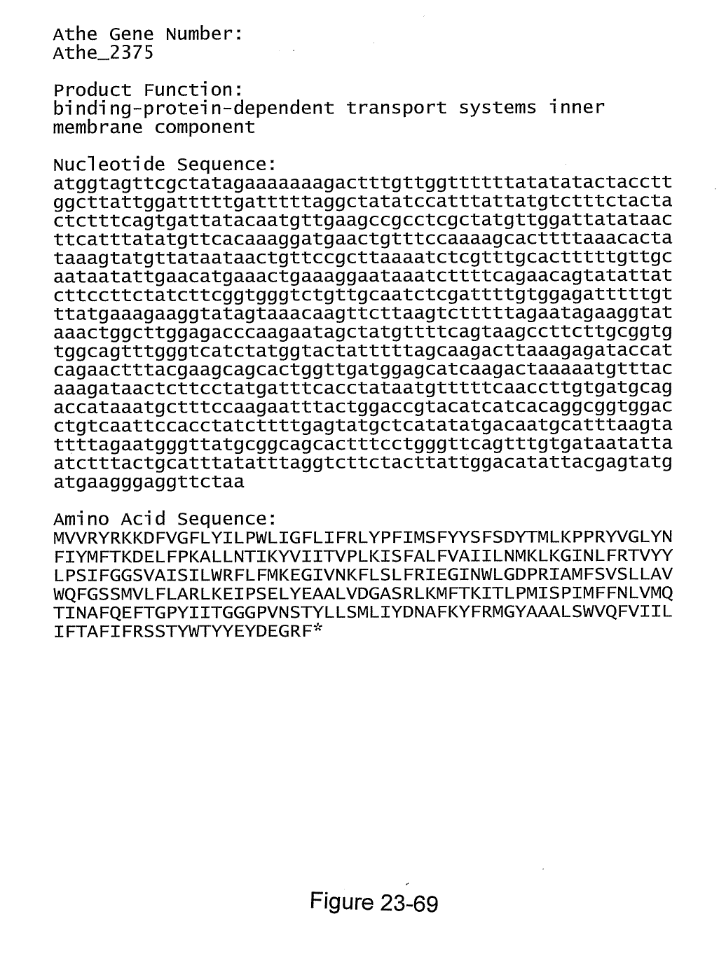

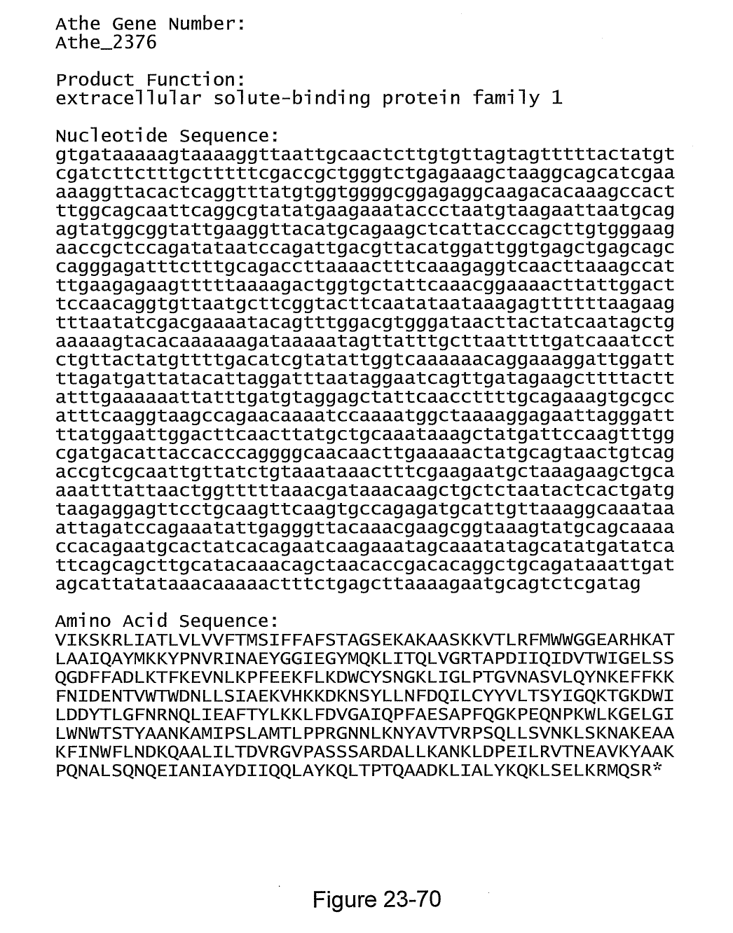

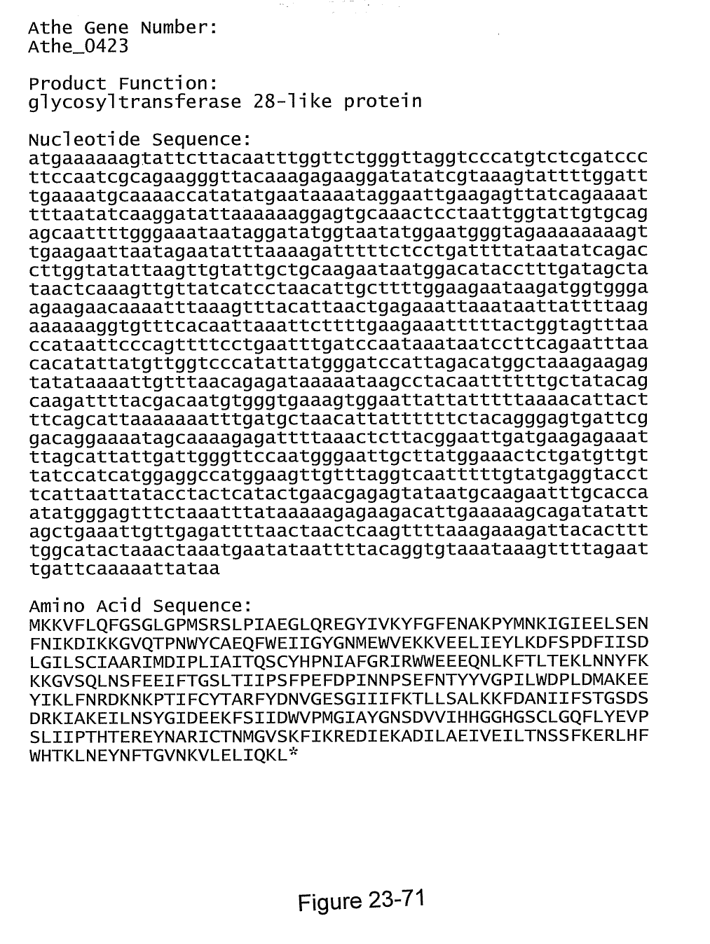

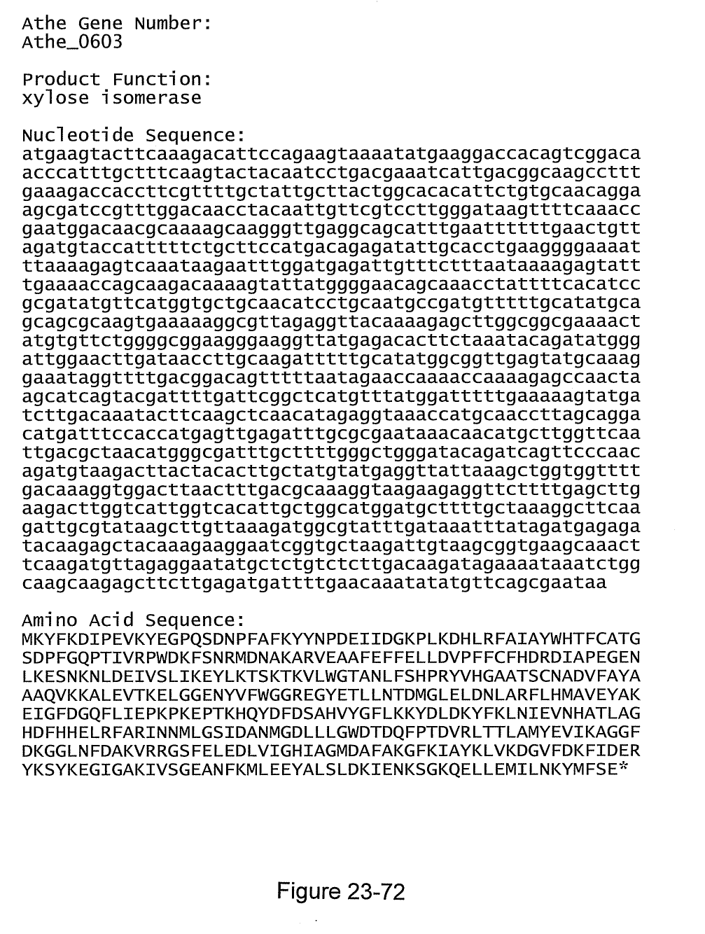

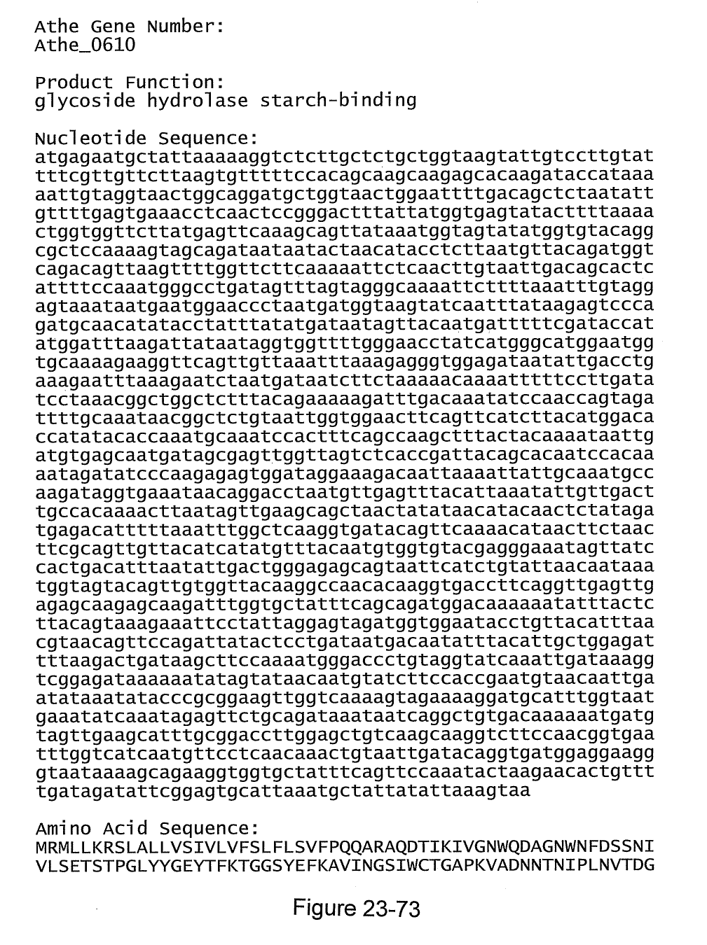

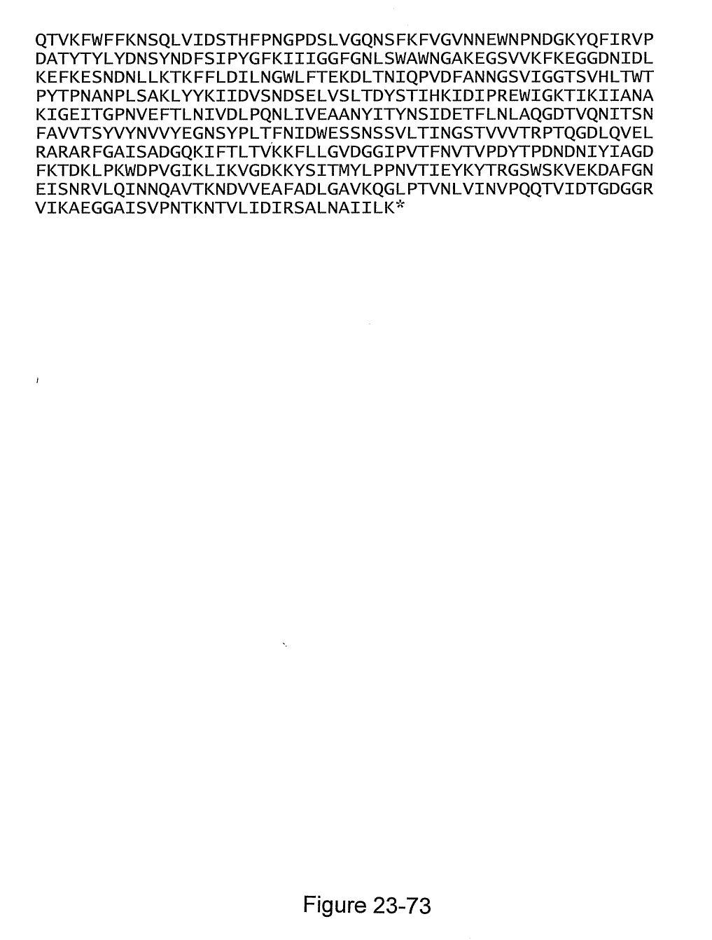

Thus, modification of a nucleotide sequence encoding an A. thermophilum polypeptide may provide the synthesis of a polypeptide that is substantially similar to the A. thermophilum polypeptide. The term “substantially similar” to the A. thermophilum polypeptide refers to a non-naturally occurring form of the A. thermophilum polypeptide. Such a polypeptide may differ in some engineered way from the A. thermophilum polypeptide isolated from a native source—e.g., the variant may differ in specific activity, thermostability, pH optimum, or the like. The variant sequence may be constructed on the basis of the nucleotide sequence presented as the polypeptide encoding region of any one of the nucleotide sequences depicted in FIG. 23, a subsequence thereof, and/or by introduction of nucleotide substitutions which do not give rise to another amino acid sequence of the A. thermophilum polypeptide encoded by the nucleotide sequence, but which correspond to the codon usage of the recipient microorganism, or by introduction of nucleotide substitutions which may give rise to a different amino acid sequence. For a general description of nucleotide substitution, see, e.g., Ford et al., 1991, Protein Expression and Purification 2: 95-107.

In some embodiments, a A. thermophilum polynucleotide can include the nucleotide sequence of one or more PHR coding regions such as, for example, Athe_{—}0423 (or2161) (SEQ ID NO:158), Athe_{—}0603 (or1720) (SEQ ID NO:160), or Athe_{—}0610 (or1727) (SEQ ID NO:162). As used herein, the Athe_#### coding region designations refer to the locus tag associated with the identified coding region, as provided in GenBank Accession No. CP001393, version 1 for the A. thermophilum chromosome, CP001394, version 1 for pATHE01, and CP001395 for pATHE02 (SEQ ID NO:1). The or#### designations refer to the coding region identifiers used in the draft A. thermophilum sequence. Table 1 correlates both designations. Consequently, the A. thermophilum polynucleotide can encode a PHR polypeptide—including, as defined herein, a biologically active analog, subunit, or derivative—such as, for example, a PHR polypeptide that includes the amino acid sequence of one or more of: Athe_{—}0423 (or2161) (SEQ ID NO:159), Athe_{—}0603 (or1720) (SEQ ID NO:161), or Athe_{—}0610 (or1727) (SEQ ID NO:163).

As described in more detail below, many of the coding regions, including PHR coding regions, that confer the ability of A. thermophilum to grow efficiently on plant biomass that cannot be utilized by C. saccharolyticus are present as gene clusters (106 clusters, defined as two or more adjacent coding regions, most of which are likely to be present as operons). Consequently, in certain embodiments, an A. thermophilum polynucleotide can include one or more coding regions from one or more of gene clusters such as, for example, SYb004 (e.g., one or more of Athe_{—}0052-Athe_{—}0061 (or1895-or1905), SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, and SEQ ID NO:52), SYb007 (e.g., one or more of Athe_{—}0088-Athe_{—}0090 (or2788-or2790), SEQ ID NO:56, SEQ ID NO:58, and SEQ ID NO:60), SYb012 (e.g., one or more of Athe_{—}0153-Athe_{—}0160 (or1387-or1394), SEQ ID NO:62, SEQ ID NO:64, SEQ ID NO:66, SEQ ID NO:68, SEQ ID NO:70, SEQ ID NO:72, SEQ ID NO:74, and SEQ ID NO:76), SYb032 (e.g., one or more of Athe_{—}0450-Athe_{—}0452 (or2132-or2130), SEQ ID NO:78, SEQ ID NO:80, and SEQ ID NO:82), SYb059 (e.g., one or more of Athe_{—}1853-Athe_{—}1856 (or2888-or2885, and or2910), SEQ ID NO:88, SEQ ID NO:90, SEQ ID NO:92, and SEQ ID NO:94), SYb063 (e.g., one or more of Athe1989-Athe_{—}1994 (or1187-or1182), SEQ ID NO:96, SEQ ID NO:98, SEQ ID NO:100, SEQ ID NO:102, SEQ ID NO:104, and SEQ ID NO:106), SYb067 (e.g., one or more of Athe_{—}2076-Athe_{—}2094 (or1093-or1071), SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, and SEQ ID NO:144), and SYb082 (e.g., one or more of Athe_{—}2371-Athe_{—}2376 (or1921-or1926), SEQ ID NO:146, SEQ ID NO:148, SEQ ID NO:150, SEQ ID NO:152, SEQ ID NO:154, and SEQ ID NO:156). Thus, the A. thermophilum polynucleotide can encode a PHR polypeptide-including, as defined herein, a biologically active analog, subunit, or derivative-such as, for example, a PHR polypeptide that includes the amino acid sequence of one or more of: SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, and SEQ ID NO:145, SEQ ID NO:147, SEQ ID NO:149, SEQ ID NO:151, SEQ ID NO:153, SEQ ID NO:155, and SEQ ID NO:157.

In some embodiments, an A. thermophilum polynucleotide can include the nucleotide sequence of one or more of the remaining PBU coding regions such as, for example, Athe_{—}0077 (or2776), SEQ ID NO:54). Consequently, the A. thermophilum polynucleotide can encode a PBU polypeptide-including, as defined herein, a biologically active analog, subunit, or derivative-such as, for example, a PBU polypeptide that includes the amino acid sequence of SEQ ID NO:55.

Here again, many of the remaining PBU coding regions are present as gene clusters. Consequently, in certain embodiments, an A. thermophilum polynucleotide can include one or more coding regions from one or more of gene clusters such as, for example, SYb001 (e.g., one or more of Athe_{—}0010-Athe_{—}0017 (or1851-or1859), SEQ ID NO:18, SEQ ID NO:20, SEQ ID NO:22, SEQ ID NO:24, SEQ ID NO:26, SEQ ID NO:28, SEQ ID NO:30, and SEQ ID NO:32) and SYb037 (e.g., one or more of Athe_{—}0607-Athe_{—}0608 (ori1724-or1724), SEQ ID NO:84 and SEQ ID NO:86). Thus, an A. thermophilum polynucleotide can encode a PBU polypeptide—including, as defined herein, a biologically active analog, subunit, or derivative—such as, for example, a PBU polypeptide that includes the amino acid sequence of one or more of SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:85, and SEQ ID NO:87.

Some methods described herein exploit the PBU coding regions of A. thermophilum to convert plant biomass into water soluble or water insoluble product. A water soluble product may have value in itself, or as a starting material from which some other material may be prepared in one or more subsequent processes. For example, in some embodiments, the water soluble product can include an alcohol such as, for example, ethanol, n-butanol, 1,4-butanediol, sec-butanol, and/or methanol. In other embodiments, the water soluble product can include, for example, hydrogen gas (H₂). In still other embodiments, the water soluble product can include one or more small organic (e.g., C1-C8) acids such as, for example, succinic acid, lactic acid, citric acid, oxaloacetic acid, malic acid, adipic acid, fumaric acid, pyruvic acid, or a salt thereof). In still other embodiments, the water soluble product can include simple saccharides such as, for example, monosaccharides and/or disaccharides. Small organic acids and/or simple saccharides can serve as metabolic intermediates for the production of other organic compounds such as, for example, alcohols, fatty acids, and polymers. Ethanol, methanol, a butanol, and/or hydrogen gas may be used as biofuels. Ethanol, methanol, a butanol, or an organic acid or a salt thereof may be used as a commodity chemical. In still other embodiments, the water soluble product can include a water soluble polymer material such as, for example, a soluble lipid such as, for example, a fatty acid or a polyisoprenoid. In other embodiments, the product may be water insoluble, such as, for example, the production of a biodiesel (alkyl fatty acid esters), which may be used as a biofuel.

In some embodiments, the product, whether water soluble or water insoluble, may be released by the A. thermophilum into the culture medium, from which the product may be isolated, purified, or otherwise recovered using a method or process appropriate for the product. In this context, “isolated” refers to increasing the proportion (e.g., concentration, w/v%, etc.) of the product to any degree regardless of the way in which the product is isolated. Thus, in some cases, a product may be isolated by, for example, removing at least a portion of the product from the culture medium. In other cases, a product may be isolated by, for example, removing one or more components (e.g., cells, spent biomass, medium components, etc.) of the culture medium, leaving behind an increased proportion of the product compared to the sum of non-product constituents of the culture medium. In other embodiments, the product, whether water soluble or water insoluble, may be sequestered within the A. thermophilum. In such cases, the methods described herein can further include solubilizing the A. thermophilum before the product may be recovered. As used herein, the term “solubilizing” refers to dissolving cellular materials (e.g., polypeptides, nucleic acids, carbohydrates) into the aqueous phase of a buffer in which the microbe was disrupted, and the formation of aggregates of insoluble cellular materials. Methods for solubilizing cells are routine and known to those skilled in the art.

The chromosomal genome of A. thermophilum is 2.97 Mb in size and is predicted to contain 2,824 genes, of which 2,654 are predicted to be protein coding regions. The A. thermophilum genome further includes two native plasmids: pATHE01 (approximately 8.3 Kb in size and containing eight coding regions) and pATHE02 (approximately 3.7 Kb in size and containing four coding regions, SEQ ID NO:1). A preliminary bioinfoiniatics analysis of the A. thermophilum DSM 6725 coding regions revealed that the closest homologs for 2,284 coding regions in the A. thermophilum genome are found in the genome of Caldicellulosiruptor saccharolyticus (DSM 8903). C. saccharolyticus was discovered in 1994 and, like A. thermophilum, is a strict anaerobe that grows optimally near 75° C. Its genome sequence was reported in 2007 and contains 2,679 coding regions (2.97 Mb). C. saccharolyticus and A. thermophilum appear to be close relatives and may be members of the same bacterial genus. Indeed, it has been proposed that A. thermophilum DSM 6725 be reclassified as Caldicellulosiruptor bescii. Thus, as used herein, the term A. thermophulim DSM 6725 refers to the bacterial strain deposited Aug. 12, 2009 with the American Type Culture Collection (ATCC), Manassas, Va., regardless of whether the microorganism is classified as A. thermophilum or C. bescii. The deposit will be maintained under the terms of the Budapest Treaty on the International Recognition of the Deposit of Microorganisms for the Purposes of Patent Procedure. The deposit was made merely as a convenience for those of skill in the art and is not an admission that a deposit is required under 35 U.S.C. §112.

Despite the apparent relatedness of A. thermophulim DSM 6725 and C. saccharolyticus, only one of the species, A. thermophilum, is able to grow efficiently on certain forms of plant biomass. The coding regions that confer this property to A. thermophilum DSM 6725 are termed PBU for plant biomass utilization. Certain A. thermophilum DSM 6725 coding regions that are not specific to A. thermophilum may, in conjunction with one or more PBU coding regions, also be involved in plant biomass utilization. Many of the PBU coding regions are present in A. thermophilum DSM 6725 as gene clusters.

Biomass utilization in C. saccharolyticus has been partially characterized and C. saccharolyticus may grow on a variety of polysaccharides, including crystalline cellulose and xylan. However, growth on untreated biomass has not been reported. C. saccharolyticus can grow on soluble and insoluble heat-treated switchgrass (i.e., after heat treatment; FIG. 13). However, in contrast to A. thermophilum, C. saccharolyticus cannot utilize either the soluble or insoluble material derived from poplar (FIG. 14), and it grows much less efficiently than A. thermophilum on insoluble material derived from heat-treated pine (FIG. 15). A. thermophilum has also been shown to grow efficiently on both washed and unwashed peanut shells (FIG. 24).

The ability of A. thermophilum to grow efficiently on untreated and treated biomass that cannot be utilized by C. saccharolyticus is a consequence, at least in part, of coding regions present in A. thermophilum that lack homologs in C. saccharolyticus.

Table 1 lists a total of 550 such coding regions. Many of these coding regions are present as gene clusters (106 clusters, defined as adjacent coding regions, most of which are likely to be present as operons). The 106 gene clusters are labeled SYa001-SYa106 and contain 436 coding regions. The remaining 114 coding regions that lack close homologs in C. saccharolyticus that are not part of gene clusters SYa001-SYa106 are labeled FPa001-FPa114. More than 30 of the clusters contain five or more coding regions, with one cluster containing 19 coding regions (SYa067; Table 2). The 550 coding regions also include nine coding regions encoding transposases. These are similar to those found in both Gram negative bacteria and other Gram positive bacteria, suggesting that at least some of the gene clusters were acquired by A. thermophilum through lateral gene transfer. Of the 550 coding regions found in A. thermophilum DSM 6725 that are not found in C. saccharolyticus, 332 of them are annotated as conserved/hypothetical/unknown function proteins, leaving 218 coding regions with a proposed function. These include 21 DNA binding proteins (11 putative transcriptional regulators/10 containing helix-turn-helix motifs) indicating that many of these coding regions may respond to and regulate carbon source utilization for growth on substrates such as plant biomass.

TABLE 1
PBU Coding Regions

| | GenBank | |
|---|---|---|
| Cluster/Single | CP001393.1 | Draft sequence |
| Number | locus tag | locus tag |
| FPb001 | Athe_0002 | or1843 |
| FPb002 | Athe_0007 | or1848 |
| SYb001 | Athe_0010 | or1851 |
| SYb001 | Athe_0011 | or1852 |
| SYb001 | Athe_0012 | or1853, or1854 |
| SYb001 | Athe_0013 | or1855 |
| SYb001 | Athe_0014 | or1856 |
| SYb001 | Athe_0015 | or1857 |
| SYb001 | Athe_0016 | or1858 |
| SYb001 | Athe_0017 | or1859 |
| FPb003 | Athe_0020 | or1862 |
| SYb002 | Athe_0022 | or1865 |
| SYb002 | Athe_0023 | or1866 |
| SYb002 | Athe_0024 | or1867 |
| SYb002 | Athe_0025 | or1868 |
| SYb003 | Athe_0028 | or1870 |
| SYb003 | Athe_0029 | or1871 |
| FPb004 | Athe_0035 | or1877 |
| SYb004 | Athe_0052 | or1895 |
| SYb004 | Athe_0053 | or1896 |
| SYb004 | Athe_0054 | or1897 |
| SYb004 | Athe_0055 | or1898 |
| SYb004 | Athe_0056 | or1899 |
| SYb004 | Athe_0057 | or1900 |
| SYb004 | Athe_0058 | or1901 |
| SYb004 | Athe_0059 | or1902, or1903 |
| SYb004 | Athe_0060 | or1904, or1903 |
| SYb004 | Athe_0061 | or1905 |
| SYb005 | Athe_0066 | or1910 |
| SYb005 | Athe_0067 | or1911, or1912 |
| SYb005 | Athe_0068 | |
| SYb005 | Athe_0069 | or1914 |
| SYb005 | Athe_0070 | |
| SYb006 | Athe_0072 | or2770 |
| SYb006 | Athe_0073 | or2771 |
| SYb006 | Athe_0074 | or2772 |
| FPb005 | Athe_0077 | or2776 |
| SYb007 | Athe_0088 | or2788 |
| SYb007 | Athe_0089 | or2789 |
| SYb007 | Athe_0090 | or2790 |
| FPb006 | Athe_0092 | |
| SYb008 | Athe_0109 | or2529 |
| SYb008 | Athe_0110 | or2530 |
| SYb008 | Athe_0111 | or2531 |
| SYb009 | Athe_0130 | or2555 |
| SYb009 | Athe_0131 | or1363 |
| SYb010 | Athe_0135 | or1368 |
| SYb010 | Athe_0136 | or1369 |
| SYb011 | Athe_0139 | or1372 |
| SYb011 | Athe_0140 | |
| FPb007 | Athe_0142 | or1376, or1374, or1375 |
| SYb012 | Athe_0153 | or1387 |
| SYb012 | Athe_0154 | or1388 |
| SYb012 | Athe_0155 | or1389 |
| SYb012 | Athe_0156 | or1390 |
| SYb012 | Athe_0157 | or1391 |
| SYb012 | Athe_0158 | or1392 |
| SYb012 | Athe_0159 | or1393 |
| SYb012 | Athe_0160 | or1394 |
| FPb008 | Athe_0188 | or1208, or1423 |
| FPb009 | Athe_0201 | or1436 |
| SYb013 | Athe_0204 | or1440 |
| SYb013 | Athe_0205 | or1441 |
| FPb010 | Athe_0224 | or1460 |
| FPb011 | Athe_0229 | or1465 |
| SYb014 | Athe_0235 | or1471 |
| SYb014 | Athe_0236 | or1472 |
| SYb014 | Athe_0237 | or1473 |
| FPb012 | Athe_0241 | |
| SYb015 | Athe_0247 | or1482 |
| SYb015 | Athe_0248 | or1483, or1484 |
| SYb016 | Athe_0252 | or2645, or2646 |
| SYb016 | Athe_0253 | or2647 |
| SYb016 | Athe_0254 | or2648 |
| SYb017 | Athe_0258 | or2652 |
| SYb017 | Athe_0259 | |
| SYb018 | Athe_0261 | or2655 |
| SYb018 | Athe_0262 | or2656 |
| SYb019 | Athe_0266 | or2661 |
| SYb019 | Athe_0267 | or2662 |
| SYb019 | Athe_0268 | or2663 |
| SYb019 | Athe_0269 | or2664 |
| SYb020 | Athe_0271 | or2665 |
| SYb020 | Athe_0272 | or2666 |
| SYb020 | Athe_0273 | or2667 |
| SYb021 | Athe_0279 | or2673 |
| SYb021 | Athe_0280 | or2674 |
| SYb021 | Athe_0281 | or2675 |
| SYb022 | Athe_0285 | or2680 |
| SYb022 | Athe_0286 | or2681 |
| SYb022 | Athe_0287 | or2682 |
| SYb023 | Athe_0310 | or2367 |
| SYb023 | Athe_0311 | or2368 |
| FPb013 | Athe_0328 | or2385 |
| SYb024 | Athe_0330 | or2387 |
| SYb024 | Athe_0331 | |
| SYb025 | Athe_0336 | or2394 |
| SYb025 | Athe_0337 | or2395 |
| SYb025 | Athe_0338 | or2396 |
| SYb026 | Athe_0347 | |
| SYb026 | Athe_0348 | or2920 |
| SYb026 | Athe_0349 | or2919 |
| SYb026 | Athe_0350 | or2918 |
| SYb026 | Athe_0351 | or2917 |
| SYb026 | Athe_0352 | |
| SYb026 | Athe_0353 | or2916 |
| SYb026 | Athe_0354 | or2915 |
| SYb026 | Athe_0355 | or2914 |
| SYb026 | Athe_0356 | |
| SYb026 | Athe_0357 | or0501 |
| FPb014 | Athe_0366 | or0510 |
| SYb027 | Athe_0375 | or0520 |
| SYb027 | Athe_0376 | or0521 |
| SYb027 | Athe_0377 | or0522 |
| SYb027 | Athe_0378 | or0523 |
| SYb027 | Athe_0379 | or0524 |
| SYb028 | Athe_0384 | or0529 |
| SYb028 | Athe_0385 | or0530 |
| SYb029 | Athe_0406 | or2843 |
| SYb029 | Athe_0407 | or2842 |
| SYb029 | Athe_0408 | or2841 |
| SYb029 | Athe_0409 | or2840 |
| SYb029 | Athe_0410 | or2839 |
| SYb029 | Athe_0411 | or2838 |
| SYb029 | Athe_0412 | or2837, or2836 |
| SYb029 | Athe_0413 | or2835, or2836 |
| SYb030 | Athe_0416 | or2168 |
| SYb030 | Athe_0417 | or2167 |
| SYb031 | Athe_0419 | or2165 |
| SYb031 | Athe_0420 | or2164 |
| SYb031 | Athe_0421 | or2163 |
| FPb015 | Athe_0423 | or2161 |
| SYb032 | Athe_0450 | or2132 |
| SYb032 | Athe_0451 | or2131 |
| SYb032 | Athe_0452 | or2130 |
| FPb016 | Athe_0456 | or2126 |
| FPb017 | Athe_0464 | or2118 |
| SYb033 | Athe_0481 | or2097, or2098, |
| | | or2099, or2599 |
| SYb033 | Athe_0482 | or2600 |
| SYb033 | Athe_0483 | or2601 |
| SYb034 | Athe_0485 | or2604 |
| SYb034 | Athe_0486 | or2605 |
| SYb034 | Athe_0487 | or2606 |
| SYb034 | Athe_0488 | or2607, or2608 |
| FPb018 | Athe_0490 | or2611 |
| SYb035 | Athe_0492 | or2614 |
| SYb035 | Athe_0493 | or2615 |
| SYb036 | Athe_0496 | or2618 |
| SYb036 | Athe_0497 | or2619 |
| SYb036 | Athe_0498 | or2620 |
| FPb019 | Athe_0506 | or2629 |
| FPb020 | Athe_0549 | or1663 |
| FPb021 | Athe_0590 | |
| FPb022 | Athe_0603 | or1720 |
| SYb037 | Athe_0607 | or1724 |
| SYb037 | Athe_0608 | or1725 |
| FPb023 | Athe_0610 | or1727 |
| SYb038 | Athe_0644 | or2728, or2729 |
| SYb038 | Athe_0645 | or1835, or2729 |
| SYb039 | Athe_0673 | or1805 |
| SYb039 | Athe_0674 | or1804 |
| SYb039 | Athe_0675 | or1803 |
| SYb039 | Athe_0676 | or1802 |
| SYb039 | Athe_0677 | or1801 |
| SYb039 | Athe_0678 | or1800 |
| FPb024 | Athe_0681 | or1796 |
| SYb040 | Athe_0718 | or1754 |
| SYb040 | Athe_0719 | or1753 |
| SYb040 | Athe_0720 | or1752 |
| SYb040 | Athe_0721 | or1751 |
| SYb040 | Athe_0722 | or1750 |
| SYb040 | Athe_0723 | or1749 |
| SYb040 | Athe_0724 | or1748 |
| SYb040 | Athe_0725 | or1747 |
| SYb040 | Athe_0726 | or1746 |
| FPb025 | Athe_0729 | or1742 |
| FPb026 | Athe_0732 | or1739 |
| SYb041 | Athe_0737 | or1734 |
| SYb041 | Athe_0738 | or1733 |
| SYb042 | Athe_0744 | or1362 |
| SYb042 | Athe_0745 | or1361 |
| SYb042 | Athe_0746 | or1360 |
| FPb027 | Athe_0759 | |
| FPb028 | Athe_0768 | or1338 |
| FPb029 | Athe_0864 | or1239 |
| FPb030 | Athe_0868 | |
| FPb031 | Athe_0871 | or1230 |
| FPb032 | Athe_0888 | or1212 |
| SYb043 | Athe_0892 | |
| SYb043 | Athe_0893 | or1207 |
| SYb043 | Athe_0894 | |
| FPb033 | Athe_0896 | or1204 |
| SYb044 | Athe_0899 | or1202 |
| SYb044 | Athe_0900 | or1201 |
| SYb044 | Athe_0901 | or1200 |
| SYb045 | Athe_0903 | or1197 |
| SYb045 | Athe_0904 | or1196 |
| FPb034 | Athe_0906 | or1195 |
| FPb035 | Athe_0908 | or1193 |
| SYb046 | Athe_0911 | or0498 |
| SYb046 | Athe_0912 | or0497 |
| SYb046 | Athe_0913 | or0496 |
| FPb036 | Athe_0916 | or0492, or0493 |
| FPb037 | Athe_0923 | or0485 |
| FPb038 | Athe_0945 | or0463 |
| SYb047 | Athe_0947 | or0460 |
| SYb047 | Athe_0948 | or0459 |
| SYb047 | Athe_0949 | or0458 |
| SYb047 | Athe_0950 | or0457 |
| FPb039 | Athe_0956 | or0450, or0451 |
| FPb040 | Athe_0965 | or0440 |
| SYb048 | Athe_1024 | or0379 |
| SYb048 | Athe_1025 | or0378 |
| SYb048 | Athe_1026 | or0377 |
| SYb048 | Athe_1027 | |
| SYb049 | Athe_1106 | or0296 |
| SYb049 | Athe_1107 | or0295 |
| SYb049 | Athe_1108 | or0294 |
| SYb049 | Athe_1109 | or0293 |
| SYb049 | Athe_1110 | or0292 |
| SYb049 | Athe_1111 | or0291 |
| SYb049 | Athe_1112 | or0290 |
| FPb041 | Athe_1122 | or0279 |
| FPb042 | Athe_1130 | or0271 |
| FPb043 | Athe_1146 | or0255 |
| FPb044 | Athe_1165 | or0236 |
| FPb045 | Athe_1174 | or0227 |
| SYb050 | Athe_1178 | |
| SYb050 | Athe_1179 | or0222 |
| FPb046 | Athe_1203 | or0197 |
| FPb047 | Athe_1256 | or0142 |
| FPb048 | Athe_1317 | or0080 |
| FPb049 | Athe_1329 | or0068 |
| SYb051 | Athe_1351 | or0046 |
| SYb051 | Athe_1352 | or0045 |
| SYb052 | Athe_1364 | or0033 |
| SYb052 | Athe_1365 | or0032 |
| SYb052 | Athe_1366 | or0029 |
| SYb052 | Athe_1367 | or0030 |
| SYb052 | Athe_1368 | or0031 |
| SYb052 | Athe_1369 | or0028 |
| SYb052 | Athe_1370 | or0027 |
| FPb050 | Athe_1383 | or0014 |
| FPb051 | Athe_1392 | or0005 |
| SYb053 | Athe_1394 | or0004 |
| SYb053 | Athe_1395 | or0003 |
| SYb053 | Athe_1396 | or0002 |
| SYb053 | Athe_1397 | or0001 |
| FPb052 | Athe_1408 | or0853 |
| FPb053 | Athe_1431 | |
| FPb054 | Athe_1468 | or0792 |
| FPb055 | Athe_1519 | or0739 |
| FPb056 | Athe_1572 | or0685 |
| SYb054 | Athe_1581 | or0675 |
| SYb054 | Athe_1582 | or0674 |
| SYb055 | Athe_1590 | or0666 |
| SYb055 | Athe_1591 | or0665 |
| SYb055 | Athe_1592 | or0664 |
| SYb056 | Athe_1597 | or0658 |
| SYb056 | Athe_1598 | or0657 |
| SYb056 | Athe_1599 | or0656 |
| SYb056 | Athe_1600 | or0655 |
| SYb056 | Athe_1601 | or0654 |
| SYb056 | Athe_1602 | or0653 |
| SYb056 | Athe_1603 | or0652 |
| SYb056 | Athe_1604 | or0651 |
| SYb056 | Athe_1605 | or0650 |
| SYb056 | Athe_1606 | or0649 |
| SYb056 | Athe_1607 | or0648 |
| FPb057 | Athe_1621 | or0634 |
| FPb058 | Athe_1633 | or0622 |
| SYb057 | Athe_1658 | or0596 |
| SYb057 | Athe_1659 | or0595 |
| SYb057 | Athe_1660 | or0594 |
| SYb057 | Athe_1661 | or0593, or0592 |
| SYb057 | Athe_1662 | or0591 |
| SYb057 | Athe_1663 | or0590 |
| SYb057 | Athe_1664 | or0589 |
| SYb057 | Athe_1665 | or0588 |
| SYb058 | Athe_1683 | |
| SYb058 | Athe_1684 | or0570 |
| FPb059 | Athe_1768 | or1570 |
| FPb060 | Athe_1771 | or1567 |
| FPb061 | Athe_1776 | or1562 |
| FPb062 | Athe_1817 | or1519 |
| FPb063 | Athe_1845 | or1490 |
| SYb059 | Athe_1853 | or2887, or2888 |
| SYb059 | Athe_1854 | or2886 |
| SYb059 | Athe_1855 | or2885 |
| SYb059 | Athe_1856 | or2910 |
| FPb064 | Athe_1858 | or2856 |
| FPb065 | Athe_1869 | or2230 |
| FPb066 | Athe_1907 | or2192 |
| FPb067 | Athe_1931 | or2508 |
| SYb060 | Athe_1933 | or2506 |
| SYb060 | Athe_1934 | or2505 |
| SYb060 | Athe_1935 | or2504 |
| SYb060 | Athe_1936 | or2503 |
| SYb060 | Athe_1937 | or2502 |
| FPb068 | Athe_1957 | or2482 |
| SYb061 | Athe_1962 | or2477 |
| SYb061 | Athe_1963 | or2476, or2475 |
| SYb061 | Athe_1964 | or2474, or2475 |
| SYb061 | Athe_1965 | or2473 |
| SYb061 | Athe_1966 | or2472 |
| SYb061 | Athe_1967 | or2471 |
| SYb061 | Athe_1968 | or2470 |
| SYb061 | Athe_1969 | or2469 |
| SYb061 | Athe_1970 | or2468 |
| FPb069 | Athe_1977 | or2899 |
| SYb062 | Athe_1985 | or1191 |
| SYb062 | Athe_1986 | or1190 |
| SYb063 | Athe_1989 | or1187 |
| SYb063 | Athe_1990 | or1186 |
| SYb063 | Athe_1991 | or1185 |
| SYb063 | Athe_1992 | or1184 |
| SYb063 | Athe_1993 | or1183 |
| SYb063 | Athe_1994 | or1182 |
| SYb064 | Athe_1996 | or1180 |
| SYb064 | Athe_1997 | or1179 |
| SYb064 | Athe_1998 | or1178 |
| SYb064 | Athe_1999 | or1177 |
| SYb064 | Athe_2000 | or1176 |
| FPb070 | Athe_2005 | or1171 |
| FPb071 | Athe_2013 | or1159 |
| SYb065 | Athe_2022 | or1149 |
| SYb065 | Athe_2023 | or1148 |
| FPb072 | Athe_2025 | or1146 |
| SYb066 | Athe_2029 | or1142 |
| SYb066 | Athe_2030 | or1141 |
| SYb066 | Athe_2031 | or1140 |
| FPb073 | Athe_2033 | or1138 |
| FPb074 | Athe_2063 | or1107 |
| SYb067 | Athe_2076 | or1093 |
| SYb067 | Athe_2077 | or1092 |
| SYb067 | Athe_2078 | or1091 |
| SYb067 | Athe_2079 | or1090, or1088, or1089 |
| SYb067 | Athe_2080 | or1087 |
| SYb067 | Athe_2081 | or1086 |
| SYb067 | Athe_2082 | or1085 |
| SYb067 | Athe_2083 | or1084, or1083 |
| SYb067 | Athe_2084 | or1082, or1083 |
| SYb067 | Athe_2085 | or1081 |
| SYb067 | Athe_2086 | or1080 |
| SYb067 | Athe_2087 | or1079 |
| SYb067 | Athe_2088 | or1078 |
| SYb067 | Athe_2089 | or1077 |
| SYb067 | Athe_2090 | or1076 |
| SYb067 | Athe_2091 | or1075 |
| SYb067 | Athe_2092 | or1074 |
| SYb067 | Athe_2093 | or1073 |
| SYb067 | Athe_2094 | or1071, or1072 |
| FPb075 | Athe_2103 | |
| FPb076 | Athe_2145 | or1018 |
| FPb077 | Athe_2153 | or1010 |
| SYb068 | Athe_2187 | or0975 |
| SYb068 | Athe_2188 | or0974 |
| FPb078 | Athe_2194 | or0968 |
| FPb079 | Athe_2196 | or0966 |
| SYb069 | Athe_2200 | or0962 |
| SYb069 | Athe_2201 | or0961 |
| FPb080 | Athe_2203 | or0959 |
| FPb081 | Athe_2209 | or0953 |
| FPb082 | Athe_2212 | or0950 |
| SYb070 | Athe_2216 | or0946 |
| SYb070 | Athe_2217 | or0944 |
| SYb071 | Athe_2223 | or0937 |
| SYb071 | Athe_2224 | or0936 |
| SYb072 | Athe_2230 | or0930 |
| SYb072 | Athe_2231 | or0929, or0930 |
| SYb072 | Athe_2232 | or0928 |
| SYb072 | Athe_2233 | or0927 |
| SYb072 | Athe_2234 | or0926 |
| SYb072 | Athe_2235 | or0925 |
| SYb072 | Athe_2236 | or0923, or0924 |
| SYb072 | Athe_2237 | or0922 |
| SYb072 | Athe_2238 | or0921 |
| SYb072 | Athe_2239 | or0920 |
| SYb073 | Athe_2247 | or0912 |
| SYb073 | Athe_2248 | or0911 |
| SYb073 | Athe_2249 | or0910 |
| SYb073 | Athe_2250 | or0909 |
| SYb074 | Athe_2257 | or0901 |
| SYb074 | Athe_2258 | or0900 |
| SYb074 | Athe_2259 | or0899 |
| SYb075 | Athe_2261 | |
| SYb075 | Athe_2262 | or0896 |
| SYb075 | Athe_2263 | or0895 |
| FPb083 | Athe_2275 | or0883 |
| FPb084 | Athe_2290 | or0866 |
| SYb076 | Athe_2292 | or0863, or0864, or2908 |
| SYb076 | Athe_2293 | or2096 |
| SYb077 | Athe_2300 | or2088 |
| SYb077 | Athe_2301 | or2087 |
| SYb078 | Athe_2312 | or2075 |
| SYb078 | Athe_2313 | or2074 |
| SYb078 | Athe_2314 | or2073 |
| SYb078 | Athe_2315 | or2072 |
| FPb085 | Athe_2320 | or2067 |
| FPb086 | Athe_2325 | or2060, or2061 |
| SYb079 | Athe_2328 | or2057 |
| SYb079 | Athe_2329 | or2056 |
| SYb080 | Athe_2331 | or2054 |
| SYb080 | Athe_2332 | or2053 |
| FPb087 | Athe_2344 | or2041 |
| SYb081 | Athe_2349 | or2036 |
| SYb081 | Athe_2350 | or2035 |
| FPb088 | Athe_2353 | or2032 |
| SYb082 | Athe_2371 | or1921 |
| SYb082 | Athe_2372 | or1922 |
| SYb082 | Athe_2373 | or1923 |
| SYb082 | Athe_2374 | or1924 |
| SYb082 | Athe_2375 | or1925 |
| SYb082 | Athe_2376 | or1926 |
| FPb089 | Athe_2379 | or1930 |
| FPb090 | Athe_2382 | or1933 |
| FPb091 | Athe_2404 | or1956 |
| SYb083 | Athe_2407 | or1959 |
| SYb083 | Athe_2408 | or1960 |
| SYb083 | Athe_2409 | or1961 |
| SYb083 | Athe_2410 | or1962 |
| SYb084 | Athe_2412 | or1964 |
| SYb084 | Athe_2413 | or1965 |
| SYb084 | Athe_2414 | or1966 |
| SYb084 | Athe_2415 | or1967 |
| SYb085 | Athe_2417 | or1969 |
| SYb085 | Athe_2418 | or1970 |
| SYb085 | Athe_2419 | or1971 |
| SYb085 | Athe_2420 | or1972 |
| SYb085 | Athe_2421 | or1973 |
| SYb085 | Athe_2422 | or1974 |
| SYb085 | Athe_2423 | or1975 |
| SYb085 | Athe_2424 | or1976 |
| SYb085 | Athe_2425 | or1977 |
| SYb085 | Athe_2426 | or1978 |
| SYb085 | Athe_2427 | or1979 |
| SYb085 | Athe_2428 | or1980 |
| SYb085 | Athe_2429 | or1981 |
| SYb086 | Athe_2431 | or1983 |
| SYb086 | Athe_2432 | or1984 |
| SYb086 | Athe_2433 | or1985 |
| SYb086 | Athe_2434 | or1986 |
| SYb087 | Athe_2436 | or1988 |
| SYb087 | Athe_2437 | or1989 |
| SYb087 | Athe_2438 | or1990 |
| SYb087 | Athe_2439 | or1991 |
| SYb087 | Athe_2440 | or1992, or1993 |
| SYb088 | Athe_2442 | or1996 |
| SYb088 | Athe_2443 | or1997 |
| SYb088 | Athe_2444 | or1998 |
| SYb088 | Athe_2445 | or1999 |
| SYb088 | Athe_2446 | or2000 |
| FPb092 | Athe_2462 | or2016 |
| SYb089 | Athe_2468 | or2913 |
| SYb089 | Athe_2469 | or2912 |
| SYb090 | Athe_2471 | |
| SYb090 | Athe_2472 | or2834 |
| SYb090 | Athe_2473 | or2833 |
| SYb091 | Athe_2475 | or2831 |
| SYb091 | Athe_2476 | or2830 |
| SYb091 | Athe_2477 | or2829 |
| SYb091 | Athe_2478 | or2828 |
| SYb091 | Athe_2479 | or2827 |
| SYb091 | Athe_2480 | or2826 |
| FPb093 | Athe_2484 | or2822 |
| SYb092 | Athe_2486 | or2820 |
| SYb092 | Athe_2487 | or2818, or2819 |
| SYb092 | Athe_2488 | or2817 |
| SYb092 | Athe_2489 | or2816 |
| SYb092 | Athe_2490 | or2815 |
| SYb092 | Athe_2491 | or2814 |
| SYb092 | Athe_2492 | or2813 |
| SYb093 | Athe_2494 | or2811 |
| SYb093 | Athe_2495 | or2810 |
| SYb093 | Athe_2496 | or2809 |
| SYb093 | Athe_2497 | or2808 |
| SYb093 | Athe_2498 | or2807 |
| SYb093 | Athe_2499 | or2806 |
| SYb093 | Athe_2500 | or2805 |
| SYb094 | Athe_2504 | or2801 |
| SYb094 | Athe_2505 | or2800 |
| SYb094 | Athe_2506 | or2799 |
| SYb094 | Athe_2507 | or2798 |
| SYb094 | Athe_2508 | or2797 |
| SYb094 | Athe_2509 | or2796 |
| SYb094 | Athe_2510 | or2795 |
| SYb095 | Athe_2512 | |
| SYb095 | Athe_2513 | |
| SYb095 | Athe_2514 | or2464 |
| SYb095 | Athe_2515 | or2463 |
| SYb095 | Athe_2516 | or2462 |
| FPb094 | Athe_2518 | or2460 |
| FPb095 | Athe_2525 | or2453 |
| FPb096 | Athe_2527 | or2451 |
| SYb096 | Athe_2530 | or2448 |
| SYb096 | Athe_2531 | or2447 |
| SYb096 | Athe_2532 | or2446 |
| SYb096 | Athe_2533 | or2445 |
| SYb097 | Athe_2536 | or2442 |
| SYb097 | Athe_2537 | or2441 |
| SYb097 | Athe_2538 | or2440 |
| SYb097 | Athe_2539 | or2439 |
| SYb097 | Athe_2540 | or2438 |
| FPb097 | Athe_2545 | or2432, or2433 |
| SYb098 | Athe_2547 | or2430 |
| SYb098 | Athe_2548 | or2429 |
| FPb098 | Athe_2556 | or2421 |
| SYb099 | Athe_2586 | or2248 |
| SYb099 | Athe_2587 | or2249 |
| SYb099 | Athe_2588 | or2250 |
| FPb099 | Athe_2604 | or2267 |
| FPb100 | Athe_2613 | or2276 |
| FPb101 | Athe_2622 | or2286 |
| SYb100 | Athe_2628 | or2292 |
| SYb100 | Athe_2629 | or2293 |
| SYb101 | Athe_2634 | or2557 |
| SYb101 | Athe_2635 | or2558 |
| FPb102 | Athe_2637 | or2560 |
| FPb103 | Athe_2647 | or2572 |
| SYb102 | Athe_2653 | or2579, or2580 |
| SYb102 | Athe_2654 | or2581, or2582 |
| FPb104 | Athe_2665 | or2591 |
| FPb105 | Athe_2667 | or2593 |
| FPb106 | Athe_2672 | or2598 |
| FPb107 | Athe_2678 | or2346 |
| SYb103 | Athe_2686 | or2336 |
| SYb103 | Athe_2687 | or2335 |
| SYb103 | Athe_2688 | or2334 |
| SYb103 | Athe_2689 | or2333 |
| SYb103 | Athe_2690 | or2332 |
| SYb104 | Athe_2692 | or2329 |
| SYb104 | Athe_2693 | or2328 |
| SYb104 | Athe_2694 | or2327 |
| SYb104 | Athe_2695 | or2326 |
| SYb104 | Athe_2696 | or2325 |
| SYb104 | Athe_2697 | or2324 |
| FPb108 | Athe_2706 | or2315 |
| FPb109 | Athe_2709 | or2311 |
| SYb105 | Athe_2711 | or2309 |
| SYb105 | Athe_2712 | or2308 |
| SYb105 | Athe_2713 | or2307 |
| FPb110 | Athe_2716 | or2304 |
| SYb106 | Athe_2718 | or2299 |
| SYb106 | Athe_2719 | or2298, or2877 |
| SYb106 | Athe_2720 | or2876 |
| SYb106 | Athe_2721 | |
| FPb111 | Athe_2728 | or2767 |
| FPb112 | Athe_2743 | or2752 |
| FPb113 | Athe_2764 | or2730 |
| FPb114 | Athe_2768 | or1841 |

TABLE 2
Exemplary PBU Gene Clusters

| Cluster/Single | GenBank |
|---|---|
| Number | CP001393.1 locus tag |
| SYb001 | Athe_0010 |
| SYb001 | Athe_0011 |
| SYb001 | Athe_0012 |
| SYb001 | Athe_0013 |
| SYb001 | Athe_0014 |
| SYb001 | Athe_0015 |
| SYb001 | Athe_0016 |
| SYb001 | Athe_0017 |
| SYb004 | Athe_0052 |
| SYb004 | Athe_0053 |
| SYb004 | Athe_0054 |
| SYb004 | Athe_0055 |
| SYb004 | Athe_0056 |
| SYb004 | Athe_0057 |
| SYb004 | Athe_0058 |
| SYb004 | Athe_0059 |
| SYb004 | Athe_0060 |
| SYb004 | Athe_0061 |
| SYb012 | Athe_0153 |
| SYb012 | Athe_0154 |
| SYb012 | Athe_0155 |
| SYb012 | Athe_0156 |
| SYb012 | Athe_0157 |
| SYb012 | Athe_0158 |
| SYb012 | Athe_0159 |
| SYb012 | Athe_0160 |
| SYb026 | Athe_0347 |
| SYb026 | Athe_0348 |
| SYb026 | Athe_0349 |
| SYb026 | Athe_0350 |
| SYb026 | Athe_0351 |
| SYb026 | Athe_0352 |
| SYb026 | Athe_0353 |
| SYb026 | Athe_0354 |
| SYb026 | Athe_0355 |
| SYb026 | Athe_0356 |
| SYb026 | Athe_0357 |
| SYb029 | Athe_0406 |
| SYb029 | Athe_0407 |
| SYb029 | Athe_0408 |
| SYb029 | Athe_0409 |
| SYb029 | Athe_0410 |
| SYb029 | Athe_0411 |
| SYb029 | Athe_0412 |
| SYb029 | Athe_0413 |
| SYb040 | Athe_0718 |
| SYb040 | Athe_0719 |
| SYb040 | Athe_0720 |
| SYb040 | Athe_0721 |
| SYb040 | Athe_0722 |
| SYb040 | Athe_0723 |
| SYb040 | Athe_0724 |
| SYb040 | Athe_0725 |
| SYb040 | Athe_0726 |
| SYb056 | Athe_1597 |
| SYb056 | Athe_1598 |
| SYb056 | Athe_1599 |
| SYb056 | Athe_1600 |
| SYb056 | Athe_1601 |
| SYb056 | Athe_1602 |
| SYb056 | Athe_1603 |
| SYb056 | Athe_1604 |
| SYb056 | Athe_1605 |
| SYb056 | Athe_1606 |
| SYb056 | Athe_1607 |
| SYb057 | Athe_1658 |
| SYb057 | Athe_1659 |
| SYb057 | Athe_1660 |
| SYb057 | Athe_1661 |
| SYb057 | Athe_1662 |
| SYb057 | Athe_1663 |
| SYb057 | Athe_1664 |
| SYb057 | Athe_1665 |
| SYb061 | Athe_1962 |
| SYb061 | Athe_1963 |
| SYb061 | Athe_1964 |
| SYb061 | Athe_1965 |
| SYb061 | Athe_1966 |
| SYb061 | Athe_1967 |
| SYb061 | Athe_1968 |
| SYb061 | Athe_1969 |
| SYb061 | Athe_1970 |
| SYb067 | Athe_2076 |
| SYb067 | Athe_2077 |
| SYb067 | Athe_2078 |
| SYb067 | Athe_2079 |
| SYb067 | Athe_2080 |
| SYb067 | Athe_2081 |
| SYb067 | Athe_2082 |
| SYb067 | Athe_2083 |
| SYb067 | Athe_2084 |
| SYb067 | Athe_2085 |
| SYb067 | Athe_2086 |
| SYb067 | Athe_2087 |
| SYb067 | Athe_2088 |
| SYb067 | Athe_2089 |
| SYb067 | Athe_2090 |
| SYb067 | Athe_2091 |
| SYb067 | Athe_2092 |
| SYb067 | Athe_2093 |
| SYb067 | Athe_2094 |
| SYb072 | Athe_2230 |
| SYb072 | Athe_2231 |
| SYb072 | Athe_2232 |
| SYb072 | Athe_2233 |
| SYb072 | Athe_2234 |
| SYb072 | Athe_2235 |
| SYb072 | Athe_2236 |
| SYb072 | Athe_2237 |
| SYb072 | Athe_2238 |
| SYb072 | Athe_2239 |
| SYb085 | Athe_2417 |
| SYb085 | Athe_2418 |
| SYb085 | Athe_2419 |
| SYb085 | Athe_2420 |
| SYb085 | Athe_2421 |
| SYb085 | Athe_2422 |
| SYb085 | Athe_2423 |
| SYb085 | Athe_2424 |
| SYb085 | Athe_2425 |
| SYb085 | Athe_2426 |
| SYb085 | Athe_2427 |
| SYb085 | Athe_2428 |
| SYb085 | Athe_2429 |

Of the 218 functionally-annotated coding regions (rather than having an unknown function) found in A. thermophilum that are not found in C. saccharolyticus, 20 of them encode polysaccharide hydrolases and related (PIM) enzymes (Table 3). Several of the coding regions that encode PHR enzymes are part of eight so-called PHR gene clusters (Table 4). These include clusters of six (SYb082), 19 (SYb067), six (SbYb063) eight (SYb012) and 10 (SYb004) coding regions (see Table 4). The PHR clusters contain almost 60 coding regions (including the 20 PHR coding regions).

TABLE 3
PHR Coding Regions

| | GenBank |
|---|---|
| Cluster/Single | CP001393.1 |
| Number | locus tag |
| SYb004 | Athe_0058 |
| SYb004 | Athe_0059 |
| SYb004 | Athe_0061 |
| SYb007 | Athe_0089 |
| SYb012 | Athe_0154 |
| SYb012 | Athe_0156 |
| SYb012 | Athe_0157 |
| FPb015 | Athe_0423 |
| SYb032 | Athe_0452 |
| FPb022 | Athe_0603 |
| FPb023 | Athe_0610 |
| SYb059 | Athe_1853 |
| SYb059 | Athe_1854 |
| SYb059 | Athe_1855 |
| SYb063 | Athe_1993 |
| SYb067 | Athe_2076 |
| SYb067 | Athe_2086 |
| SYb067 | Athe_2089 |
| SYb067 | Athe_2094 |
| SYb082 | Athe_2371 |

TABLE 4
PHR Gene
Clusters

| | GenBank |
|---|---|
| Cluster/Single | CP001393.1 |
| Number | locus tag |
| SYb004 | Athe_0052 |
| SYb004 | Athe_0053 |
| SYb004 | Athe_0054 |
| SYb004 | Athe_0055 |
| SYb004 | Athe_0056 |
| SYb004 | Athe_0057 |
| SYb004 | Athe_0058 |
| SYb004 | Athe_0059 |
| SYb004 | Athe_0060 |
| SYb004 | Athe_0061 |
| SYb007 | Athe_0088 |
| SYb007 | Athe_0089 |
| SYb007 | Athe_0090 |
| SYb012 | Athe_0153 |
| SYb012 | Athe_0154 |
| SYb012 | Athe_0155 |
| SYb012 | Athe_0156 |
| SYb012 | Athe_0157 |
| SYb012 | Athe_0158 |
| SYb012 | Athe_0159 |
| SYb012 | Athe_0160 |
| SYb032 | Athe_0450 |
| SYb032 | Athe_0451 |
| SYb032 | Athe_0452 |
| SYb059 | Athe_1853 |
| SYb059 | Athe_1854 |
| SYb059 | Athe_1855 |
| SYb059 | Athe_1856 |
| SYb063 | Athe_1989 |
| SYb063 | Athe_1990 |
| SYb063 | Athe_1991 |
| SYb063 | Athe_1992 |
| SYb063 | Athe_1993 |
| SYb063 | Athe_1994 |
| SYb067 | Athe_2076 |
| SYb067 | Athe_2077 |
| SYb067 | Athe_2078 |
| SYb067 | Athe_2079 |
| SYb067 | Athe_2080 |
| SYb067 | Athe_2081 |
| SYb067 | Athe_2082 |
| SYb067 | Athe_2083 |
| SYb067 | Athe_2084 |
| SYb067 | Athe_2085 |
| SYb067 | Athe_2086 |
| SYb067 | Athe_2087 |
| SYb067 | Athe_2088 |
| SYb067 | Athe_2089 |
| SYb067 | Athe_2090 |
| SYb067 | Athe_2091 |
| SYb067 | Athe_2092 |
| SYb067 | Athe_2093 |
| SYb067 | Athe_2094 |
| SYb082 | Athe_2371 |
| SYb082 | Athe_2372 |
| SYb082 | Athe_2373 |
| SYb082 | Athe_2374 |
| SYb082 | Athe_2375 |
| SYb082 | Athe_2376 |

The PHR coding regions and particularly the PHR clusters together with other coding regions in the 550 gene set found in A. thermophilum that are not found in C. saccharolyticus form what are referred to herein as the plant biomass utilization, or PBU, coding regions. The PBU coding regions are directly and indirectly involved in enabling A. thermophilum to efficiently utilize untreated, treated, and spent plant biomass. Thus, the ability to confer to other microorganisms the ability to utilize untreated and/or spent biomass can be achieved by directly transferring certain PBU polynucleotides to microorganisms known to utilize, for example, cellulose and xylan. Since A. thermophilum grows at moderate temperatures (75° C. optimum, but remain viable at, for example 90° C.), the microorganisms receiving an A. thermophilum PBU polynucleotide can include thermophilic microorganisms, including extreme thermophiles, as well as microorganisms that grow at more moderate temperatures (mesophiles).

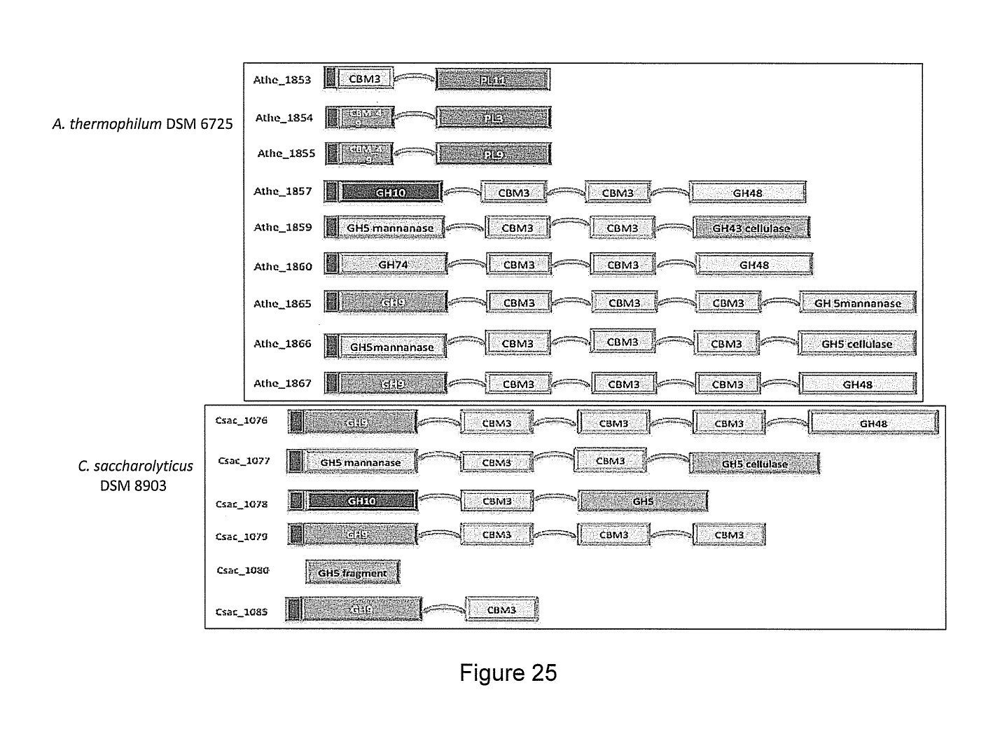

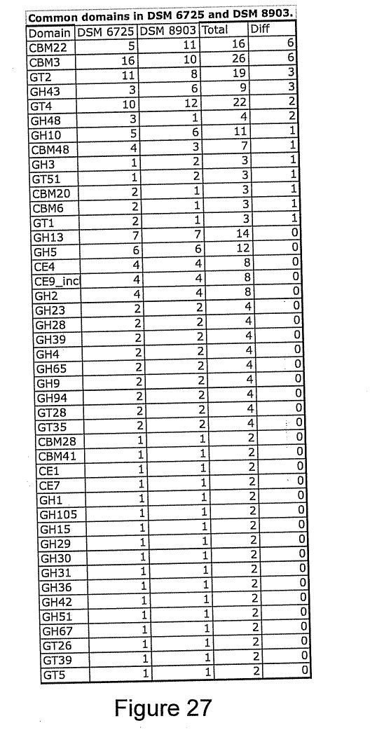

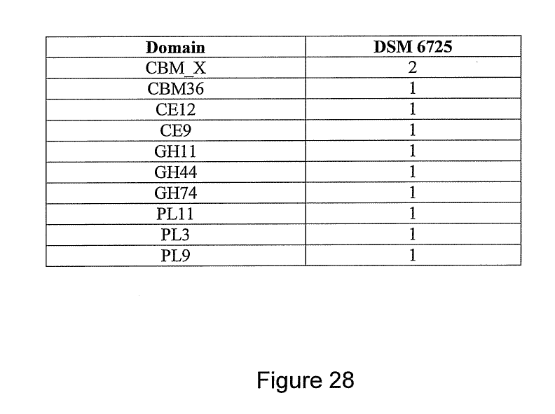

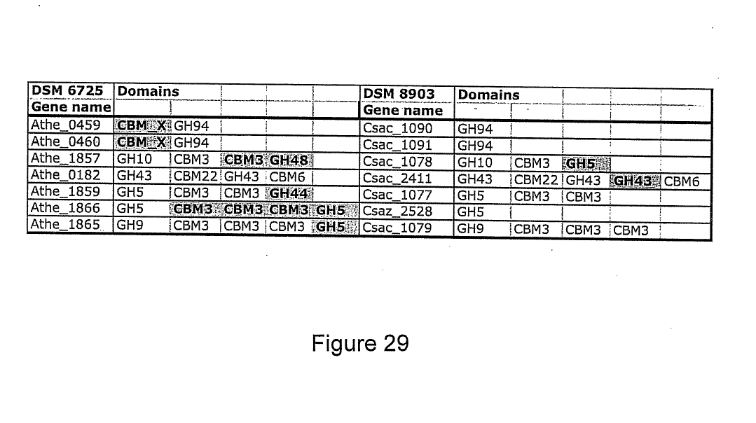

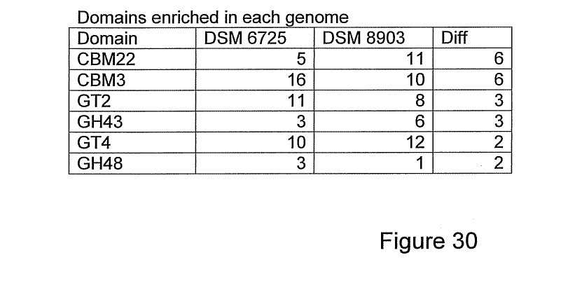

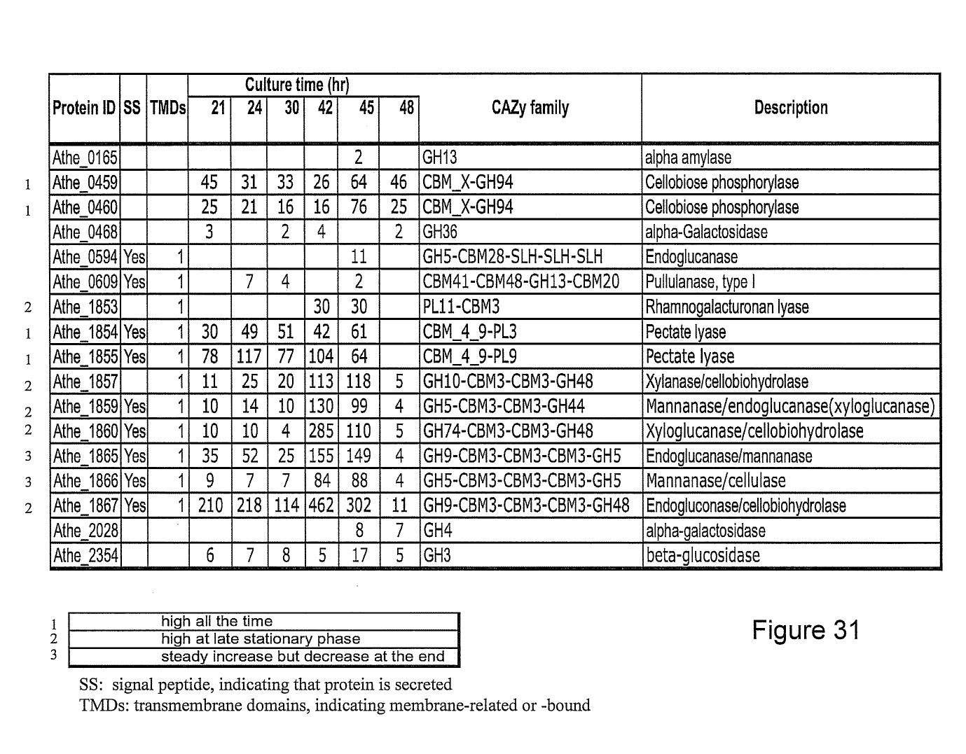

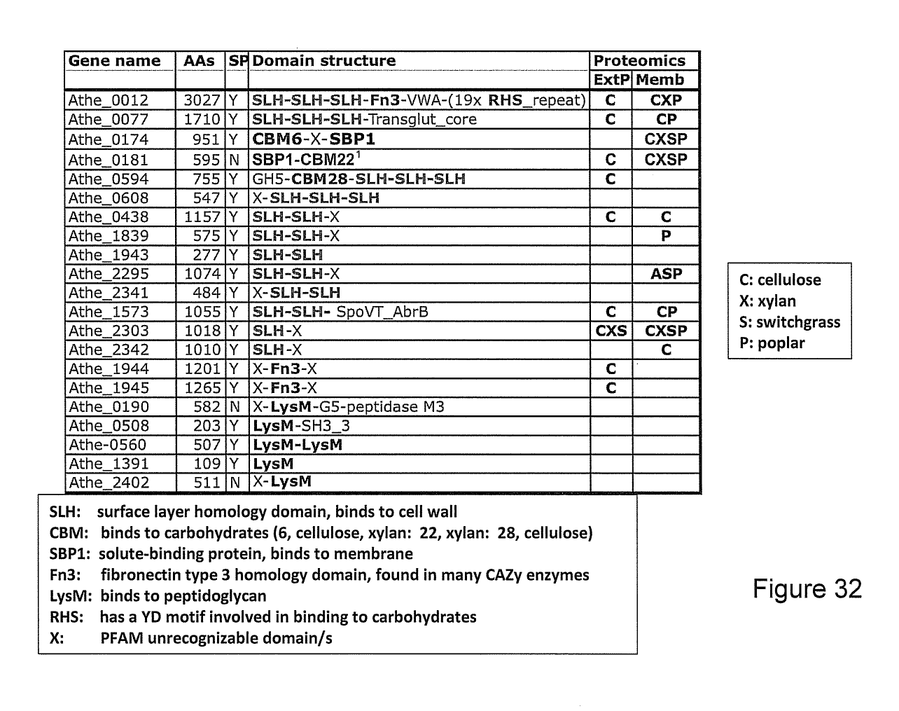

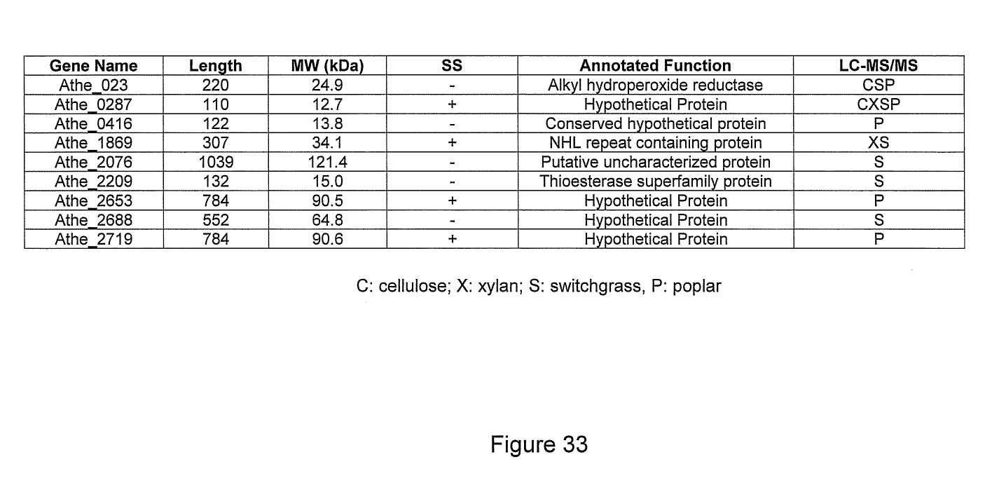

Coding regions that enable A. thermophilum to efficiently breakdown plant biomass encode various types of proteins, including what are referred to herein as carbohydrate-active enzymes (CAZy) as well as proteins that may not be catalytic but allow the microorganism to attach to the insoluble biomass prior to and during degradation. FIG. 27 lists CAZy-related domains—found in enzymes such as glycoside hydrolases, glycosyl transferases, and carbohydrate esterases—that are present in the genomes of A. thermophilum and C. saccharolyticus. Such domains can be highly conserved between functionally related proteins and between species. Thus, the structure and function of many CAZy-related domains are well characterized. FIG. 28 lists CAZy-related domains that are uniquely present in A. thermophilum. In addition, A. thermophilum has some unique combinations of these domains that are not present in C. saccharolyticus (FIG. 25 and FIG. 29). Some of these and other CAZy-related coding regions are expressed at different times throughout the growth phase when A. thermophilum is grown on crystalline cellulose, as shown by proteomic identification of the proteins released by the microorganism into the growth medium (FIG. 31). Numerous non-catalytic extracellular and membrane-associated proteins were also identified in the A. thermophilum genome that could potentially mediate its attachment to biomass (FIG. 32). Using the same proteomics analyses, several of these have been measured in either the extracellular fraction or the membrane fraction of A. thermophilum when grown on cellulose, xylan, switchgrass, and/or poplar (FIG. 32). FIG. 33 lists some other proteins, measured by proteomic analysis, that are not encoded in the genome of C. saccharolyticus but are produced by A. thermophilum when the microorganism is grown on cellulose, xylan, switchgrass, and/or poplar.

An A. thermophilum PBU polynucleotide can include one or more of the PBU coding regions identified in Table 1. In some embodiments, the A. thermophilum PBU polynucleotide can include one or more coding regions of a PBU gene cluster as identified in Table 2. In certain embodiments, the A. thermophilum PBU polynucleotide may be an A. thermophilum PHR polynucleotide—i.e., include one or more of the A. thermophilum PHR coding regions identified in Table 3. In some embodiments, the A. thermophilum PHR polynucleotide can include one or more coding regions of a PHR gene cluster as identified in Table 4. The complete nucleotide sequence—and the predicted amino sequence encoded by the nucleotide sequence—of every remaining A. thermophilum PBU coding region is accessible via GenBank Accession No. CP001395 (version 1, created Feb. 5, 2009).

An A. thermophilum polynucleotide can include one or more A. thermophilum coding regions that encode products that are involved in plant biomass utilization, but may not necessarily be specific to A. thermophilum compared to C. saccharolyticus. Such coding regions can include, for example, Athe1867 (SEQ ID NO:6). Consequently, the A. thermophilus polynucleotide can encode a polypeptide having the amino acid sequence of, for example, SEQ ID NO:7.

Thus, in another aspect, the present invention provides methods of transferring one or more polynucleotides of A. thermophilum to a recipient microorganism. In some cases, such methods can include the cloning and direct transfer of one or more polynucleotides from A. thermophilum to the recipient microorganism. Such methods are routine and known to those skilled in the art. (See, e.g., Sambrook et al, (1989) Molecular Cloning: A Laboratory Manual., Cold Spring Harbor Laboratory Press or Ausubel, R. M., ed. (1994). Current Protocols in Molecular Biology).

When direct cloning methods are used to transfer one or more polynucleotides from A. thermophilum to a recipient microorganism, the recipient microorganism may be any microorganism suitable for cloning transfer of polynucleotides. Suitable recipient microorganisms include, for example, members of the family Enterobacteriaceae such as, for example, members of the genus Escherichia or Salmonella. In certain embodiments, a suitable recipient microorganism may include E. coli. In other embodiments, the recipient microorganism can include a eukaryote such as, for example, a yeast such as, for example, Saccharomyces cerevisiae.

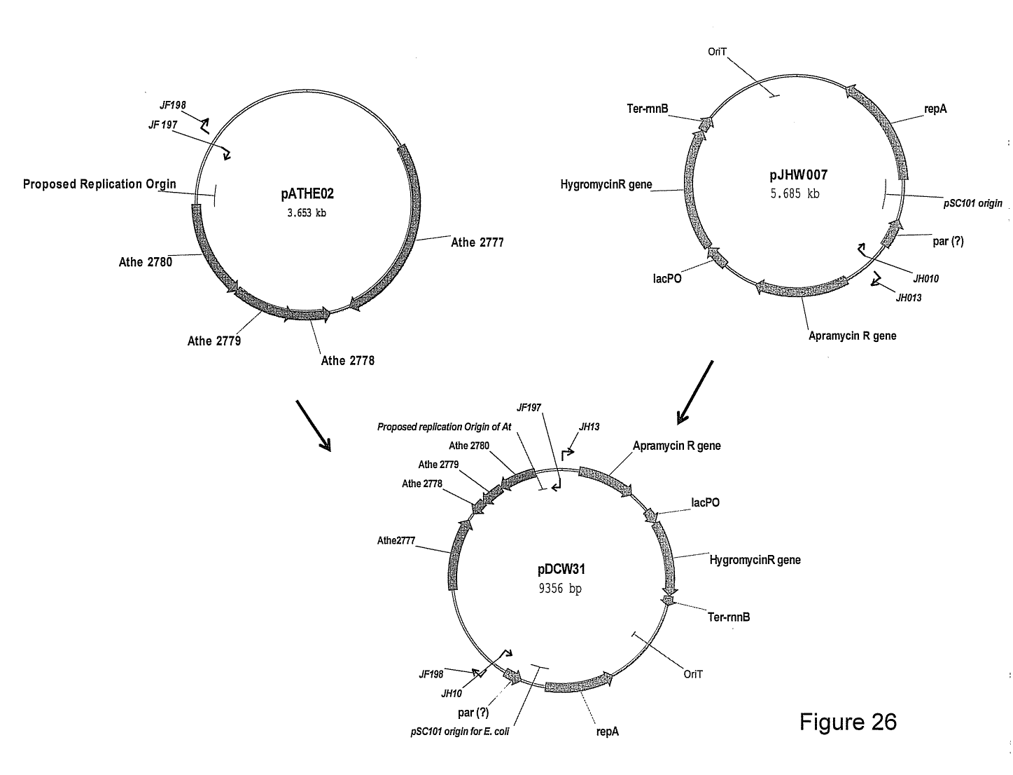

In other cases, such methods can include the cloning and transfer of one or more polynucleotides from A. thermophilum to an intermediate, or “vector,” microbe, followed by transfer of the one or more A. thermophilum polynucleotides from the vector microbe to the recipient microorganism. The cloning of the one or more A. thermophilum polynucleotides into the vector microbe may be accomplished using routine methods referred to in the immediately preceding paragraph. Alternatively, the cloning of one or more A. thermophilum polynucleotides into the vector microbe may be accomplished using a shuttle vector that permits the movement of nucleotide sequences cloned into the shuttle vector to be shuttled between A. thermophilum and another microorganism. One such shuttle vector is pDCW 31, the construction of which is described in Example 5 and is shown in FIG. 26. The pCDW 31 shuttle vector contains elements from the naturally-occurring A. thermophilum plasmid pAthe02 (SEQ ID NO:1) and the pSC101-based plasmid pJHW007. While components of the pJHW007 plasmid were used to construct pCDW 31, analogous components of any pSC101-based plasmid can be used to construct a similar shuttle vector.

The subsequent transfer of the one or more A. thermophilum polynucleotides to a recipient microorganism may be accomplished by any method appropriate for transferring a polynucleotide to the particular recipient microorganism. In some cases, an appropriate method may include routine cloning methods already described. In other cases, an appropriate method may include methods described in U.S. Provisional Patent Application Ser. No. 61/000,338, filed, Oct. 25, 2007, entitled “METHODS FOR GENETIC MANIPULATION OF EXTREMOPHILES,” which describes the transfer of polynucleotides by conjugation. Conjugation is a polynucleotide transfer process in which a donor microbe (e.g., a vector microbe) makes contact with and transfers a polynucleotide to a recipient (Frost et al., Microbiol. Rev., 1994, 58:162-210); Willets and Skurray, In: Escherichia coli and Salmonella typhimurium: cellular and molecular biology, Neidhardt et al. (eds.), 1987, American Society for Microbiology, Washington, D.C., 1110-1133). Generally, such methods include co-cultivating a vector microbe and a recipient microorganism, wherein the vector microbe includes a conjugative polynucleotide, and wherein the co-cultivation is under conditions suitable for conjugative transfer of at least a portion of the conjugative polynucleotide from the vector microbe to the recipient microorganism, and identifying a recipient microorganism exconjugant. Conjugation from a vector microbe to a recipient microorganism can result in the transfer of a plasmid or in the transfer of part of the vector microbe's chromosome. Preferably, the methods described herein result in transfer of a plasmid from vector microbe to the recipient microorganism.

In particular, conjugative methods may be appropriate if the recipient microorganism is, for example, an extremophile or a mesophile. Examples of extremophiles include, but are not limited to, thermophiles and extreme thermophiles (microorganisms that grow in environments at temperatures of between 50° C. and 100° C., and between 70° C. and 100° C., respectively), hyperthermophiles (microorganisms that grow in environments at temperatures above 80° C.), acidophiles (microorganisms that grow in environments at low pH, such as less than pH 3), and halophiles (microorganisms that grow in environments of at least 1 M NaCl). The extremophile may be an obligate anaerobe. The extremophile may be a member of the kingdom Archaea such as, for instance, a member of phylum Crenarchaeota, Euryarchaeota, Korarchaeota, or Nanoarchaeota, preferably Crenarchaeota or Euryarchaeota, more preferably, Euryarchaeota. Examples of such microorganisms include, but are not limited to, Pyrococcus spp., such as P. furiosus, Sulfolobus spp, such as S. solfataricus, and Thermococcus spp., such as T kodakaraensis. The extremophile may be a member of the family Thermotogaceae, such as, for example, Thermotoga spp. such as, for example, T. maritima, or a member of the family Aquificaceae, such as, for example, Aquifex spp such as, for example, A. aeolicus. Examples of thermophiles that are not extreme thermophiles include, for example, A. thermophilum, Caldicellulosiruptor saccharolyticus, and Clostridium thermocellum. Examples of mesophiles include, for example, members of the family Enterobacteriaceae such as, for example, members of the genus Escherichia or Salmonella. In certain embodiments, a suitable mesophile may include E. coli.

The vector microbe may be a member of the family Enterobacteriaceae and may be, but is not limited to, E. coli and Salmonella spp. The member of the family Enterobacteriaceae is one that is able to transfer polynucleotides by conjugation with the recipient microorganism. Alternatively, the vector microbe may be a member of the family Bacillaceae such as, for example, Bacillus spp.

In some embodiments, the polynucleotide to be transferred to the recipient microorganism (e.g., the cloning vector or conjugative polynucleotide) can include an A. thermophilum PBU coding region as defined above. The transfer of a polynucleotide that includes an A. thermophilum PBU coding region can permit the recipient microorganism (e.g., the cloning recipient or the exconjugant) to express an A. thermophilum polypeptide—as defined above—encoded by the A. thermophilum PBU coding region. Exemplary PBU polypeptides are encoded by A. thermophilum PBU coding regions identified in Table 1. The amino acid sequences of PBU polypeptides encoded by the exemplary PBU coding regions are accessible via GenBank Accession No. CP001395 (version 1, created Feb. 5, 2009).

In some embodiments, the polynucleotide to be transferred to the recipient microorganism (e.g., the cloning vector or conjugative polynucleotide) can include a PHR coding region as defined above—i.e., a member of a subset of PBU coding regions. The transfer of a polynucleotide that includes an A. thermophilum PHR coding region can permit the recipient microorganism (e.g., the cloning recipient or the exconjugant) to express an A. thermophilum polypeptide—as defined above—encoded by the A. thermophilum PHR coding region. Exemplary PHR coding regions are identified in Table 3. The amino acid sequences of PHR polypeptides encoded by the exemplary PHR coding regions are accessible via GenBank Accession No. CP001395 (version 1, created Feb. 5, 2009).

The recombinantly expressed A. thermophilum polypeptide (e.g., a PBU polypeptide or a PHR polypeptide) may be isolated from the recipient cell—whether a cloning recipient or an exconjugant—using methods well-known in the art. Consequently, in another aspect, the present invention provides an isolated polypeptide encoded by an A. thermophilum PBU polynucleotide or a PHR polynucleotide.

In another aspect, the present invention provides a genetically-modified microorganism that includes one or more Anaerocellum thermophilum plant biomass utilization (PBU) polynucleotides. The genetically-modified microorganism may be derived from one of the recipient microorganisms described above with respect to methods of transferring at least a portion of an A. thermophilum polynucleotide to a recipient microorganism. Also, the genetically-modified microorganism may include one or more PBU coding regions, PHR coding regions, or one or more coding regions from a gene cluster identified above.

In some embodiments, the genetically-modified microorganism may be modified in a way to promote the production and/or accumulation of a particular metabolic product. As noted above, such genetic modifications can include the introduction of one or more heterologous coding regions that promote the production of one or more desired products or intermediates. In other cases, such genetic modifications can include disrupting the activity of one or more endogenous coding regions in a way that inhibits the production of non-desired metabolic products and/or redirects the metabolism of intermediates toward the production of desired metabolic products.

For example, metabolic pathways that supply or are supplied by the citric acid cycle are well known to those skilled in the art. Thus, disrupting—either by reducing or eliminating the activity of products encoded by certain coding regions—a metabolic pathway that is, at least in part, supplied by the citric acid cycle can shunt metabolism away from the disrupted pathway (and its product) in favor of accumulating other intermediates of the citric acid cycle and/or pathways supplied by those alternative intermediates. Examples of modifications that disrupt a metabolic pathway include, for example, “knock out” mutations that significantly reduce or eliminate biological activity of the mutated coding region (and/or the polypeptide encoded by the mutated coding region). Methods for introducing knock out mutations in many cellular models are routine and known to those skilled in the art. In other words, one may direct metabolism toward pathways that produce desired products by reducing or eliminating metabolism via pathways that compete with the desired pathway for metabolic resources.

For example, modifications that disrupt one or more metabolic enzymes involved in a pathway supplied by the citric acid cycle can promote the accumulation of, for example, succinate that would otherwise be metabolized—either directly by the disrupted pathway or indirectly to form the citric acid cycle intermediate that would be directly metabolized by the disrupted pathway. Disrupting activity in other well known metabolic pathways can promote production of, for example, ethanol, acetate, lactate, hydrogen gas, etc. Exemplary targets for such knock out mutations in A. thermophilum include, for example, Athe_{—}1918 (SEQ ID NO:8), Athe_{—}2388 (SEQ ID NO:10), Athe_{—}1493 (SEQ ID NO:12), Athe_{—}1494 (SEQ ID NO:14), Athe_{—}1223 (SEQ ID NO:16), but those skilled in the art can readily determine additional targets in A. thermophilum by identifying coding regions in A. thermophilum that correspond to known components of known and conserved metabolic pathways other microorganisms.

Such modifications may be provided alone or in combination with one or more additional modifications such as, for example, introduction of a heterologous coding region that promotes the conversion of an intermediate (e.g., an intermediate accumulated due to a knock out modification) to a desired product (e.g., a metabolic product not produced—or produced inefficiently—by the wild type of the genetically-modified microorganism. In some cases, the production of one or more butanols may be promoted in A. thermophilum by a combination of disrupting one or more A. thermophilum metabolic pathways and introducing one or more heterologous coding regions that promote the production of butanol from. In one exemplary embodiment, a knock out modification in one or more of SEQ ID NO:8, SEQ ID NO:10, SEQ ID NO:12, SEQ ID NO:14, or SEQ ID NO:16 may be combined with introducing one or more coding regions of Clostridium acetobutylicum that are known to confer the ability to produce 1-butanol in E. coli such as, for example, the coding region for C. acetobutylicum thiolase (Atsumi et al., Metab. Eng. 2008, 10:305-311.

In yet another aspect, the present invention provides a method of processing plant biomass. In this aspect, the method includes growing genetically-modified microorganisms comprising one or more A. thermophilum PBU polynucleotides on a substrate that comprises plant biomass under conditions effective for the microorganism to convert at least a portion of the plant biomass to a water soluble product.

Generally, the plant biomass, the cultivation conditions, the microorganisms, and PBU polynucleotides may be those described above in connection with various embodiments of other aspects of the present invention. In some embodiments, the genetically-modified microorganism may be A. thermophilum. In other embodiments, the genetically-modified microorganism may be a microorganism other than A. thermophilum.

Another utility of A. thermophilum and/or the genetically-modified microorganisms described above may be for the production of one or more A. thermophilum polypeptides that possesses acellular plant biomass degrading activity—i.e., is able to degrade plant biomass when isolated from A. thermophilum. Thus, in another aspect, the present invention provides a method of making an isolated A. thermophilum polypeptide. Generally, the method includes growing a microorganism comprising at least one polynucleotide encoding an Anaerocellum thermophilum polypeptide possessing plant biomass degrading activity under conditions effective for the microorganism to produce the A. thermophilum polypeptide, and isolating the A. thermophilum polypeptide.

In some embodiments, the microorganism may be A. thermophilum. In other embodiments, the microorganism may be genetically engineered to include one or more A. thermophilum PBU polynucleotides, PHR polynucleotides, or one or more coding regions from a gene cluster identified above. Methods for isolating polypeptides produced by microorganisms in culture are well known to those skilled in the art. Polypeptides and fragments thereof useful in the present invention may be produced using recombinant DNA techniques, such as an expression vector present in a cell. Such methods are routine and known in the art. The polypeptides and fragments thereof may also be synthesized in vitro, e.g., by solid phase peptide synthetic methods. The solid phase peptide synthetic methods are routine and known in the art. A polypeptide produced using recombinant techniques or by solid phase peptide synthetic methods may be further purified by routine methods, such as fractionation on immunoaffmity or ion-exchange columns, ethanol precipitation, reverse phase HPLC, chromatography on silica or on an anion-exchange resin such as DEAE, chromatofocusing, SDS-PAGE, ammonium sulfate precipitation, gel filtration using, for example, Sephadex G-75, or ligand affinity.

In some cases, the isolated polypeptide may be used to directly for biomass conversion. Thus, in yet another aspect, the present invention provides a method of processing plant biomass. Generally, the method includes providing an isolated A. thermophilum polypeptide possessing plant biomass degrading activity, and contacting the A. thermophilum polypeptide with plant biomass under conditions effective for the A. thermophilum polypeptide to at least partially degrade the plant biomass.

In certain circumstances, it may be desirable to have the A. thermophilum utilization of plant biomass result in the production of an product that A. thermophilum is not naturally capable of producing. In such cases, the water soluble product produced by methods described herein may be recovered and subsequently processed to produce a desired end product. In other cases, the desired end product may be a product of a metabolic process native to another microorganism that is made possible by expression of one or more coding regions from that microorganism. Transfer of a polynucleotide that includes one or more such coding regions to A. thermophilum may permit the A. thermophilum to perform one or more additional metabolic steps to convert the water soluble product to the desired product.

Thus, in yet another aspect, the present invention provides methods of transferring one or more polynucleotides that include heterologous coding regions—e.g., carbohydrate metabolism coding regions or butanol synthesis coding regions—to A. thermophilum. Metabolic pathways in E. coli for producing, for example, various biofuels are known and coding regions of the E. coli genome that promote the production of the various biofuels are similarly known. (See, e.g., Connor et al., Curr. Opin. Biotech. 2009, 20:307-315 and Atsumi et al., Metab. Eng. 2008, 10:305-311).

One or more heterologous coding regions may be introduced into A. thermophilum using any suitable method including, for example, routine cloning and direct transfer of polynucleotides containing the heterologous coding region, cloning and transfer of one or more polynucleotides to A. thermophilum via an intermediate, or “vector,” microbe, or the transfer of polynucleotides by conjugation, as described above. In addition, a polynucleotide that includes one or more heterologous coding regions may be introduced into A. thermophilum by, for example, electroporation as described in Example 6, below.

Generally, the plant biomass, the processing conditions (e.g., temperature), and the A. thermophilum polypeptide may be those described above in connection with various embodiments of other aspects of the present invention.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

Anaerocellum thermophilum strain DSM 6725 (Deutsche Sammlung von Mikroorganismen and Zellkulturen GmbH (DSMZ), Braunschweig, Germany) was grown in 0.5% modified 516 medium (DSMZ). The medium was modified by adding vitamins and trace minerals solutions and the method to reduce the medium. The modified medium contained, per liter: 0.5 g yeast extract, 0.33 g NH₄C1, 0.33 g

KH₂PO₄, 0.33 g KCl, 0.33 g MgCl₂×6 H₂O, 0.33 g CaCl₂×2 H₂O, 0.5 mg resazurin, 5 mL vitamin solution, and 1 mL trace minerals solution. The vitamin solution contained: 4 mg/L biotin , 4 mg/L folic acid, 20 mg/L pyridoxine-HCl, 10 mg/L thiamine-HCl, 10 mg/L riboflavin, 10 mg/L nicotinic acid, 10 mg/L calcium panthothenate, 0.2 mg/L vitamin B₁₂, 10 mg/L p-aminobenzoic acid, and 10 mg/L lipoic acid. The trace minerals solution contained: 2 g/L FeCl₃, 0.05 g/L ZnCl₂, 0.05 g/L MnCl₂×4H₂O, 0.05 g/L H₃BO₃, 0.05 g/L CoCl₂×6H₂O, 0.03 g/L CuCl₂×2H₂O, 0.05 g/L NiCl₂×6H₂O, 0.5 g/L Na₄EDTA (tetrasodium salt), 0.05 g/L (NH₄)2MoO₄, and 0.05 g/L AlK(SO₄)₂.12H₂O. Both vitamin and trace minerals solutions were filtered through 0.22 pm membrane and stored at 4° C. The reducing system was composed of 0.5 g cysteine, 0.5 g N₂S, and 1 g NaHCO₃. The final pH was 7.2. The medium was filtered through 0.22 μM membrane and prepared anaerobically under 80% N₂ +20% CO₂ (N₂/CO₂) gas atmosphere. Soluble growth substrates were added into the medium prior to filtration. Insoluble growth substrates were weighed and added into sterilized culture bottles individually.

The growth substrates and their sources were: D-(+)-cellobiose (cat. C7252) and oat spelts xylan (cat. X0627) were from Sigma Chemical Company, St. Louis, Mo., and Avicel PH-101 (cat. 11365) was from Fluka, Switzerland), Poplar and switchgrass (sieved, −20/+80 mesh fraction) were provided by Dr. Brian Davison of Oak Ridge National Laboratory (Oak Ridge, Tenn.), Tifton 85 bermuda grass and napier grass (sieved, −20/+80 mesh fraction) were provided by Dr. Joy Peterson (Department of Microbiology, University of Georgia, Athens, Ga.), and the pine wood was provided by Dr. Alan Darvill (Department of Biochemistry and Complex Carbohydrate Research Center, University of Georgia, Athens, Ga.).

A. thermophilum was grown at 75° C. with shaking at 150 rpm unless specified otherwise. To test the ability of A. thermophilum to grow on untreated plant biomass, A. thermophilum was grown in 50 mL 0.5% modified 516 medium in sealed 100-mL serum bottles without shaking. For the kinetic analyses, A. thermophilum was grown in either 0.5 L or 0.25 L cultures in 1 L or 0.5 L sealed bottles, respectively. “Flushed” cultures were grown in the same conditions, but the cultures were purged with N₂/CO₂. For growth on “spent” insoluble substrates (from poplar, switchgrass and Avicel), the insoluble material that was left over after cells had grown on that substrate was collected in late stationary phase (when cell growth had stopped). The residual insoluble substrate was separated from the cells by filtering through glass filters with a pore size 40-60 μm. The material was washed with distilled water and dried at 50° C. overnight. This was then used as the growth substrate for new cultures.

During growth of A. thermophilum on different complex and defined substrates, samples were removed from the cultures at various time intervals (FIGS. 1-4). Some or all of the following parameters were measured: pH, cell density, cell protein, hydrogen, acetate, lactate, ethanol, and in some cases, reducing sugars. The cell count was determined using a phase-contrast microscope with 40× magnification. Cell protein was determined by the Bradford method. For cell protein assay in cultures growing on insoluble substrate, the cells were separated from the substrate by a low speed centrifugation. To measure protein, the cell pellet resuspended in 50 mM Tris-HCl (pH 7.0) buffer with lysozyme (0.2 mg/ml) was incubated at 10° C. for 6 hours and then subjected to three freeze-thaw cycles. Acetate and lactate were measured in the growth medium after removing cells (and the insoluble substrate if present) by HPLC (Waters 2690 Separations Module, Waters Corp., Milford, Mass.) equipped with a Aminex HPX-87H column (300 mm 7.8 mm, Bio-Rad Corp., Hercules, Calif.) at 40° C. with 5 mM H₂SO₄ as the mobile phase at a flow rate of 0.6 ml min⁻¹ with a refractive index detector (Waters 2410, Waters Corp., Milford, Mass.). Ethanol was measured enzymatically using the Ethanol Kit (Megazyme International Ireland Ltd., Wicklow, Ireland). Hydrogen producing during cell growth was determined by gas chromatography (Shimadzu GC-8A, Shimadzu Scientific Instruments, Inc., Columbia, Md.) equipped with a thermal conductivity detector and a molecular sieve column (Alltech 5A 80/100, Grace Davison Discovery Sciences, Waukegan, Ill.) with argon as the carrier gas. Reducing sugars were determined with dinitrosalicylic acid (DNS) reagent as previously described (Miller, G. L., 1959, Anal. Chem., 31:426-428).

The data shown in FIGS. 12-15 used the defined medium that we developed for A. thermophilum (DSMZ 6725). The same medium was also used to grow Caldicellulosiruptor saccharolyticus (DSMZ 8903). Both microorganisms were grown in 50 mL culture volumes in a medium containing: 0.33 g/L MgCl₂, 0.33 g/L KCl, 0.25 g/L NH₄Cl, 0.14 g/L CaCl₂, trace minerals (Na₄EDTA, FeCl₃, ZnCl₂, MnCl₂, H₃B0₃, CoCl₂, CuCl₂, NiCl₂, (NH₄)₂MoO₄, AlK(SO₄)), vitamin mix (0.02 mg/L biotin, 0.02 mg/L folic acid, 0.1 mg/L pyridoxine-HCl, 0.05 mg/L thiamine, 0.05 mg/L riboflavin, 0.05 mg/L nicotinic acid, 0.05 mg/L D-Ca-pantothenate, 0.001 mg/L vitamin B₁₂, 0.05 mg/L p-aminobenzoic acid, 0.05 mg/L lipoic acid), 20 amino acids (0.076 g/L alanine, 0.124 g/L arginine, 0.1 g/L asparagine, 0.048 g/L aspartic acid, 0.2 g/L glutamic acid, 0.048 g/L glutamine, 0.2 g/L glycine, 0.1 g/L histidine, 0.1 g/L isoleucine, 0.1 g/L leucine, 0.1 g/L lysine, 0.076 g/L methionine, 0.076 g/L phenylalanine, 0.125 g/L proline, 0.076 g/L serine, 0.1 g/L threonine, 0.076 g/L tryptophan, 0.012 g/L tyrosine, 0.052 g/L valine, 0.5 g/L cysteine), 0.25 mg/mL resazurin, 1 mM KH₂PO₄, 0.5 g/L Na₂S, and 1.0 g/L NaHCO₃. The heat-treated biomass samples were prepared by taking switchgrass, poplar or pine (100 mg) and extracting them for 2 minutes with 2 mL sterile water at 98° C. The soluble material was removed and used as a growth substrate for one culture and the insoluble solid was used as the growth substrate for a separate culture. Cultures were grown in triplicate at 75° C. without stirring or shaking. The cell density was measured as described above.

### Example 2

CelA (Athe_{—}1867, or2232, SEQ ID NO:6) encodes a cellulase coding region in A. thermophilum with an activity not present in the hyperthermophile P. furiosus , a microorganism that grows optimally at 100° C. The CelA coding region contains two cellulase enzymatic domains intermixed with carbohydrate binding domains. Two forms of the CelA coding region from A. thermophilum are generated and introduced into P. furiosus by mating as described in U.S. Provisional Patent Application Ser. No. 61/000,338, entitled “METHODS FOR GENETIC MANIPULATION OF EXTREMOPHILES,” filed Oct. 25, 2007. The first form consists of part of the native CelA nucleotide sequence itself (a single cellulase enzymatic domain and a single carbohydrate binding domain adjacent to it). This truncated form of CelA is cloned by PCR amplification from A. thermophilum into E. coli in a vector for mating into P furiosus. The second form of CelA consists of these domains proceeded by a signal sequence for protein localization. The signal sequence is from the P. furiosus alpha amylase coding region.

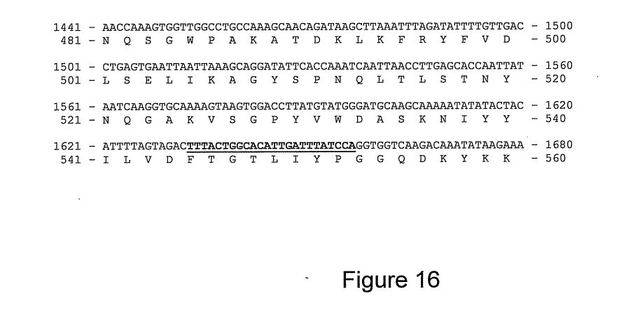

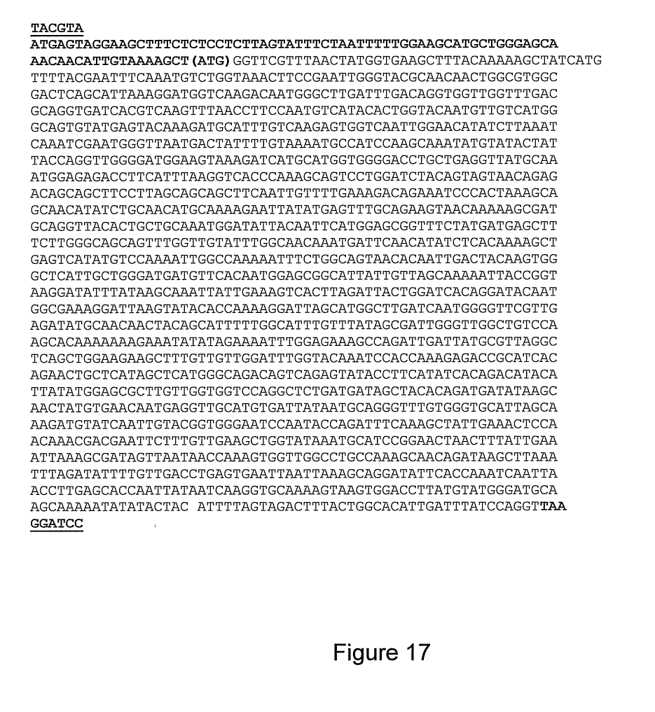

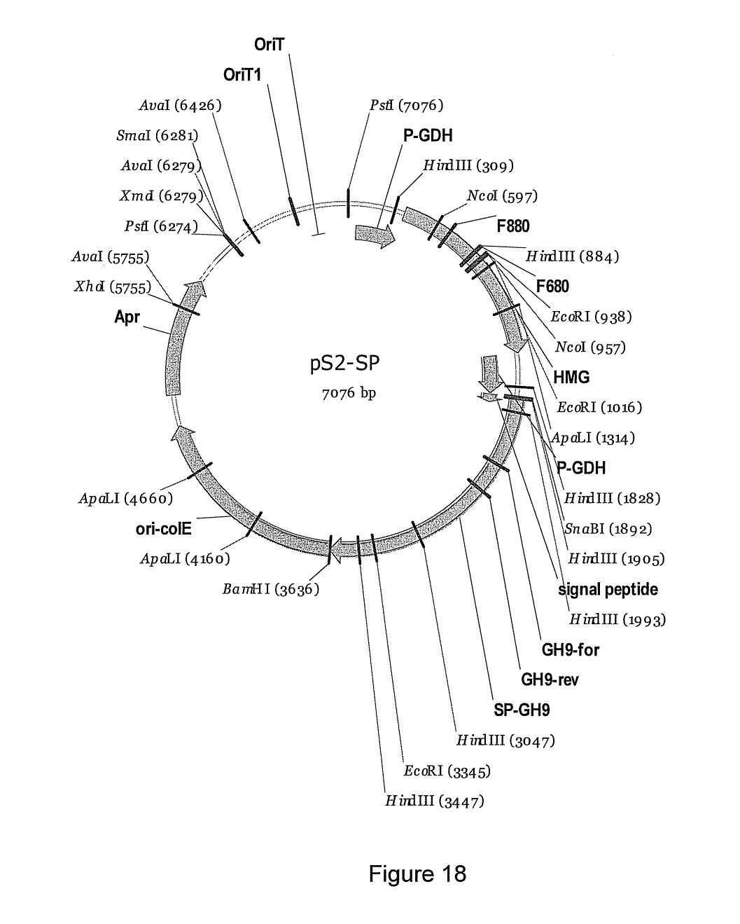

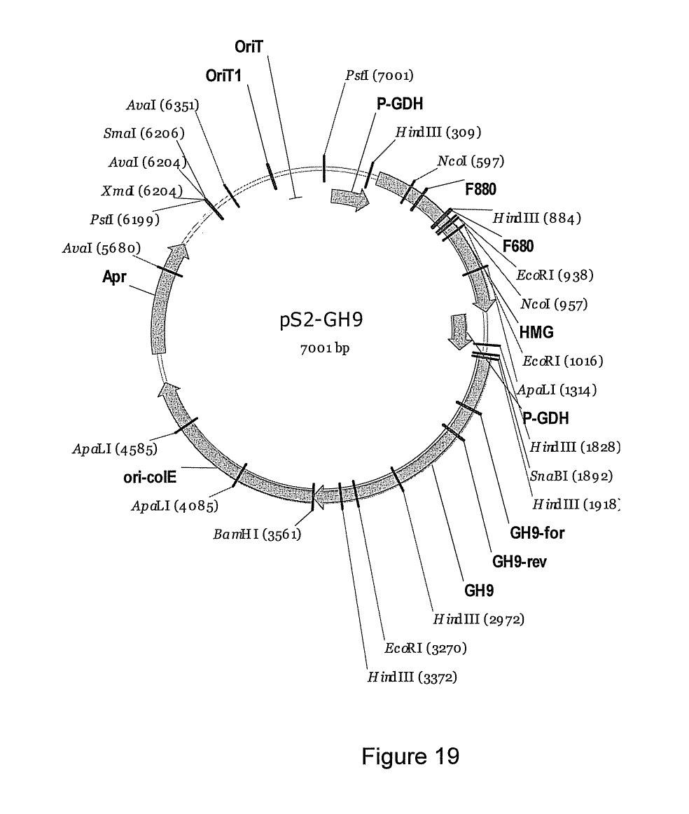

The DNA sequence of the CelA coding region and signal sequence are shown in FIGS. 16 and 17 respectively. Plasmid maps of these constructions are shown in FIGS. 18 and 19.

These plasmids are mated into P. furiosus and exconjugants are selected on simvastatin using methods described as follows:

### Media Components

1000× (1 mL/L) Trace Minerals Solution: 1.00 mL/L HCl (concentrated), 0.50 g/L
Na₄EDTA (tetrasodium), 2.00 g/L FeCl₃, 0.05 g/L H₃BO₃, 0.05 g/L ZnCl₂, 0.03 g/L

### CuCl₂.2H₂O, 0.05 g/L MnCl₂.4H₂O, 0.05 g/L (NH₄)₂MoO₄, 0.05 g/L

### AlK(SO₄).2H₂O, 0.05 g/L CoCl₂.6H₂O, and 0.05 g/L NiCl₂.6H₂O.

5× Base Salts: 140.00 g/L NaCl, 17.50 g/L, MgSO₄.7H₂O, 13.50 g/L MgCl₂.6H₂O, 1.65 g/L KCl, 1.25 g/L NH₄Cl, 0.70 g/L CaCl₂.2H₂O.
Liquid complex cellobiose (CC) media (pH 6.8): 200 mL/L 5× Base salts, 1 mL/L 1000× Trace minerals, 100 μL/L 100 mM Na₂WO₄*2H₂O, 50 μL/L Resazurin (5 mg/mL), 5 mL/L 10% w/v Yeast Extract, 50 mL/L 10% w/v Casein hydrolysate, 35 mL/L 10% w/v Cellobiose, 0.5 g/L Cysteine, 0.5g Na₂S, 1 g/L NaHCO₃, 1 mL/L 1M K₂HPO₄ buffer.
Solid complex cellobiose (CC) media: 1× media +1% phytagel solution (Sigma Chemical Company, St. Louis, Mo.).
CC plates containing 5-fluoroorotic acid (5-FOA): to ensure complete 5-FOA solvation, 1M NaOH is dripped into the solution until a murky consistency is reached at around pH 10, cysteine is then used to lower the pH to 7, where the solution turns transparent.
Simvastatin plates: solid complex cellobiose plates with the indicated amount of simvastatin added.
A. thermophilum is sensitive to 8 millimolar (mM) 5-FOA, 30 mM hygromycin, 8 micromolar (μM) simvastatin, and 50 μM apramycin.

### Growth Conditions.

P. furiosus strain (DSM 3638) (DSMZ, Braunschweig, Germany) is grown in liquid complex cellobiose (CC) media and on solid CC plates containing 1% phytagel. 50 mL liquid cultures are incubated in serum bottles and phytagel-containing plates of solid media are cultivated in anaerobic jars. Both types of media are grown at 90° C. under an argon atmosphere introduced through a vacuum manifold. Single crossover mutants containing an up-regulated HMG CoA reductase coding region are selected for on CC plates containing 8 μM Simvastatin (Sigma Chemical Company, St. Louis, Mo.). PyrF deletion mutants are selected for on CC plates containing 0.25% 5-FOA (Zymo Research Corp., Orange, Calif.). P. furiosus cells are plated on solid media by adding 50 μL of cell suspension to a pool of 800 μL 1× base salts. The plates are then spun by hand to spread the cells by centrifugal force. E. coli strains XL10 (Stratagene, LaJolla, Calif.) and ET12576 (Beirman et al., Gene 1992, 116L43-49) are grown in both liquid LB media and on solid LB plates at 37° C.

### Growth Measurements.

Cell counts are estimated by direct observation 2 μL of cell sample using a Petroff-Hauser counting chamber under 40× magnification. Viable cell count is determined by plating 1/100 and 1/1000 dilutions of cell culture and recording the number of colony forming units.

### Conjugation Procedure.

P. furiosus strain (DSM 3638) (DSMZ, Braunschweig, Germany) is used as the recipient strain in the conjugation experiments. 100 mL of a 1% v/v inoculum P. furiosus are incubated for nine hours to a cell density of approximately 10⁸ cells/mL. The cells are then pelleted at 5100 rpm for 15 minutes and washed twice with 1× base salts before resuspending in a final volume of 3 mL 1× base salts. E. coli strain ET12576, carrying the helper plasmid PUZ8002 and the conjugation plasmid, was used as the donor. An E. coli culture of 50 mL LB media containing 50 μg/mL kanamycin (selection for PUZ8002) and 50 μg/mL apramycin (selection for conjugation plasmid) is incubated overnight until a cell density of approximately 10⁹ cells/mL is reached. The E. coli is then pelleted at 2500 rpm for 10 minutes and washed twice with LB. 1 mL of the P. furiosus cell suspension is used to resuspend the E. coli control pellet, carrying only the PUZ8002 plasmid. The remaining 2 mL of P. furiosus are combined with the pellet of E. coli cells containing both the PUZ8002 plasmid and the conjugation plasmid. Once the E. coli cells have been resuspended with P. furiosus cells, the mixture is allowed to shake at 37° C. at 200 rpm for one hour. The cells are then plated on CC media containing Simvastatin as previously described and incubated aerobically at 37° C. for two hours to allow conjugation to occur. After the two hour incubation, the plates are transferred to anaerobic jars. Additional reductants, in the form of solid Na₂S and cysteine crystals, are added directly to the anaerobic jar as it is filled with the plates. Once the jars have been degassed and filled with an argon atmosphere, they are transferred to 90° C. incubators and allowed to grow for 40 hours.

### Mutant Selection.

After incubating for 40 hours, the anaerobic jars are placed in water baths to cool to room temperature before opening. Colonies growing on plates with selection are restreaked on fresh selective plates and incubated for another 40 hours to test for stability of transformation. In concert with the restreaks, mutants are inoculated into 5 mL of liquid CC cultures with no selection to create cell stocks. Genomic DNA is isolated from the cell stocks for further analysis by PCR after examination of the restreaked selective plates to identify potential transformants demonstrating stability with new growth. To select for double crossover mutants, exconjugants demonstrating resistance to the first selection (8 μM Simvastatin) are passaged through non-selective liquid CC media and plated on media containing the second selective reagent (0.25% 5-FOA). Colonies growing on the second selection are restreaked and inoculated into liquid cultures as previously described.

DNA isolation. Pyrococcus Furiosus Genomic DNA Mini Prep Protocol

1-2 mL of P. furiosus cell culture is pelleted at 5000 rpm for 10 minutes and resuspend in 200 μL of buffer A (25% w/v sucrose, 50 mM Tris-HCl pH 7.8, 40 mM EDTA) w/RNase A by vortexing. 250 μL of 6M guanidinium pH 8.5 is added to the pellet, mixed by gentle inversion, and allowed to sit for 5 minutes. The pellet is washed twice with 200 μL phenol/chloroform. The aqueous layers are combined and washed with 200 μL chloroform/isoamylalcohol (24:1). 20 μL of 3M sodium acetate is added and mixed by gentle inversion. 0.6 volumes of isopropanol is added and allowed to sit at −80° C. for 15 minutes after mixing by inversion. The sample is centrifuged at 14,000 rpm for 30 minutes. The supernatant is carefully removed and the pellet washed with 70% ethanol. The pellet is centrifuged at 5000 rpm for 2 minutes. The supernatant is removed and the pellet is allowed to air dry. The pellet is resuspened in 50 μL dH₂O or an appropriate buffer.

### Example 3

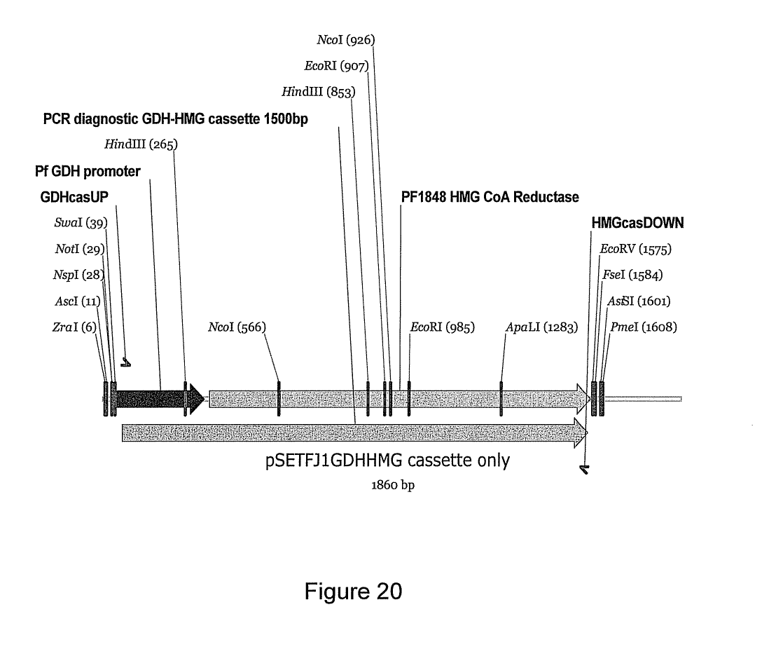

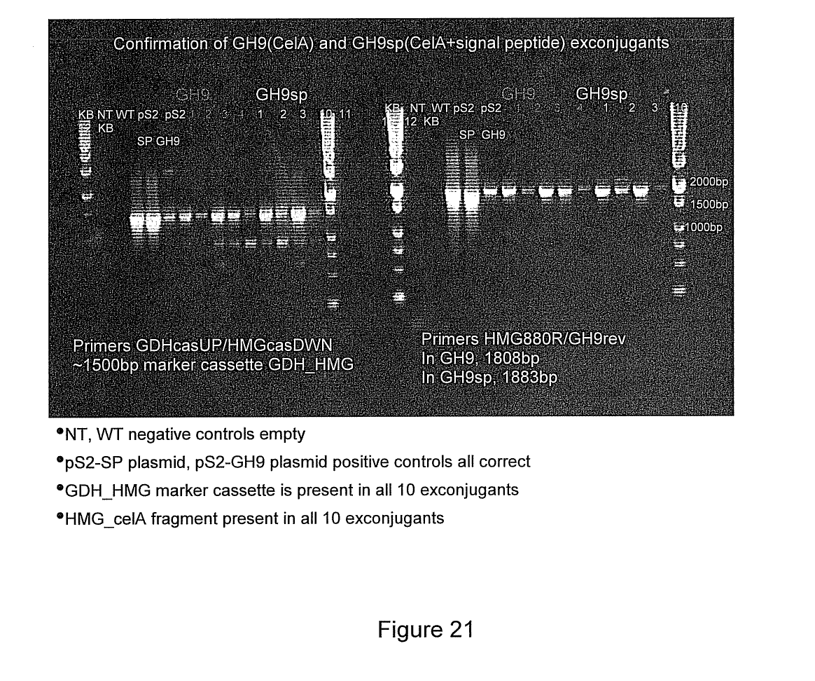

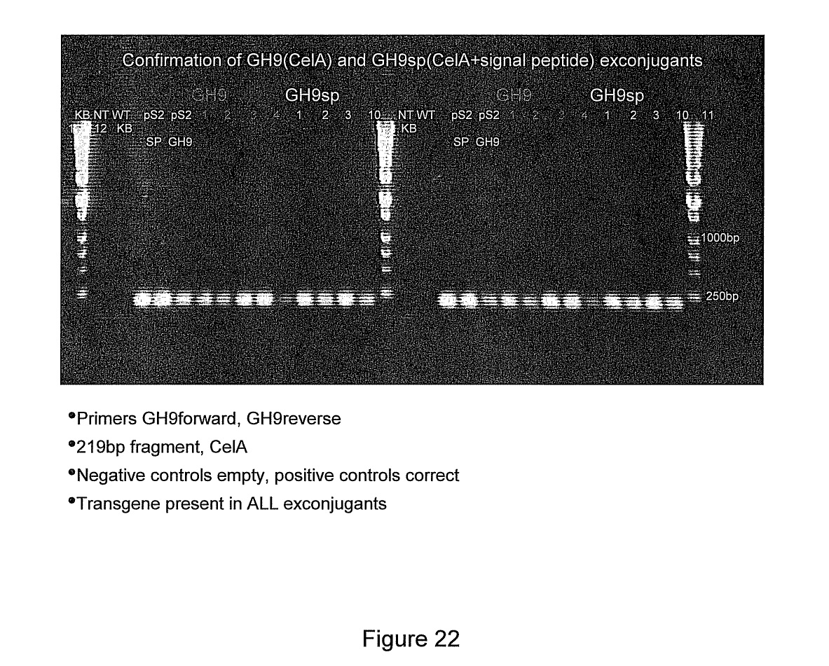

The presence of the celA coding region in the P. furiosus chromosome was confirmed by PCR. Primers for PCR were designed to amplify the GDH-CelA cassette with and without a signal sequence upstream of the CelA coding region (FIG. 20). The expected products were obtained from the P. furiosus exconjugants but not wild type P. furiosus strain (FIGS. 21 and 22). These results indicate that the GDH-CelA construction is integrated into the P. furiosus chromosome. As these plasmids do not replicate in P. furiosus , it is expected that the cassette integrated at either the GDH or HMG locus. The plasmid also contains a GDH-HMG cassette for simvastatin selection and as both these coding regions are from P. furiosus they provide an area of homology for crossing over.

In addition, quantitative PCR assays (qPCR) were performed on the P. furiosus exconjugants to detect the presence of A. thermophilum CelA specific transcript. These assays detect relative transcript levels as compared to an internal standard. In this case the constitutively expressed POR transcript was used as an internal control. CelA transcript was clearly detected in the exconjugants but not in the wild type strain. Since there is no “wild type” level of CelA transcript to compare it to there is no “x-fold” level of increase in this case. The detection of the CelA transcript confirms the presence of the coding region in P. furiosus and indicates that it is in fact expressed at the level of transcription.

### Example 4

A. thermophilum was grown as described in Example 1, except that the growth substrate was peanut shells (0.5%, w/v) that were used either with or without prior washing at 75° C. for 18 hours. Results are shown in FIG. 24.

### Example 5

Construction of pDCW 31, Anaerocellum-E. coli Shuttle Vector

The native A. thermophilum plasmid pAthe02 (SEQ ID No:1) has been sequenced (GenBank Accession No. CP001395, version 1, created Feb. 5, 2009) and is described in Kataeva et al. (2009), J. Bact., 191(11):3760-3761. The entire 3.653 kb pAthe02 plasmid was amplified by PCR using the primers JF 197 and JF198:

| | |
|---|---|
| JF197 5′-CAGCGTTAGCAAAGTGTTGT-3′ | (SEQ ID NO: 2) |
| JF198 5′-AGCTAACGGACAGCTCAACGT-3′ | (SEQ ID NO: 3) |

A 5.601 kb fragment from the pJHW007 plasmid was amplified by PCR using the primer set JH010 and JH013:

```
(SEQ ID NO: 4)
JH10 5′-AGAGAGATGCATACCAGCCTAACTTCGATCATTGGA-3′
                              Nsi I
(SEQ ID NO: 5)
JH13 5′-AGAGAGGGTACCAGGATCTCAAGAAGATCCTTTGAT-3′
                              Kpn I
```

All PCR amplifications were performed using the High Fidelity Pfu DNA polymerase (Stratagene, La Jolla, Calif.) as described in the manufacturer's direction. The two amplified DNA fragments were treated with FAST-LINK DNA ligase (Epicentre Biotechnologies, Madison, Wis.) to construct pDCW 31 (9.356 kb) by blunt-end Ligation. The pDCW 31 plasmid includes the pSC101 origin of replication and the apramycin resistance coding regions that function in E. coli, and a replication origin and hygromycin resistance cassette that function in Anaerocellum. It also contains an oriT. Construction of pDCW 31 is shown in FIG. 26.

### Example 6

Anaerocellum thermophilum (At) Electroporation Protocol

0.1 mL of an Anaerocellum thermophilum culture (approximately 2 10 ⁸ cells per mL) is inoculated into a bottle with 50 mLs of defined At medium+uracil. Growth medium components are prepared as separate sterile stock solutions. Stock solutions are as follows: 50× salts prepared in a final volume of 1 L, 16.5 g of MgCl₂.6H₂O, 16.5 g of KCl, 12.5 g of NH₄Cl, 7.0 g of CaCl₂.2H2O; 1000× trace minerals prepared in a final volume of 1 L, 1.0 ml of HCl (25%: 7.7M), 0.5 g of Na₄EDTA tetrasodium, 2.0 g FeCl₃.4H₂O, 0.05 g of ZnCl₂, 0.05 g of MnCl₂.4H₂O, 0.05 g of H₃BO₃, 0.05 g of CoCl₂.6H₂O, 0.03 g of CuCl₂.2H₂O, 0.05 g of NiCl₂.6H₂O, 0.05 g of (NH₄)₂Mo0₄, 0.05 g of AlK(SO₄).2H₂O; 500× vitamin solution prepared in a final volume of 1 L, 0.010 g of biotin, 0.010 g of folic acid, 0.50 g of pyridoxine-HCl, 0.025 g of thiamine-HCl, 0.025 g of riboflavin (cocarboxylase), 0.025 g of nicotinic acid, 0.025 g of D-Ca-pantothenate, 0.50 g of vitamin B12, 0.025 g of p-aminobenzoic acid, 0.025 g of lipoic acid (6,8-thioctic acid); 25 amino acid solution in a final volume of 1 L, 1.9 g of L-alanine, 3.1 g of L-arginine, 2.5 g of L-asparagine, 1.2 g of L-aspartic acid, 5.0 g of L-glutamic acid, 1.2 g of L-glutamine, 5.0 g of glycine, 2.5 g of L-histidine, 2.5 g of L-isoleucine, 2.5 g of L-leucine, 2.5 g of L-lysine, 1.9 g of L-methionine, 1.9 g of L-phenylalanine, 3.1 g of L-proline, 1.9 g of L-serine, 2.5 g of L-threonine, 1.9 g of L-tryptophan, 0.3 g of L-tyrosine, 1.3 g of L-valine; 5 mg/ml resazurin sodium salt; 10% (w/v) D-(+)-cellobiose consisting of 100 g in a final volume of 1 L; 1 M KH₂PO₄, adjusted to pH 6.8 with 10 M NaOH; 0.142 M MgSO₄.7H₂O; 0.544 M CaCl₂.2H₂O; 10% (w/v) yeast extract (Difco, BD Diagnostic Systems, Sparks, Md.) consisting of 100 g in a final volume of 1 L; 10% (w/v) casein hydrolysate (enzymatic; USB Corp., Cleveland, Ohio) consisting of 100 g in a final volume of 1 L.

Each liter of defined liquid medium is composed of 20 ml of 50× salts, 2 ml of 500× vitamin mix, 1 ml of 1000× trace minerals, 40 ml of 25× amino acid solution, 50 μl of 5 mg/ml resazurin, 50 ml of 10% cellobiose, and 2.4 ml of 1 M KH₂PO₄. When complex medium is desired, 5 ml of 10% yeast extract and 50 ml of 10% casein hydrolysate is added. The medium is brought to 1 L with distilled water. To reduce the oxygen in the medium, 3 g of L-cysteine HCL, 1 g of Na₂S, and 2 g of NaHCO₃ is added and adjusted to pH 6.4 with 1 N NaOH at room temperature. The medium is filtered through a 0.2 μm filter, distributed into smaller bottles, and the headspace flushed with at least three times with argon. To make 1 L of solid medium, the medium is prepared the same as above except the final volume is adjusted to 500 ml, and 2.5 ml of 0.142 M MgSO₄.7H₂O and 1 ml of 0.544 M CaCl₂.2H₂O are added to aid in polymerization. The headspace of the bottle is flushed with argon and placed at 95° C. Another bottle of 500 ml of distilled water with 10 g of phytagel is autoclaved and immediately combined with the first bottle. The medium is poured into polystyrene Petri dishes and inoculated immediately after solidification. The plates are put in modified paint tanks which are flushed with four to five times with argon before incubating.

The culture is incubated at 75° C. for 16 hours. Following the incubation, the culture is centrifuged at 3500 g for 15 minutes at 23° C. The supernatant is discarded and the pelleted cells are resuspended cells in 25 mL of room temperature 10% glycerol. The cells are washed twice by repeating the centrifugation and resuspension in 10% glycerol. After the final wash, the cell pellet is resuspended in 1 mL of 10% glycerol.

50 μL of cells are transferred to room temperature tubes for each electroporation. 30 ng of either replicating or non-replicating plasmid DNA in a total volume of 5 μL is added to each tube and mixed with the cell suspension. The cell/plasmid mixture is transferred to a 1 mm gap electroporation cuvette (to get 18 kV/cm). The cells are electroporated using an electroporator (Bio-Rad Gene Pulser, Bio-Rad Laboratories, Hercules, Calif.)) set to 1.80 V, 400 Ω resistance, 125 F capacitance, and 25 F capacitance at bottom.

The electroporated cells are transferred to 10 mL of complex medium with uracil and cytosine (described above) and incubated at 75° C. overnight. Following the overnight incubation, the cells are centrifuged at 3500 g for 15 minutes. The cell pellet is washed once by resuspension in 5 mL of 1× At salts (see above) and then recentrifuged. The washed cells are resuspended in 300 μL of 1× At salts.

The cells are plated by adding 100 μL of the cell suspension to a 4 mL tube containing 0.3% agar, then overlaying the cell/agar suspension onto either defmed medium with uracil (one plate) or defmed medium with uracil and 20 μg/mL hygromycin (two plates). The plates are placed in a jar and degassed by flushing the headspace with argon three to five times, then incubated at 75° C. for 60 hours. After 60 hours incubation, growth on plates with and without hygromycin is observed.

The efficiency of transformation is 1000 transformants per μg of replicating plasmid DNA and 100 transformants per μg of non-replicating plasmid DNA based on an average of at least three independent transformation experiments. The replicating plasmid is stably maintained after approximately 100 generations without selection.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

1. A method of processing plant biomass, the method comprising:
growing Anaerocellum thermophilum on a substrate that comprises plant biomass under conditions effective for the A. thermophilum to convert at least a portion of the plant biomass to a water soluble product or a water insoluble product; and
isolating at least a portion of the water soluble product or water insoluble product.

2.-3. (canceled)

4. The method of claim 1 wherein the conditions comprise a temperature of at least 70° C.

5. (canceled)

6. The method of claim 1 wherein the plant biomass comprises spent biomass.

7.-11. (canceled)

12. The method of claim 1 wherein the water soluble product comprises methanol, ethanol, butanol, fatty acids, hydrogen gas, succinic acid, citric acid, oxaloacetic acid, malic acid, adipic acid, fumaric acid, pyruvic acid, a monosaccharide, or a disaccharide.

13. (canceled)

14. The method of claim 1 wherein the water soluble product or water insoluble product comprises a biofuel.

15-17. (canceled)

18. The method of claim 1 wherein the A. thermophilum produces a water insoluble product that comprises alkyl fatty acids.

19.-21. (canceled)

22. A method of transferring one or more polynucleotides of A. thermophilum to a recipient microorganism, the method comprising:
providing an expression vector appropriate for the recipient microorganism comprising an A. thermophilum PBU polynucleotide; and
introducing the expression vector into the recipient microorganism.

23. The method of claim 22 wherein the recipient microorganism comprises Saccharomyces cerevisiae.

24.-26. (canceled)

27. The method of claim 22 wherein the recipient microorganism comprises an extremophile.

28.-34. (canceled)

35. The method of claim 22 wherein the recipient microorganism comprises a thermophilic microbe.

36.-39. (canceled)

40. The method of claim 22 wherein the A. thermophilum polynucleotide comprises a nucleotide sequence having at least 80% identity to the nucleotide sequence of a plant biomass utilization (PBU) polynucleotide.

41.-43. (canceled)

44. The method of claim 40 wherein the PBU polynucleotide comprises a polysaccharide hydrolases and related enzymes (PHR) polynucleotide.

45.-76. (canceled)

77. A genetically-modified microorganism comprising one or more A. thermophilum plant biomass utilization (PBU) polynucleotides.

78. The genetically-modified microorganism of claim 77 wherein the PBU polynucleotide comprises a nucleotide sequence having at least 80% identity to the nucleotide sequence of a PBU polynucleotide.

79. (canceled)

80. The genetically-modified microorganism of claim 78 wherein the PBU polynucleotide comprises one or more coding regions from a gene cluster chosen from: SYb001 and SYb037.

81. (canceled)

82. The genetically-modified microorganism of claim 78 wherein the PBU polynucleotide comprises a polysaccharide hydrolases and related enzymes (PHR) polynucleotide.

83-85. (canceled)

86. The genetically-modified microorganism of claim 77 wherein the microorganism comprises a eukaryote.

87. (canceled)

88. The genetically-modified microorganism of claim 77 wherein the microorganism comprises an extremophile.

89. The genetically-modified microorganism of claim 77 wherein the microorganism comprises a thermophilic bacterium.

90. The genetically-modified microorganism of claim 77 wherein the microorganism comprises a mesophilic microbe.

91. An isolated polypeptide comprising an amino acid sequence that is at least 80% identical to the amino acid sequence of a PBU polypeptide.

92. (canceled)

93. The isolated polypeptide of claim 91 wherein the PBU polypeptide comprises a PHR polypeptide.

94.-114. (canceled)

115. A method of processing plant biomass, the method comprising:
growing Anaerocellum thermophilum on a substrate that comprises plant biomass under conditions effective for the A. thermophilum to convert at least a portion of the plant biomass to a water soluble product or a water insoluble product; and
converting at least a portion of the water soluble product or water insoluble product to a biofuel or commodity chemical.

116. The method of claim 115 wherein the conditions comprise a temperature of at least 70° C.

117. The method of claim 115 wherein the plant biomass comprises spent biomass.

118. The method of claim 115 wherein the biofuel or commodity chemical comprises methanol, ethanol, butanol, fatty acids, hydrogen gas, succinic acid, citric acid, oxaloacetic acid, malic acid, adipic acid, fumaric acid, pyruvic acid, a monosaccharide, or a disaccharide.


SEQUENCE LISTING (163 sequences)

SEQ ID NO: 1 - DNA, 3653 bp, Anaerocellum thermophilum DSM 6725
aaaaaaattagagcaggatttgagcaagaggggcgctgcaaccccctttgaaaccgctttcataacccgaaaagctcttcacccaccgcatcaatattcatacccgcttgcataattattcatccctgattgtaccacaaaacccagaaaagtcctgggaaaatcatgaagattcttccattttccaatttccaggaagcgaattgaaattcccagggattttttgcctttttccagggaatgaactggaatttccagcggtttttccagggacttaaccacacgaaaggtactttccgccatcaaaggcttgcaaacccataggaaactactggcaggcaaaaccttccacccagtagcaaatctcttctaccttgttttgcgagcgttagcagggagctgtccgttagctttgtgttagcatccttctgttagcctttggcacgttagtagacttttgttagttctctcttcttgtttttatcttcattgtgattgttttccagaagactgcttgacattttgttattgtagtgttatgatctatttcagaagcgaaaagtaaaccgcggtagcgggttactttttgcgtaatatttggaattgtcagtttggcacgggaaatggaatcgggcaggtatagggaaaggcaatagccgtggacggcgttccgattccgctttgtttgtgcgttagcacatgagtcttgctgttatcacgctctgctctgtggtagtacaccgcaaggggtacaccatttggagttttgccgcatctgagagtttagcagtttgcaaaacaaagttcgcagttagaaaagttaggcaaggggcgatgacgtgcacgtctgacacgcttgacgtgcaccgagccgagagtagagcgtgatgacatagaaaaagcccagtagcaggttgtgcgtaccgtgacatgctactgtggaagacaagtaaatagtcaaaaaacggtgcgcttactgttcgtgggctcatgggaataccgtgagcattctggacaggttcaacagcctgaaagggcaaaaccccccttgaaagggtttaaatacacacgtcctttttgtcgttttttgtcggtttaaatatttatgcaggggatgatagggaatgggaaagccgtccataatcaagcaagtactcaatgaattcgaaaagcagataaggttcggggaaagtaagcatgaagcaaagagagaagagcgggagagatgtgaagttactggagagacgtggaatcctgctcgtgtggaaggcatttttagtttctcaacctacagggagtacgttaaggaggcgttagaatttgcgaattgggctcgtactgaaaaggggtgtaaggatttagaacaagcacgggcctatgtgtctgaatatttgcagtcgcatatagacaaagggtatagcgcgtggactgttaagaaagaagcagcagccctggcgaaactgtatcattgtcgtacaactgactttaaagtagagcttcccgcaagacacagggaagagattgagagaagcaggggatacaaagaccacgatagggagtttagcaaagagaggaatagagacattatcatcttttcaaaagctactgggctgagaagaagggaattggaaagagtgagttctcgggatatctttcgtgggcctgacggaagattatatgtgcacgtgagcaacggcaagggcggtagagaaagggatgttcatgttttgcagaaatacgagagagaggttgagaggatagtcagagagcgggaaggaagagacaggctgtttgacagggtccccataaggatggacgtacacagctataggagggaatatgcaagagagcgttacagagaagttgagcgtgagataagtcgtgagagaaagcttttcgacagagttgaggatctcgttcgtagtaggcttacaaggctctatcctgacaggtttagagaaattggcgaaagacaacttactcgtgaactcacaagagctgatgggctttatcatcgcagtgatggtagggagtttgaccgcctggcattgtgggaagtttcaaacgacttgggacataatcgaattgacgttgttgcaagacactatctggattaagcgaataaaggctcaagaaagtggataaaaaaacagggggtatattgatatatcccccctgttttttgtgcgtctacaggaccttatttgcgtttcaaggctattcttgtggggtagtgcagtaaaaaaggttgcctttgtatcttgcctgtagaatcttgccgttttcaaaaacaaattcaacgttgcacttatcagcaagtttcttgattattccctgctcaaattcgtcgaacctgtacccttccattgctcttgctgtgaaataacccagaaggtacattgcttcttcacgtgtaaactcaattgttagctttttcactctcttttaccccctttacgagactgagatattctttgtacaatctgtcaatacgttctgtatcgtcaggttgtacgtttttgagtttttcgtagagattatgaaacaacaggttctctgctgttccaatagggtatttctttctttccgcgtggactagccttttcagttggtttagctttacgttccggatcttgctcagtaggtcttcgacgatattttcaatctgttttgcttcgtcttcttcgatgtagaagcatagttgtttgatttcaagtagcctctgaataagttcctgctttgttaacattccttgtcacccttccccttttcagtcttttttattttgtcgtattctgattcgaactttctcagttggtcagctctgccgtaacgtatgcgtcgaggatttagcatgaatcgtccacggcctattttaatcaacaaatctttctcgattagctttttgattgcaagatggtattgcttttttgacatgtcgatgtttcctttgatgtcgtattcgtatgaaagtgcaaacgtgtagttgttaaacgagattttgtgttgtacaatgtatgagagaattcgcattgctgattttgcaaggtctttatcttccattatgagtttgatagaaatgaatggaagtttgacaaagtctgtgtcttcaaaatcatgtccgatcagtatgaaagtttgtacttcgcctgtttcagggtcgaccatttttttgtaccctatcttcttcatttgaatccccctttttttacctttttccaattctaaagccattataatacacttaaagtaacttgtcaagtaacttcaggtgtaaaaaattacacttcaggtgtaataaattacactttccattcagtattttcaaggctttgtgggtaactttattcttatctatgtatatatcgcctgcgttagcaggcttgaaaatttccagttaggataagcaggaacaacggtcgctgacgctgaacactgacgaaatagctgacgccccaaagtccacaacagtgccaaaccgataacaaaaacatgctaacgcaaacatagactaacgcacgactgacgtcgtgatgtgtgtgtgggcctacctacacacaaaaagaactaacaacagctgactaacgtctgaagagctctaacaacactttgctaacgctgagctaacggacagctcaacgttaacacccgct

SEQ ID NO: 2 - DNA, 20 bp, Artificial sequence
  source: /note=“Description of Artificial Sequence Synthetic primer”
cagcgttagcaaagtgttgt

SEQ ID NO: 3 - DNA, 21 bp, Artificial sequence
  source: /note=“Description of Artificial Sequence Synthetic primer”
agctaacggacagctcaacgt

SEQ ID NO: 4 - DNA, 36 bp, Artificial sequence
  source: /note=“Description of Artificial Sequence Synthetic primer”
agagagatgcataccagcctaacttcgatcattgga

SEQ ID NO: 5 - DNA, 36 bp, Artificial sequence
  source: /note=“Description of Artificial Sequence Synthetic primer”
agagagggtaccaggatctcaagaagatcctttgat

SEQ ID NO: 6 - DNA, 5280 bp, Anaerocellum thermophilum DSM 6725
atgaagcgttacagaagaattattgccatggttgtaaccttcatatttattttaggagtggtatatggagttaaaccatggcaagaggttagggctggttcgtttaactatggggaagctttacaaaaagctatcatgttttacgaatttcaaatgtctggtaaacttccgaattgggtacgcaacaactggcgtggcgactcagcattaaaggatggtcaagacaatgggcttgatttgacaggtggttggtttgacgcaggtgatcacgtcaagtttaaccttccaatgtcatacactggtacaatgttgtcatgggcagtgtatgagtacaaagatgcatttgtcaagagtggtcaattggaacatatcttaaatcaaatcgaatgggttaatgactattttgtaaaatgtcatccaagcaaatatgtatactattaccaggttggggatggaagtaaagatcatgcatggtggggacctgctgaggttatgcaaatggagagaccttcatttaaggtcacccaaagcagtcctggatctacagtagtagcagagacagcagcttccttagcagcagcttcaattgttttgaaagacagaaatcccactaaagcagcaacatatctgcaacatgcaaaagaattatatgagtttgcagaagtaacaaaaagcgatgcaggttacactgctgcaaatggatattacaattcatggagcggtttctatgatgagctttcttgggcagcagtttggttgtatttggcaacaaatgattcaacatatctcacaaaagctgagtcatatgtccaaaattggcccaaaatttctggcagtaacacaattgactacaagtgggctcattgctgggatgatgttcacaatggagcggcattattgttagcaaaaattaccggtaaggatatttataaacaaattattgaaagtcacttagattactggactacaggatacaatggcgaaaggattaagtatacaccaaaaggattagcatggcttgatcaatggggttcgttgagatatgcaacaactacagcatttttggcatttgtttatagcgattgggttggctgtccaagcacaaaaaaagaaatatatagaaaatttggagaaagccagattgattatgcgttaggctcagctggaagaagctttgttgttggatttggtacaaatccaccaaagagaccgcatcacagaactgctcatagctcatgggcagacagtcagagtataccttcatatcacagacatacattatatggagcgcttgttggtggtccaggctctgatgatagctacacagatgatataagtaactatgtgaacaatgaggttgcatgtgattataatgcagggtttgtgggtgcattagcaaagatgtatcaattgtacggtgggaatccaataccagatttcaaagctattgaaactccaacaaacgacgaattctttgttgaagctggtataaatgcatccggaactaactttattgaaattaaagcgatagttaataaccaaagtggttggcctgccagagcaacagataagcttaaatttagatattttgttgacctgagtgaattaattaaagcaggatattcaccaaatcaattaaccttgagcaccaattataatcaaggtgcaaaagtaagtggaccttatgtatgggatgcaagcaaaaatatatactacattttagtagactttactggcacattgatttatccaggtggtcaagacaaatataagaaagaagtccaattcagaattgcagcaccacagaatgtacagtgggataattctaacgactattctttccaggatataaagggagtttcaagtggttcagttgttaaaactaaatatattccactttatgatggagatgtgaaagtatggggtgaagaaccaggaacttctggagcaacaccgacaccaacagcaacagcaacaccaacaccaacgccgacagtaacaccaacaccgactccaacaccaacatcaactgctacaccaacaccgacaccaacaccgacagtaacaccaaccccgactccgacaccgactgctacaccaacagcaacgccaacaccaacatcgacgccgagcagcacacctgtagcaggtggacagataaaggtattgtatgctaacaaggagacaaatagcacaactaatacgataaggccatggttgaaggtagtgaacactggaagcagcagcatagatttgagcagggtaacgataaggtactggtacacggtagatggggacaaggcacagagtgcgatatcagactgggcacagataggagcaagcaatgtgacattcaagtttgtgaagctgagcagtagcgtaagtggagcggactattatttagagataggatttaagagtggagctgggcagttgcaggctggcaaagacacaggggagatacagataaggtttaacaagagtgattggagcaattacaatcaggggaatgactggtcatggatgcagagcatgacgaattatggagagaatgtgaaggtaacagcgtatatagatggtgtattggtatggggacaggagccgagtggagcgacaccaacaccgacagcgacaccagcaccgacagtgacaccgacacctacaccaacaccaacgtcaacaccaactgctacaccaacagcaacgccaacaccaacaccgacgccgagcagcacacctgtagcaggcgggcagataaaggtattgtatgctaacaaggagacaaatagcacaacaaacacgataaggccatggttgaaggtagtgaacactggaagcagcagcatagatttgagcagggtaacgataaggtactggtacacggtagatggggacaaggcacagagtgcgatatcagactgggcacagataggagcaagcaatgtgacattcaagtttgtgaagctgagcagtagcgtaagtggagcggactattatttagagataggatttaagagtggagctgggcagttgcaggctggtaaagacacaggggagatacagataaggtttaacaagagtgactggagcaattacaatcaggggaatgactggtcatggatgcagagcatgacgaattatggagagaatgtgaaggtaacagcgtatatagatggtgtattggtatggggacaggagccgagtggagcgacaccaacaccgacagcgacaccagcaccgacagtgacaccgacacctacaccagcaccaactccaaccccgacaccaacaccaactgctacaccaacaccaacgccaacaccaaccccaaccgcgacaccaacagtaacagcaacaccaacaccgacgccgagcagcacaccgagtgtgcttggcgaatatgggcagaggtttatgtggttatggaacaagatacatgatcctgcgaacgggtattttaaccaggatgggataccatatcattcggtagagacattgatatgcgaagcacctgattatggtcatttgaccacgagtgaggcattttcgtactatgtatggttagaggcagtgtatggtaagttaacgggtgactggagcaaatttaagacagcatgggacacattagagaagtatatgataccatcagcggaagatcagccgatgaggtcatatgatcctaacaagccagcgacatacgcaggggagtgggagacaccggacaagtatccatcgccgttggagtttaatgtacctgttggcaaagacccgttgcataatgaacttgtgagcacatatggtagcacattaatgtatggtatgcactggttgatggacgtagacaactggtatggatatggcaagagaggggacggagtaagtcgggcatcatttatcaacacgttccagagagggcctgaggagtctgtatgggagacggtgccgcatccgagctgggaggaattcaagtggggcggaccgaatggatttttagatttgtttattaaggatcagaactattcgaagcagtggagatatacggatgcaccagatgctgatgcgagagctattcaggctacttattgggcgaaagtatgggcgaaggagcaaggtaagtttaatgagataagcagctatgtagcgaaggcagcgaagatgggagactatttaaggtatgcgatgtttgacaagtatttcaagccattaggatgtcaggataagaatgcggctggaggaacggggtatgacagtgcacattatctgctatcatggtattatgcatggggtggagcattggatggagcatggtcatggaagatagggagcagccatgtgcactttggatatcagaatccgatggcggcatgggcattagcgaatgatagtgatatgaagccgaagtcgccgaatggagcgagtgactgggcaaagagtttgaagaggcagatagaattttacaggtggttacagtcagcggagggagcgatagcaggaggcgcgacaaattcatggaatggcagatatgagaagtatccagcagggacagcaacattttatggaatggcatatgaaccgaatccggtatatcatgatcctgggagcaacacatggtttggattccaggcatggtcgatgcagagggtagcggagtattactatgtgacaggagataaggacgcaggagcactgcttgagaagtgggtaagctgggttaagagtgtagtgaagttgaatagtgatggtacgtttgcgataccgtcgacgcttgattggagcggacaacctgatacatggaacggggcgtatacagggaatagcaacttacatgttaaggtagtggactatggtactgacttaggaataacagcgtcattggcgaatgcgttgttgtactatagtgcagggacgaagaagtatggggtatttgatgagggagcgaagaatttagcgaaggaattgctggacaggatgtggaagttgtacagggatgagaagggattgtcagcgccagagaagagagcggactacaagaggttctttgagcaagaggtatatataccggcaggatggatagggaagatgccgaatggagatgtaataaagagtggagttaagtttatagacataaggagcaagtataaacaagatcctgattggccgaagttagaggcggcatacaagtcagggcaggcacctgagttcagatatcacaggttctgggcacagtgcgacatagcaatagctaatgcaacatatgaaatactgtttggcaatcaataa

SEQ ID NO: 7 - protein, 1759 aa, Anaerocellum thermophilum DSM 6725
MKRYRRIIAMVVTFIFILGVVYGVKPWQEVRAGSFNYGEALQKAIMFYEFQMSGKLPNWVRNNWRGDSALKDGQDNGLDLTGGWFDAGDHVKFNLPMSYTGTMLSWAVYEYKDAFVKSGQLEHILNQIEWVNDYFVKCHPSKYVYYYQVGDGSKDHAWWGPAEVMQMERPSFKVTQSSPGSTVVAETAASLAAASIVLKDRNPTKAATYLQHAKELYEFAEVTKSDAGYTAANGYYNSWSGFYDELSWAAVWLYLATNDSTYLTKAESYVQNWPKISGSNTIDYKWAHCWDDVHNGAALLLAKITGKDIYKQIIESHLDYWTTGYNGERIKYTPKGLAWLDQWGSLRYATTTAFLAFVYSDWVGCPSTKKEIYRKFGESQIDYALGSAGRSFVVGFGTNPPKRPHHRTAHSSWADSQSIPSYHRHTLYGALVGGPGSDDSYTDDISNYVNNEVACDYNAGFVGALAKMYQLYGGNPIPDFKAIETPTNDEFFVEAGINASGTNFIEIKAIVNNQSGWPARATDKLKFRYFVDLSELIKAGYSPNQLTLSTNYNQGAKVSGPYVWDASKNIYYILVDFTGTLIYPGGQDKYKKEVQFRIAAPQNVQWDNSNDYSFQDIKGVSSGSVVKTKYIPLYDGDVKVWGEEPGTSGATPTPTATATPTPTPTVTPTPTPTPTSTATPTPTPTPTVTPTPTPTPTATPTATPTPTSTPSSTPVAGGQIKVLYANKETNSTTNTIRPWLKVVNTGSSSIDLSRVTIRYWYTVDGDKAQSAISDWAQIGASNVTFKFVKLSSSVSGADYYLEIGFKSGAGQLQAGKDTGEIQIRFNKSDWSNYNQGNDWSWMQSMTNYGENVKVTAYIDGVLVWGQEPSGATPTPTATPAPTVTPTPTPTPTSTPTATPTATPTPTPTPSSTPVAGGQIKVLYANKETNSTTNTIRPWLKVVNTGSSSIDLSRVTIRYWYTVDGDKAQSAISDWAQIGASNVTFKFVKLSSSVSGADYYLEIGFKSGAGQLQAGKDTGEIQIRFNKSDWSNYNQGNDWSWMQSMTNYGENVKVTAYIDGVLVWGQEPSGATPTPTATPAPTVTPTPTPAPTPTPTPTPTATPTPTPTPTPTATPTVTATPTPTPSSTPSVLGEYGQRFMWLWNKIHDPANGYFNQDGIPYHSVETLICEAPDYGHLTTSEAFSYYVWLEAVYGKLTGDWSKFKTAWDTLEKYMIPSAEDQPMRSYDPNKPATYAGEWETPDKYPSPLEFNVPVGKDPLHNELVSTYGSTLMYGMHWLMDVDNWYGYGKRGDGVSRASFINTFQRGPEESVWETVPHPSWEEFKWGGPNGFLDLFIKDQNYSKQWRYTDAPDADARAIQATYWAKVWAKEQGKFNEISSYVAKAAKMGDYLRYAMFDKYFKPLGCQDKNAAGGTGYDSAHYLLSWYYAWGGALDGAWSWKIGSSHVHFGYQNPMAAWALANDSDMKPKSPNGASDWAKSLKRQIEFYRWLQSAEGAIAGGATNSWNGRYEKYPAGTATFYGMAYEPNPVYHDPGSNTWFGFQAWSMQRVAEYYYVTGDKDAGALLEKWVSWVKSVVKLNSDGTFAIPSTLDWSGQPDTWNGAYTGNSNLHVKVVDYGTDLGITASLANALLYYSAGTKKYGVFDEGAKNLAKELLDRMWKLYRDEKGLSAPEKRADYKRFFEQEVYIPAGWIGKMPNGDVIKSGVKFIDIRSKYKQDPDWPKLEAAYKSGQAPEFRYHRFWAQCDIAIANATYEILFGNQ

SEQ ID NO: 8 - DNA, 945 bp, Anaerocellum thermophilum DSM 6725
atgagaaaaccgggtaaaattgtaattattggaactggctttgtaggctcatcaactgcttttgctcttgtagatgccgggcttgcaacagaacttgttttaattgatgtaaaccgtgcaaaagccgaaggtgaagccatggatttaaatcacggaatatcctttgtaaaacccgtcaagatatgggcaggtgattatgaagattgcaaagatgctgatataataataatcactgctggtgccaaccaaaagcctggtgaaacaaggcttgatttgacttataaaaatgcacaaattacaaagtcgataattgaaaatattatcaaatatacgcatgatgcaatacttttaatggtaacaaaccctgttgatgttctcacgtatgtaatgtataaagtttcaggcctgccaaaaaatcaggttataggttctggaacagtcttagactcatcacgatttagataccttttggcacaacactgccaggttgatgtgagaaatgttcatgcatatatattgggcgaacatggagacagtgaaattgctgcctggtctcttacaaatataggcggcgtgaattttatgcaggagtgtctattatgcgggaaaaattgctcacctgaagtaaaagagcaaatattcaacaaagtaaaaaatgctgcatacgaaataattgaaagaaaaggagcaacatattacgccattgcattggctgttagaagaattgttgaagctataatcagagatgaaaattctatactgcctgtatcatcaatagttgatgacgtatatggtgtaaaagacgttgcaatttcccttcctgcaattatcaacaaaagcggagttgtaaaagtatttgatataccactgacagatgaggaaaaagaaaagcttaaaaactctgctcaggtaataaaaagtgtgatagagtctttaaaactataa

SEQ ID NO: 9 - protein, 314 aa, Anaerocellum thermophilum DSM 6725
MRKPGKIVIIGTGFVGSSTAFALVDAGLATELVLIDVNRAKAEGEAMDLNHGISFVKPVKIWAGDYEDCKDADIIIITAGANQKPGETRLDLTYKNAQITKSIIENIIKYTHDAILLMVTNPVDVLTYVMYKVSGLPKNQVIGSGTVLDSSRFRYLLAQHCQVDVRNVHAYILGEHGDSEIAAWSLTNIGGVNFMQECLLCGKNCSPEVKEQIFNKVKNAAYEIIERKGATYYAIALAVRRIVEAIIRDENSILPVSSIVDDVYGVKDVAISLPAIINKSGVVKVFDIPLTDEEKEKLKNSAQVIKSVIESLKL

SEQ ID NO: 10 - DNA, 972 bp, Anaerocellum thermophilum DSM 6725
atgaagatacttgtaacaagaagaataatggaacctgcgattgagcttttgaaaaaatatggtgaggttgaagtaaatccacacgacagaccaatgacaagagaagaacttctgtcagcaataaaagacaaggatgcggttttaacccagcttgttgacaaggttgacaaagagtttttcgaccatgcaccaaatgtcaagattgttgcaaactatgcagtggggtacgataacatagatattgaagaggcaacaagaagaggtgtttatgtcacaaacacacctgatgttcttaccaacgcaacagctgagcttgcatgggcgctgttgtttgctgcggcaagaagaatagttgaagctgacaagttcatgagaggtggacattacaaaggctggggaccaatgctgtttttgggcaaaggtgtgacaggtaaaacgcttggtgtgattggtgcaggtagaattgggcaggcttttgcaagaatgtcaagagggtttaatatgaagattttgtactatgactttgaaagaaaagaaagttttgaaaaagagcttggtgcccagtatgtgccgcttgatgaactattaaaagaagctgattttatttcaatacatgtacctctcacaccacagacaaggcatttaattggtgaaagagaattttctctcatgaaaccatcggcaattttaattaacacagcacgtggaccaattgtagatgaaaaggctttagtcaaagcgcttaaagaaaagaagatttatgctgcaggacttgacgtgtacgagagagaacctgagtttgagccagaactggctgagcttgacaatgttgtaatgcttcctcatattggttctgcaacagaagagtcgaggcttgacatggcaatgcttgcagcaaacaatatagtagatttcattgaaggaagggttccaagaacacttgtcaataaagaggttttgaacaagaaataa

SEQ ID NO: 11 - protein, 323 aa, Anaerocellum thermophilum DSM 6725
MKILVTRRIMEPAIELLKKYGEVEVNPHDRPMTREELLSAIKDKDAVLTQLVDKVDKEFFDHAPNVKIVANYAVGYDNIDIEEATRRGVYVTNTPDVLTNATAELAWALLFAAARRIVEADKFMRGGHYKGWGPMLFLGKGVTGKTLGVIGAGRIGQAFARMSRGFNMKILYYDFERKESFEKELGAQYVPLDELLKEADFISIHVPLTPQTRHLIGEREFSLMKPSAILINTARGPIVDEKALVKALKEKKIYAAGLDVYEREPEFEPELAELDNVVMLPHIGSATEESRLDMAMLAANNIVDFIEGRVPRTLVNKEVLNKK

SEQ ID NO: 12 - DNA, 1200 bp, Anaerocellum thermophilum DSM 6725
atgaaggttttagtattaaattcaggaagttcatctttaaagtatcaatttattgataccgatacagaggttgctctttgtaaaggtgttgttgacaggattggtttgccgggggcatttattagacatcaaaagaatggtcaagagattgtaaaagaacaggaaataaatgaccacaatgttgctattaagcttgtgttagagatgcttacacatgaagaagctggtattatccattccatggatgaaattgatgcaattggtcacagggttgttcatggtggggaatattttagtgatgcggtaattgtcaatgaagaggtaaagaaagcaataagggaatgtattgaacttgcgcctcttcacaatcctgctaatttaatggggattgaagcatgtgaaaaagagattcctgggaaacctaatgtggctgtatttgatacagcatttcatcaaacaatgccaagatatgcgtatatgtattcgcttccatatgaggtgtatgaaaaatataaaattagaaaatatggattccatggaacatcacacaaatatgttgcaattaaagctgcagagtacttaagaagacctcttgaggagttaaaacttataacatgtcatcttggaaatggttcgagtgtatgtgcaataaagtacggaaaatcagttgatacaagcatgggatttactcctttggctggtcttgcaatgggaacaagaagcggaacaattgaccctgctgtgatactctatcttatggaaaaagaaaaaatggatgtaaagcagatgaatgattttctgaataaaaagtcgggtgtgcttggtatatcaggtgtgagcagtgactttagagatttagaaaaagctgcaaatgagggtaatgaaagagcacagcttgcaattgacatgttctgttacagggttaaaaagtatattggtgagtacgcagcggtcttgggtggagtagatgcaataatatttactgctggaataggtgaaaataatgctcttgtgagagataaatgtttgactgatttagagtatatgggtgtcctgtacgatagagaaagaaacttcaatgtagaaaaaggcaaggtttttgaaataaacaaacctgagagtaaggtaaaggttttaatagttcctacaaatgaggaacttatgattgcaagagagacaaaaagacttctttcaaaataa

SEQ ID NO: 13 - protein, 399 aa, Anaerocellum thermophilum DSM 6725
MKVLVLNSGSSSLKYQFIDTDTEVALCKGVVDRIGLPGAFIRHQKNGQEIVKEQEINDHNVAIKLVLEMLTHEEAGIIHSMDEIDAIGHRVVHGGEYFSDAVIVNEEVKKAIRECIELAPLHNPANLMGIEACEKEIPGKPNVAVFDTAFHQTMPRYAYMYSLPYEVYEKYKIRKYGFHGTSHKYVAIKAAEYLRRPLEELKLITCHLGNGSSVCAIKYGKSVDTSMGFTPLAGLAMGTRSGTIDPAVILYLMEKEKMDVKQMNDFLNKKSGVLGISGVSSDFRDLEKAANEGNERAQLAIDMFCYRVKKYIGEYAAVLGGVDAIIFTAGIGENNALVRDKCLTDLEYMGVLYDRERNFNVEKGKVFEINKPESKVKVLIVPTNEELMIARETKRLLSK

SEQ ID NO: 14 - DNA, 999 bp, Anaerocellum thermophilum DSM 6725
atggcatttattgatacaatcatagaaaaagcaaaatcagatataaaaacaattgtactacccgaaagctacgaagaaagaaatttaaaggctgcttctatagctttgaaagaaaagatagctaagatagttttgattggcaaagaagatgagataaaaaaagaggcaacaaagtttggtgcagatgtagatgaagctatttttattgacccagacaattttgatagatttgatgaatttgtaaatgaattttatgaactaagaaaaaacaagggtgtaacattggaagatgcaaaaaagtttatgaaagacccgatgtattttggtgttatgcttgtgtacaaaggtttggcagatggtatggtgtctggtgctattcactcaacagcagatacattaagaccggctctgcagatattaaaaactgcacctggggtaaaacttgtttcaagcttctttattatggttgtaccaaactgcgaatatggtgaaaatggagtttttgtatatgctgatgcaggtttgaatccaaatccaacagcagaagagcttgctgatatagctattacatctgcaaagagctttgaagctttagttggtaaaactccaaaagtagcaatgctttcatattcaaccaaaggttctgcaaagtctgagatggttgacaaggttgttgaggcaacaaggattgcaaaagagaaagcaccagacattttaatagatggcgaacttcaagcagacgcagcaatagttccttctgttgcaaagctgaaagcgccaggaagtcctgttgcaggacaagcaaatgttctaatcttccctgatttggatgctggcaacattgcatacaaacttacagaaaggcttgcaaaagcagaagcgtacggacctattacccagggaatagcaaaacctgtaaatgatttgtcccgaggttgcaaagctgaagacattgtgggggttattgctattactgctgtacaggctatgatgaaataa

SEQ ID NO: 15 - protein, 332 aa, Anaerocellum thermophilum DSM 6725
MAFIDTIIEKAKSDIKTIVLPESYEERNLKAASIALKEKIAKIVLIGKEDEIKKEATKFGADVDEAIFIDPDNFDRFDEFVNEFYELRKNKGVTLEDAKKFMKDPMYFGVMLVYKGLADGMVSGAIHSTADTLRPALQILKTAPGVKLVSSFFIMVVPNCEYGENGVFVYADAGLNPNPTAEELADIAITSAKSFEALVGKTPKVAMLSYSTKGSAKSEMVDKVVEATRIAKEKAPDILIDGELQADAAIVPSVAKLKAPGSPVAGQANVLIFPDLDAGNIAYKLTERLAKAEAYGPITQGIAKPVNDLSRGCKAEDIVGVIAITAVQAMMK

SEQ ID NO: 16 - DNA, 1641 bp, Anaerocellum thermophilum DSM 6725
atgaaatttttaactgcagatgtattaaaagatatgttaaaagctgcaaataattatttaaaattgcacatagataagataaactcattaaacgtctttccagtaccagatggtgacacaggcaccaatatgtctgccactcttgacagcagcataaaagaaataaatggaaagactttcgaaaatgtggacaaacttatgaatgcagttgcgtttggcagcttaaaaggtgcacgcggtaattctggtgttattctttctcagcttttacgcggatttgccaaagagctaaaaggcaaagatgttatagatataccaacatttgttgcttgtttaaaatctgcgtctgcaagtgcttacaaagcagtgatgaagcctacagaaggcactatgctcacagttgcacgcgggattgcagaggatgttgaaaaagaagtggcagaaggcattgtgagtgaaatagaggatttgctggaagtgtgtgtttcaagcgggaagaagtggcttgcaaagacaccagagatgctttctattttaaaagaggcaaatgtagttgacagtggcggtatgggtcttgtaataatttttgaagggatgtataaattcttaaaagaaggaatggtatttgaagagccatcacagcaggaagtttatacagtcctcacttttaaacctgaagatattaagtttacttactgtacagagttttttattaccggtttgaaaaagaatattgaaaaagaatttaaggaatatcttgagacaattggtgattcaattattgtaatccaagatggcgacattctcaaaacacacgttcacacaaattcacctggcaaggtaatagaaaaggctttgaaatatggtgagcttataaatataaagattgataatatgaaatatcagcaccaggagtttataagtaaaagagaaaaccatgagacagaacttcaaacacaggctgaagttattataaaagaatatggttttgtagctgtatcacaaggagaaggattcaatgaaatattaaaaggcttgggtgttgattttgtaattgaaggcggacagactatgaatccaagcgctgaggactttgtaaatgctataaagaatgtaccagccaaaaatgtatttattttcccgaacaataaaaacgtgattatgtcagcagagctttctttacagcttattaatacaaataaaaatatagtgattatgaagacaaccaatattcctgagtgcattactgcaatgataaagtttgatttgaacaagagtattgaagaaaatataaagctcatgcagcaagctataaactcagtaaaggttgtagaaataactaaggcagtgagaaatacaaaaataaacgggtttgagattgaagaaggcgattttatagggatttccaaaaaggaaataattgcatgtgacaaagatatgttaaaagtagctttggcttgtgtcgaaaagattgttgattctacaacccagattttgagtatttactatggcaaaggtgtagccttagaagatatagaggtgcttgttaaaaacatacaagaaatatacccgaaaattgacattgagagctatgaaagtggaaatgaaatttatcaattaattattgtagctgagatgtga

SEQ ID NO: 17 - protein, 546 aa, Anaerocellum thermophilum DSM 6725
MKFLTADVLKDMLKAANNYLKLHIDKINSLNVFPVPDGDTGTNMSATLDSSIKEINGKTFENVDKLMNAVAFGSLKGARGNSGVILSQLLRGFAKELKGKDVIDIPTFVACLKSASASAYKAVMKPTEGTMLTVARGIAEDVEKEVAEGIVSEIEDLLEVCVSSGKKWLAKTPEMLSILKEANVVDSGGMGLVIIFEGMYKFLKEGMVFEEPSQQEVYTVLTFKPEDIKFTYCTEFFITGLKKNIEKEFKEYLETIGDSIIVIQDGDILKTHVHTNSPGKVIEKALKYGELINIKIDNMKYQHQEFISKRENHETELQTQAEVIIKEYGFVAVSQGEGFNEILKGLGVDFVIEGGQTMNPSAEDFVNAIKNVPAKNVFIFPNNKNVIMSAELSLQLINTNKNIVIMKTTNIPECITAMIKFDLNKSIEENIKLMQQAINSVKVVEITKAVRNTKINGFEIEEGDFIGISKKEIIACDKDMLKVALACVEKIVDSTTQILSIYYGKGVALEDIEVLVKNIQEIYPKIDIESYESGNEIYQLIIVAEM

SEQ ID NO: 18 - DNA, 120 bp, Anaerocellum thermophilum DSM 6725
atgaaagttaagaaaaagaagataggtagtgttattatgatttttgcaatattgttgagcgttattgttccagttttaacggctacatatcaaagtaaaagttatttagaaactacataa

SEQ ID NO: 19 - protein, 39 aa, Anaerocellum thermophilum DSM 6725
MKVKKKKIGSVIMIFAILLSVIVPVLTATYQSKSYLETT

SEQ ID NO: 20 - DNA, 93 bp, Anaerocellum thermophilum DSM 6725
atggtttttaaaagtgataaaaacttaatttacttggttacaggaaaagtgaagaaaaatgaccttggcatgggaatttacatttgcgaataa

SEQ ID NO: 21 - protein, 30 aa, Anaerocellum thermophilum DSM 6725
MVFKSDKNLIYLVTGKVKKNDLGMGIYICE

SEQ ID NO: 22 - DNA, 9084 bp, Anaerocellum thermophilum DSM 6725
gtgagatttccaaagaaattgagagtattgttaacatttcttattcttttaatcttcacccttaatacaaatctatatgtcttttcacaaagtagtaatctcaagaaccaagagtcaaattacaaagatttaataggacactgggcagaggatgaatttagatggttgatagagaagggaataattagtggagtgaaggcaaatggagtaatgttagcaaagccagatgagaagttaacaagagcggaagctgttgtgattatactaagaactatatttgagaaggagcttcttgaggaggaaataaagaaattaaaagaggatagttttaaagatatttcaaatcattggagcagagattacataaatgtagctgcgagatatggtttggttaaaggttattcagacaagacatttagaccaaatcagagaattacacgtgaagagtttgttttaatggtgataagggtgagcaaatatagggagcaagcagcaagtgagggcaagcaaaacgataagaaggtaaaatttaaagatgtgagtgttaacaattttggatataacgaaataatttttgcagttgagaaaggcttgatcaaaggctatagcgatggtacatttagaccaagaagttatataagccgagcagaagcagcagtaatagttgcaagggcattgaaagcggatttattcattacctatagagcaaagaagcctcaaataactaaagaaaatgcgagcgaattttttgaacttgttgttgataaagaggaagtaggtataatagaaaaggcgaagctcacactaaagagcaaagtaaagggtgtcaagtttacagcagagtggaaggcaagtggtggtaaacttgaggtagctaaagataatcagagtgcgatttggagcccgacagatgcagaagagggtaagaattatattgtgactgttgaagtaaccatcctgacagataatggggaaaagataaaaatacaaaaggttgcaaggatacgagtgaacgagggcgtccaactgacaaagaatgaaaacatagaagaaccggtgaataaaatagatccattatttacccaaagagaatcaaacaatttagttcaagaaaattctagttatagcccattagaagaaaaactttcagcgccaatattgaaaatagcaaaaaaaggtgagtatgtagagcttacttggaatgaggtaaaaggtgcaaggagttacattgtaaaaagagggaaggtaagcggggagtatgaggtattggcaagcggagttattactacggcgtatattgatggtcctatggatggaaaaacaacgtattactatgtggttgcagcagtgggagcaaatggtacgaccagtatgaattcaaatgaggtagtgtacaaagcactgccacaatcaccaaagttatttgggacatacaatgggacaagtgcacgtcttacatggacgaaagcgaatggagcagagaggtatactctttacaggagcacagtcagtggaggaccatactatgcaattgcagaaaacttacttaccaatgtgtttgaagatactaatttgacatcaggtacagtgtattactatgtagtaaaagccattaacgaattaggagaaagtgaatactcaaatgaggtagcgatgggggaaggagttacagttgcagctaaatttaatcctgatgacgacgaggatggagatgggttgacaaatgtagatgagttaaaatatgcaacaagtttgaagaagaatgatagtgatggagatgggttgagtgacggttatgaagtaaagaaaggaacaaatccattagtaccggatacagataatgacgggatatatgatggagcagaggttgtgatgggaacagacccattgacaaagaatcctttgacaagtgccgagaaatatgcggtatcggaagatggtaaagtttttgtaagagcactttctgatgcaaatattttaatagctcccttacaggtaaagaggtctgacaatgtgtttattaactcattgaaaggaatagtaggtaaagcgatagagataactgcagggggatttgatataaagaaggcagagatagtagtgaactatgatgaagcagaacttaatggtgtagaagagaataatttaatgctgtattatgtcaattatgacaagaagatattagagccgttagaagatgttgtagtagatacagtgtataatcgagtttcgggtaagacagagcattttagcacatttttacttggtgataagaatatgccagttgatctttccaaagtagatattgtgtttgtacttgacaactcaggtagtatgtcgtctaatgacccaaattattacagaatagaagcaacaaagaaatttatacaaaatatagatgaacttaacaatagagttgggttagtagatttcgatagctctgtaagtgttagatcaaatttgacatctgacaagagcaaattgttacaagccctaaatgcgatgagatggacaggtggttctacgaatataggaggaggattgaaagctgcgctgggattattcgaccaagagcaatctaaaaagataatagtacttttatcagacggatatcacaacacaggaattcatccaaacgatgtactaccagaattaataaaacaagaaatagtagtaaataccatcgcattgggtaaagattgtgatagggagttattacatgatatagctgataagacaaaaggaggctatttttacgttgataatacaggaggactttctcaagaagatgtagacaagcaaatagagctaatatacgagaaactaactaaatggataacccttcaaaaggaagcagaaaagaatctcaaacctcaagaagtgttaagtatagagtacaatgatgtaggtttagacaatgaagagtttcataaatggataaccacagctatgactaatttacttacgggaaattatatggaagagtttgatgatattagtatagaggggaatggtcctgagtttaaatttgtgaggtattacaattcttttgctaatcaacaaaagacgataataggtaaaggttttagaaccaattttgacagcaagcttacaaaggtagcgggagtcggaatagtccaagcaggggtattgaatgtaagagaaggaccgaatgtaaataccaaaaagatagggtatttgactaaggggacaaaggtgcagattgaagaggatggcaacaagaacggcagtggttggcacagaattgtgtataagggtaaatctgcatatatttgcgcagcatatgtcaaagagctaaacaatggaattgaagtaacatatccaagtggtagtatgatagtatttgtggatgataatggagatgggatttacttgtcagacagtaacaaagctgacaaggtagttttgtcaggtaatgaatatatattgatacaaagagatttgacgagatatgtttttgacaagagtggaaaacttatcaaaattggtgatagaaatgaaaattacattactattgagtattctggtgaaaagatatctgcggcaagagatgtttttggcagaaaattagaatttatctttcaaggggataacctggtatgtataagagagaacatcaagggtaaaataggtaggaaagtagaatttgtatatgatgacaaggatagactaataaaagtaattggggttgatggcgcagagacaagatatgaatatgatgagaaagacagactcaagaggataattgatgcgaacgggcaccaggtggtaaggaatgaatatgatattttaggtaggatagtgaggcaatacgacggggaagatataatcagatttttcatatacgatgatgaagacagagtaagatattatatagacgaaaatggtaatgagagtatggtagtattcaatgaagagctaaaaccaataaaagagagaaatgcattgggtgggggttgtgactacaagtatgagattaatgacggttcaaagtggatagacgtgacaactcctgatttggataaggatgtagttgtgagcggtcttacacgggaaaaatatcaagagctgaaagagaaaggtagcatgacaaagcaggtcacaattcagataataaagacttctcctagtttagaaacagcaaagactacacaaatatacgatggacgtggcaatattatccaagtgatagatgcttatggtaattcaataaaaatgaaatatgataacaataataatttaattgagcaaacagacaggattggtgctactaccaaatatatttatgatgccgagggtataaatttaattgagaaaattgatccacttggaaacaaggaaagatatgaatattattctataaattcgggcataaaactcaatggtttacttgcaaagtatatagataaaaatgggaatgagacgagatactattatgaagatgaatataacaatttaacacgagtggttgacgcagaaggttatgagacaaaatatgaatatgaccagttaggcagaaaaataaaagaaataaatgaaagaggatatgtaacaaggtttgagtatgatccagaaggcagaattacaaaagaaatagacgcgtttggtaaaacaaaagtatatgtatatgacaaagtaggtaatttaatagaagaaatcgatcgccttggcaacaaaacaagatatgtatacgatgataaaaacaggcttataaaagaaattgatgcgatgggtggagagtatcaatacttttatgatcctgtagggaacaagataaaggagatagaccctgaaggaagagtaaccaaatatacttatgatgaactgaacagacttgttgagatagaagatgcagaaggcaataaaaccaaattcaagtatgatttagcaggcaggaaaatatcagaggtaaatgccttaggaaaagagacaagatatgaatatgatttattaggaagacttacgaaagtgattgatcctttaggtaaaacccgaagttaccaatacaatgctgaagggtataaaatatcagagacgaataaaaatggtgccactacttcatatgtatatgatttagcaggaaggttaataacagcatattatccagatggaactagaaggagctataactacgataataatggcaatgtaatcagtatcatcaatccaaaaggatatgtgacaaaatactactacgacaaattaaacagggtaatcaaggtagaagacagcaacggcaaagcagtaacatatgagtatgacggttgtggaaatgtaatttgctttaaagacaaaaaaggaagagaaacaaggtacgaatatgatgctcttgacagagtaaaaagagtaatagcaccaaacggagcacaaacagagtatgagtacgatgcagagggcagagttgtcaaggtcacagatgcaaaaggaagaagcgaggaatatatgtatgatgagcttggtagagttgttgtatataaggacaagttaggaaatgtaatcaaatacgcatatgacaaggtgggtaatagaacacagcttattgatagaagaggaaatgcaacaaagtatgagtatgataaattgaatagggtagtaaaggtgattgacgcgtatggaaatgaaagtaggctggagtatgatgctgttgggaataatatagcaaagacagacagaagaggaaacacaacaaagtatgaatatgatgcaaataacagactagtgacaataattgatccatatggaaataagattagatttgagtatgatggagaaggcaatgtaatatgcagaatagatgcaagagggaacagaatgtattatagttatgatggattgaacaggttgaggactgtacaagacaacgatggcaggaaaactgtttatgaatatgatgagaatgggaatatagtaaagataatacgaccagacggcaagtatgtaacctacaggtatgacagtttggacaggttggtaagagttacacaagagaatggagctgtgacagagtataggtatgacgaagaggataatttaattgaggtcaaggatgggaatggcaatataacaaggtatgaatacaatgagatagacagacctgtcaaagtaatagatgcaatcggcaatgaagaaagatatagctatgacttagtagggaatatagtatatgcgatagacaagaatggagttaggatagagtacagctatgaccaacttgacagggtagtgcatgttaaagctggcggagtagaggtgagatatagttatgatgaggaaggcaacagagtccagatgtcagacaaaacagggataaacaaatatgaatatgataagctaaataggttgataaggaaaatatacccagatggcaagagtatagaatatgaatacgaccaagaaggtaatgtaataaaggtaaaagacccgagcggatatgtgacgcaatacaagtatgataagatgaatcggcttgaagaggtgataacctcagatggtagcacaagatattcatacgatgagaatggaaatgtgaaatcaatagagtatccgaataagctgaagtttgagtacgaatatgatagcaggaacttacttaaaggattagtggttacagcgagagatgttgtaaggaatgagatagataagtattatactcctagcagtgtgatagaacaaggtggtggcacaagcacatatatttacaagtatgaatatggatatgatgacaatggcaatatgatatacaaacaggagcctataggtaggacagagtacaagtatgatgaggtaggtagagtagctgaagtcaaagatagatttggacgattaactcgatatgaatatgacaatgtaggcaatagaataaaggagatagtagaggtcccgacaggtataagcagagatagtcttaattctgaagggctgaatattagatacgattatggagaagtatacagcgttgagaagacttacacatatgataaattaaatagactattgagcattgaatctcgagacagagaagggaatattgtaggtgttaacagctacggctatgacaacaatgggaatttgataagagcagaggaaagatggcgcacaagagtatacaagggtcagggaagcaaagtagttgcaaaagagatagaaaagcaagatttacagactgtatatcaagatgcatatactgcagagaatgttcaggtacagacaagcgtatataactcagcatatagtttggacaatagcacagtagtgatttcagtatatgatagtgtgtacaatagtgcatacaatttacaaaattcaagttctgagcagagtattactgtaaaagacgtagtttacgagacggtatatgaagatgtttatgagatagaagagaagattaaagtaagtgaatttagatatgatgagcttggcaggatggtatgggcaaaggttgacaataatgtggtagaatttgaatacgacggagatgggctcagaacaaagaagataacagcaaatgatgtaaaaacatactactggtctggcagcaatcttatttatgaaagcgatgcaacgggcaaaggattcagcagcatatggggcttgagtatgataggaaggacggacggtagtaatacagagtattttatgaaagatgggcatggagatgtacttatcacgtttgacaaaagtgggcagaggaagaatatatatgaatatgatttgtatggtaatgtaataaaagaaatagagacagggcaagagaacccgataagatatgctggatattactatgacaaggaattagaatggtattatttaaagacaaggtattacgatagtaggatagggaggtttgtgaaagaggatggcataaagggagatataacagacccagaaagtttgaacctatacacttactgtacaaacaatcctgtgaatttatatgatccggatggagagtttgcgatagtgccactgttagtaggattagcagtgcagactgtaagtggagtattgttagattatataatagataggaaaaatttcaatctttggaagagtattggaacaaatttagtagtgtcaatggttggtatagtaacaggaggaatagcaagttcaataaagcttggtacaaaaataactaaagttgcaccgaaagtagcaaaactaacagaaagattagtaagatggggaagtgaaaaagctgttaaaaaacctaactctaaggttgcaaaatttataaataaatttacagaagatgttgcaaaaccttttattgataaattaggcactccaaagacatgggttatggctgggttaaaagcactgacccaaacaggtgcggatgcgacgatagatatgatatctggtgaaaaagttaccgcaggatcaatttctctcgatatacttttgagaactgcatttggtgcgtttggctcagcaacaggaaacgtgaaactatttggaggtggattgaggagaatatttaaattacctccctctaagaaagcggaaagttgggatgctgctgctaaaagagtagttgcaaaatatttgatagaagatttagaggcgggattatcaaattttggcaaaaagtgttcacttgatgcattaaagggtattaacttaggaaggatattgggacgagcaggaagaaagtttacaaaatatatagtaaataaattggaaatgcaaaagtattcaataacaaatgtttcagggatccaaggggaaagtggtggaggagtattagacgagtttattgttcaaaataataaaaaaacaggcgaggatgtaaaatggacaagaggaagtggagtaagtagtccagcgacaattggtatctggggcactgcaggtgaacatgaggttggacaaataaaaggagtagcagcacaaggattgaattttagctggagcatacaatcaaagatgggagctgatttaaacttaggggcggtaagaggaggcgagccgaggacgccgacagtagatttaaaattcggctatatggcacctatgagaaggatgatagtgccgagggtaggcagggtattagcatattga

SEQ ID NO: 23 - protein, 3027 aa, Anaerocellum thermophilum DSM 6725
VRFPKKLRVLLTFLILLIFTLNTNLYVFSQSSNLKNQESNYKDLIGHWAEDEFRWLIEKGIISGVKANGVMLAKPDEKLTRAEAVVIILRTIFEKELLEEEIKKLKEDSFKDISNHWSRDYINVAARYGLVKGYSDKTFRPNQRITREEFVLMVIRVSKYREQAASEGKQNDKKVKFKDVSVNNFGYNEIIFAVEKGLIKGYSDGTFRPRSYISRAEAAVIVARALKADLFITYRAKKPQITKENASEFFELVVDKEEVGIIEKAKLTLKSKVKGVKFTAEWKASGGKLEVAKDNQSAIWSPTDAEEGKNYIVTVEVTILTDNGEKIKIQKVARIRVNEGVQLTKNENIEEPVNKIDPLFTQRESNNLVQENSSYSPLEEKLSAPILKIAKKGEYVELTWNEVKGARSYIVKRGKVSGEYEVLASGVITTAYIDGPMDGKTTYYYVVAAVGANGTTSMNSNEVVYKALPQSPKLFGTYNGTSARLTWTKANGAERYTLYRSTVSGGPYYAIAENLLTNVFEDTNLTSGTVYYYVVKAINELGESEYSNEVAMGEGVTVAAKFNPDDDEDGDGLTNVDELKYATSLKKNDSDGDGLSDGYEVKKGTNPLVPDTDNDGIYDGAEVVMGTDPLTKNPLTSAEKYAVSEDGKVFVRALSDANILIAPLQVKRSDNVFINSLKGIVGKAIEITAGGFDIKKAEIVVNYDEAELNGVEENNLMLYYVNYDKKILEPLEDVVVDTVYNRVSGKTEHFSTFLLGDKNMPVDLSKVDIVFVLDNSGSMSSNDPNYYRIEATKKFIQNIDELNNRVGLVDFDSSVSVRSNLTSDKSKLLQALNAMRWTGGSTNIGGGLKAALGLFDQEQSKKIIVLLSDGYHNTGIHPNDVLPELIKQEIVVNTIALGKDCDRELLHDIADKTKGGYFYVDNTGGLSQEDVDKQIELIYEKLTKWITLQKEAEKNLKPQEVLSIEYNDVGLDNEEFHKWITTAMTNLLTGNYMEEFDDISIEGNGPEFKFVRYYNSFANQQKTIIGKGFRTNFDSKLTKVAGVGIVQAGVLNVREGPNVNTKKIGYLTKGTKVQIEEDGNKNGSGWHRIVYKGKSAYICAAYVKELNNGIEVTYPSGSMIVFVDDNGDGIYLSDSNKADKVVLSGNEYILIQRDLTRYVFDKSGKLIKIGDRNENYITIEYSGEKISAARDVFGRKLEFIFQGDNLVCIRENIKGKIGRKVEFVYDDKDRLIKVIGVDGAETRYEYDEKDRLKRIIDANGHQVVRNEYDILGRIVRQYDGEDIIRFFIYDDEDRVRYYIDENGNESMVVFNEELKPIKERNALGGGCDYKYEINDGSKWIDVTTPDLDKDVVVSGLTREKYQELKEKGSMTKQVTIQIIKTSPSLETAKTTQIYDGRGNIIQVIDAYGNSIKMKYDNNNNLIEQTDRIGATTKYIYDAEGINLIEKIDPLGNKERYEYYSINSGIKLNGLLAKYIDKNGNETRYYYEDEYNNLTRVVDAEGYETKYEYDQLGRKIKEINERGYVTRFEYDPEGRITKEIDAFGKTKVYVYDKVGNLIEEIDRLGNKTRYVYDDKNRLIKEIDAMGGEYQYFYDPVGNKIKEIDPEGRVTKYTYDELNRLVEIEDAEGNKTKFKYDLAGRKISEVNALGKETRYEYDLLGRLTKVIDPLGKTRSYQYNAEGYKISETNKNGATTSYVYDLAGRLITAYYPDGTRRSYNYDNNGNVISIINPKGYVTKYYYDKLNRVIKVEDSNGKAVTYEYDGCGNVICFKDKKGRETRYEYDALDRVKRVIAPNGAQTEYEYDAEGRVVKVTDAKGRSEEYMYDELGRVVVYKDKLGNVIKYAYDKVGNRTQLIDRRGNATKYEYDKLNRVVKVIDAYGNESRLEYDAVGNNIAKTDRRGNTTKYEYDANNRLVTIIDPYGNKIRFEYDGEGNVICRIDARGNRMYYSYDGLNRLRTVQDNDGRKTVYEYDENGNIVKIIRPDGKYVTYRYDSLDRLVRVTQENGAVTEYRYDEEDNLIEVKDGNGNITRYEYNEIDRPVKVIDAIGNEERYSYDLVGNIVYAIDKNGVRIEYSYDQLDRVVHVKAGGVEVRYSYDEEGNRVQMSDKTGINKYEYDKLNRLIRKIYPDGKSIEYEYDQEGNVIKVKDPSGYVTQYKYDKMNRLEEVITSDGSTRYSYDENGNVKSIEYPNKLKFEYEYDSRNLLKGLVVTARDVVRNEIDKYYTPSSVIEQGGGTSTYIYKYEYGYDDNGNMIYKQEPIGRTEYKYDEVGRVAEVKDRFGRLTRYEYDNVGNRIKEIVEVPTGISRDSLNSEGLNIRYDYGEVYSVEKTYTYDKLNRLLSIESRDREGNIVGVNSYGYDNNGNLIRAEERWRTRVYKGQGSKVVAKEIEKQDLQTVYQDAYTAENVQVQTSVYNSAYSLDNSTVVISVYDSVYNSAYNLQNSSSEQSITVKDVVYETVYEDVYEIEEKIKVSEFRYDELGRMVWAKVDNNVVEFEYDGDGLRTKKITANDVKTYYWSGSNLIYESDATGKGFSSIWGLSMIGRTDGSNTEYFMKDGHGDVLITFDKSGQRKNIYEYDLYGNVIKEIETGQENPIRYAGYYYDKELEWYYLKTRYYDSRIGRFVKEDGIKGDITDPESLNLYTYCTNNPVNLYDPDGEFAIVPLLVGLAVQTVSGVLLDYIIDRKNFNLWKSIGTNLVVSMVGIVTGGIASSIKLGTKITKVAPKVAKLTERLVRWGSEKAVKKPNSKVAKFINKFTEDVAKPFIDKLGTPKTWVMAGLKALTQTGADATIDMISGEKVTAGSISLDILLRTAFGAFGSATGNVKLFGGGLRRIFKLPPSKKAESWDAAAKRVVAKYLIEDLEAGLSNFGKKCSLDALKGINLGRILGRAGRKFTKYIVNKLEMQKYSITNVSGIQGESGGGVLDEFIVQNNKKTGEDVKWTRGSGVSSPATIGIWGTAGEHEVGQIKGVAAQGLNFSWSIQSKMGADLNLGAVRGGEPRTPTVDLKFGYMAPMRRMIVPRVGRVLAY

SEQ ID NO: 24 - DNA, 1248 bp, Anaerocellum thermophilum DSM 6725
atggagagtagagagagggtagtgaaagcgctgaataagctaataaaggcattggcagtaatttggttagtgttaattgttctgaggcttatatggtattttgcagacataacttatatatttgagagattatcgatagatacgacagatgttctatcattgttcttggaacaaacaataggttcttttttggcttatagtgtggctatattgattgtagctttatttactgtagtgttattttttttattaagttcaataattacggtgttgattgctggtttcacaacaacaatttcagaaaagattgaagcaatttttgtcaagaaggaatttggtgcgatgcaggtaaatgaagaagtaattgaagctagtttagatatagatgaagagatttatattgataggagtaagaggaaaataagcgtatttggcagatatgaaaaggccaaaaaaacggattcacttttaggagcggagcacaggattgatggagagttttttatttttaagtgtattaaagtgaaaggagaaaaattatatttaccgtactcattaattgaaaatagatatgtaaacggatatgttgtaggtaagaatatatcatactactgtgatggcaatgaatcagaaagaatgttattggtagacgcaattaaagaagaaataatagaaaatgaaaaagagcatgttgaagaaaaaatttgtggtgaaattctagcagaaaaatgtgctaaagctgctgcaaagttaattaagagtataacagaagaaagttttgatactgaaccaagatttaaggatatagtactaacgttattagttacaggggtatttttaataacaaattttgtgtcttggttgatatctaagatagcaagtagtgagataaatttgcaaggaatattatggattataaatataatgattttagtgttaatattagtactttacgtagttaagagattttatgaaaaagaaaaaccgcttaagtttgatatagagatagataaggtacatgtaatattttgtgaaagttctgaaggaataatagaaacagtaataaaagaaaaagaagcatttgtaaaagatgaatacatagtgttagtaaaaaaaggagatgaagaaggcgagagatttttggggaaattaaaagaagaaaaagaaaaaggattgtatgaagtaagattaatatatggtaccaaaaaagagaacaaaataaagtattgggtaaaattaaggagttga

SEQ ID NO: 25 - protein, 415 aa, Anaerocellum thermophilum DSM 6725
MESRERVVKALNKLIKALAVIWLVLIVLRLIWYFADITYIFERLSIDTTDVLSLFLEQTIGSFLAYSVAILIVALFTVVLFFLLSSIITVLIAGFTTTISEKIEAIFVKKEFGAMQVNEEVIEASLDIDEEIYIDRSKRKISVFGRYEKAKKTDSLLGAEHRIDGEFFIFKCIKVKGEKLYLPYSLIENRYVNGYVVGKNISYYCDGNESERMLLVDAIKEEIIENEKEHVEEKICGEILAEKCAKAAAKLIKSITEESFDTEPRFKDIVLTLLVTGVFLITNFVSWLISKIASSEINLQGILWIINIMILVLILVLYVVKRFYEKEKPLKFDIEIDKVHVIFCESSEGIIETVIKEKEAFVKDEYIVLVKKGDEEGERFLGKLKEEKEKGLYEVRLIYGTKKENKIKYWVKLRS

SEQ ID NO: 26 - DNA, 783 bp, Anaerocellum thermophilum DSM 6725
atgagcaagatattagtgaaaggtgtaaaaaatccaatatataacagattgcgtattaagtcatatttggtaagtttcaaagttttgatagtagttgttactataattattgcttttttgcttattaactatgcaactgattgggttaatagaattggattggatgtagatgaaaaaacaaggtcgtttataaatacattggcttttagtttatcgctagatatcttatccccatgctctatcgatgacactattttgggataccctattacagaagctattttcacaccaataatatacatggttacttggtttaagggtgatagcaaagaaaaaatagaatatttttcaagaaataaatcagtaatagaactgctgttgagcaagcttcaatatgttttaatggacaactctccagaggaaaattttatgaattgggtgaaatcgaaagatatagatttaaaaaaatatgaaaaggaagatattttaacgttttattgggcattccactttttagatgtcaatgtaatagtagtgatattcagtactgtagtgttgataggatgtattggagcaccagtaagtgtattagtatttacattaatattcagcatatctacgttaatagaggatataagagatttggtctttaagagaagagaagcaattttaaggggagaggaaggaattgaagatgaagatgaaaggaagatatatgagaaaataaaagaagagataaaaatagcaatttataaggaagaagaagcaagaaatcaaagttag

SEQ ID NO: 27 - protein, 260 aa, Anaerocellum thermophilum DSM 6725
MSKILVKGVKNPIYNRLRIKSYLVSFKVLIVVVTIIIAFLLINYATDWVNRIGLDVDEKTRSFINTLAFSLSLDILSPCSIDDTILGYPITEAIFTPIIYMVTWFKGDSKEKIEYFSRNKSVIELLLSKLQYVLMDNSPEENFMNWVKSKDIDLKKYEKEDILTFYWAFHFLDVNVIVVIFSTVVLIGCIGAPVSVLVFTLIFSISTLIEDIRDLVFKRREAILRGEEGIEDEDERKIYEKIKEEIKIAIYKEEEARNQS

SEQ ID NO: 28 - DNA, 192 bp, Anaerocellum thermophilum DSM 6725
atgtatgtatcggcatttatattgttaatatattctttgtatccatttttacatgaacatgttattaatatacttaaaaagggaataaaagaagagtcaaggctaataagtatatggcttgtaatggttataatatcttatcttcttaaaaggtatgacattataactaggtctgatgaaggggatttatga

SEQ ID NO: 29 - protein, 63 aa, Anaerocellum thermophilum DSM 6725
MYVSAFILLIYSLYPFLHEHVINILKKGIKEESRLISIWLVMVIISYLLKRYDIITRSDEGDL

SEQ ID NO: 30 - DNA, 1311 bp, Anaerocellum thermophilum DSM 6725
atggagagtagagagagggtagtgaaagcgctgaataagctaataaaggcattggcagtaatttggttagtgttgattgttctgaggcttatatggtattttgcagatatagcgtatgtgagcgaaagaataacaggggattcatcagatggtattatagtattttttaagcagatatttttgtacattctttttttgggtttagtaggagtaatattaccgtttttcattttttctgggggagggtatattccatatttagcattgtatgaactgttgacaatttttaaaataatagttgtagtaactgtagcagttgtggtaaggaagttttgggttgtaaataaatattttgagacaacagcgtttaagagagattttttagtgaatagtagcaaaaaagaaatagtactaatattaaaatgtgacgaatcatattgtaaatataaaacagaagaagagttttatatttttaagagaattgtgactgaaggaggaggagagatatatttaccttactctgtaatagaagatagatatgtagatggatatgttataggcaaaaatatatggtatcatgaagaagattcaggtagaaaaatattgatagaagagttattagaagaaaaaatagtagaaaataaagaaaataaaaaggaacaagctgaagagagaattaatgaagaaatattagcagaaaaagtggcaagagctgctataaaattgcttaaattaagaaggcaagagaagacaatggtcagttgtagtaatgtttcacccatttattatgtaacagctttaggtaattcaagaggtttttttgcgaaaataattccatcgttaattgtaggctcgttaattgcggggatatttgtaacaatagaatttacatattggttaatacaggatataacgaatagtagtcgtataaccgtacacaagatcatatggattatgagtatgctactgatggtaatgctattagcaatttttgtggtcaaaggtttttataaaagagaaaaaaaagatgaagaaaaacttaacatagaaatagtaaaaataccagtgatactatacagggaaaataagaaagctgaggtaaaagaaggaggtatatttgtaaaagacgacgaaaatatagtgttagtgaaaaagggagacaaagaagttgaagagcttttgagtgaattaaaaataaaaaaagaaagagtagaaggattgtatgaggtgagattaataagtaagtattcttctgatgaagaagtaaaaagcacgctatggtgcttggtaagaataggtggtgaagcttaa

SEQ ID NO: 31 - protein, 436 aa, Anaerocellum thermophilum DSM 6725
MESRERVVKALNKLIKALAVIWLVLIVLRLIWYFADIAYVSERITGDSSDGIIVFFKQIFLYILFLGLVGVILPFFIFSGGGYIPYLALYELLTIFKIIVVVTVAVVVRKFWVVNKYFETTAFKRDFLVNSSKKEIVLILKCDESYCKYKTEEEFYIFKRIVTEGGGEIYLPYSVIEDRYVDGYVIGKNIWYHEEDSGRKILIEELLEEKIVENKENKKEQAEERINEEILAEKVARAAIKLLKLRRQEKTMVSCSNVSPIYYVTALGNSRGFFAKIIPSLIVGSLIAGIFVTIEFTYWLIQDITNSSRITVHKIIWIMSMLLMVMLLAIFVVKGFYKREKKDEEKLNIEIVKIPVILYRENKKAEVKEGGIFVKDDENIVLVKKGDKEVEELLSELKIKKERVEGLYEVRLISKYSSDEEVKSTLWCLVRIGGEA

SEQ ID NO: 32 - DNA, 774 bp, Anaerocellum thermophilum DSM 6725
atgaataggatagtagtaagagacatagaagacaaaaaaggaaaaagcactattacaaagattagtacattgcaaaaagctgtagtgttagttgttgctgtaatttttactttcttattattcaagtacgcaaaagaggtagttaataggaatgagctaaatgtggatgataggataaggcctattacaaaaacattagtttttggattatcaatagaacctgggtttataggtaatgaactttttgactttcctactgtgggatgttatagtttgattgcatttatatggcggcacgtaacggaagatgacaaaattactctttcagaaaatttaatagaattgacctcaaaagaaaaacaggcaatagaaatgatgttgaaaaaacttaactatattttattggaaaatccaaaagaagcttttatgagatggattaaatcagagaatattaaaatgaacagaaaagagtataaatatgatattttagaagatttttgggcatttcattttttagatatgaatgtaatatttatcggagttagtattataagttttattttttggactacatatgcattttgcaatatgttatttttaattatagtaactatggtagagtttgtaataaacttgtgggtagtaattttgaagaaagaagaaggagtagaagatgaagaagaaagagagatatatgagatgataaaagaagaagtaagaaaaataatttatgatggaaaagaagcaagaaatttaagacaggaaagcaattaa

SEQ ID NO: 33 - protein, 257 aa, Anaerocellum thermophilum DSM 6725
MNRIVVRDIEDKKGKSTITKISTLQKAVVLVVAVIFTFLLFKYAKEVVNRNELNVDDRIRPITKTLVFGLSIEPGFIGNELFDFPTVGCYSLIAFIWRHVTEDDKITLSENLIELTSKEKQAIEMMLKKLNYILLENPKEAFMRWIKSENIKMNRKEYKYDILEDFWAFHFLDMNVIFIGVSIISFIFWTTYAFCNMLFLIIVTMVEFVINLWVVILKKEEGVEDEEEREIYEMIKEEVRKIIYDGKEARNLRQESN

SEQ ID NO: 34 - DNA, 792 bp, Anaerocellum thermophilum DSM 6725
ttgaatagatttttggcaaactttctgaaatacaaagatcttttatatgagcttgttttgagagatataaaaattaaatatagacggtctattttaggtatgttttggagcttgctaaatcctcttttgatgatgattgttttaacgattgtattttctcaccttttcagatttgacataaaaaattatcctatttacttattgaccggacaaattatgtttgcttttttttctgaatcaacctcaagtgcaatgagggcaattatagataacgcttcactcattaaaaaggtatatataccaaaatatatatttcctgtttccaaagttttgtcatctttttttaatctaattttctcacttttggcaattgttttagttacaataggaatggtagtattgggcaataatgttaatttaacgtggacatttttgttatttcctattcctttaatattcatattaattttttcaataggcataggcttaattctgtcttgctatgcagtatttttcagagatttgattcatctatactcggttggattaactgcttggatgtatttgacacccatattttatcctgtgagtataattccacaaaaatatctgatattgattaagctaaatccgatgtattattttatagagtactttagaaaagttacattttatggtacactacctacactaaaggaaactctaatttgcattttggtagacataatatttttagtaataggacttttagttttttatcgaaaacaaaataaatttatcttgtatgtgtaa

SEQ ID NO: 35 - protein, 263 aa, Anaerocellum thermophilum DSM 6725
LNRFLANFLKYKDLLYELVLRDIKIKYRRSILGMFWSLLNPLLMMIVLTIVFSHLFRFDIKNYPIYLLTGQIMFAFFSESTSSAMRAIIDNASLIKKVYIPKYIFPVSKVLSSFFNLIFSLLAIVLVTIGMVVLGNNVNLTWTFLLFPIPLIFILIFSIGIGLILSCYAVFFRDLIHLYSVGLTAWMYLTPIFYPVSIIPQKYLILIKLNPMYYFIEYFRKVTFYGTLPTLKETLICILVDIIFLVIGLLVFYRKQNKFILYV

SEQ ID NO: 36 - DNA, 735 bp, Anaerocellum thermophilum DSM 6725
atggacaaaaacatagctgtaaagattgaaaatgtttcaatgatgtttaatatggcatctgaaaagatttatagtattaaagagtactttataaaacttgtgtcaggtaagctatactttagagaattttgggccttgaaggatataagttttaaaatcaaaaaaggtgaaatttttggtataataggcttaaacggggcaggcaaaagtaccctgctcaaaataattgcaggagttttaaagccgacaatgggtagagtctatgtaaacggtactatggcaccattgattgaacttggagcaggttttgactttgaacttactgcaagagagaatatattcttaaacggcgctattttgggctattcaagaaagttcatgaaagaaaaatttgacgagatagtagagtttgcagaactgagagattttttagatgttcctctaaaaaacttttcgtctggtatgcaggcaagacttggttttgctattgctacaattgttgaccctgacattttaattgttgatgagattctggcagtaggagactttcattttcaggaaaaatgcgaaagaagaattaatagtatgcttgaaaaagggacaactattgtgatggtgtcccattcaatagatcaaattgagagaatgtgccaaagagttttgtggcttgaaaaaggcagaatgaaaatgataggcgatgcaaaagaggtttgtgaggcttacaggaactcttga

SEQ ID NO: 37 - protein, 244 aa, Anaerocellum thermophilum DSM 6725
MDKNIAVKIENVSMMFNMASEKIYSIKEYFIKLVSGKLYFREFWALKDISFKIKKGEIFGIIGLNGAGKSTLLKIIAGVLKPTMGRVYVNGTMAPLIELGAGFDFELTARENIFLNGAILGYSRKFMKEKFDEIVEFAELRDFLDVPLKNFSSGMQARLGFAIATIVDPDILIVDEILAVGDFHFQEKCERRINSMLEKGTTIVMVSHSIDQIERMCQRVLWLEKGRMKMIGDAKEVCEAYRNS

SEQ ID NO: 38 - DNA, 732 bp, Anaerocellum thermophilum DSM 6725
atgaaaggaatagttttggcaggaggaacaggttcacgtttatatccgttaactaaggttacaaacaagcatttgttgcctgttggaaaatatccaatgatttattatccaatttttaaattaaaacaagcaggtatcaaagagataatgataataactggtaaagaacacatgggagctgttgtcaatttattaggaagtggtcgtgagtttggattagaatttacttataggattcaagatgaagctggtggaattgctcaagctctgggactgtgtagcttttttgctgggaatgataagtgtgttgtgatactgggcgacaatgtttttgaggatgatataactgagtatgtaaggaatttcgaaaagcaggagcggggcgctaggattcttcttaaggaagttccggacccgcatagatttggagttgctgaactgaaagatgggaaaatagtctcgattgaagaaaagcctaagaatccgaaaagtaattttattgtcacaggaatctatatgtatgatagccaagtttttgatattatcaagacacttaagccttcacagaggggtgaattggaaattacggatgtaaataacgagtacataagaagaggagagttgtattttgactttttaaagggctggtggactgatgcagggacttttgagtcactaaaaagagcaaatgagcttgctgaaaatatggtgttagattttaatggaatataa

SEQ ID NO: 39 - protein, 243 aa, Anaerocellum thermophilum DSM 6725
MKGIVLAGGTGSRLYPLTKVTNKHLLPVGKYPMIYYPIFKLKQAGIKEIMIITGKEHMGAVVNLLGSGREFGLEFTYRIQDEAGGIAQALGLCSFFAGNDKCVVILGDNVFEDDITEYVRNFEKQERGARILLKEVPDPHRFGVAELKDGKIVSIEEKPKNPKSNFIVTGIYMYDSQVFDIIKTLKPSQRGELEITDVNNEYIRRGELYFDFLKGWWTDAGTFESLKRANELAENMVLDFNGI

SEQ ID NO: 40 - DNA, 462 bp, Anaerocellum thermophilum DSM 6725
atggagcttattgaaggtgttaaggtaaaaagactcgagaaatttgctgatgatagaggttttttcatggagattttgagagacgaagatggctttttagaaaaatttggacaggcttcaatgtctttgacttatccaggggtaataaaagcttttcactatcataagttgcaagacgatgtttggtttttcccaaaaggcaatgcacaggttgttctttacgatttgcggccagattcgcctacatacaaaaaaacaaatgtattctacatgggagaacacaaccctatagtacttttaatacccagaatggttgctcatggctatagggtattggggaatgagccggcaataattgtgtattttacaacagagcattataatagagaaaatccagatgaatacagaattccttgggatgacaaggaaattaattttgattggacaacaaaatttagataa

SEQ ID NO: 41 - protein, 153 aa, Anaerocellum thermophilum DSM 6725
MELIEGVKVKRLEKFADDRGFFMEILRDEDGFLEKFGQASMSLTYPGVIKAFHYHKLQDDVWFFPKGNAQVVLYDLRPDSPTYKKTNVFYMGEHNPIVLLIPRMVAHGYRVLGNEPAIIVYFTTEHYNRENPDEYRIPWDDKEINFDWTTKFR

SEQ ID NO: 42 - DNA, 957 bp, Anaerocellum thermophilum DSM 6725
atggagacaattcttgtagcaggtggggcaggttttataggaagcaattttgttaagtacatgattagcaaagaagaatacaaaataatcaattatgatgcattaacctatgcaggaaatcttgagaatttgaaagaggtagaaaaccacccttattatacatttattaaaggagatattgttgatagatctaaagttgaagaggtttttaaaaattatcaaattgactatgtaataaactttgccgcagagtcgcatgtggacagaagtataaaggaccctgatatattcgttaaaacaaatgttttgggaacacaagttttattagatgtgtcgaggaaattcgggataaaaaagtttattcaaatttcaacagatgaagtatatggttccttagggcctgaaggatattttacagaagaaagtccgcttgcaccaaacagtccttattctgccagcaaagcaggggctgatatgcttgtgagagcatattttaagacatatggtctgcctgtgaacataacaaggtgttcaaacaattttggtccacatcaacacccagaaaagtttataccgaccgtaattttgaatgcgctgcaaaacaagccgataccaatttatggtgacgggcaaaatataagagactggctatatgtagaagaccactgcagagcaattgagcttgtgctcaaaaaaggtagaataggtgaagtatacaatattggcgggaataatgagtggaggaatatagatatagccaaattgattttaaaactactcgggaaaccagagaatctaatacaatttgtggctgacaggccaggacatgataggagatatgcaattgactcgagtaagattcaaaaggaattggggtggaaggttgaatataagtttgatgaagctattagaaagactatagaatggtataaaaatgaatttttcaaaggagaatga

SEQ ID NO: 43 - protein, 318 aa, Anaerocellum thermophilum DSM 6725
METILVAGGAGFIGSNFVKYMISKEEYKIINYDALTYAGNLENLKEVENHPYYTFIKGDIVDRSKVEEVFKNYQIDYVINFAAESHVDRSIKDPDIFVKTNVLGTQVLLDVSRKFGIKKFIQISTDEVYGSLGPEGYFTEESPLAPNSPYSASKAGADMLVRAYFKTYGLPVNITRCSNNFGPHQHPEKFIPTVILNALQNKPIPIYGDGQNIRDWLYVEDHCRAIELVLKKGRIGEVYNIGGNNEWRNIDIAKLILKLLGKPENLIQFVADRPGHDRRYAIDSSKIQKELGWKVEYKFDEAIRKTIEWYKNEFFKGE

SEQ ID NO: 44 - DNA, 2172 bp, Anaerocellum thermophilum DSM 6725
atgagaagtataaaccaattaaatattggtctaacagagacaaagtacggcaaattctattatttaaagagtgatgcaataataggcaaatctttagaaatatacggagaatgggcggtaccagaaatagagttgttaacaagttttatagaagaaggggatatagtaattgatgtaggcgcatacataggaactcatactattccatttgctcaaaagttgaatggaaaaggttttgtaattgcattcgaaccccaagagattatattcaatatactagcaaagaatataaggacaaataacgcagataatgtgcaaatatttaacaaagctgttttagataaaaatacaataacctacatagaaacttttaactacaatgaaactaataattttggcagtgctaaaataataactgatgatattgaaactcaaggaattatcaaaaaaattgaagcaatcactattgatagtttagaattgaataattgtaaattaataaaaattgacgtagaaggacaagaagaatttgtattgagaggctcagaaaagacaataaaaagttttatgccaattatttattttgaaagtaatgagcttgagaaaacatggaatagtatttgcttagtaaagaaatggggatacgatactttcttgtttagatttccagcatttaatccatttaacattaaaggtacacaagataatatttttggttatgcatgtgaaacaggtattttggggattcataaatcaaaactgagtacttatggaaatgatactttgaaatggcgaaaattaaattatctatttgaaattaccacgcttgacgatttagcttttttattaatagaaacttctcgtggacatgatcccattatacctgatattaaatataatgaaatcagaaacataaagtttcttcaaaattatataattgaggtggtaaaagaaattgaaaaaaaagataaactattacaaacaattaaagaagatttaaaaataaaagaacaacacttacaaagtatttattgttcggaaggttggaaactgttaaccaagtgttataaaataagagataagatattcccaccgaatagtaagagaagagaattagcaaagtttattttatttattatgaaaaaactgaatttaaaatttgttcaggaaataataagatttgcaaggatgtatggtattaaagctctttataatcgaagtcgaattgctttgcactctatgaaaagttattcgagtaaccaagaaaattattttgttcctgaacaatttgaaagagattatcttttaagtgaaatcagtgttgatataattgtacctatttataatgcttatgaggatttaaagaggtgtgtagaaagtattcttaagcacactgatttgaaaaaacatagattggtattaattaatgattgttcgactgatgagagaatatattctttcttaaaaaatcttgagaacgaaagaaccgaagaaaatttacttgttattcataataaggagaatttgggttttgtaaaaacagttaataaaggaattagtttgagtaataaagatgttattattcttaactctgatacaattgtaactgcgagatgggtagaaaagcttatcagagcagcgtattcaaggtctaatgttgcaacagttacacctttttcaaataatgccacaatttgttcattaccaataatgttaaaagataattcgcttcctttggattgggatattgattattttgctaaagttgtagataggatttcacttttaaaatatcctgaaattccaacagcagttggattttgtatgtatattaaaagagaagttattgaaaaaataggaatgtttaacgaagagaaatttggcaaaggttatggagaagaaaatgatttttgtatgcgagcgctaaacgaagggtatgtaaatattttatgtgataatttatttatttatcacaaaggaagtcaaagctttacggaagaagttaaaaggaaaagagaaatggagagcttaaaggttatcaatcaattacatccattttactctgagatggttaaggcctttatagaaaaaaatcctttaaaatattatcattctacacttgaagaaatcatgtccctttataacttattaaaaagtgaggagaaaaagtga

SEQ ID NO: 45 - protein, 723 aa, Anaerocellum thermophilum DSM 6725
MRSINQLNIGLTETKYGKFYYLKSDAIIGKSLEIYGEWAVPEIELLTSFIEEGDIVIDVGAYIGTHTIPFAQKLNGKGFVIAFEPQEIIFNILAKNIRTNNADNVQIFNKAVLDKNTITYIETFNYNETNNFGSAKIITDDIETQGIIKKIEAITIDSLELNNCKLIKIDVEGQEEFVLRGSEKTIKSFMPIIYFESNELEKTWNSICLVKKWGYDTFLFRFPAFNPFNIKGTQDNIFGYACETGILGIHKSKLSTYGNDTLKWRKLNYLFEITTLDDLAFLLIETSRGHDPIIPDIKYNEIRNIKFLQNYIIEVVKEIEKKDKLLQTIKEDLKIKEQHLQSIYCSEGWKLLTKCYKIRDKIFPPNSKRRELAKFILFIMKKLNLKFVQEIIRFARMYGIKALYNRSRIALHSMKSYSSNQENYFVPEQFERDYLLSEISVDIIVPIYNAYEDLKRCVESILKHTDLKKHRLVLINDCSTDERIYSFLKNLENERTEENLLVIHNKENLGFVKTVNKGISLSNKDVIILNSDTIVTARWVEKLIRAAYSRSNVATVTPFSNNATICSLPIMLKDNSLPLDWDIDYFAKVVDRISLLKYPEIPTAVGFCMYIKREVIEKIGMFNEEKFGKGYGEENDFCMRALNEGYVNILCDNLFIYHKGSQSFTEEVKRKREMESLKVINQLHPFYSEMVKAFIEKNPLKYYHSTLEEIMSLYNLLKSEEKK

SEQ ID NO: 46 - DNA, 1242 bp, Anaerocellum thermophilum DSM 6725
atgagcacaattgtttatttagtccattcaatacccaaatatgaaaagagtggaaccccaatagctgcttggagagtggcaaagggtgtaaaagaaaaatataatcaaaatgtagcatttataattccttcacctgatggagaagagggaaaagaaaaggttgacgatattttggtttacaaagtaaaaagaatagattggcatgaaaatttttttcatgattttgatatagatagagaaacgtatattagaaaaataaagaatattttaaaagaagttaattgtaatattttgcatatttataatttagtatttagctcttatcaggtaatgaaacttaggaaagaaggaatcagaattgttcgtacgataacacatactgaggatatttgttttaatgttgatccttttgttaaagttggtgataaaattgagatatgtagtgggcctgatccgatagcaaaatgcgcttgtcattataaacagatgtatggtggtaataatttgatggagtttataattaaaaagatatctaagcattttacgagcgtagaattattatattctaatttttgtgatataataacctttactaatgaagaatttgctaaatattttaccaactacgtaaatattccaagagatgtaatccgaataataccacatggagtagaaaataaattagaaaaatatatattgccgaatatgccaaaaaacgaaggatttagatttttatatcttggaggagataattttagaaaaggatttgttatattagataatgcgttaaattctttaaatggggagttattcaacaagattaaggaaataactatagtgggtaaaacaacaaaggaatttagagaaaggtttaataatgataaatatatgtttaagggggtattgccagaagaagaattatataacgaaataagtaatgctgatcttgtgatattgcccacatttttcgaaacatataacatatctttaagagaagctatcaagttaggaaaacctgttataactactaaaacttttggttcgaatattgtagtggatggatataatgggtttagatttgatattggtgatagcttacaattaaaaaacataatagaattaatattaagcaatccccaaatactggtagatatgagcaaaaattgtttaaatactcatataactgatattgaagaagaaataaagttatttatgaaagtttacaatgaattaaaagattag

SEQ ID NO: 47 - protein, 413 aa, Anaerocellum thermophilum DSM 6725
MSTIVYLVHSIPKYEKSGTPIAAWRVAKGVKEKYNQNVAFIIPSPDGEEGKEKVDDILVYKVKRIDWHENFFHDFDIDRETYIRKIKNILKEVNCNILHIYNLVFSSYQVMKLRKEGIRIVRTITHTEDICFNVDPFVKVGDKIEICSGPDPIAKCACHYKQMYGGNNLMEFIIKKISKHFTSVELLYSNFCDIITFTNEEFAKYFTNYVNIPRDVIRIIPHGVENKLEKYILPNMPKNEGFRFLYLGGDNFRKGFVILDNALNSLNGELFNKIKEITIVGKTTKEFRERFNNDKYMFKGVLPEEELYNEISNADLVILPTFFETYNISLREAIKLGKPVITTKTFGSNIVVDGYNGFRFDIGDSLQLKNIIELILSNPQILVDMSKNCLNTHITDIEEEIKLFMKVYNELKD

SEQ ID NO: 48 - DNA, 1008 bp, Anaerocellum thermophilum DSM 6725
atgagaaatttaacagttataatacctacatataatcaaaaagatttattagaaagagcaataaattcgttaatatctaagtgcaatgacgaaataaatatatatgtattagtcaacaatacggaaagtaattacatcgtaagaaataaaaattatgcaaatctacatgttgaatatttgaacacaaactgtggtttttgtaaagcagtcaattatggattacgattaataaaaaaatcaagatttatttttcttttaaacgatgatactgaagtaataaatcagatagatatagataatattattcatgaattgattgaaaaaggcaacattttttctatatcgttgaaaatgctgaagggtaattatccaaatcttttagacgacgcaggtgatatgtacactatcttaggttggcagtttaaaagaggcaatggtcttccaaaagaactttatgatagaccgtgtgaaattatttccgcatgtggtggtgctgcaatctataacaaaaaaattcttgatgagataggttacttcgatgaagatttttttgcatatcttgaggatgtagatttaggtttgagagcgctcatgaggggatataaaaatttatattatccttacataagcgtattgcatgttggaagtgcgacaacaggaggaaaatataacgatattactatcagacttacagcaagaaactcgatatatgttatatacaaaaatcttcccttaccccttttaataattaattttccctttattttattgggatacttaatcaaatttatattctttgctaaaaaaggaaaaggaaaaatttacataagtggagttcttgaaggactaaaaaatttgcccaaatttaaagaaaaaagaagagaaaatatgagaaaaaagaaaatttctaacataaagcttgaatggattcttattaaagctacatttgaatattttcaccaatatataaaaagagcattttacactttaagaggtgcaaagaaatga

SEQ ID NO: 49 - protein, 335 aa, Anaerocellum thermophilum DSM 6725
MRNLTVIIPTYNQKDLLERAINSLISKCNDEINIYVLVNNTESNYIVRNKNYANLHVEYLNTNCGFCKAVNYGLRLIKKSRFIFLLNDDTEVINQIDIDNIIHELIEKGNIFSISLKMLKGNYPNLLDDAGDMYTILGWQFKRGNGLPKELYDRPCEIISACGGAAIYNKKILDEIGYFDEDFFAYLEDVDLGLRALMRGYKNLYYPYISVLHVGSATTGGKYNDITIRLTARNSIYVIYKNLPLPLLIINFPFILLGYLIKFIFFAKKGKGKIYISGVLEGLKNLPKFKEKRRENMRKKKISNIKLEWILIKATFEYFHQYIKRAFYTLRGAKK

SEQ ID NO: 50 - DNA, 846 bp, Anaerocellum thermophilum DSM 6725
atgatactaatcacaggttccaaaggccaacttggaagcgaatttataaagcagtttgaaaataaatataatgtgaaaggtattgacattgaacaagttgatattactgatcttgacagtacagttagttatatttcagccacaaagcccaacatcatcatccactgtgcggcttacaccaatgtagatgggtgtgagagtgacaaagacactgcttttaaagtaaatgccattgggacacgaaatgttgcaatggctgcagaaaaagttggtgcaaagcttgtatatatatctaccgattatgtatttgacggtgaaaaagagaagccatataatgaatttgatagaccaaatccgataagcatatatggcctttctaagcttgcgggagaggaatttgtaaaaactttttgtagcagatattttatagtgaggattgcctggctgtatggtgaaaatggtaacaactttgtaaaaacaattgtaaaacttgctaaagaaaaaggcgagattgatgttgttaatgatcagagaggcaatcccacatttaccaaagatgttgttcaagctgtagaagtaataatgaactcagagaaatatggaacataccatgtaacaaatgaaggcataacttcttggtacgattttgcatataagattgtaagcacgtttgggatagactgcaaagtcaatccaactacaagtgataaatttattcgcccggcaaaacgtcctaagaactcagcacttgacaagatgatgttaagacttgaatttggatataaaatgagacattgggaagaggcttttgaagagtttgctatgctgatgaaaggaagaatatag

SEQ ID NO: 51 - protein, 281 aa, Anaerocellum thermophilum DSM 6725
MILITGSKGQLGSEFIKQFENKYNVKGIDIEQVDITDLDSTVSYISATKPNIIIHCAAYTNVDGCESDKDTAFKVNAIGTRNVAMAAEKVGAKLVYISTDYVFDGEKEKPYNEFDRPNPISIYGLSKLAGEEFVKTFCSRYFIVRIAWLYGENGNNFVKTIVKLAKEKGEIDVVNDQRGNPTFTKDVVQAVEVIMNSEKYGTYHVTNEGITSWYDFAYKIVSTFGIDCKVNPTTSDKFIRPAKRPKNSALDKMMLRLEFGYKMRHWEEAFEEFAMLMKGRI

SEQ ID NO: 52 - DNA, 897 bp, Anaerocellum thermophilum DSM 6725
atggatttgtctataattatagttaattacaataccaggaatttgctcagaaaaactcttgagtcaatatataagaatcctacctatagagaatttgaaataattgtggtagacaatgcatcaagtgatggcagccaagagatggtaaaaaaagagtttccaaatgttattttgattaaaaataaacaaaatttaggatttgccaaagccaacaatatagggataagaattgcaaagggaaagtacatattgcttttaaattcagatacagaagttttaaggggtacacttgatagttgtatagattttttggagaaagatgaagctaaagaaattggaattcttggatgtaaagtggtgcttccagatggcaagcttgatttagcatgtagaagagggtttcctactcctaagaattcgttttttaagatatttgggcttgcgaagctgtttcctaaaagtcgattttttgcaggttacaatcttacatatcttgatgaaaatcaatcttatgaagttgattcagttgttggggcgtttatgttaataaggcgcgaagttatagataaaataggtttacttgatgaggattatttcatgtttggcgaggacatagatttttgttttagagccaaacaaaatggatttaaagtttactactatgctgatgcaaaaatcattcatcacaaaagaggttctggcaggaatttgaaggttttgtcagctttttatgactcaatgtggatattttataaaaaacattactacaacagatacccaaaaaccttagccttattaatatttataacaattaagttgattaaatcattaaagcttgcccatgcgaaagtacggaactttcctattagaaagggaaaaaaaccatgcatagcctaa

SEQ ID NO: 53 - protein, 298 aa, Anaerocellum thermophilum DSM 6725
MDLSIIIVNYNTRNLLRKTLESIYKNPTYREFEIIVVDNASSDGSQEMVKKEFPNVILIKNKQNLGFAKANNIGIRIAKGKYILLLNSDTEVLRGTLDSCIDFLEKDEAKEIGILGCKVVLPDGKLDLACRRGFPTPKNSFFKIFGLAKLFPKSRFFAGYNLTYLDENQSYEVDSVVGAFMLIRREVIDKIGLLDEDYFMFGEDIDFCFRAKQNGFKVYYYADAKIIHHKRGSGRNLKVLSAFYDSMWIFYKKHYYNRYPKTLALLIFITIKLIKSLKLAHAKVRNFPIRKGKKPCIA

SEQ ID NO: 54 - DNA, 5133 bp, Anaerocellum thermophilum DSM 6725
ttgaattcaaagaaagcttttagactgctatcatgggtagtgataatatcttttgtattgggttttataaacccagctgtttttgcaaaaggttttaaagacacatcaaatcactgggcaaaagatgtaattgagagatgggcaaatacatacaatgtggcaaatggttattctgatggcacttttaaaccctcaaatgctattacaagggctgaatttgctcagcttgtgagcagagtaattggtgaggctttagtcaggtctgaaattaactttaaggatgtaaaagaaaatgattggttttactcagccgtcaaaaatcttgctgattatataagtgggtatccagatggtacatttaaaccaaagaacagtataacaagagaagaagcagcatgtattttggcaaaggtatttggtattgacaaatcacagagcaatgttctttcgaagttttctgattataaacaagtgtcagagtgggcaaaagagtatttagcagctatggttgaaaatggatatattaatggctataaagataaaacattaagacctaaaaattacattacaaaagctgaggcactgactattttagataacattgttgggcttttgttatcaaaaaagggtatgtatgtaggaagagaagtcaagggaaatttaattataagtgcaccttatgttagtgtgtcaggatttaatgtaagtaaaaattgcttgataacagaaggagtaaaagatggaaatgttacaattactaatacgcaggtagacggtaatatcattgttcgtggcggaggagaacacagtgtcgttttgaaaaatgtcaaagcttctgcagttatagttgtgaacaaacaagcagctacaaaagtaaatattagtggaaattcaaagatagataaggttgtaattgaaaagccagctaatattttagttgagaagactgcagaagtgaacacagtggatatcagagctgacagaacaatttttaaggcagaaggcaaaatagagaggatagcagtaaatgcaaaagatgtaaaagtaaatgataaggaggtccagcaaggcgctatactaacaattactaataccaatgaaaatgagaaaaagactcaatctacaaataccaatacttcgcaggagtcaacacaaaatgaaactacgtctcaaacatctaataatacttcaagtgtgcaaacaaatactggaacatcaactggaaatatatcttcaggcacttcaactggtggctatacaggtggaggaagtgtatatagtggcggttcttcgggttctggaacaagtgtccaatatggacctgtaagtagagtagaggttgacaaagaaaccatccctattggtcagaacgtccaagtaacagttacagtgaaggatgcagcggggaatttattgccaaacaaggttgtaaggattgaaggacaggctgcttatacaaattccttaggtagtgcaacatttactttatcagttcaggatagcaaagaaatagttataaatgtagatggcaatgactattatggtcttttgtacgcaattaaaccaactgaaggagttttgacatttaagttaaagacggcaagtggtaactttttaagtggatttaatgtgaaactagtaaatcaagctaagcaattttctgagttaaagtctgccacagctgaacaagtgtcattcattgtaccagcagttgatggatacaaagctttaatatggtcatatgatagtcaaaatgggcttatatataccatcttagataatctgtcagttggaaacggtattgtgagacttgttgcagacacaacattaccaaattatgtacaagctacactagattttagcataaataaccaacccctcaataactatgagttttcaattatcaatagctcaaaatctaatactttttcaattgatgatgtaaagctaaatatttcttcaaatcagcttaaaattacagcagatgcgggcagttattcaatcaaggtagcaaaagatactcaggatggtaagctatattttatgagagattttaatctccaacagtcaggacaagctttttcatttaatttcagctcttttaaaagagttatatttaatttttctggtattggcactttacaaaagagtgtatctgtaaaattaaatggtagctggtttgaactctccggggaaaatgatatatatctgcaaagaggagtttataacctggaagaagtttttataaagacatacgatgagaacagtaaagaggttgattattcatataaggtgccagcaggcagtagtccaattgtagttgatgcgacaggaagtaacgaagagtgctctgtgaatgtagacctttcaatagataatgtctcaagcagtgtatatgctactaatatagatggaaatgaatttgcttctataaaggcaggttcacttatagattttgtagtggttttaaagacgaaaagtgggctgaacttagctttgttaaagccatctgaaaagatgtcagatacagcaccattgtttaaagcaagtgatattgtggcatacatatcaagtggaaatgtacagaataatattgaacttctttgtacatcggtagaagataataatatttcgcatgtactcggatatttgccaaggaatcttcaagatggtactgttagcgtagaatttacggcagaaataggtttgctttatggaagctctattacaagcagtatttcgctttctatagacaatcaaaatggtgacagtagaggttatgtatatccacaaaatacaactacaagtgctgtatactttgtatgtgataaaattgacagtggagcaagtgactttttagcacttagtcttccggcaagtgaaacttgtgacgtcaatcttattgccaacaaaatatattttgaactcatgacagaaagaccgcttacttttgaatacggattgaatggaatgtttggactgagctttaatctcaccagtgaaaacctctacatgatatggcctgtttatcttttcagcaaagacgagggtcttacaagaagacaagctgttgaacaaaagtccttgcaaatagtatctacagttgtagaccaaacttacaatgactatgataaagttttaggtttgcatgactggcttgttttgcacacccagtacgatttggaagggtatcttaacaacaacataccttatgagtcacatacagcttatggtgcactaattaatggtattgcagtttgcaacggttatgcaacagctatgcttgcgcttttagaggatgcaggtatagactcaaaagagatctatggtatggcaggtgtaggtaattcaaaagaagcccatgcatggaacatggtaagcttagaaaataattggtatcatttagatgcaacatgggatgacccagactggggcaattatgttgaacatgcctttttcaatgttccagatagtaaaattgagttaacgcatgattgggaaagaaacctttatcctgctgctacagctatagattacagctatggtaactattacaatgtagaaaccattccacaggatgtttattacaacgaagacaatattgtaacaatagttgtcaaaaactataaaggtgaactacaagccaataaacttattacaataacgaaatttaaccaagcaggagttgaagatattgtatttactggctatacttctgctgatggtacaataacattcaatttaaagcctactgagatgatatcttaccagatttatataacctcttttatggaagacaaaggatacattaaagttgttgaaaaactgaagccggtgacattaagtttaaatattgacaatcaaccaattgatgaattttatatctcagatggtttgaacttcttgaaagtcacgggtggtcagcgacaggttggacttagcaggtggcgcgagaatgatctaattttctatggtttaggttttgtaataaagaaaagtttaacatttgacagctatgacccgatccaacttacagtttgtaacgatgcatacgattcttatgtacttttaagtgtgtataacagtgcttatgcagttgtcggtgcagacgtttatgtgattgacaaagataccttcaaggagctgtttgtaggcagcacagactcagaaggaaatcttccgcttgttcttacaaacggagaatacattttaaaagttgctaattataattcagaaacaaattctcaagatttttactattcgattttacaggtaacacaagatagtgtaacttcaattgatttgtctcagtttaatcagattcaatatataccaggatttaccgaaggcggcgttggaatgggagacaagctgttacttggtttcgactttgactatgatggaaaatttgttgtgtcatcgatggggcactcgagaaaggcttattttgcaccgggtgattatggttgggtatggattttggcccataacccgtatgatatagaagataatgttgctacatcattagtgtataaacctttgcaaaaactaattattccagaaggtacgcttcagcaatatactgttgatttgagagttgacagaagcaaaatagtcttgaactgtgaacgtcgaccagaaggaagctgggattttcttccaacaacgcttgatatggtatatgaaaaggatgacattataattgatatttatattccaaccaatagtcagagctacgttgtgggtggggatactgtacctcagcagcttatagaaaataatggagatatgcatattatgtggatgggtattgctggcatttatccaattagaacgtatactatttcaaatagtactccatatgaattgtttacaggacatcttggttttattgacaatactaatagttggcgtataattgtacttgatgtagtaggtgatctaaattcaacatttagaatagtattaccactatcaccgtatgacattgatgttattatcgacaaagaaattacaatttacccacttactgcaggatcaggtggtttgcgaaaagagtcatatattcaaaaggctaagatttacaatcaaataatcaacacacagcttattaaaaaagagaataaaaaataa

SEQ ID NO: 55 - protein, 1710 aa, Anaerocellum thermophilum DSM 6725
LNSKKAFRLLSWVVIISFVLGFINPAVFAKGFKDTSNHWAKDVIERWANTYNVANGYSDGTFKPSNAITRAEFAQLVSRVIGEALVRSEINFKDVKENDWFYSAVKNLADYISGYPDGTFKPKNSITREEAACILAKVFGIDKSQSNVLSKFSDYKQVSEWAKEYLAAMVENGYINGYKDKTLRPKNYITKAEALTILDNIVGLLLSKKGMYVGREVKGNLIISAPYVSVSGFNVSKNCLITEGVKDGNVTITNTQVDGNIIVRGGGEHSVVLKNVKASAVIVVNKQAATKVNISGNSKIDKVVIEKPANILVEKTAEVNTVDIRADRTIFKAEGKIERIAVNAKDVKVNDKEVQQGAILTITNTNENEKKTQSTNTNTSQESTQNETTSQTSNNTSSVQTNTGTSTGNISSGTSTGGYTGGGSVYSGGSSGSGTSVQYGPVSRVEVDKETIPIGQNVQVTVTVKDAAGNLLPNKVVRIEGQAAYTNSLGSATFTLSVQDSKEIVINVDGNDYYGLLYAIKPTEGVLTFKLKTASGNFLSGFNVKLVNQAKQFSELKSATAEQVSFIVPAVDGYKALIWSYDSQNGLIYTILDNLSVGNGIVRLVADTTLPNYVQATLDFSINNQPLNNYEFSIINSSKSNTFSIDDVKLNISSNQLKITADAGSYSIKVAKDTQDGKLYFMRDFNLQQSGQAFSFNFSSFKRVIFNFSGIGTLQKSVSVKLNGSWFELSGENDIYLQRGVYNLEEVFIKTYDENSKEVDYSYKVPAGSSPIVVDATGSNEECSVNVDLSIDNVSSSVYATNIDGNEFASIKAGSLIDFVVVLKTKSGLNLALLKPSEKMSDTAPLFKASDIVAYISSGNVQNNIELLCTSVEDNNISHVLGYLPRNLQDGTVSVEFTAEIGLLYGSSITSSISLSIDNQNGDSRGYVYPQNTTTSAVYFVCDKIDSGASDFLALSLPASETCDVNLIANKIYFELMTERPLTFEYGLNGMFGLSFNLTSENLYMIWPVYLFSKDEGLTRRQAVEQKSLQIVSTVVDQTYNDYDKVLGLHDWLVLHTQYDLEGYLNNNIPYESHTAYGALINGIAVCNGYATAMLALLEDAGIDSKEIYGMAGVGNSKEAHAWNMVSLENNWYHLDATWDDPDWGNYVEHAFFNVPDSKIELTHDWERNLYPAATAIDYSYGNYYNVETIPQDVYYNEDNIVTIVVKNYKGELQANKLITITKFNQAGVEDIVFTGYTSADGTITFNLKPTEMISYQIYITSFMEDKGYIKVVEKLKPVTLSLNIDNQPIDEFYISDGLNFLKVTGGQRQVGLSRWRENDLIFYGLGFVIKKSLTFDSYDPIQLTVCNDAYDSYVLLSVYNSAYAVVGADVYVIDKDTFKELFVGSTDSEGNLPLVLTNGEYILKVANYNSETNSQDFYYSILQVTQDSVTSIDLSQFNQIQYIPGFTEGGVGMGDKLLLGFDFDYDGKFVVSSMGHSRKAYFAPGDYGWVWILAHNPYDIEDNVATSLVYKPLQKLIIPEGTLQQYTVDLRVDRSKIVLNCERRPEGSWDFLPTTLDMVYEKDDIIIDIYIPTNSQSYVVGGDTVPQQLIENNGDMHIMWMGIAGIYPIRTYTISNSTPYELFTGHLGFIDNTNSWRIIVLDVVGDLNSTFRIVLPLSPYDIDVIIDKEITIYPLTAGSGGLRKESYIQKAKIYNQIINTQLIKKENKK

SEQ ID NO: 56 - DNA, 660 bp, Anaerocellum thermophilum DSM 6725
gtgacagacttatatataaccattttatttgtctcgataattatagcatttgggattgaaattttacaaagtaaaacctttcataaaagttgtttaacaggagacaaaaatttatttctgctcatagggatgacttttttaggaattcatttgtttataacatttccgggcatgatttcgataaagatatggatgatgctaaatcttattgtattgtggatcttgtcatattacagagaaactataaaaaaagaaatattattcaagaatatttctaaacttttaaaatctcaaagaagaaaaatattttttataagtagcatattgtgttacataattgtctgtcttcttttaaattattacatagtttacaagttcatatattcatctgcagtttcaaaattaacaataacattcactatttcctttcttatactaacagaagctaaaaatatagcttgttttattacaattttaaaattttattgcatatctttggtaacgatttggggagaaaatttagtgaaatatgagggatggcttattggagcaacaagagattattacatattaaaagaaaaattttcggggaaattaataacagtaaaaaaagatatagttaatcaaatagtgataatgggaaaagtttttgaaagataa

SEQ ID NO: 57 - protein, 219 aa, Anaerocellum thermophilum DSM 6725
VTDLYITILFVSIIIAFGIEILQSKTFHKSCLTGDKNLFLLIGMTFLGIHLFITFPGMISIKIWMMLNLIVLWILSYYRETIKKEILFKNISKLLKSQRRKIFFISSILCYIIVCLLLNYYIVYKFIYSSAVSKLTITFTISFLILTEAKNIACFITILKFYCISLVTIWGENLVKYEGWLIGATRDYYILKEKFSGKLITVKKDIVNQIVIMGKVFER

SEQ ID NO: 58 - DNA, 1074 bp, Anaerocellum thermophilum DSM 6725
atgaggtttaaaaagtttttaaaagttttgatagctgttttaatgtgctttatgcttggaaatccgttttatgcgcaagctgcaataaccctcacatccaatgctagtggaacttatgatggctattactatgaattgtggaaggattctggaaatacaacaatgacagttgacacaggaggaagatttagctgtcaatggagtaacattaacaatgcacttttcagaacaggtaaaaaattcagtacagcatggaaccagctcggaacagtgaaaatcacctactctgctacctacaatccaaatggtaattcatatctatgtatctatggatggtcaagaaatccactggttgaattttacattgtcgaaagctggggcacatggcgtccgcccggggcaacatcattgggtactgtaacgattgatggaggaacatacgatatttacaagacaactcgtgtaaatcaaccatctatagaagggacaactacatttgatcagtattggagtgttagaacatcaaaaagaacaagtggcactgttactgtgactgatcattttaaagcatgggctgcaaaaggcctgaacttgggtacaattgaccagattacactctgtgtagaaggttaccagagcagtggttcagctaatataacacaaaatacattttctataacaagtgcctcttcaggtggaacaacacccactaccacaaaggtagagtgtgaaaatatgtcactgagtgggccgtatgcatcaaaaattacaagtccattttacggcatggctctttatgcaaacggagataaagcaacaacaaatataaacttttcagcaagccgtaactatacttttaaattacgaggatgtggaaataacaataatttagcatcagttgatttactaatagatgggaagaaagtaggttcgttctattatcgggggacatatccttgggaagctcctatagagaatgtgtatgtgagtgcaggttcgcacaaagtggaaattgtagtttctgctgataatggtacatgggatgtttatgcagattatttgttaatacaatga

SEQ ID NO: 59 - protein, 357 aa, Anaerocellum thermophilum DSM 6725
MRFKKFLKVLIAVLMCFMLGNPFYAQAAITLTSNASGTYDGYYYELWKDSGNTTMTVDTGGRFSCQWSNINNALFRTGKKFSTAWNQLGTVKITYSATYNPNGNSYLCIYGWSRNPLVEFYIVESWGTWRPPGATSLGTVTIDGGTYDIYKTTRVNQPSIEGTTTFDQYWSVRTSKRTSGTVTVTDHFKAWAAKGLNLGTIDQITLCVEGYQSSGSANITQNTFSITSASSGGTTPTTTKVECENMSLSGPYASKITSPFYGMALYANGDKATTNINFSASRNYTFKLRGCGNNNNLASVDLLIDGKKVGSFYYRGTYPWEAPIENVYVSAGSHKVEIVVSADNGTWDVYADYLLIQ

SEQ ID NO: 60 - DNA, 363 bp, Anaerocellum thermophilum DSM 6725
atgtttggcggaagtacaatatccaaatatgttgaaggttatacaacattaaagaatatctatggaacagaaatcaattcttcgaccattcaatcttgtgataggaatattggtcaggctacaatttatgtaagcccttatgtaactgcatttacacatactttaactcagcgaagaggtattttaatgcatgagatgggacacgctatgggcttaagacatcctaattattcggactcaagaaatagctattcagctgatagttatggaagtattatggattacagttataccgaagaatacccaactatacatgatatatgtgacattgaaataatgtatggttttaactgttacaattaa

SEQ ID NO: 61 - protein, 120 aa, Anaerocellum thermophilum DSM 6725
MFGGSTISKYVEGYTTLKNIYGTEINSSTIQSCDRNIGQATIYVSPYVTAFTHTLTQRRGILMHEMGHAMGLRHPNYSDSRNSYSADSYGSIMDYSYTEEYPTIHDICDIEIMYGFNCYN

SEQ ID NO: 62 - DNA, 930 bp, Anaerocellum thermophilum DSM 6725
atgtttaaagatttcttaaaagactcatttttcttcacaaaaagatactatggatacatatttggtctattggttatatcatttattcttggtactttggcagttgtggttttactaatacttggtattttattggctatgttaatgggtgtggatatgtctacattaggtttattaaatgataaaaatgtttttccatttttaagtagcaaatttattttctatatgattttaatgattctactttttataatgtgggtattgttagtattagcttttatccaatatccacttgtaaagacctttattgaaataacacgagaaaaggatatatacaaaaaaccttttgaaattttctttgccggaataaaagaaaagaatctgatgatgggcttgaaaatagttggactgggatttttattaacacttatagttgggtcaatcctttttataggtattataggtatagcttttagtaccaatactgtcgataatttagcacgctttgctttgattataattggtattgttggtatagtattaggtgtttacctgttattaaggttaatgtttgctaatgcagcgttggtggataaagatataggagttatagaaagtattaaagagagtttaaagcttacaaaggggaaaatgggatttgtaatagcagcttttgtttatagtattttggtatccatagttttccaaataccagtttatattgttgatacattatttaaaacagaaacatctcaagagagtatattatttattattttaagtttgataggatttgttttttaccttatagcagtgccttactttattgtgttacagtatctaccgtataataccttgaaaagccatgttgaaaatgcaaaccatatagatagccagttttacggaacttataataatattattgggtaa

SEQ ID NO: 63 - protein, 309 aa, Anaerocellum thermophilum DSM 6725
MFKDFLKDSFFFTKRYYGYIFGLLVISFILGTLAVVVLLILGILLAMLMGVDMSTLGLLNDKNVFPFLSSKFIFYMILMILLFIMWVLLVLAFIQYPLVKTFIEITREKDIYKKPFEIFFAGIKEKNLMMGLKIVGLGFLLTLIVGSILFIGIIGIAFSTNTVDNLARFALIIIGIVGIVLGVYLLLRLMFANAALVDKDIGVIESIKESLKLTKGKMGFVIAAFVYSILVSIVFQIPVYIVDTLFKTETSQESILFIILSLIGFVFYLIAVPYFIVLQYLPYNTLKSHVENANHIDSQFYGTYNNIIG

SEQ ID NO: 64 - DNA, 807 bp, Anaerocellum thermophilum DSM 6725
atgagtgcaaaagaatttccaaaaaacgaactaataagcgttataattccctgttataacgaggcacaaaatattggacaaacactcaaagaaatttatgattatttagatgagtttgtacctaactatgaagtaattgttgtagttgaaaaaagcacagacaatactttggaagtaataaactcaagaaaaaatcaaaaaactatcgtattagagaatacaaaaaaatatggcaagggatatagtttaaaaagaggtatatattttgcaaaaggacaatacatacttacctgcgatgcggaccttcctgtggacataaaaaaatattttcttcctatgttagaactattaaagagagatgaaaaggttgctgcagtttttgccactgccttagctataaaaacatgcaggaaagaaagaggctttgtaagaagtattgtttcactgatattttttgtttttagacagttgttattacagtttcctgtaagtgatacacaattaggttttaaattgtttagagcagatgtagcaaaaaagttctgtcaaaaagttaatgagaatgggtttttgtttgacttgatattaacagatttgatgttaaatgaaggatatcaaattgaagaagtaaatgtaaaagttgtagaaaggaaaatcaagtcttctgtttctgtcgctgaaataataaaaactacttataagttttgcaaatatatattgtttacgcgttcaaaattacttagaaaatgtacagataaagttatgataaatgataaaagtaaaagcataaaagctacaagtatttga

SEQ ID NO: 65 - protein, 268 aa, Anaerocellum thermophilum DSM 6725
MSAKEFPKNELISVIIPCYNEAQNIGQTLKEIYDYLDEFVPNYEVIVVVEKSTDNTLEVINSRKNQKTIVLENTKKYGKGYSLKRGIYFAKGQYILTCDADLPVDIKKYFLPMLELLKRDEKVAAVFATALAIKTCRKERGFVRSIVSLIFFVFRQLLLQFPVSDTQLGFKLFRADVAKKFCQKVNENGFLFDLILTDLMLNEGYQIEEVNVKVVERKIKSSVSVAEIIKTTYKFCKYILFTRSKLLRKCTDKVMINDKSKSIKATSI

SEQ ID NO: 66 - DNA, 1116 bp, Anaerocellum thermophilum DSM 6725
gtgaacgaacgaaataaagaaattgcatatattattcttgacttattcgttgctgccgttccatttttgtatattacatacctttacagattaggagttacagaattagaaagtttaaaagaagttgtatatgctaattcccttttaagtacacttttttatatttctaacatagttatgtttctcaaaatagaatataatattagcttctacgcaactgcaaaagtaataagtgtaatttcattaattttaggaattatattgcaagtcttatatccatcttatggatgggcaataagtatacttttattaatggacagttttgtagtttttctcagaggaactatgttgagagagaaaaattacatgaaagcggctttaattaattttctgttgcacacaagtagatttgggatattaatactgtttcctttagatggtcttttgacaagatggattgtatctaatatcggtggatatttgctggtattggcaatgtatttatctttgataaagtggatttttgaaagagaagataaaaatagcattatgagtttaaacatagatgaaattagaaaaagagcttattatttactttttgtaatgataatttttgtaattcaaaatattgatctattagcactaaagggaatgaatttttataacttgcttgttttgtcaaggccgtggggcttagttgcttatgttgttacagtatcaattggaaatttagtcttgattaataaaaaaacatatccctattggatttatttaactgtagcatgggctggaattttcattggatatatttctttgggtaaacacataaattactatcttttcaaaattccatattcgtttgatttaagtatattaccaattttattttaccttttaatggctacgatattatttacaaattacttaaattatgcaaaaaagaagcagttaataattgaacctttgtgcttattcctattaattgaagtcttaaaaaataaaatctctctaatgagagaattgaaatttgaggtattaattttaattttaacatttttgataattgttcacttgtttttaatttttttatattcaacttataacaaatgtatgtag

SEQ ID NO: 67 - protein, 371 aa, Anaerocellum thermophilum DSM 6725
VNERNKEIAYIILDLFVAAVPFLYITYLYRLGVTELESLKEVVYANSLLSTLFYISNIVMFLKIEYNISFYATAKVISVISLILGIILQVLYPSYGWAISILLLMDSFVVFLRGTMLREKNYMKAALINFLLHTSRFGILILFPLDGLLTRWIVSNIGGYLLVLAMYLSLIKWIFEREDKNSIMSLNIDEIRKRAYYLLFVMIIFVIQNIDLLALKGMNFYNLLVLSRPWGLVAYVVTVSIGNLVLINKKTYPYWIYLTVAWAGIFIGYISLGKHINYYLFKIPYSFDLSILPILFYLLMATILFTNYLNYAKKKQLIIEPLCLFLLIEVLKNKISLMRELKFEVLILILTFLIIVHLFLIFLYSTYNKCM

SEQ ID NO: 68 - DNA, 1080 bp, Anaerocellum thermophilum DSM 6725
atgaagactattttagtgttaacgcaaagggatatataccataaaaaagctggtggtgcagaaagatatttgtttaatgtgctaaaagggttgagtgatcattacaaaatcacttgtctgtgtcaaaatgatggtactcagaaagattatgagatatatgataatataacgtttattcgaattaaaactaatttaattagcctaatttttaaagcaatgttctattataaaagaaataaggaaaacattgacttaataattgaccatacaaatacacatcagttttttacttttttatacgttccgaaaaataagagactattaatagttcaccagcttgcacttgagatttgggaatattattttccaaagtacattggaaaagcgctaaaattacttgaaaaacttctttggcgtctatcatccggaatggcagtaacagttagccggtcaactaaggaggatttacagaggtttggttttaaagaagaatttatatggattgtaaaaaattcattaaagcataaatatacttcacttccacatatggagaaagaagattttcttgttagtgtcgggagattagttccatataaaagatttgaagatgcaatttatttagcaaaaaaagttaacaaaaaaatatttattataggagaaggtcaggaaaattataaaagaaaattaagaaattatgcaaaaaaaattaacgcagatgtgatttttactggatatatttctgaagagaaaaaacaagacatagtagagaaagcctatatgcacatttttccatctataagagaaggttggggacttgtaataagcgaagcagctaacttagggacaccttccttagtatatcctgttcctggctgcctggatgcagtaaattacggaaaagctggttttgtaacaaagagaataggcaaagaacatttgttggaaagatttctaactattaatagggaagaatatgaaaggatgagaatttgtgcatttgaatttacttcacagctaaactataataaacaatgtgaagagtttcaacaggttatacaaagtataatagaaaatacatag

SEQ ID NO: 69 - protein, 359 aa, Anaerocellum thermophilum DSM 6725
MKTILVLTQRDIYHKKAGGAERYLFNVLKGLSDHYKITCLCQNDGTQKDYEIYDNITFIRIKTNLISLIFKAMFYYKRNKENIDLIIDHTNTHQFFTFLYVPKNKRLLIVHQLALEIWEYYFPKYIGKALKLLEKLLWRLSSGMAVTVSRSTKEDLQRFGFKEEFIWIVKNSLKHKYTSLPHMEKEDFLVSVGRLVPYKRFEDAIYLAKKVNKKIFIIGEGQENYKRKLRNYAKKINADVIFTGYISEEKKQDIVEKAYMHIFPSIREGWGLVISEAANLGTPSLVYPVPGCLDAVNYGKAGFVTKRIGKEHLLERFLTINREEYERMRICAFEFTSQLNYNKQCEEFQQVIQSIIENT

SEQ ID NO: 70 - DNA, 852 bp, Anaerocellum thermophilum DSM 6725
atgaagctgggaattttgataacaacatataatgatgactatattattttaagatgcttagattctatctataaccaacttgatgagatagattttccaatttatgttgtatgtgttgatgatggttcagatttgcctcttacatatcctcattttgatattctaagaactgaacataggggaagaagctatgcgagaattgagggactgaaaaaaattttagctgaaaattgcacacatttcttatttttagatagcgatatggtccttccaccaggctttctcaaaaagttgaagacagtagttgaaaattacgatagtgatgctttcattatccctgaagtggcttttagtagttataacaatttttggacaaaagttaaggtctttgaaagaaatttatatagagtaagttattgcaaagaaagtggaaatattgaagctgccagattatggaaaacaaatgcatttccgggttttgttgaaggattagaagcatttgaagagattcagccaacaatattgggtgttaaaaaaggtttaaagattttaaaaatccaagagatttttattcttcatgatgaaaagaaggtaactttcaaagacttaataagaaaaaagaatacttacttttgttgtatgattggttctgaaaaatgttcaaagtgggagataatgaaaaggtattatttctttcgtccccatctttaccataaggaaaatttaaagaaatacataagacatcccattttagctattggagttgtgtttatgtaccttgtattgacattgaattttatatggacgagtatatcaataaatttaattagaaaaggattgatggaaaaatga

SEQ ID NO: 71 - protein, 283 aa, Anaerocellum thermophilum DSM 6725
MKLGILITTYNDDYIILRCLDSIYNQLDEIDFPIYVVCVDDGSDLPLTYPHFDILRTEHRGRSYARIEGLKKILAENCTHFLFLDSDMVLPPGFLKKLKTVVENYDSDAFIIPEVAFSSYNNFWTKVKVFERNLYRVSYCKESGNIEAARLWKTNAFPGFVEGLEAFEEIQPTILGVKKGLKILKIQEIFILHDEKKVTFKDLIRKKNTYFCCMIGSEKCSKWEIMKRYYFFRPHLYHKENLKKYIRHPILAIGVVFMYLVLTLNFIWTSISINLIRKGLMEK

SEQ ID NO: 72 - DNA, 237 bp, Anaerocellum thermophilum DSM 6725
atgagaagaagggggagaaatgttctatataaggtaagacgaagggaaaattatgcaaaaatagttataatagctgtattaatagggtgccttattttattaggatggcatacggttgtaaagtcaagtaattggtttaaatcaattattatcgagaacatggactacaactatggaaaagattcaaatagtatccagccttatgcacctaaaaaaacacatcctcgaggagaataa

SEQ ID NO: 73 - protein, 78 aa, Anaerocellum thermophilum DSM 6725
MRRRGRNVLYKVRRRENYAKIVIIAVLIGCLILLGWHTVVKSSNWFKSIIIENMDYNYGKDSNSIQPYAPKKTHPRGE

SEQ ID NO: 74 - DNA, 4224 bp, Anaerocellum thermophilum DSM 6725
atgaaacaagaagagatattgcaaagacaaaggaagatagcaaagactatttcgttgattgctgtagtagttataatcctcatagatacaaaaaatatttggttgaataatggctttattattttcagtgatcttgactttagtggaattaatgataaaaggtatattgagagaatatggggtgtttttaatccgcatttttcttcaaccaatttttttaatttatctcgtttgttttttatatttcctttttatgccctaaactttttggtctcttctttttacaatcaagcaatgcttaagttgataattttgtcttgtttactaatatcaggaataggtatgtttttattatgtgagtatctattgataaaaaccttcagaggttttcctgattatgtgcattattttggattaataataccggctgtgtattatgcattaaatccatgggttatattcagaattcagcatatatttcttttgagcgggtacagtgtatttccgcttataataaaagaatttttgaggttgtatgaaataaagatttttgactttcaaaaagatattgaagaatatcaacaaagaagaagagttccgaaagagcatattattatagatgaaaaaataaatcttaaggacttcttcaaaatagttttgtactccatgattggttctgcagctattcactattttttcttttatgtcattttccttaccatattatttataggcattatgccaagaatatggaaattaaataggtattctaaagttgtacttttgttttatctaaaaaagtactttttaatgatttttttgatgatagttggaaatatgtattggtttttgacatattctatagcactattatttgttaatattgaacctcaaaatgtaaatgtggttgatacaattcaattattttcacgaaatagctctgtacagaatgtactctatttaatttcctattggcttccattttttaacttagaaatgtttttagacaaaatgttttgggttgcaggaggagtcttcctgcttataattgcatacataatattttatcgctttgggtggcatttttatgtaagacttttcaccctagtaacaagtctggtaatcttactctctttaggaacaaacgttgaccccttagctgatgtatatgttaaagtagtaacaaacgttccgataataggtcatctctttagagaccctaacaaaattgtaggtgtactggcttgttttttagctataatcctgggttttggagtagacagaatagtatttttcctgttaaatgcaggctttggcagaaaatttgcagtagcttttgttttagtcatgttgctttgtttctatttctatcaaagaccagttaaaattttctacatcgataattattacaaaggtgtgccaattccaaaagaatataaagaaataagtaaatattatacaaaagatggtaaaatattatggatacctactatggacaacatggtactttcaaatggcatttcggcttatagatggaatataaacaaggaaatgcctggttcaatgaaagcggctggggattttcatgtatatgcatcttctaaaaatacaatatttcaacatgaaaataatgtagggacagtttcttatttttattcttatttacagcatctattggataaaggtggaactgacaaaataggcaggttattaaaacttacaggtttcaatcaagtggcttatcacagagatgtccaaaatcaagaagagagacaaaagtttaatctttttgttctggaaagacagagaggaattgttctgagaaagaaattaggatttattcacctttacaatgtaaagaagaattctgataacgctaaggtggacatttacaataccaagggattaaaccattttgttcagctctttgactttgaaaatatactgggatcaaaaggatataatataatctggtctcagggtaaaagggagaaatttgatcttaacaaggtaaatttagtggtaggcgataataaatttgatatttttcaacaaaatatacctgagaaatatataatttcattatttgacaagataaatacaggaaatccatatgtaggttgggctaagtcaatgtgcaaggagtcagattggttttggattcacaaagtaaacggtttaaatcgtctgccatgggattatgattatggtaaaggatttatattcacatatacaccctataaagtggaacttcctccatataagttaaataagaatgttggggaggttatactcaactccaatgatattgtaaaaacagatttttttgaaattgacaaagaggagacaaattttaaacttgagattacaaaagatgtaatgggcaatagcatcttagcagcggaagttgctcccaaaaaatttattggtaatgttttctggaaaattgcaaggtcaaaacttatgtcgataaaacctgggtttgtaaatcttcgggcagtggtttccggtgtaaatgctggaaaagttcattttaaactcaaatttttcaatcaaaacatgaatgaattgggagtggtctatggtaatgttccgggagaaatagctgaatttaataaagcacttataactgctaatgctgttgtaccgcctgcaacaaagtatatgagggtggaactttggctacttgaagatcaaaaaacaccagtatatttttggattcatgacattgagttaagatatttttcaagcatttcatctaatgaactgagaataaaaattccttcatatattaaaggagaataccatctttacgtgagagctctggtaaatgagaatggtgggaaggtcaagatcaatgataccaaaataaacttaaaaggtgatgagaaccagtttaaatggttgtatgttggtaaatataccagagatactattgttataaaaccagaagaagggtttgtactattgaaccttctttgtgctttacctgattggttatataaaaaaattcaaaacgaagatttgaagagtaaacaagcaatagtattgtttagcaatgaatttgcattaaaagaaggttttaaagttaaaaaaatggatgattacattattcatccaaattttgttgatagtcttttagacgtaataagtgatgggatttatactaaaaaaatagatatattaagaagtgatcactataaactatattttataggaaacgttacaagtcaagttacaataacattgaagaataaagacgataaagttgtaagatttggatatttgaaattttttaaactaattaataataactttaatggtataagattttatgtttctaacaagccttactcgtactttttaaatattgaaaaagaagaaaatccaaaatggcgttcacaattgtattatattgaccttggttttttgaaaaaagggcaatatactttagagataaagtttgaaaatggaataaaatctttaacagaacctaatgatattcatgttttacgtccagaagaggttaaagttaatttaaaagttgacgacactctttatatattaatttcaaacattcttcaagaaaatgtgacgactcaaaagaaagaaaataaaatttttagcagcaagtattcagacatatattctgataaagtaaaatactggctcatctatggaacaaaacaggtagagtcaaaaaagaatgagttatattacgtgaactttgatattgaaacgaaaggtttggcagaggttagtgctaagattttgtttatggataagaataaagtactttacaatagccaatatattgacatatttaacaataaaggacaaagtatattttcagctaaaaaaaatggctttatccaaatagtattttttgtgcgtagtaacggtaaagaagatggtgtgtttgaattaaagcaaattagattactagagataagtaaaatgaacaaattttcatcaatagtaattttaccaagtagtatacaaaaaggtagtaaattgaatatactgaatgaaacctataatcctctatgggaaaacaatggtgaaaaagcgaaacaagtaaacattttcttaaatggtttttacactcaaagcaacaaatatgtatttggtaaaaaaatatggatagcttatatatttggtttactaatatcaattttatatattacagtaaatttgtggttgctattaagaagaaaaaaataa

SEQ ID NO: 75 - protein, 1407 aa, Anaerocellum thermophilum DSM 6725
MKQEEILQRQRKIAKTISLIAVVVIILIDTKNIWLNNGFIIFSDLDFSGINDKRYIERIWGVFNPHFSSTNFFNLSRLFFIFPFYALNFLVSSFYNQAMLKLIILSCLLISGIGMFLLCEYLLIKTFRGFPDYVHYFGLIIPAVYYALNPWVIFRIQHIFLLSGYSVFPLIIKEFLRLYEIKIFDFQKDIEEYQQRRRVPKEHIIIDEKINLKDFFKIVLYSMIGSAAIHYFFFYVIFLTILFIGIMPRIWKLNRYSKVVLLFYLKKYFLMIFLMIVGNMYWFLTYSIALLFVNIEPQNVNVVDTIQLFSRNSSVQNVLYLISYWLPFFNLEMFLDKMFWVAGGVFLLIIAYIIFYRFGWHFYVRLFTLVTSLVILLSLGTNVDPLADVYVKVVTNVPIIGHLFRDPNKIVGVLACFLAIILGFGVDRIVFFLLNAGFGRKFAVAFVLVMLLCFYFYQRPVKIFYIDNYYKGVPIPKEYKEISKYYTKDGKILWIPTMDNMVLSNGISAYRWNINKEMPGSMKAAGDFHVYASSKNTIFQHENNVGTVSYFYSYLQHLLDKGGTDKIGRLLKLTGFNQVAYHRDVQNQEERQKFNLFVLERQRGIVLRKKLGFIHLYNVKKNSDNAKVDIYNTKGLNHFVQLFDFENILGSKGYNIIWSQGKREKFDLNKVNLVVGDNKFDIFQQNIPEKYIISLFDKINTGNPYVGWAKSMCKESDWFWIHKVNGLNRLPWDYDYGKGFIFTYTPYKVELPPYKLNKNVGEVILNSNDIVKTDFFEIDKEETNFKLEITKDVMGNSILAAEVAPKKFIGNVFWKIARSKLMSIKPGFVNLRAVVSGVNAGKVHFKLKFFNQNMNELGVVYGNVPGEIAEFNKALITANAVVPPATKYMRVELWLLEDQKTPVYFWIHDIELRYFSSISSNELRIKIPSYIKGEYHLYVRALVNENGGKVKINDTKINLKGDENQFKWLYVGKYTRDTIVIKPEEGFVLLNLLCALPDWLYKKIQNEDLKSKQAIVLFSNEFALKEGFKVKKMDDYIIHPNFVDSLLDVISDGIYTKKIDILRSDHYKLYFIGNVTSQVTITLKNKDDKVVRFGYLKFFKLINNNFNGIRFYVSNKPYSYFLNIEKEENPKWRSQLYYIDLGFLKKGQYTLEIKFENGIKSLTEPNDIHVLRPEEVKVNLKVDDTLYILISNILQENVTTQKKENKIFSSKYSDIYSDKVKYWLIYGTKQVESKKNELYYVNFDIETKGLAEVSAKILFMDKNKVLYNSQYIDIFNNKGQSIFSAKKNGFIQIVFFVRSNGKEDGVFELKQIRLLEISKMNKFSSIVILPSSIQKGSKLNILNETYNPLWENNGEKAKQVNIFLNGFYTQSNKYVFGKKIWIAYIFGLLISILYITVNLWLLLRRKK

SEQ ID NO: 76 - DNA, 405 bp, Anaerocellum thermophilum DSM 6725
atgccaggtaaaaaaagatctgttgtgggtgtgttgatattctcaataatcactttgggtatctattacttatactggatttatgtaacctcaaaagaaacccaacggtacttagaaaagaatactacaagcccaggtcttgaacttttactatgtataattacatgtggtctttattggttttattggatttttaaatatagcaaaattgctgttgaatgccaacaaaaagctggtttgcctacagaagataatgctgttataaaccttattcttagcatcattggacttggaattataagttctatgattctccaatcaagtttaaacaaagtttgggaatttgaaagttctaaaaatagtccagttatttcaacaactaatacttctgaaaacaataagtaa

SEQ ID NO: 77 - protein, 134 aa, Anaerocellum thermophilum DSM 6725
MPGKKRSVVGVLIFSIITLGIYYLYWIYVTSKETQRYLEKNTTSPGLELLLCIITCGLYWFYWIFKYSKIAVECQQKAGLPTEDNAVINLILSIIGLGIISSMILQSSLNKVWEFESSKNSPVISTTNTSENNK

SEQ ID NO: 78 - DNA, 762 bp, Anaerocellum thermophilum DSM 6725
atgatagttaaaaatgtgttgattgcagatgaaaatgaatacataagaaaagcaataattgaaaagtttgaaaattcttttgattcagtacatcactttgtattttttgaggctgctgatggtgaagaagctttgaacaaaattggtgagaacagtattcatgttgcaataattgatacaaacttgcctaaaattagtggatttgaagttttgagaactatcaaaaaaagttctgttaaagcttacattccagtaattttgctcagtagcaatattcatcgtgcaacaagagcaaaggcatacgagcttggagctattggagttattccaaagcctttttcaactttagaagtttacaatatggtaaggtcacttttgtacactcaagatgaatatttacatattaatgaagttgtacatcttatttcttttttgaatgaaatggcaaatgaacatatcattgtagttcccaaaattatagatgaatttttatcagaatattttgtttcatatcacttgttggctttaaaagatactacaaaagaggtgatttataacagaggttttgatgaatatgaaattaaaaaggttattggctatttaaatgatgagaacagaataaattcaagctgttattcgattataaaacttaacaataaggaagaaacttattactttatattcaaaattctgaatgataataaaatattaattcttctaaagaaagttttagaactttggagtgaattaaatgatagataa

SEQ ID NO: 79 - protein, 253 aa, Anaerocellum thermophilum DSM 6725
MIVKNVLIADENEYIRKAIIEKFENSFDSVHHFVFFEAADGEEALNKIGENSIHVAIIDTNLPKISGFEVLRTIKKSSVKAYIPVILLSSNIHRATRAKAYELGAIGVIPKPFSTLEVYNMVRSLLYTQDEYLHINEVVHLISFLNEMANEHIIVVPKIIDEFLSEYFVSYHLLALKDTTKEVIYNRGFDEYEIKKVIGYLNDENRINSSCYSIIKLNNKEETYYFIFKILNDNKILILLKKVLELWSELNDR

SEQ ID NO: 80 - DNA, 1233 bp, Anaerocellum thermophilum DSM 6725
atgatagataatataaattttgttatcttgtgtattattattgttattcttttagttataacttcgggagttgtttttttatacaaagttataaatagtatccgtcaagaaaaaataaagcagatttttaaaaattacagtaaaaatgtctatgatgtgataacaggaaagaaaaatgttattgattcttcaaacatttacattctgagtgatgttataaatatatattattcatgggtacatggagaagaaaaaaaacggctatatatagctttgaagaaattgaatttttttgacattgctatcaagatggttcaaaaagggaacaaagttcgaaaacttaggtttgcaaaagttattagtatagtgggcgaagaagatgagctaaaaacgcttttaaaaataagcattaaagagccgtatctgatagatacaactgctgaagccattttcaaaaacatagatgagatacagaatttaagtgtttttaaaccttatttaaagactatttttctaaacattgataaatatccagattcggttagaaaaagaatagaattttttacagtttatggtggcgaaaaaataaaagatataattttatacgtgataaaagaaaacccgtcggataaagttctgatatcatgtttgaatatattttcagagattgcggcattagaagatttagaacacattgattttcttataaatcatccatcaccagaagtcagatcagctttctgcagagtgattgaaaaaataggatgtagaaactgcaaagaaaaacttgaaactctgataaaaaacgaaaatataaattttgtgaagttacgagccttaagggcgttgagcaatgtttcatctaagggttctttaaaatatttacttgcctcccttgaagatgactggttctacatgagagactttgcaagaaagatgctatctgagtttggaccagttattttaaatgatcttctaaaattttactatacaacaaatgataaatttgcaagggacaaaataagagaggttttttatagtccagtgaattttgattatattatcaagagtgctttgaattatagaactgaacaggagaaagatttagctttagaaataattaaaatacttaaatcgtctaacccccactgtttttatcaaagaatgaaagatgaaggatttgaatatcttataaccgagaaagaggtagtgaataacgaatgcgacatataa

SEQ ID NO: 81 - protein, 410 aa, Anaerocellum thermophilum DSM 6725
MIDNINFVILCIIIVILLVITSGVVFLYKVINSIRQEKIKQIFKNYSKNVYDVITGKKNVIDSSNIYILSDVINIYYSWVHGEEKKRLYIALKKLNFFDIAIKMVQKGNKVRKLRFAKVISIVGEEDELKTLLKISIKEPYLIDTTAEAIFKNIDEIQNLSVFKPYLKTIFLNIDKYPDSVRKRIEFFTVYGGEKIKDIILYVIKENPSDKVLISCLNIFSEIAALEDLEHIDFLINHPSPEVRSAFCRVIEKIGCRNCKEKLETLIKNENINFVKLRALRALSNVSSKGSLKYLLASLEDDWFYMRDFARKMLSEFGPVILNDLLKFYYTTNDKFARDKIREVFYSPVNFDYIIKSALNYRTEQEKDLALEIIKILKSSNPHCFYQRMKDEGFEYLITEKEVVNNECDI

SEQ ID NO: 82 - DNA, 1395 bp, Anaerocellum thermophilum DSM 6725
atgcgacatataataaactggttttcatggtttgtatcttattatgttttagttttgaatactgtttatgccattttaattttaatatctctttttggtattgttagttactggagaaataagataaaaggaaggattgtggaggttgtctcatcagactttgcactccctgtatcactgctggtgccggcatacaatgaagaagaaacaatagcaaaatctgtaaaatcttttttgcagattgaatatccagaatatgaagtagtagtaattaatgatgggtcaaaagatgggacattggatgtattaaaaaacgaatttgacctttacattgtagataggaaatttagaaagattttgtcaacaaaagagataaaggtcatatactattccaaaaagtattcaaatttaatcgtagtggataaagaaaatgggggaaaagctgatgctctcaatgcagggataaacgtatgtacatacccatatgtttgttcacttgatgccgattcaattttagaaagagattcaatagccaaggttatgcaaccattttttgataacccttatgaagttgtagccacaacgggtattgttaggattgtaaatggaaccgaattggattcctttggcaatataaaaaaattaaagctaccaagttcaagtcttgcaagatttcagataatagaatacttgagagcttttttgggtgcgagaaaaggtctttctatgataggaagtcttgttattgcatctggagcatttgcagcatttaataaaaatgctattataaaggttggagggttttctgatagaactgttggcgaggatatggagattgttgtaaagctcagaaaaaactcttataaagaaggcgcactgggaagagttgaatttgtaccagacccgattgtatggactcaatgtccagagactttaaaagacctttcaaaacaaagaagaagatggcaaagaggtctttgccaagttattttcatgcacaaagatattctatttaatcctaagtatggcatattaggtctttttgctatgccatatcagcttatgtttgaactattggggccgtttgtggagatgttgggttatatttttatacctatatcgtattttgctcacataatcaatttagaagtggccttatttttctttgcagttgagataatgtacggaatagttatttcgattttagcagttcttcttggagaattttctgatagaaaatatgaagggtggagagagttcggtatacttgtattgtttgcgatattagaaaattttggctacagacagatgacaatgttattcagaattgttggtacatttgaagctatacttagaaagaaaggttgggcaaagcctgagagaaagaagttataa

SEQ ID NO: 83 - protein, 464 aa, Anaerocellum thermophilum DSM 6725
MRHIINWFSWFVSYYVLVLNTVYAILILISLFGIVSYWRNKIKGRIVEVVSSDFALPVSLLVPAYNEEETIAKSVKSFLQIEYPEYEVVVINDGSKDGTLDVLKNEFDLYIVDRKFRKILSTKEIKVIYYSKKYSNLIVVDKENGGKADALNAGINVCTYPYVCSLDADSILERDSIAKVMQPFFDNPYEVVATTGIVRIVNGTELDSFGNIKKLKLPSSSLARFQIIEYLRAFLGARKGLSMIGSLVIASGAFAAFNKNAIIKVGGFSDRTVGEDMEIVVKLRKNSYKEGALGRVEFVPDPIVWTQCPETLKDLSKQRRRWQRGLCQVIFMHKDILFNPKYGILGLFAMPYQLMFELLGPFVEMLGYIFIPISYFAHIINLEVALFFFAVEIMYGIVISILAVLLGEFSDRKYEGWREFGILVLFAILENFGYRQMTMLFRIVGTFEAILRKKGWAKPERKKL

SEQ ID NO: 84 - DNA, 1005 bp, Anaerocellum thermophilum DSM 6725
atgttctatcggcaggacccagatatcgaaaagaatatagtgagtgttcaaagattagatattacaacagaggtagggcaaaagccacagcttccgccaactgtcatggtggtttatgatgatgatacatcctctgaggcaaatgttacatgggaagctattgatgagagtaaatactcagcgattggtgaatttactgtgactggtactgtagaaggcacaactctaaaagcatatgccaaagtcaaggttatcaacagcaaaaaccttgtaaaaaattacagttttgaagaaggactaaagtattggataagcgaaggaaatacaaatgcagtcaaaactgaaagtggtgggcattctgggacagcgcagcttactcactggagtgacaaaccttacaaggtggtcacatatcaaacaattgagaatataccaaatggtatatacattttcagagcctttgcaaatggaggaggcggtcagaatgctaactacctgtttgtaaaagactatggtggagaagaattgagaataaatatgccaactacatgggtcgctgagtggcacagaatggtgattgcaaacattaaggttacgaccggacgtgttacaatagggctttattcagattcagagaatgcaggaggtacatggtgcaaccttgacgatgttgaatttttcaaggttgctgacagtgaatttgaaattttaaatgcaaatctgcaaaaggataaaggaattgttgcgagtgttacagtgaaacaatcagaaggtatccagcatgaggggaaagaggccattgtatttgagctacttaaagggacaactcctgtttcaattgttgctattgaaaaagatataatagatgctgaagatttcaaggcatactttaatgtaaatgattatcttagccctgattacagggtaaaagtatttgtatttgacaaatttgacacaagcctttctgttccaaatagtcttgctgagcctgttgagcttcgatga

SEQ ID NO: 85 - protein, 334 aa, Anaerocellum thermophilum DSM 6725
MFYRQDPDIEKNIVSVQRLDITTEVGQKPQLPPTVMVVYDDDTSSEANVTWEAIDESKYSAIGEFTVTGTVEGTTLKAYAKVKVINSKNLVKNYSFEEGLKYWISEGNTNAVKTESGGHSGTAQLTHWSDKPYKVVTYQTIENIPNGIYIFRAFANGGGGQNANYLFVKDYGGEELRINMPTTWVAEWHRMVIANIKVTTGRVTIGLYSDSENAGGTWCNLDDVEFFKVADSEFEILNANLQKDKGIVASVTVKQSEGIQHEGKEAIVFELLKGTTPVSIVAIEKDIIDAEDFKAYFNVNDYLSPDYRVKVFVFDKFDTSLSVPNSLAEPVELR

SEQ ID NO: 86 - DNA, 1644 bp, Anaerocellum thermophilum DSM 6725
atgaaaaaaagaacaaggatagcttcaagtctattgcttgtttttgtgtttttgttctctttattgtctgtgggatattctgcccaggaagttattttaaattccatacctgataaaaatccaggtgaagacgttgtaatttctgggcaaacgatgtttgatgagattgttatcaaagtgttgagaccaaattctaccatactttatataaatacagtgaaggggaaaaacttcagtgacaaatttactttaccagcagacttgcctgagggtacttacacagttgttactggaaaaggttctattgtcgatataaaaactttcaatgtagtaaggaaacaaccatctttacctaattcgtttcctgagattttaatccctaatttcaaagaagctgaaaaacaggcaggtgtacagccatctcaaaaggaagatattaaacaaactgataaagctgctttgcctgaaattacaattgataatagtgggataattaggcccaaaatatatcagctgtcagagggaagattagacgttagattagatgagtctttagtacaaaaagcactgcagatgcaggtgtcaaaaaacaaaattttaacgttggatttaaaaaacagcaagcctttagtacagtacaatattgtgcttccatcgaaagtatttacagcttcttttgatgtgcaggaagttttagttgatacagatgaattagatttgaaacttccagctgacctattaaaggaaaagaacatagacaagaacaagcaaatagagatattgataaaaaaagtagaaaattctaatataccttcagagattcgtgcggcagttgggaaaagaccaatatatgaaattgggttttatcaagatttgaaaaaggtgaatgttgaaagacctacaaattctataaaaatatctattgcttatactcctggaatagaagagctgagcggtattgaaaacttggttttgtttcatataaaagaggacgggaaaacagagattttatcaaatagtaaatatgtaaggacatcgaaatgtgtgattgcaaatgtgaatggttttagcaagtttgcaatagggtatgttaccaaaaaatttgacgacttaaaaaattattcgtgggctgaaaaagctgtgtcttccctggccgcaagaaatataataagtggtgttgacaaaagtagttttagacctcaagagtatataaagcgtggagagtttgtcaagtggctggtaaatgctcttggccttgatgctcagtattcttcgaactttgaggatgtcaaaaaagatagcagctactggcgtgaagttgcaattgcaaaagcacttggcattgcaaaaggttatgcgaataagtttaaaccagaagattatataacaaggcaagatatgatggttcttgttaaaagagcattggaggttgcaaacaaaccaattgtaaaaacaaaaagtagccttgcaactaaattttctgattcatctgatatatctttgtatgctcaagatagtatttctattttggttgcaaatgatttaataaaaggaaatagcaaaaatcaaatactgcctcgaaagtttgcaactcgtgcagaagctgctcagcttttgttcaggatatttttcaaatgggaatag

SEQ ID NO: 87 - protein, 547 aa, Anaerocellum thermophilum DSM 6725
MKKRTRIASSLLLVFVFLFSLLSVGYSAQEVILNSIPDKNPGEDVVISGQTMFDEIVIKVLRPNSTILYINTVKGKNFSDKFTLPADLPEGTYTVVTGKGSIVDIKTFNVVRKQPSLPNSFPEILIPNFKEAEKQAGVQPSQKEDIKQTDKAALPEITIDNSGIIRPKIYQLSEGRLDVRLDESLVQKALQMQVSKNKILTLDLKNSKPLVQYNIVLPSKVFTASFDVQEVLVDTDELDLKLPADLLKEKNIDKNKQIEILIKKVENSNIPSEIRAAVGKRPIYEIGFYQDLKKVNVERPTNSIKISIAYTPGIEELSGIENLVLFHIKEDGKTEILSNSKYVRTSKCVIANVNGFSKFAIGYVTKKFDDLKNYSWAEKAVSSLAARNIISGVDKSSFRPQEYIKRGEFVKWLVNALGLDAQYSSNFEDVKKDSSYWREVAIAKALGIAKGYANKFKPEDYITRQDMMVLVKRALEVANKPIVKTKSSLATKFSDSSDISLYAQDSISILVANDLIKGNSKNQILPRKFATRAEAAQLLFRIFFKWE

SEQ ID NO: 88 - DNA, 2502 bp, Anaerocellum thermophilum DSM 6725
atgttaactaataaaaagcttagggagtatgtatattttgtatcgattatttttttcatgactattatattgttaaatggacagataaatgtgagcaaaagtaatttagcaatggcggcaacaggaagtcaaattgagaagttaaatagaggattaattgcaataaaagtaaccaacggagtatttttgagttggagaatgtttggttcagatttagcgaatatagggtttaatatttacagaaatggagtgaagataactaatacgccgattcagaacagcacaaactatgttgatactgctgggacagctgcttcgaagtattatgtaaaagcagtgataaatggtgtagaggtagagcaatctgaagaagtaagtgtgcttagtagcaattatattgaaataagattaaataaaccagcgaattctccactcggtgcttcatattcgccaaatgatgcaagtgttggcgatttagatggtgatggggaatatgagattgttctgaaatgggatccaagtgattcaaaggataactcacaatctggatacacaagcaatgtgtatttagacgcttacaaattaaatggcaagtttttatggagaattgatttgggtaggaatataagagcaggagcacattatacacaatttatagtatatgatttagatggtgacggaaaagctgaagttgcatgtaaaacagctgatggaacaatagatggacaagggaatgtgataggtgatccaaatgctgattggaggaattcctctggttatattttatcagggcctgaatatttgactatatttgaaggtgcgacaggacgagcaataaagacggttaattatattccgccacgagggaatgtttcatcatggggtgattcttatggaaacagggttgacaggtttttagcagcagtagcttatttggatgggaatagacctagcttaattatgtgccgaggatattacacaaaaacatatatagttgcttggaattggcgaaatggtgagttaacaaagttatggcaatttgacacaggagagattagagatggatatagagatgattacgaaggacaaggaaatcacaatttgagtgtggctgatgttgacaatgatggtaaagatgagataatatatggtgctatggtagtagatgataatggagcaccattatattcaactaaattaggtcatggtgatgcaatgcatgtgacagatattgatccagacaggccaggattagaagtttggcagtgtcatgaaggaagtacaggagcgagtttaagggatgcacgaactggacagatattagtgagagttttaacatctggagatagtggacgtgctttgacggcagatattaacccgcgatatagaggattagaaatgtgggcggcaggtggaataagtgttagagattgtagaggtaatgtaatcagtaatgcgacaccaccaattaattttgcaatatggtgggatggagatttaggtagagaattgttggataatgtatatatttataaatgggattataacaataataggagtaatactatattcacagcaagtggatgttcatcaaacaatggcacaaaagcaacaccttgcttgagtgcagatatacttggggattggcgagaagaggtcatatttaggactagtgataacaatgcaatcaggatatatacaaccaccacattgacagattataagatacctacgcttatgcataacaggcaatatagggtgtctatagcatggcagaacgttgcatataatcaaccacctcacgtaagtttttatttagggtatgagactaatgtgaataacatatatcaatattttgaaggttatgggcaacaaccaattgttacaccgtcgctaaccccgacaaaaacaccaacgcctacatcaactccattgccaacatcaactgcaacatctacgccaactccaacagcaacagcaacaccaacaccgacaccaacagcaacaccaacaccgacgccgagcagcacacctgtagcaggtggacagataaaggtattgtatgctaacaaggagacaaatagcacaacaaacacgataaggccatggttgaaggtagtgaacactggaagtagcagcatagatttgagcagggtaacgataaggtactggtacacggtagatggggacaaggcacagagtgcgatatcagactgggcacagataggagcaagcaatgtgacattcaagtttgtgaagctgagcagtagcgtaagtggagcggactattatttagagataggatttaagagtggagctgggcagttgcaggctggtaaagacacaggggagatacagataaggttcaacaagagtgattggagcaattacaatcaagggaatgactggtcatggatgcagagcatgacgaattatggagagaatgtgaaggtaacagcgtatgtagatggggtgctggtatgggggcaagaaccaaaataa

SEQ ID NO: 89 - protein, 833 aa, Anaerocellum thermophilum DSM 6725
MLTNKKLREYVYFVSIIFFMTIILLNGQINVSKSNLAMAATGSQIEKLNRGLIAIKVTNGVFLSWRMFGSDLANIGFNIYRNGVKITNTPIQNSTNYVDTAGTAASKYYVKAVINGVEVEQSEEVSVLSSNYIEIRLNKPANSPLGASYSPNDASVGDLDGDGEYEIVLKWDPSDSKDNSQSGYTSNVYLDAYKLNGKFLWRIDLGRNIRAGAHYTQFIVYDLDGDGKAEVACKTADGTIDGQGNVIGDPNADWRNSSGYILSGPEYLTIFEGATGRAIKTVNYIPPRGNVSSWGDSYGNRVDRFLAAVAYLDGNRPSLIMCRGYYTKTYIVAWNWRNGELTKLWQFDTGEIRDGYRDDYEGQGNHNLSVADVDNDGKDEIIYGAMVVDDNGAPLYSTKLGHGDAMHVTDIDPDRPGLEVWQCHEGSTGASLRDARTGQILVRVLTSGDSGRALTADINPRYRGLEMWAAGGISVRDCRGNVISNATPPINFAIWWDGDLGRELLDNVYIYKWDYNNNRSNTIFTASGCSSNNGTKATPCLSADILGDWREEVIFRTSDNNAIRIYTTTTLTDYKIPTLMHNRQYRVSIAWQNVAYNQPPHVSFYLGYETNVNNIYQYFEGYGQQPIVTPSLTPTKTPTPTSTPLPTSTATSTPTPTATATPTPTPTATPTPTPSSTPVAGGQIKVLYANKETNSTTNTIRPWLKVVNTGSSSIDLSRVTIRYWYTVDGDKAQSAISDWAQIGASNVTFKFVKLSSSVSGADYYLEIGFKSGAGQLQAGKDTGEIQIRFNKSDWSNYNQGNDWSWMQSMTNYGENVKVTAYVDGVLVWGQEPK

SEQ ID NO: 90 - DNA, 1383 bp, Anaerocellum thermophilum DSM 6725
atgataaaatctaagaataaaaaagaggaggtttgggtgatgagtaacaggaagattttagccattgtagtcagtttgataatggttgtttcattgtttacagggattgggttgcgtaatgaagttgcaaaggcagcgacacttttaacagatgattttgaagatggcaacagagatggatggtcgacatcgaacggtagttggagtgtagtagtggatgggagcaaggttttaaagcaggctagcacaggttctgaggcgagagcatatactggttcatctgattggagtgattatacagttgaagcgaaagttaaagtattaaatgtgaaggattcgagttcaggtgcgggagtgatagtgagatataaaaactcaggtaacttttatgcgttggtgctaaggggttcaaagatagaaatagggaagaaattaaacagtaactggagtacattggcgttcaagtcatttacgttggatcaggatacctggtataatgtgaaattagaagtaaatgggagcaagttagttggatatgttaatgggagtcaagtattaagtgcaagtgatttatcgattacgacaggaaaagcaggtttaatagctgacaggtgtgttgctgaatttgatgatgttgttgtaaattcaagtgtgagcggtacagcacctactccgacaccaacaccgacttcatcagtgacaccaacaccgacatcgactccaacgccaaccaaaacacctactccaacttccacaccagtaccaacacagaccccagcagtaacaccgacgccgaccccaaatacgggtggtgttttagttattacagatacaataattgtaaaatccggtcaaacatatgatggtaaaggaataaaaataatagctcaaggaatgggtgacggaagtcaatctgaaaatcaaaagcccatatttaaacttgaaaaaggggcaaatttgaaaaatgtaataattggagcgccaggttgtgacgggatacattgttatggtgataatgtggttgaaaatgttgtatgggaagatgttggagaggatgcgttgactgtaaaaagtgagggggtagtggaagttattggtggttcagcaaaagaagctgctgacaaggtgttccaacttaatgcaccgtgtacattcaaagtaaaaaacttcacagctacaaatataggaaagcttgtaagacaaaatggtaatactactttcaaagtagttatttatcttgaagatgtaacattaaacaatgtaaaaagctgtgttgcaaaatctgatagtccagtatcagaactgtggtatcataacttgaatgtaaacaattgtaaaacattatttgaatttccgtcccaatcacagatacatcaatactaa

SEQ ID NO: 91 - protein, 460 aa, Anaerocellum thermophilum DSM 6725
MIKSKNKKEEVWVMSNRKILAIVVSLIMVVSLFTGIGLRNEVAKAATLLTDDFEDGNRDGWSTSNGSWSVVVDGSKVLKQASTGSEARAYTGSSDWSDYTVEAKVKVLNVKDSSSGAGVIVRYKNSGNFYALVLRGSKIEIGKKLNSNWSTLAFKSFTLDQDTWYNVKLEVNGSKLVGYVNGSQVLSASDLSITTGKAGLIADRCVAEFDDVVVNSSVSGTAPTPTPTPTSSVTPTPTSTPTPTKTPTPTSTPVPTQTPAVTPTPTPNTGGVLVITDTIIVKSGQTYDGKGIKIIAQGMGDGSQSENQKPIFKLEKGANLKNVIIGAPGCDGIHCYGDNVVENVVWEDVGEDALTVKSEGVVEVIGGSAKEAADKVFQLNAPCTFKVKNFTATNIGKLVRQNGNTTFKVVIYLEDVTLNNVKSCVAKSDSPVSELWYHNLNVNNCKTLFEFPSQSQIHQY

SEQ ID NO: 92 - DNA, 1962 bp, Anaerocellum thermophilum DSM 6725
atgagtaacaggaagattttagccattgtagtcagtttgataatggttgtttcattgtttacagggattgggttgcgtaatgaagttgcaaaggcagcgacacttttaacagatgattttgaagatggcaacagagatggatggtcgacatcgaacggtagttggagtgtagtagtggatgggagcaaggttttaaagcaggctagcacaggttctgaggcgagagcatatactggttcatctgattggagtgattatacagttgaagcgaaagttaaagtattaaatgtgaaggattcgagttcaggtgcgggagtgatagtgagatataaaaactcaggtaacttttatgcgttggtgctaaggggttcaaagatagaaatagggaagaaattaaacagtaactggagtacattggcgttcaagtcatttacgttggatcaggatacctggtataatgtgaaattagaagtaaatgggagcaagttagttggatatgttaatgggagtcaagtattaagtgcaagtgatttatcgattacgacaggaaaagcaggtttaatagctgacaggtgtgttgctgaatttgatgatgttgttgtaaattcaagtgtgagcggtacagcacctactccgacaccaacaccgacttcatcagtgacaccaacaccgacatcgactccaacgccaaccaaaacacctactccaacttccacaccagtaccaacacagaccccagcagtaacaccgacgccgaccccaacgccgacgacagttccaacacctgccccgacacctgtacctggggtgaatgctatttatgtggcaccaagtgggagctcagataatcctggtaccattgatcgacctactacattagaaaaagcaatcacgatagtacaacctgggcagataatctacatgagaggtgggacgtataagtattctgcgcagatcacaattgaaagaaataatagtggtacaagcaatgcaagaaaatgtatttatgcatttccaaatgaaaggccaatattggacttttcatctcaaacatatgggagtgtggactcaaatccaagaggattacagattaatgggaactattggcacataaaaggattagaagtcatgggagctgcggacaacggaatcttcgtaggaggcagctacaatataattgaacaatgtgaaattcatcataacagagattcaggtttgcagataagcaggtatataagttctgcaaccagagatgagtggccaagttataacttgatattgaattgtacatcacatgacaatatggatccagataacggtgaagatgcagatggttttgcatgcaaactaacagcaggaccaggaaatgtattccgaggttgtgtagcgtactacaatgttgatgatggttgggatttatacacaaaaagtgagacaggagctattggtgaagtattaattgaggattgtgtggcatatggtcacgggcaaacatcaaccgggagtgccacatctagcagtgatggaaatggctttaagctaggaggcagtaatataaaggtcaatcatacagtgagaagatgtatagcatttaataacaacaaacatggatttacttataatagtaatccgggtagcataacagtggaaaattgtacgggctataataacggtttaaaggtaagtggaaggaacttttattttgaagaaggtacacacgtgttgaagaattgtttatcctacaaagagagtgcatcgagtgatttggtaagtggaacgataattaattgtgttttgtggagtaataggcaagcaataaagctaaatggtcaactggtaaccgataatgacttttacagcttaacaccaaccataacaaggaatagtgatgggggtttaaacttaggagactttttaaagccaaagcctggtagtggtttagaaggaataggagcaaggtaa

SEQ ID NO: 93 - protein, 653 aa, Anaerocellum thermophilum DSM 6725
MSNRKILAIVVSLIMVVSLFTGIGLRNEVAKAATLLTDDFEDGNRDGWSTSNGSWSVVVDGSKVLKQASTGSEARAYTGSSDWSDYTVEAKVKVLNVKDSSSGAGVIVRYKNSGNFYALVLRGSKIEIGKKLNSNWSTLAFKSFTLDQDTWYNVKLEVNGSKLVGYVNGSQVLSASDLSITTGKAGLIADRCVAEFDDVVVNSSVSGTAPTPTPTPTSSVTPTPTSTPTPTKTPTPTSTPVPTQTPAVTPTPTPTPTTVPTPAPTPVPGVNAIYVAPSGSSDNPGTIDRPTTLEKAITIVQPGQIIYMRGGTYKYSAQITIERNNSGTSNARKCIYAFPNERPILDFSSQTYGSVDSNPRGLQINGNYWHIKGLEVMGAADNGIFVGGSYNIIEQCEIHHNRDSGLQISRYISSATRDEWPSYNLILNCTSHDNMDPDNGEDADGFACKLTAGPGNVFRGCVAYYNVDDGWDLYTKSETGAIGEVLIEDCVAYGHGQTSTGSATSSSDGNGFKLGGSNIKVNHTVRRCIAFNNNKHGFTYNSNPGSITVENCTGYNNGLKVSGRNFYFEEGTHVLKNCLSYKESASSDLVSGTIINCVLWSNRQAIKLNGQLVTDNDFYSLTPTITRNSDGGLNLGDFLKPKPGSGLEGIGAR

SEQ ID NO: 94 - DNA, 2382 bp, Anaerocellum thermophilum DSM 6725
atgggaagaaccatatgtattaaaaaaagtgcaaatatgttcttaatatttttagttatggtttttctttttcattttctaatgtcagttttcttattatatatgactaaagaacttattttgagagacataattaatgataagtcacttcaattagataaacttcgcgaaaaaatagacaatgaactaaagaaaatgaatgagatagttacacgtattcttactgatgatgaattaaaatgggtagggaatctaagggggttaagagaaaattctttagatttatgggagtactttgaatattataaacactttaaagacataggcatgattaatggagagttaaaacctatcattgtactttttttacgcgagggagaaatagtatattttgcttccgaagaatttggctctttttttacatttggatttgaaaatttttgtaattacttttcacctgacagaatgaactgccgcgtttggctcaataaaatattcgataaaaacaacagtaaatgcaaaaataagttaatatcacaagaatattcaattagtggccaaagaatattggcattgcatgaaacatattactttccatttgataacattggaaatcaattggcaattcttctggtaattattgatgttgctaaattttgtaagctactaaaggaaaacaaattaaacagccaagattcaataattttaatttatgataggtgtgagaaaaaaatattaacttcaaataaagcagatattaataatacaataaataacatattagaaaaacttagcaaaaataagaaatatatttctaatctgtataatgtaataacaatacgaaataaaaagtatatatttttacggttggcttcaaatgtatatgattgggattatgtttatctagtaccatataacagcataaaaagtgaaatatacatttcaagaactgtttatacattgtatctaacggagtttacacttttcataattattataagtttatatgctatttatatcaaattatataaagtaaggacatataaaataactaagggcttaaaaaatgtaaacgaggagaatgagaataaaatacttacttttaggtgcataaacaagttaaaggagaaagaccgtaggttaatacacactaaaattacaagttacaggattcttaataaaataaccagtaaacaagaattattaaacgatatgataatagaaaagcttatttatggttggtcttatagcaaaggggcaatagaagaaaaaatccaaagtataggcttaaaaattggaggtaaaaaatttttagttgccataatcaaaatgctctctcttaccaaacaagttagaacaagagatataataaaaaatgagttagaaagtattagggttgactcaaaaattaacttggtattttattatgtttaccaacttgcagatagtgatcttgctttaattttagcatttgatgaagatgaagatgaaaaagtaagtcaaaatgtaaatctatggctgagattgataggtgatagattagtaattaaaatatgtcacaaatatttaattgcagttggtcgtatagttaataacattgacgaatgtagaatttcatttttagatgcaaaagaaattattgaagctaataaattaatcacaacagagaacgttgagaatggcatactgtggtattataatgttgttaagaaagataataatatatggtatccaatagaaatagaagagagattaatgttgctagttaatcttgggaaaatatctgagattgaagaaattctacacctattatattataaaaactttgaagaaaaagatatatcactgagtttaaagtatgtgctaattagtgagttaattggaacaattattaaaattgcaaatatgacaaaagtaaatattgacaatctatttgacattaagaactttatttttattgaagaaaatgactttgacaggttatttgaagatataaagcaagtatttattaatataacagaacaaatcaagagaaacaggaaggcttctaaggaaaaattaatagaggagatattagaatttataaacaaaaacttatttaatccaaacatgagtatatcccttgtcgctgaaagatttaatttatctgaatcttatttttctaatatttttaaaaatgcagtaggtataaagttcagtgattatgtagaaaagttaagaatagaagaagcatataaattgataaagcaaaaaaagtggaatttagacgatattagtaaaatggtaggatatactaacattaaaacctttagaagagcttttaaaagggtaaaaggttgtttacccagtgaaattttaaacataaatgaacatgatatatag

SEQ ID NO: 95 - protein, 793 aa, Anaerocellum thermophilum DSM 6725
MGRTICIKKSANMFLIFLVMVFLFHFLMSVFLLYMTKELILRDIINDKSLQLDKLREKIDNELKKMNEIVTRILTDDELKWVGNLRGLRENSLDLWEYFEYYKHFKDIGMINGELKPIIVLFLREGEIVYFASEEFGSFFTFGFENFCNYFSPDRMNCRVWLNKIFDKNNSKCKNKLISQEYSISGQRILALHETYYFPFDNIGNQLAILLVIIDVAKFCKLLKENKLNSQDSIILIYDRCEKKILTSNKADINNTINNILEKLSKNKKYISNLYNVITIRNKKYIFLRLASNVYDWDYVYLVPYNSIKSEIYISRTVYTLYLTEFTLFIIIISLYAIYIKLYKVRTYKITKGLKNVNEENENKILTFRCINKLKEKDRRLIHTKITSYRILNKITSKQELLNDMIIEKLIYGWSYSKGAIEEKIQSIGLKIGGKKFLVAIIKMLSLTKQVRTRDIIKNELESIRVDSKINLVFYYVYQLADSDLALILAFDEDEDEKVSQNVNLWLRLIGDRLVIKICHKYLIAVGRIVNNIDECRISFLDAKEIIEANKLITTENVENGILWYYNVVKKDNNIWYPIEIEERLMLLVNLGKISEIEEILHLLYYKNFEEKDISLSLKYVLISELIGTIIKIANMTKVNIDNLFDIKNFIFIEENDFDRLFEDIKQVFINITEQIKRNRKASKEKLIEEILEFINKNLFNPNMSISLVAERFNLSESYFSNIFKNAVGIKFSDYVEKLRIEEAYKLIKQKKWNLDDISKMVGYTNIKTFRRAFKRVKGCLPSEILNINEHDI

SEQ ID NO: 96 - DNA, 1818 bp, Anaerocellum thermophilum DSM 6725
gtgggtgatgtaaattctgtcaattccgctttaaaaaacttcaatattgtagttgcttctgccaacaacaaagctgtggaaaatgtcaccaaagaaataccagttctaaattcagttgaccaatcctgtcttgaaaaatatgaactccattatttcaaagatggagccgaactggtatacgactacagaggggaaaacaatgaaagcagtgagaaaactgaatctttagtatcagttgaaaatgggcagcaatgctgggcattgatttcagcagttttgggtaaaaaagaaaacagagaaaagtttttcagtgctctggaaaagtatattaatgaattattttcttctataccgccagtcaagtgggaacagtgtaaaagaagtttcaatcatgtcttcaagaagtttagacatgtccagaaactttacaggactatggaactttttctgaaactcgaagaaaacaggtttaaatttacccgtatgatagctgctgcggggaagatttgcctaaaaaaattttttgccagatattttcccgatgacatgaaacttccttcctcagagttctggtcccaaaatgagcacgaaattcacaaatcatctccatggatgagcaggtatctcaatgatatacgcaatgaagtttttgcaagagctcttcagttacatcaggcagctattgctgcaaacaaagagaagttcggggagaatttagaaaaattcattaaatatatgagaaaaaacgaatcactgcctgcagaaaaggcaaaagaactgtggcatactttctttatgattgttccagttgtctcaacaacatttgcgtcgctgagcaatatgtttaaagatgttgacgacgaaatcattgactggctgattgttgatgaggctgggcaggcactaccccagcattttatcggcgcactcctcagaagtaaaagagcaataatagtgggagaccctctacaaattccacctgttgttaaaataccaccatttgttattaatgatgtgttcaaagcatatgggattttcaaatggagacaggaagacagtaattccagtacaccacgtataactgaaactgattccgtacaaattgtagccgatagagccaacaaattcggtgcaaagattggcgatatgtgggttgggtgtcctctgagagtacataagcgttgtacggaaccaatgtttacagtagcgaatagaattgcttataaaaaccttatgatttttgatgtgaataaacctgaaggattgcaaactgtttttaaagatagcttctgggtagatgtaaagggtaagtgtgtttacaagcattatgtaagagaacagggaaaagtagtgaaagctatcgtacaagaatttttgcagcgaaccttgacaactcaaaatgaggttaaattaacagaagagctttttatcatttcgccttttaaagcagtcaaaagtagtatatcttctatactgaaaagaatgcctctgtataacacaaatttaaagaaagaagaatgggagaatatagttaatgaaatagttggcacaattcacagttttcagggcaagcaagccaacaatgtaatcatatgccttggtgccgatgaaagtaatgaaggagctgtaagatgggcatcttcagaaccgaatattttaaatgtggcgctgaccagagctaaatatagagtcatagtaataggtgataaagacctgtggggaaagcataaatattttgatacactcttagaagaactaggtgaaaaagtgattgagtataccacagaaaaagatctggtcaacaagatttttgcttag

SEQ ID NO: 97 - protein, 605 aa, Anaerocellum thermophilum DSM 6725
VGDVNSVNSALKNFNIVVASANNKAVENVTKEIPVLNSVDQSCLEKYELHYFKDGAELVYDYRGENNESSEKTESLVSVENGQQCWALISAVLGKKENREKFFSALEKYINELFSSIPPVKWEQCKRSFNHVFKKFRHVQKLYRTMELFLKLEENRFKFTRMIAAAGKICLKKFFARYFPDDMKLPSSEFWSQNEHEIHKSSPWMSRYLNDIRNEVFARALQLHQAAIAANKEKFGENLEKFIKYMRKNESLPAEKAKELWHTFFMIVPVVSTTFASLSNMFKDVDDEIIDWLIVDEAGQALPQHFIGALLRSKRAIIVGDPLQIPPVVKIPPFVINDVFKAYGIFKWRQEDSNSSTPRITETDSVQIVADRANKFGAKIGDMWVGCPLRVHKRCTEPMFTVANRIAYKNLMIFDVNKPEGLQTVFKDSFWVDVKGKCVYKHYVREQGKVVKAIVQEFLQRTLTTQNEVKLTEELFIISPFKAVKSSISSILKRMPLYNTNLKKEEWENIVNEIVGTIHSFQGKQANNVIICLGADESNEGAVRWASSEPNILNVALTRAKYRVIVIGDKDLWGKHKYFDTLLEELGEKVIEYTTEKDLVNKIFA

SEQ ID NO: 98 - DNA, 330 bp, Anaerocellum thermophilum DSM 6725
ttgaaagatacttttgaacagttaattaaaaatcagaaaaatattagagaaatctatatgaatttactccagaaattttgttacgatatttatccagtcaggaacaaagaatttacaaattcattcggtgttacatggcttaattccaaaaaaggcaaacaacacgaacaaagaagaaaagtcgagatagaaaacgaaatagaggaagaaattgaaacgccactgcaagacctttttgccagtttttatctaaaagacattgaagaagcacttaaacgccttgagaatgataatctaaaccctgtcttgtttcgcttctttactgagtaa

SEQ ID NO: 99 - protein, 109 aa, Anaerocellum thermophilum DSM 6725
LKDTFEQLIKNQKNIREIYMNLLQKFCYDIYPVRNKEFTNSFGVTWLNSKKGKQHEQRRKVEIENEIEEEIETPLQDLFASFYLKDIEEALKRLENDNLNPVLFRFFTE

SEQ ID NO: 100 - DNA, 474 bp, Anaerocellum thermophilum DSM 6725
atgataaatacaattgcaaaagatgagatgaaaagaatactgctggcataccagaaacttgaattgttgcagcctgctcctattcccaatggagtttttataaacgactttactgaaatctggagtcctaactatttcacagattgcagagtctatcttggaatttatgattaccaggaagtaaaagaggcttttgaaaaaatttataaccagcaatatgaccatcccgaaaaagaagatgaaaaacaaaacagaaaacaaaaactttactggttctatttcgatattgatgagaacgggaaatataaagatgacagtttaaaaatttcttcaactttgtgggctcttaaacaactggaactggaaaatacaaacaaaagaaggctgtcagaaactgcggagaaaaaagatgaagaaaagcaaactgaagattatgatcttggtaaaaatttacagagtttgctcaaaacttga

SEQ ID NO: 101 - protein, 157 aa, Anaerocellum thermophilum DSM 6725
MINTIAKDEMKRILLAYQKLELLQPAPIPNGVFINDFTEIWSPNYFTDCRVYLGIYDYQEVKEAFEKIYNQQYDHPEKEDEKQNRKQKLYWFYFDIDENGKYKDDSLKISSTLWALKQLELENTNKRRLSETAEKKDEEKQTEDYDLGKNLQSLLKT

SEQ ID NO: 102 - DNA, 186 bp, Anaerocellum thermophilum DSM 6725
atggctgactataagaaagcatatacattacgaatagatgaaactttgcttgataagattagagtaatcgccgagaaaaataaacgctcaattaatgctcaaattgaatatttgattcaacagtgtgtcgaagaatttgaagctgagcacggtgagataaaaattgaagaagaaaatactgaatga

SEQ ID NO: 103 - protein, 61 aa, Anaerocellum thermophilum DSM 6725
MADYKKAYTLRIDETLLDKIRVIAEKNKRSINAQIEYLIQQCVEEFEAEHGEIKIEEENTE

SEQ ID NO: 104 - DNA, 843 bp, Anaerocellum thermophilum DSM 6725
atggatatatttaccttaataagaacaaaatataatactttatctcaaactcaaaagaaaattgcagattggattttagaacatggaaatgaaattattttaatgtcaataggcgaaatcgcagcaaagtgttcaacaagtgaagctactgtaatgagatttttaagaaaacttggttttgattcatatcagctctttaaggtaaaagtggctcaagatattgttgatgtgactccccgagccgtttatgaggatgtaagtagtgaagatagtgttaatgagataaaacagaaagtaattcaatctacaattgatgctatcagagatatagataagttaattgaagatagcacaatagaaaaagctatagagataatggctaatgcaaaaagaatctttttctttggagtgggagcatcaggagcaatagcaaaggacgcatttcacaagtttttaagattaggcataaataccatatattgtagtgattctcatattatgagcataatgtgtagccatatgacagaaaaagatgccgttttggcgatttcacactcaggagaaagtcgagaaataattgatgcaatagaacttgcaaaagaaaacaaagcaaaagtaatttcgtttacaagttacccgaattctaccttggcaaaactttcagatgtggtttttctaagtgctaccaaagagactaaattcagatctgacgcgatggtttcaagaattgtgcaatgtgtgattatagacattctctatgtttctctagttttaaagttaggaagtgaagcaattgtaaatgtgaataaatcaagattagcagtcgcaaaaaagaaaaaataa

SEQ ID NO: 105 - protein, 280 aa, Anaerocellum thermophilum DSM 6725
MDIFTLIRTKYNTLSQTQKKIADWILEHGNEIILMSIGEIAAKCSTSEATVMRFLRKLGFDSYQLFKVKVAQDIVDVTPRAVYEDVSSEDSVNEIKQKVIQSTIDAIRDIDKLIEDSTIEKAIEIMANAKRIFFFGVGASGAIAKDAFHKFLRLGINTIYCSDSHIMSIMCSHMTEKDAVLAISHSGESREIIDAIELAKENKAKVISFTSYPNSTLAKLSDVVFLSATKETKFRSDAMVSRIVQCVIIDILYVSLVLKLGSEAIVNVNKSRLAVAKKKK

SEQ ID NO: 106 - DNA, 1098 bp, Anaerocellum thermophilum DSM 6725
atgagtaaaaaaatgaaaataatggtaattggagacgcaatgataccgggtaaagattttgaatcagcagctaaaaaatatttatctgattatgtggaagaaataattacaggagattgggaaaataattgggacaatttacaaagaagaagattggaagtagaaaagaaagggcccgagattgaggaagtagttcctttaataaaggaaaaagggcaagatgtttcaatgttgtttggtttatttgttcccatttccaaagaaacattcaattacttgccaaaggtaaagattattggggtttcgcgagcaggcttagaaaatgtaaacgtaaaagaagcaacccaacgaggagttttagtgttcaatgtccagggaagaaatgcagaagctgtttctgactttgcaataggtttgcttttggcagaatgtagaaacattgcgagagcccactatgcaataaagaatggccagtggcggaaagaattttctaattctgattggattccggaactaaaaggcaaaacagttggtattattggttttggatatattggtagactggtagcaaaaaaactctctggatttgaagttagaagacttgtgtacgatccttatgtaagtgaagaggaaattagagaatgcggatgtataccagtagacaaagagactttgttcaaagaaagtgattttattactctccatgcacgcctcacagaagagaataaaaatttggttggcaaatatgagatttcattgatgaaaccaacagcatacattattaacactgcacgggcaggtctaattgataaagaagcattaatagaggctctaaagacaaagagaatagcaggagcagcactggatgtgttctgggaagaacctattccttcggacagtgagttgttagaattggacaatgttactcttacaagtcatttagcaggaacaaccaaagaagcacttacaagatcacctgagcttttaatggaggatgtcaagaagtttattgaagggcagaaagcaagatttattgtgaatccagaggttttggaaaaccaagagttcaagaaatggctggagggtgtgaagaaatga

SEQ ID NO: 107 - protein, 365 aa, Anaerocellum thermophilum DSM 6725
MSKKMKIMVIGDAMIPGKDFESAAKKYLSDYVEEIITGDWENNWDNLQRRRLEVEKKGPEIEEVVPLIKEKGQDVSMLFGLFVPISKETFNYLPKVKIIGVSRAGLENVNVKEATQRGVLVFNVQGRNAEAVSDFAIGLLLAECRNIARAHYAIKNGQWRKEFSNSDWIPELKGKTVGIIGFGYIGRLVAKKLSGFEVRRLVYDPYVSEEEIRECGCIPVDKETLFKESDFITLHARLTEENKNLVGKYEISLMKPTAYIINTARAGLIDKEALIEALKTKRIAGAALDVFWEEPIPSDSELLELDNVTLTSHLAGTTKEALTRSPELLMEDVKKFIEGQKARFIVNPEVLENQEFKKWLEGVKK

SEQ ID NO: 108 - DNA, 3120 bp, Anaerocellum thermophilum DSM 6725
atgaacatcaactttaaaataaaaccattctggttttggaatgggaaaatggaaaatgatgaaatagcagatcaaatagctcagatgcatgaaaaaggaattggtggattcttcattcatccacggcaaggtctcgaaataccctatctttctcacgagtggtttgaaaaggtttctgttgcaattgaatgtgcaaaaaaatataacatggaagtgtggctttatgatgaatatccttatccaagtggaatttcagctggtgaagtagttgttcagcatcctgaatatcaagcttttatattagattataaagtgtttgaggcaaaagataatgaagaaatttgcattgaaattcccatgtgtgaagtgttattagcaagagcttatagaataaggaacaatattatagtatggaatgaatacatagatttgattgattatattggtgtaatctacaaagaacacatttaccaagaaagtgggcttacgttttacaataggaaaagatactttgttggagatggggcaaaagcacttaaatggaaagtaccaaaaggaaagtggaagatatttttgttttatcagtatccattaaaaaattttaaatatttcgggacatttattgatcctctaaataaagatgctgtaagactgtttattcaaaccactcatgaaaaatataaaaaatacttgggtcatgagtttggaaaaacaattaaaggaatttttacagatgaaacagctccagttgctggcaaacttccttggtcaaaattgcttcctaagctttttgagcaaacatatggggaaaatcttattgaaaaattacctcaaattatttgcacagacatatttgatacagccggttcaaaaattaggtatcagttttggaaacttgttgtggacacatttattgagagctacgacaaacaaattcttgaatggtgtcatcagaacaatcttttgtatgtgggcgaaaagccaattttgagaagctcacaattagcttttatggacattcccggaatagatgcagggcaccaaaaggctggtgacattccacaggttgtatctgaaaactacagagcaaatccaaagatagcatcatctgccgctcacttttataaaaaggaaagggttttgtgtgaatgctttcacagtattggctggagcatgacaatgcaagatatgaaatggatatttgactggctaatattgcagggaatagatatgtttgtcccccatgccttttattatagtgcagatggactcaaaaaacacgatgccccaccttcagcctttttccaaatgccttggtggaaacatcaaaaaatattgtctgagtatgtagaaaatgtaactaaaatgcttaaaaattgtaaaagaaaagttgatgtacttatagtagatccaattacaagccagtggacctgttttaacgacaaagaagtaaaagagaagatttcgatggatttctgtagaattcagcaaattcttttagaagaaaatgtagactattatgtgattgaccagtcattagtaggaagtttggaatgcaggaatcagaagatttattatgacaatgaaaaatttgaattattgatcattccacctgtgaccaatttagaaaaagaggcttacatgaagataaaagatctaatattgaaaggatgtaaagttgtatttattggctgtttgccattccaaaccatcgaagattttgatgttgccaaagatattagcaattttcttggagtaaattctatggacatcgcaaaagcatataaaacaggttctaaattgaacaatacagtttttcttaacagttgtatcttcattggtaatatagaagatttagtagcaaaaattgacaagatttgtaaaaagcctgtaagtatatcatatgaatcttctaatgaccgtggtattctctgtgcttactttgaggacgcagaacacgactttttatttatgattaacccaactaatgaaaagaagatctgcaaagtatacttgcggtacaggcctgatgaaataaacaaaatttattcggttccattgacatcgcaagaacttgataaggaaataaatttcgaaaattctttagataaaaagcaaataactttttccatgaattttgaaccttttcaatcgtatttaattaaattagaaaaaagttttgttaagaaaaacgaccataaaaatgatgttgaaaagagagtttttgagtacaaaatccctcttgcaactgtttgggaattttcaattgagagtttaaatcctttaaggctcggcagatggaatttaaagttgattttcaacaacgaaaatgaacagtattcaatatcttcaaaaattccagttacaccaaaaccaataattgatcagatagaagaggcaaaaattccaataccactaaaaacaaaaagtttctttggatgtccaaaagaaataacattgccaagctttgaggcaatatacactacttcattttttatagatgcagcaagccaaaaattctggcttgtaattgaagatgaaggtataaaaggtgaatgggttgtgcttttgaacaatcatactattttaccaagagattttgtacttaaaagattttattctcatactaatttggcatatgatattagcaacttaattaaactgggggaaaatcagctttgtgtatgtgtaaagattagcaggtcttttgatggacttcttacacccatttatatcttcagcacggcaggtgtattcaaagttgatgatagctggcatatagacaaacttccaactcaaggctgctttggtaaagaccttgaaaatggcattcctttttatgctggttttataaagtacgaaaaagaagttcaaatgccatcttttgaaaatggttttgtggaattctttattgaagataacataaatcagtgtgtaagtctttatataaatgatgaatttataggtacaaggtgctggcagccttatagatggaaggtagattctgatttactttcttcaaagaaggtaaagctcacacttgaagtatcaacttcgagcctgcagctgttcgaaggtgaagttattgaaccaataacacataaaattaagacaatataa

SEQ ID NO: 109 - protein, 1039 aa, Anaerocellum thermophilum DSM 6725
MNINFKIKPFWFWNGKMENDEIADQIAQMHEKGIGGFFIHPRQGLEIPYLSHEWFEKVSVAIECAKKYNMEVWLYDEYPYPSGISAGEVVVQHPEYQAFILDYKVFEAKDNEEICIEIPMCEVLLARAYRIRNNIIVWNEYIDLIDYIGVIYKEHIYQESGLTFYNRKRYFVGDGAKALKWKVPKGKWKIFLFYQYPLKNFKYFGTFIDPLNKDAVRLFIQTTHEKYKKYLGHEFGKTIKGIFTDETAPVAGKLPWSKLLPKLFEQTYGENLIEKLPQIICTDIFDTAGSKIRYQFWKLVVDTFIESYDKQILEWCHQNNLLYVGEKPILRSSQLAFMDIPGIDAGHQKAGDIPQVVSENYRANPKIASSAAHFYKKERVLCECFHSIGWSMTMQDMKWIFDWLILQGIDMFVPHAFYYSADGLKKHDAPPSAFFQMPWWKHQKILSEYVENVTKMLKNCKRKVDVLIVDPITSQWTCFNDKEVKEKISMDFCRIQQILLEENVDYYVIDQSLVGSLECRNQKIYYDNEKFELLIIPPVTNLEKEAYMKIKDLILKGCKVVFIGCLPFQTIEDFDVAKDISNFLGVNSMDIAKAYKTGSKLNNTVFLNSCIFIGNIEDLVAKIDKICKKPVSISYESSNDRGILCAYFEDAEHDFLFMINPTNEKKICKVYLRYRPDEINKIYSVPLTSQELDKEINFENSLDKKQITFSMNFEPFQSYLIKLEKSFVKKNDHKNDVEKRVFEYKIPLATVWEFSIESLNPLRLGRWNLKLIFNNENEQYSISSKIPVTPKPIIDQIEEAKIPIPLKTKSFFGCPKEITLPSFEAIYTTSFFIDAASQKFWLVIEDEGIKGEWVVLLNNHTILPRDFVLKRFYSHTNLAYDISNLIKLGENQLCVCVKISRSFDGLLTPIYIFSTAGVFKVDDSWHIDKLPTQGCFGKDLENGIPFYAGFIKYEKEVQMPSFENGFVEFFIEDNINQCVSLYINDEFIGTRCWQPYRWKVDSDLLSSKKVKLTLEVSTSSLQLFEGEVIEPITHKIKTI

SEQ ID NO: 110 - DNA, 726 bp, Anaerocellum thermophilum DSM 6725
atgatgcgaattgtagaaaaaattagacaaaaaaatgaaaattatcactccggtccaatacctacaattgcctttttgggtgacagtataacccacggttgttttgaggtcattgaaggaacaaaaagaacattagaagttgtgtgcgattttgaagctgtctatcataatcaatttaaaaaaatcttatcaatgatgtttccatttgctcaaattaatattgtcaatgcaggcataagtggtgatacagcacagggcggcttacaaagacttgaaagagatgttttaagatttaacccagaccttgtggtagtttgttatggattaaatgatagtaacaaaggaaaagagtatttgaatgaatatttagatggtttagcagggatatttatagaacttaaaaaacacgatattgaagtaatttttttaactcccaatatgaaaaatacttatatttcaccagctataaagagcttgcctttgattgagatggcaaaattaaatatggaaagtcaaatcaatggaacgttagatctttacatggattcagctaaggagctttgcaaaaaggaaaaggttgtagtttgtgattgttatgaaaaatggaaaaagctttatcacagtggcgttgacacaacaaatcttctttcaaattttataaatcaccccaatcgcccgatgcataagctctttgcatggtcattatttgagacaattatgttttaa

SEQ ID NO: 111 - protein, 241 aa, Anaerocellum thermophilum DSM 6725
MMRIVEKIRQKNENYHSGPIPTIAFLGDSITHGCFEVIEGTKRTLEVVCDFEAVYHNQFKKILSMMFPFAQINIVNAGISGDTAQGGLQRLERDVLRFNPDLVVVCYGLNDSNKGKEYLNEYLDGLAGIFIELKKHDIEVIFLTPNMKNTYISPAIKSLPLIEMAKLNMESQINGTLDLYMDSAKELCKKEKVVVCDCYEKWKKLYHSGVDTTNLLSNFINHPNRPMHKLFAWSLFETIMF

SEQ ID NO: 112 - DNA, 990 bp, Anaerocellum thermophilum DSM 6725
atgaaaatttgcatagtaggcagtagtggacactatgtatatgctttaagaggaataaaagaagaccctcatgcccaaattgtgggaatctctcctggatgtgaaggagagaatattgaaaggttacattctcaagtaaatgaaatgggattcacacctgtggtttatagcaatcctataaggatgtttgaagatctcaaacctgacattgctgtgattaatacatttttttataaaaattctgagcttgcaattgaggctatgaaaagaggaatccacgtatatatggaaaagcctgttgcactatcaatagaaaaacttgaagaactaaagagtgtgtggaggcaaacaaaagtaaaactctcatcaatgctgggattgcgctatacaccccatttttggactgcttataaacttataaatgaaaacaagataggtagaataagactgatacatgcccaaaaatcttataaacttggaactcgacctgacttttataaacatagaagaacatatggcggaacaattccctgggttggcattcatgctattgattggatttattggctaagtggcaagaaatttaaatcggtctttgcaggacattcaaaactttataataatgatcatggtgagcttgaatctactgctttttgtagttttgtaatggaagatgagatttttgcaacggtgaacattgactatctgcgtcctgctactgcccctactcatgatgatgatagaattagaattgtgggaacaagaggaatttttgaagttttaaatggaaaagttttcttgctaaatgataccactaaagagatctcagaagtctctttagaaaaaccacctattgtgtttttagatttcttaaatgaggtaagaggtacagataagtgcttagttagtagcgaggatagcttttatgtaacctttgcttcgcttttagcaaggcagtctgctgatgaggataaggtaattgaattttaa

SEQ ID NO: 113 - protein, 329 aa, Anaerocellum thermophilum DSM 6725
MKICIVGSSGHYVYALRGIKEDPHAQIVGISPGCEGENIERLHSQVNEMGFTPVVYSNPIRMFEDLKPDIAVINTFFYKNSELAIEAMKRGIHVYMEKPVALSIEKLEELKSVWRQTKVKLSSMLGLRYTPHFWTAYKLINENKIGRIRLIHAQKSYKLGTRPDFYKHRRTYGGTIPWVGIHAIDWIYWLSGKKFKSVFAGHSKLYNNDHGELESTAFCSFVMEDEIFATVNIDYLRPATAPTHDDDRIRIVGTRGIFEVLNGKVFLLNDTTKEISEVSLEKPPIVFLDFLNEVRGTDKCLVSSEDSFYVTFASLLARQSADEDKVIEF

SEQ ID NO: 114 - DNA, 843 bp, Anaerocellum thermophilum DSM 6725
gtgagaagaacaaagacaatgaaagcattattgtttcatctgctaacaattctatttggttacataatgctgtatccacttctttggatgtttttcagttccttcaaagaaaatagtgaaatttttctaaatgcgcacgaattgctacccaaaaagtggcttttcaaaaattatattgatggatggagaggttttgcgggatacccattttcagttttttttaaaaattcatttattgttactattattggtactgttggtgctgtcatttcctcagctattgtagcttatggttttgcaagatgtaagttcaaaggcaaaggattttggtttggttgtatgattataactatgcttttaccatatcaggttgttatgattccgcaatacatcatgtttcaaaagatgggatgggtaaatacgttcaagcctttacttgttccagcttttttgggtcagccattttttatctttttaatgattcaatttataagaggaattccaaacgaattagatgaagcggctaaaattgatggttgtagtaaatattcaatttttacaagaattattttgccattaatttctccggctttaattacatcagcaatcttttcatttttgtggcgttgggatgactttttagggcctcttttgtacctcagcaaacctgagttgtatacagtatctttagggttgagaatgttctctgacccgacagcagtttctaactggggagctgcctttgctatggcaacattatcgctcgttccctcatttataatatttatattcttccaacgctatctagtggaaggaattgttactacaggtttaaagggttaa

SEQ ID NO: 115 - protein, 280 aa, Anaerocellum thermophilum DSM 6725
VRRTKTMKALLFHLLTILFGYIMLYPLLWMFFSSFKENSEIFLNAHELLPKKWLFKNYIDGWRGFAGYPFSVFFKNSFIVTIIGTVGAVISSAIVAYGFARCKFKGKGFWFGCMIITMLLPYQVVMIPQYIMFQKMGWVNTFKPLLVPAFLGQPFFIFLMIQFIRGIPNELDEAAKIDGCSKYSIFTRIILPLISPALITSAIFSFLWRWDDFLGPLLYLSKPELYTVSLGLRMFSDPTAVSNWGAAFAMATLSLVPSFIIFIFFQRYLVEGIVTTGLKG

SEQ ID NO: 116 - DNA, 927 bp, Anaerocellum thermophilum DSM 6725
atgtcgaatattaatcgaaaaaagtctttcagagaacttttgatgagttcagaaaatgttgctggttatgtttttatatctccctggcttattggcttttttgtgttcactttgattcctattgcagcaaccttttatttgtcttttactcaatatgatttattatcatctcctaaatttgtaggattacaaaattatgtacaaatgtttaaagaagatccgttattttggaaatcaatgtcagtaacttttttctatgtgtttgtaactgtgccattaaagctggcttttgcattgcttcttgccctttggctttcttacaaaagcagactaacaccattttacagggctgtatactatgttccttctatgatgggtggcagtgtggctgtggcagtgctttggcaaagactttttacaagtgatggtgttataaattcaatattgaaactatttggaattcaaagtgagacttcatggataggaaatccaagaactgctatatggacgttgatattacttgcggtttggcaatttggttcgccgatgttgatatttttagcaggtttaaagcaaataccagaaagctattatgaagcagctattattgatggagcaaatagctggcaaaagtttgttaaaataaccttgccgatgctcacaccaataatatttttcaacttgattatgcagatgataggaagctttatgacttttactcaaggattcattattacaaatggcggccctgtgaacagcacactcttttacgctatttacctctacagaagagcattccaattttatgacatgggctacagctgtgctatgtcgtgggtaatgcttattatcattggaatactcacagcttttatattcaaatcatctacattttgggtatattatgagtccaaggaaggtgaataa

SEQ ID NO: 117 - protein, 308 aa, Anaerocellum thermophilum DSM 6725
MSNINRKKSFRELLMSSENVAGYVFISPWLIGFFVFTLIPIAATFYLSFTQYDLLSSPKFVGLQNYVQMFKEDPLFWKSMSVTFFYVFVTVPLKLAFALLLALWLSYKSRLTPFYRAVYYVPSMMGGSVAVAVLWQRLFTSDGVINSILKLFGIQSETSWIGNPRTAIWTLILLAVWQFGSPMLIFLAGLKQIPESYYEAAIIDGANSWQKFVKITLPMLTPIIFFNLIMQMIGSFMTFTQGFIITNGGPVNSTLFYAIYLYRRAFQFYDMGYSCAMSWVMLIIIGILTAFIFKSSTFWVYYESKEGE

SEQ ID NO: 118 - DNA, 1305 bp, Anaerocellum thermophilum DSM 6725
atgaaaagaatattgtgcattggaactattataatatttttattttcaatccttatatttccttttactaaaccttctgaaaaggcttttggttcttctcagtcaatcaaactcagagttgcgtggtggggaagccaaacgcgtcatgacagaactcaaaaggtgcttgaactataccgagcaaaggttaatcgaaaagtaagttttgtaacagaatttggaagctggtcgggatattgggataaacttacaactcaagcagctgcaaagaatttgcctgacattattcaaatggactatatgtatttagctcaatatgttcaaaaaggattgttggcagatttaacaccttacacaaaaaatggaatattgaatctgaaagatgtaagtgaagcaagtataaaaagtggatcagttggtggaaggatttatgccataagtttagggactaacgctttagcaataatttatgaccctgctgtggctcaaaaagcaggtgttaagatacctgaagatggcaactggacctggaatgactacaaagagattatcaaaaaagtttttcaaaaaaccaaaatcagagcagacttggcattaacagctgacccaaaattcttacttgaatactatgtaaggcagcaaggcaaaagcttatataaacacgatggaacgggtttgggatttactcaagaaaaatttgttattgatgtatttaatatcaatttagagttattgaaaggcggatatacagcaaaacctgatgaagtttcagcaacctccacaattgaagacagcttatttgtgaaaggcaagacatggataggatggacatggagcaacatgtttgttgcaacagcaaatgctgcaaaacgcccactggcattagctcttccacccaaggggggtataagaccagggttgtatttaaaaccttctcagttcttctctattgctgcaacctccaaatataaaactgaagcagctaaagttataaacttctttacaaatagcatagaggcgaataatattttgcttgctgaaagaggtgtgcctatttcagcaaaagtaagagaaggtatcaagaatgcagtcgaacctgcagtgagacagacatttgattatatagccctcgctgaaaagaattgttcacctattgatccaccagatccaccaggcggaactgaagtagggcagacgtttaaagatctttacgaccaagtcttgtatggtcagataaaacctgaaacagcggcaaagatgtttatgcaaaaagcaaatcaaatacttgccaaaaacaagaaataa

SEQ ID NO: 119 - protein, 434 aa, Anaerocellum thermophilum DSM 6725
MKRILCIGTIIIFLFSILIFPFTKPSEKAFGSSQSIKLRVAWWGSQTRHDRTQKVLELYRAKVNRKVSFVTEFGSWSGYWDKLTTQAAAKNLPDIIQMDYMYLAQYVQKGLLADLTPYTKNGILNLKDVSEASIKSGSVGGRIYAISLGTNALAIIYDPAVAQKAGVKIPEDGNWTWNDYKEIIKKVFQKTKIRADLALTADPKFLLEYYVRQQGKSLYKHDGTGLGFTQEKFVIDVFNINLELLKGGYTAKPDEVSATSTIEDSLFVKGKTWIGWTWSNMFVATANAAKRPLALALPPKGGIRPGLYLKPSQFFSIAATSKYKTEAAKVINFFTNSIEANNILLAERGVPISAKVREGIKNAVEPAVRQTFDYIALAEKNCSPIDPPDPPGGTEVGQTFKDLYDQVLYGQIKPETAAKMFMQKANQILAKNKK

SEQ ID NO: 120 - DNA, 1212 bp, Anaerocellum thermophilum DSM 6725
ttgaatattgcaaagattggtcttattggaattagtggttttgggagtatacacttgcggtcaatagaacagcttcaagggaagatggttgacttgagagcaattgttgcaacaagctacgaaaaaaataaagaagtgattgatagattggcttctcgaggtgttgagtattatcaggattatagattaatgcttgaaaatcataaagacttagattttgttgccatctcaacgcccattcatttacatgctccaatggcaattgatgcaatggaaagaggttttaatgtcctgcttgaaaagccgcctgctgtgacaattcaggatattgacgctataattgagacgaagagaaaaacaaaaagggtttgtagcgtgaactttcaaaacacctctggtaaagcatttagaaaacttcttgagtatataagagaaggcaggcttggcaggataaaatcaattattggtgttggacgttggaaaagggatgaaagctattatcaaagaaatgcgtgggctgggaagctaattgttgatggtaactatgtcttggatggaacaataaacaatcctcttgcacatcttttgaacaatgaattgattattgctgagacctcagaagaaaatggaggtgtacctcagaaagttactgcagaactttatcacggtcataaaattgaaggtgaagacacagcgtgtgtgagaatcatcacaaaaacaggaatcgaggtctatttttattcaacgttatgtaacagggaagaagagtcaccctatatcataatagaagctgaaaaagcaagagcttattggacatttgcaaataagtttaagatagaatattttgatggcaatacagaagagtttgatggtggacgcgaagatttgtttgtgaatatgtacattaatatggtggagcatctgtttgaaggcaaacaactttactgtcctctggaagtgactcgcaattttgttttggcatccaacggggctttcgaatcgtcagggtgtatttatgatattccagacgaatatttagagattagcaatgaaaatggtaagatatatacctatatcaaaaatataaaagaaatcatagatgaggcagctgagaatagaaaactattttctgaaataggagtaccgtgggcgaaacaaacagaagagtttgatttaattgattactgttgttttagtatgtttaaaaggtaa

SEQ ID NO: 121 - protein, 403 aa, Anaerocellum thermophilum DSM 6725
LNIAKIGLIGISGFGSIHLRSIEQLQGKMVDLRAIVATSYEKNKEVIDRLASRGVEYYQDYRLMLENHKDLDFVAISTPIHLHAPMAIDAMERGFNVLLEKPPAVTIQDIDAIIETKRKTKRVCSVNFQNTSGKAFRKLLEYIREGRLGRIKSIIGVGRWKRDESYYQRNAWAGKLIVDGNYVLDGTINNPLAHLLNNELIIAETSEENGGVPQKVTAELYHGHKIEGEDTACVRIITKTGIEVYFYSTLCNREEESPYIIIEAEKARAYWTFANKFKIEYFDGNTEEFDGGREDLFVNMYINMVEHLFEGKQLYCPLEVTRNFVLASNGAFESSGCIYDIPDEYLEISNENGKIYTYIKNIKEIIDEAAENRKLFSEIGVPWAKQTEEFDLIDYCCFSMFKR

SEQ ID NO: 122 - DNA, 1089 bp, Anaerocellum thermophilum DSM 6725
atgaaagcttatgcaatggtattagaagaatttaacaagccgttaaaagcaaaagagtttgaactaatcaagccttctgatggtgaacttcttgttaaaattgaagcggcgggtgtttgtggatctgatgtgcatatgttcagaggtaatgacccgcgtacaaaacttcccatgattttagggcatgaaggtgttggacgtgtgtatgctatttcaggtcagtggcgtgatataaatggcgagaaaattcaagagggagatttgataatttgggacaggggtgttgtgtgtggtaggtgctacttttgtgctgtcaaaaaagaaagctatctgtgtccgcacagatggacatatgggataagcgttagctgcgcagagcctccgcatttgagaggctgctattcggagtacatttatcttcacaaagatacgaaagtgataaaaataaaagagaatgttgatccagaaattttagtatctgcctcatgttctggtgcaacgtgtgctcatgcttttgacattgtttcacctgattttggtgacagtgtcctaattcaagggccaggtcctatagggctttatgcaatcatttttgcaaaacttagaggagcacgaaatataattgtgattggtggcacaaaagaaagacttaaaatgtgtgaagaatttggggcaacgcatgtgcttgatagaaattcaactacagcttgccaaagacaggaaataataatggatatcacaaatgggcgtggagtcgatttggcaattgaagctgtgggacatccatcagcagtaagtgagggaataaaacttgttcgaaatggtggaagctacttatcacttggttttggtgacccaaacggcagcgttacactcgattgttactatgatattgtgagaaaaaatttaagatatcaaggggtatgggtcagcgatacaaaacatttatatatggcagtgaatgttgtgctccagaacagggaacttttcaaaaagatgattacaaatgtttataagttgactgatgcgacaaaagctcttgaggatatggaaaacaaaaatacaataaaatctgttctaaagccttga

SEQ ID NO: 123 - protein, 362 aa, Anaerocellum thermophilum DSM 6725
MKAYAMVLEEFNKPLKAKEFELIKPSDGELLVKIEAAGVCGSDVHMFRGNDPRTKLPMILGHEGVGRVYAISGQWRDINGEKIQEGDLIIWDRGVVCGRCYFCAVKKESYLCPHRWTYGISVSCAEPPHLRGCYSEYIYLHKDTKVIKIKENVDPEILVSASCSGATCAHAFDIVSPDFGDSVLIQGPGPIGLYAIIFAKLRGARNIIVIGGTKERLKMCEEFGATHVLDRNSTTACQRQEIIMDITNGRGVDLAIEAVGHPSAVSEGIKLVRNGGSYLSLGFGDPNGSVTLDCYYDIVRKNLRYQGVWVSDTKHLYMAVNVVLQNRELFKKMITNVYKLTDATKALEDMENKNTIKSVLKP

SEQ ID NO: 124 - DNA, 1806 bp, Anaerocellum thermophilum DSM 6725
gtgatatacaaagcagatgtgctggtagtaggtagtggcggagcaggtttgagagctgcaattgcagcgtgtgaaagagcatatgagagccgaaaaagaataaaagttttgctggcaagtaaaggaaaaataggaagttgtggtactacagctcttgcatactctgatagaatggcattccatgtcacacttcccacaacagagcctaaaggtgaagataactggaaatatcatgcaaaagatatctatgagattggcggacttgtttctgattatgacttggctgagattttagcaaaaaactcagccgatgcttatttttacttagatagtctcggtgttccgtttgtaaaagaaaatggcgtacctgctcaatttgtgacagacggttctatatatgcgcgtgcatgttttacaggacctgatactgctgttcaaatagaaaaggctttgattcgaaagcttggtgagatgaaggatattgaagttttagaagatgtgatgataagtgatttgattgttgtgaataacaaagtttgcggagcaattgcttttaaagggaatcaaaatatcataatacttgcaaaagccattgttttagcaacaggtggggcaggaagtatttataaaagcaatgtgtttccaccacgcatgacaggtgatgggtatgcaatggcacttcgtgcaggggctttgcttgtgaatatggagtttattcaaataggtctttcatcccccaaaacaaaacttgcgtgttcaggaagtataatgagatgtgtccccaggtttgtaaatgaaaaaggagaagaatttttattaaattatcctatcgcgtacaatgatgtatttgaaaaaggtgcaacgtggccaataagctacgagcacaagacatgtttgatagacattgcagtgttcagagagattgcccgtgggggtaaagtgtttttagactttactcaaaatccaaaaggttttgaatttaaacacctaagagaagacttaaaacaaagatattactcagaagttaaaaatcttacaaataaaagatctactccatacgaaagactttgtgaaataaatcctcagacagttgagtggtttttgaaaagaggaattgaccttagaaaccaaatgttagaaattgcgccatcaattcagcatttccagggtggtgtaaaaattagagaaaatgcaaatacagcaattagtggcttgtatgcctgtggcgagtgtgcaggcggacagcatggagcaaacagaccaggcggaaatgcacttttggatactcaggtttttggtaagatagcaggggaaagtagctttgaatttgcctcaaacacttcgattgatgaagaatctgcaatttctgaagcaaaccgcctgtttgaaagctataaaatctatatagctgaagatggcattgatttggagaatgctatatcagaactaaatagggttatggatttgtacgcaagtgttgtgagacatcaggatggacttcaaaaagcacttatgaaaattgaggagttgaaaacaagaaaaatcaagcctgttgagtatgaatatcttttagagctaaaaaacatgcttttgtgtgcagaggcagtggtaaaaagttgcattttaagagatgaaagcagaggaccgcacttgatgttcgaaaattacagcgatttgtggccaaagccaagagatgaaagatacaacatatatcacgtatgcaagctaaacaaagagacaaatcaagttgaagtctttcctatggaaccagtaaaacccgaaaccttagggggcaaagtaaaatga

SEQ ID NO: 125 - protein, 601 aa, Anaerocellum thermophilum DSM 6725
VIYKADVLVVGSGGAGLRAAIAACERAYESRKRIKVLLASKGKIGSCGTTALAYSDRMAFHVTLPTTEPKGEDNWKYHAKDIYEIGGLVSDYDLAEILAKNSADAYFYLDSLGVPFVKENGVPAQFVTDGSIYARACFTGPDTAVQIEKALIRKLGEMKDIEVLEDVMISDLIVVNNKVCGAIAFKGNQNIIILAKAIVLATGGAGSIYKSNVFPPRMTGDGYAMALRAGALLVNMEFIQIGLSSPKTKLACSGSIMRCVPRFVNEKGEEFLLNYPIAYNDVFEKGATWPISYEHKTCLIDIAVFREIARGGKVFLDFTQNPKGFEFKHLREDLKQRYYSEVKNLTNKRSTPYERLCEINPQTVEWFLKRGIDLRNQMLEIAPSIQHFQGGVKIRENANTAISGLYACGECAGGQHGANRPGGNALLDTQVFGKIAGESSFEFASNTSIDEESAISEANRLFESYKIYIAEDGIDLENAISELNRVMDLYASVVRHQDGLQKALMKIEELKTRKIKPVEYEYLLELKNMLLCAEAVVKSCILRDESRGPHLMFENYSDLWPKPRDERYNIYHVCKLNKETNQVEVFPMEPVKPETLGGKVK

SEQ ID NO: 126 - DNA, 1146 bp, Anaerocellum thermophilum DSM 6725
atgccaaacctatcaacgacgtatgcaaagctgaatttaagaacacctgtaattgttgcatctgctggcattactggaactgtggagaggcttcaaagatgcgaagaaaacggtgctggggcagttgtgacaaaaagtctttttcaaaaggaaatatgcagaattgcacccactccacggtttaaaatagtcaagcatgaaaacacgtttacgctttactcatatgaacaggcaagcgaatttaaccctcaagagtatgctgaatttatattcaaagcaaaacaaaagctaagcattccagttattgcgagtataaactgctacacagatgatgcatggcttgagtatagcaagcttatggagcaggcaggggctgatgcgatagagctaaacctttcatgtcctcacggtgtgcatataatgtctggtatggatgtaattgaagagatggtcaacacaacaaaacttgtcaaaagcaatgttaagataccagtgatacccaaaatgactcctcaatctacaaatccgggatctgatgccttaagactcgacagtgcaggagcagacgggcttgtaatgttcaatagatttacagggcttgacattgatattgagaaagaagcacccattttgcacggcggttatgcagggcatggtggtccgtgggcaattatgtatggtttgaggtggataagcgctgtatcgccaaaagtgaaatgtagtatcagtgcaagcggcggtgccatgaatggagaagatgttgtcaaatacatattggcaggtgcgtcggctgttcaagtttgcacaactgttattttgaatggctatggggttataaaaaagataaacaagtatttagaagagtacatggagagaaaaggttacaacacaattgatgattttaaaggaaaggtgtgcagtagaattcttgacatggactctgttgacagaacgcactgggctgttgcaaggattgacaaagaaaaatgcacatcttgtggcaagtgcttcacagtttgcatatatgatgcaattgaaaaggatgatggaaagtttaaagtaaatcaaaactgcgatggctgcggactttgtgcagaactgtgcccagccaaggcaatcttaatggtaagaagaggtgaagtttaa

SEQ ID NO: 127 - protein, 381 aa, Anaerocellum thermophilum DSM 6725
MPNLSTTYAKLNLRTPVIVASAGITGTVERLQRCEENGAGAVVTKSLFQKEICRIAPTPRFKIVKHENTFTLYSYEQASEFNPQEYAEFIFKAKQKLSIPVIASINCYTDDAWLEYSKLMEQAGADAIELNLSCPHGVHIMSGMDVIEEMVNTTKLVKSNVKIPVIPKMTPQSTNPGSDALRLDSAGADGLVMFNRFTGLDIDIEKEAPILHGGYAGHGGPWAIMYGLRWISAVSPKVKCSISASGGAMNGEDVVKYILAGASAVQVCTTVILNGYGVIKKINKYLEEYMERKGYNTIDDFKGKVCSRILDMDSVDRTHWAVARIDKEKCTSCGKCFTVCIYDAIEKDDGKFKVNQNCDGCGLCAELCPAKAILMVRRGEV

SEQ ID NO: 128 - DNA, 1539 bp, Anaerocellum thermophilum DSM 6725
ttgtacatcattatattccactttcagtacatttttattttatcattaacaccttttgaagttgagattaataaatttgcccaaattggctgtgagaatagacttacaattgttgtaaacaacatcttggactggagttgtcttccaccagggtttataagggaatacaatgacccaatgcatccagaagggtataaaactcaggaatatctttttgactttttcaactattcaggtattcacagaccagttttgctctacaccacttccaaaacatatattgaggatattaagattgaaacccagattgagggtcaaaagggtatagtttgctttaaggtggctgtaagtggcgaaaaaaaggatgaatgtcagatagcagtagctttgtatgacaaagatggaaagcaaatagcaaaggtcgaagggccagagggtatgatagaggttggagatgcgatattttgggagccttcaaatccatatctttacaaactaaatgtaactttaatacacgatgaaaaggtggtagatgaatattatcttcctgtgggaataaggacagttgaggtaaaaggcaaaagacttttcctaaatggtaagccagtgtatcttaaaggtttggcaaagcatgaagacagtgatataaggggcaagggatacgaccctgtgatagctgtgaaagatttcaacctcctaaaatggataggagcaaactcattcagaacatcacattatccttacgcagaagagattttaaacttggcagacgagtatggttttttggtaattgacgaggcaccagctgttggcatgaatttctttaacaaaaacgaaaaagtgtttaccgcggagagagtaaaccaaaagacattagaacatcacttagaagttataagacaacttattgcaagggataaaaaccatccaagtgtgattatgtggagtgtggcaaatgaggctgcaacatatgaagatggggcatatgaatatttcaaaagagtaatagatgaggtgagaaagcttgacccgacaagaccggtgacgctggttgaatcctcttttccagatgagaccaaagtgggaagtcttgttgatgttatatgtgtaaacaggtactattcatggtattctgatcctggcagactggatttgatagagttccagcttgaaaaggagctgaaaaggtggtttgagctttatcaaaaaccagtgataataacagagtatggggcagatacaattgcaggatttcattcaagtcctccaatgatgttttctgaggaatatcagtgtgagatgcttgaaagatatcatagggtgtttgacaggctggattttgtgataggcgaacacatatggaactttgcagactttgcaacaaaacaagaggttcgaaggattatgggcaacaggaaaggaatctttacaaggcaaagacagccaaaagccgcagctttcttgctcaaaaaaagatggcaaaattcagagcacaaaaggctggaggaaaatgtttcagaagataaaacacgtaattaa

SEQ ID NO: 129 - protein, 512 aa, Anaerocellum thermophilum DSM 6725
LYIIIFHFQYIFILSLTPFEVEINKFAQIGCENRLTIVVNNILDWSCLPPGFIREYNDPMHPEGYKTQEYLFDFFNYSGIHRPVLLYTTSKTYIEDIKIETQIEGQKGIVCFKVAVSGEKKDECQIAVALYDKDGKQIAKVEGPEGMIEVGDAIFWEPSNPYLYKLNVTLIHDEKVVDEYYLPVGIRTVEVKGKRLFLNGKPVYLKGLAKHEDSDIRGKGYDPVIAVKDFNLLKWIGANSFRTSHYPYAEEILNLADEYGFLVIDEAPAVGMNFFNKNEKVFTAERVNQKTLEHHLEVIRQLIARDKNHPSVIMWSVANEAATYEDGAYEYFKRVIDEVRKLDPTRPVTLVESSFPDETKVGSLVDVICVNRYYSWYSDPGRLDLIEFQLEKELKRWFELYQKPVIITEYGADTIAGFHSSPPMMFSEEYQCEMLERYHRVFDRLDFVIGEHIWNFADFATKQEVRRIMGNRKGIFTRQRQPKAAAFLLKKRWQNSEHKRLEENVSEDKTRN

SEQ ID NO: 130 - DNA, 1203 bp, Anaerocellum thermophilum DSM 6725
atgcagaacttaacagcacatttaaacatgataagggcgatgaaaatgaaacctaacttttcagaacttgcaagaatatatgggatggatagaagaacagttaaaaaatattatgagggttatgaaggaaaacctaagaatagaaataaaccaagtaaattggacaaatactatgatgagataaaatcaaagcttgctatcaaaggagttacagtcaagggtgtttatgagtatttaaaatcaaaagatgagacaataggaacatattcaaacttcaataagtatgttaagaaaaaaggattaaagccagagaagaaaataaaaggtcacccaagatttgagacagatccaggtgagcaagcgcaagttgattggaaagagaatataaagcttgtctcaagaaatggagaggagtttatcattaatgttcttgattttaaattaggttattcaaggtattgctgctttgagataaacaggacaaaaactcaagaagaattaatagaaactctaataagaatattcaaagatataggcggagtaccgagagagattttatttgacaatacagcagcagttgttgatataacaggtgagaaaattaaagtaaattcaagatttaaaagttttgcaaaagactttgggtttgaagtaaaactgtgcaaaccaagacattcgtacacaaaaggaaaagttgaagcagcaaacaagtttatagattggatactgccatatcagggtgaatttgaaacagaagaggacttagtaaggataataaaagagataaacgcaaaggtcaatatgcagccaaatcaaacaactcaagttccacctgctcttctgtttcaaaaagaaaaagagtatttacaacccttgccagacaaaaggttaatagacagttacctaaattcctacaagtcagttaaagtccaaaaggactctctgatttactacaagggaagtaaatactctgttccacccgaatacataggaaagacagtccaagtaaaggaggtggaaaacaaaatttatatttattataacacaaacctgttaaggatacatgttattgatgaaaaaaatatcaattatcacgatgaagattacaagcagctaatgctaatgagagttggtcaaagagaagagcttaacaagatatgtgaggaaaacctaaagaaatttgataatctgttgaaaacctaa

SEQ ID NO: 131 - protein, 400 aa, Anaerocellum thermophilum DSM 6725
MQNLTAHLNMIRAMKMKPNFSELARIYGMDRRTVKKYYEGYEGKPKNRNKPSKLDKYYDEIKSKLAIKGVTVKGVYEYLKSKDETIGTYSNFNKYVKKKGLKPEKKIKGHPRFETDPGEQAQVDWKENIKLVSRNGEEFIINVLDFKLGYSRYCCFEINRTKTQEELIETLIRIFKDIGGVPREILFDNTAAVVDITGEKIKVNSRFKSFAKDFGFEVKLCKPRHSYTKGKVEAANKFIDWILPYQGEFETEEDLVRIIKEINAKVNMQPNQTTQVPPALLFQKEKEYLQPLPDKRLIDSYLNSYKSVKVQKDSLIYYKGSKYSVPPEYIGKTVQVKEVENKIYIYYNTNLLRIHVIDEKNINYHDEDYKQLMLMRVGQREELNKICEENLKKFDNLLKT

SEQ ID NO: 132 - DNA, 765 bp, Anaerocellum thermophilum DSM 6725
atgagcaactatgtgaaactacttaacaacttagaagagttaggtcttcacaacataaagaataaccttgacaaatacttagatttagtggcaagcggagaaaaaagtatgacagatgcattatatgaacttagtaatttagagataaaagccaaagaagaaagggcgatattaggatgtgtgagggtggcaaatttcccatttataaaaggtatagaagattttgatttttcatttcagccaagtataaacaagcaacagataatggacttaatgagtttgagatttttagagggtaatgaaaatatactatttgtcggaacaccaggggtagggaaaacgcatctagccacagcaataggtatagagtgtgcaaaacgaaggtattcaacatattttatacattttcaagagttaatagcccagctaaagaaagcattattggagaacagattagagtacagacttaagcatttttcgaaatacaaagttttaataatagatgagataggttatttgccaatagacaatgatggagcaaatttatttttccagctgatatcgagcagatatgagaagagcagtacaataataacaactaatgttgtattctcagaatggggagagatatttggtggagcgacaatagcaaatgcaattttagataggctactgcatcattcttacgtgattttcataaaaggtccttcatacagattacagtcaaaaacagcatattttagcaatacaaaccagcaaagttaa

SEQ ID NO: 133 - protein, 254 aa, Anaerocellum thermophilum DSM 6725
MSNYVKLLNNLEELGLHNIKNNLDKYLDLVASGEKSMTDALYELSNLEIKAKEERAILGCVRVANFPFIKGIEDFDFSFQPSINKQQIMDLMSLRFLEGNENILFVGTPGVGKTHLATAIGIECAKRRYSTYFIHFQELIAQLKKALLENRLEYRLKHFSKYKVLIIDEIGYLPIDNDGANLFFQLISSRYEKSSTIITTNVVFSEWGEIFGGATIANAILDRLLHHSYVIFIKGPSYRLQSKTAYFSNTNQQS

SEQ ID NO: 134 - DNA, 399 bp, Anaerocellum thermophilum DSM 6725
atgctttatccacgtgagacaagaacaagagagataaaagacttaagcggtatatggaggtttaaagttgacagggacaataaaggctatgaacagaggtggtttgaaaaaccccttgaagatgctattttgatgcctgtgccgtcaagctacaatgacataacccaggatgagagcataagagatcacataggtgatgtgtggtatgaaaggacattttacatacctgaaggttggaaggacaaaagaatagttttgagagttggaagtgctactcatcacgcaagagtatttttaaatggtaaggaaatagcccagaacaaaggtgggtttttgccttttgtaaatgtcaatatcaaaatgaacaaaaaatcgaaaataaaaatgtacaaaaaataa

SEQ ID NO: 135 - protein, 132 aa, Anaerocellum thermophilum DSM 6725
MLYPRETRTREIKDLSGIWRFKVDRDNKGYEQRWFEKPLEDAILMPVPSSYNDITQDESIRDHIGDVWYERTFYIPEGWKDKRIVLRVGSATHHARVFLNGKEIAQNKGGFLPFVNVNIKMNKKSKIKMYKK

SEQ ID NO: 136 - DNA, 2598 bp, Anaerocellum thermophilum DSM 6725
atgcaagaaataaagctaaaatggcttgtcaaaccgcaggtaggcaatattggtgcaacgttttgtattccatggaaaaaagctcagctctggaatacagaaaatgttattgtcagaaacaaggatggaagtattattcctactcaaaactgggcactttcatactggggtgatgggtcggtaaaggtgagtagccacgctgctttctttggcaccaatttacctgatgagctctttgcatgtattgaagaagataccgtaacaaaattcgaaacaatggttgaagtttatgaagacaaggagcatattagcgtgacaacaggcaagcttgagtgcaaaattagcaagaccggagataaaattattgaatatttaaaagtaggacaaaagattatttgctctaatctcacgcaaattcttattacagaaagaatttctaattttacaggatataagactaaagttgaagaagaatacattcctcaaatattaggtgcaaaagtagagtctcacggacctttgagatgtgttataagggtgtggggaagacacttgcggaataccaagatgacatttgatggctctttactcaaacaagggtggttgccttttgagttgagattatatttctatgcaaattcagataaaattaagattgttcatacatttatttacaacggaaatccccatcaagattttataaaaggattgggcttaaaatttgatcttcctttgcactctcctctgtacaatagatttgtaagatttggaggggacagtggacttttttgtgagtcaccaaaaaatttgatagatgtgtacagaaaagagagatacaaaataatgtttcaaaatcaggtcaatggacaaccagtagattttgatattgacgaggaccaccattatattagggagattaacaatactccagagtgggatgaatttattctttttcaagattcttccgagcactatgttattaaaaaacgtacatcttgccagtgcagttttgtaaaagcaacagaaggcagaagatctatggggtttgcatatattggtgatgagaatggtgggcttggagtagctttaaaggatttttggcaaaaatatccttcaggatttgaggcaaaagatttaacttcttctgaggcaaatttaatagtttggctttggccaccttatgctgaagttatggatttgagacactatgattttggtacatacacatcttcttcatatgaagggtttgatgagtatagaagtactccctatggtatagctaatacaaatgagctgtggctgtactgttttgattatgctccaacaagtgagcagcttttggaatatgcaaaaatgcaaggattttctcctcttcttgtatgtagccctgaaaggtataaagagacagagatatttggaggattaaatttaccggataatagcaactccaagaaagaaaagattgaaagaatactgacagctattgttgatttttatctaaaggaagttgagagaagaaagtggtacggtttttgggactatggtgattttatgcatagctatgatatggtaagacatatgtggaaatatgatattggtggatatgcttggcagaacacagagcttgtgccaaacatttggctttggcttatgtttttaagaacaggacgttttgatatatttaaaatggctgaggcaatgacaagacatacatcagaagtagatgtgtatcatcttggggaatacaagggactgggttcaaggcacaatgttgttcattggggatgtggttgcaaagaagtgagaattagcatggcataccttcacaggtattactattatcttactgccgatgaaagaatagggcagcttatggatgatgtaaaagatattgacaaacaaattatgcacatggaccctatgcgtgcgtattttacgaatgatcctgaaaatagagtacacataagggttggaccggatgttatgaccttttgcgccaattggtttgtaagatgggaaaggcacaaggaagaaatttacaaacaaaagataataaagatactggactttttgaagaaaaatccagcagcatttatttcgggtggggtatttgactatgatcctgaaaaaacccagttaaaacctgttgaatacactggtggttcaaattttgtattttgttttggaaatacacttgtttggcttgagatagccaagaattttgaagataaagaatttgaagaattgtgtgctcagcagggccttttttatactgaatttaaagaaaacaaaaatgagattttaaaaagctggggtgttccaagctttggatttaagttgaacatgctcaatattgggatggctgcctttgctgcaatgaagaaaaatattcctgaattgaaaaaagaaatatggcagatgtttttagaccataacaaaaatccatggctaaaatttatttgtgatggaggtataaatttacaattggcaactgtacctgcgcctgttgctgagattccttttatttcaacgaatatagcttctcaatggtcattaaacgcattgcttgcgctagaatttattggagatgagctatga

SEQ ID NO: 137 - protein, 865 aa, Anaerocellum thermophilum DSM 6725
MQEIKLKWLVKPQVGNIGATFCIPWKKAQLWNTENVIVRNKDGSIIPTQNWALSYWGDGSVKVSSHAAFFGTNLPDELFACIEEDTVTKFETMVEVYEDKEHISVTTGKLECKISKTGDKIIEYLKVGQKIICSNLTQILITERISNFTGYKTKVEEEYIPQILGAKVESHGPLRCVIRVWGRHLRNTKMTFDGSLLKQGWLPFELRLYFYANSDKIKIVHTFIYNGNPHQDFIKGLGLKFDLPLHSPLYNRFVRFGGDSGLFCESPKNLIDVYRKERYKIMFQNQVNGQPVDFDIDEDHHYIREINNTPEWDEFILFQDSSEHYVIKKRTSCQCSFVKATEGRRSMGFAYIGDENGGLGVALKDFWQKYPSGFEAKDLTSSEANLIVWLWPPYAEVMDLRHYDFGTYTSSSYEGFDEYRSTPYGIANTNELWLYCFDYAPTSEQLLEYAKMQGFSPLLVCSPERYKETEIFGGLNLPDNSNSKKEKIERILTAIVDFYLKEVERRKWYGFWDYGDFMHSYDMVRHMWKYDIGGYAWQNTELVPNIWLWLMFLRTGRFDIFKMAEAMTRHTSEVDVYHLGEYKGLGSRHNVVHWGCGCKEVRISMAYLHRYYYYLTADERIGQLMDDVKDIDKQIMHMDPMRAYFTNDPENRVHIRVGPDVMTFCANWFVRWERHKEEIYKQKIIKILDFLKKNPAAFISGGVFDYDPEKTQLKPVEYTGGSNFVFCFGNTLVWLEIAKNFEDKEFEELCAQQGLFYTEFKENKNEILKSWGVPSFGFKLNMLNIGMAAFAAMKKNIPELKKEIWQMFLDHNKNPWLKFICDGGINLQLATVPAPVAEIPFISTNIASQWSLNALLALEFIGDEL

SEQ ID NO: 138 - DNA, 1569 bp, Anaerocellum thermophilum DSM 6725
atgagtaaagttaagaagattttgaggttatttgtagttagtatctgtattgcaggtttgttgttaacatcaataggagtattgggtgcatcaaaatcaaagtattcgtttaaactcacaatcatggctcagtattttgggacagagcctgcgccatcaaatagtccagtaattttaaaagcagaacagtacctaaaaactgacctggagtttacatgggtacctgccgatggttacaatgacaaattaaacattatgttagcaagtggaaatcttccaatggtggtttatgtaccaggaaaaactgcgtctataattggtgcatgtaaagcaggagcattctgggaacttggaccgtatataaagcaatataagaatttaaaagcaatcccggatatagttctgtggaactcttccattgatggtaaaatatatggtattccacgttcaaggaccttgggaagaaatggtattgtttatagaaaagactgggctaaaaatgttggtatcactaagcttgaaactattgatgatttgtacaacatgttaaagaagtttacttacaatgatccagataaaaatggaaaaaatgacacatatggaatgattgtttgcaactacaatggaccattctatattactctcacatggtttggtggtccgaatggatggggattgaacaaaaacggacagttggtgccaactttcttgacaaatgcatatatagaaaatttaaaattctggcgaaagatgtatcaagaaaagctgtttaaccatgacttcccgagtgttccaggtgcaagatgggaggattattactcacagggtaaaggtggagtaaagattgacgttatagactctgcaaacagaatttacaatggcctcaagaacaacggtatcattccaaaagatgcaaaggatacagatataatggacgttgttgtgtctgtgaaaggtaaatatggtctaagaaatatgccgacttctggttatgcggggtatctgatggtttcaaagacaagtgtaaaagatatgaatacattcaagaaagtaatgtcgatattagataagtttggcgacagaacaatgcaggatctgtttggttatggactaccgaacagacattacaaacttgtagatggtaaaatagatcctattccaaacttgccagcagatttgtcaagagaaataagcggacttaaccaggttctacatttttatcctgcaaacggtggaacaccaagatatatgactccacttttgcagctgcaggcagatatgcaagctttgaatgaaaagctcaatattcttgtgccaaatccggcagaagcattgataggtatgtcagatacttatatcaaacgcggtgtcacacttgataatatgatagaggatgcgcgcgttaaatatattactggtcagctcaatgaccagggctttaagaaagttcttgataactggagaaggcagggaggagaccagattataaaagaagtaaatgcactgtatcgtaaatataaaaagaatattccttacaaagaagacatatataagattctcaatccttag

SEQ ID NO: 139 - protein, 522 aa, Anaerocellum thermophilum DSM 6725
MSKVKKILRLFVVSICIAGLLLTSIGVLGASKSKYSFKLTIMAQYFGTEPAPSNSPVILKAEQYLKTDLEFTWVPADGYNDKLNIMLASGNLPMVVYVPGKTASIIGACKAGAFWELGPYIKQYKNLKAIPDIVLWNSSIDGKIYGIPRSRTLGRNGIVYRKDWAKNVGITKLETIDDLYNMLKKFTYNDPDKNGKNDTYGMIVCNYNGPFYITLTWFGGPNGWGLNKNGQLVPTFLTNAYIENLKFWRKMYQEKLFNHDFPSVPGARWEDYYSQGKGGVKIDVIDSANRIYNGLKNNGIIPKDAKDTDIMDVVVSVKGKYGLRNMPTSGYAGYLMVSKTSVKDMNTFKKVMSILDKFGDRTMQDLFGYGLPNRHYKLVDGKIDPIPNLPADLSREISGLNQVLHFYPANGGTPRYMTPLLQLQADMQALNEKLNILVPNPAEALIGMSDTYIKRGVTLDNMIEDARVKYITGQLNDQGFKKVLDNWRRQGGDQIIKEVNALYRKYKKNIPYKEDIYKILNP

SEQ ID NO: 140 - DNA, 879 bp, Anaerocellum thermophilum DSM 6725
atgagacagaataagacagtagcaagtactatttttgatgtttttaatcatatattccttggaatatgggcgataataactgttttacctttcctatacgttttggctgcatcttttgctccagattcagaaataaaaacgaggacattctttataattcctcataatcctactcttctcacctataaatttatttttgcttcaaactattttcttcgtagtatgctcaacagtgtgattatcacggtaggaggtaccttggtaaatctgtttttcacatttacaatggcatatgctctttcaaaaaagcactttataggcagaagtattgtcttaaatggcataatatttacaatgctttttggtggaggaatgattcctacatatttgctggtaaaaagcctgggtctcttaaattcttattgggcactatggttgccaggtgcaattagtccctttaacttctttgttgtcaaaaactttttccaggaaatgccacaggatttagaggatgcagcaagaattgatggatgtacagaagctcaagtgttgtggaagataatacttcctctttcaaagcctattattgcgacatttgctttgttctatggagtggggcattggaattcatggtttggagcgcttttgtatataaacgatgctgagaaatggcctgtgcagctaattttaaggcaaatagtaatgttgtcaaccacgcttgcttcggacctaacacaatttgatcctaatttccaacctccacaggagtcgctgaagatggcaataattgtcgtagcaactttaccaataatgcttttgtatccatggcttcagaagtacttcataaaaggtatgtttattggttcattgaaggagtga

SEQ ID NO: 141 - protein, 292 aa, Anaerocellum thermophilum DSM 6725
MRQNKTVASTIFDVFNHIFLGIWAIITVLPFLYVLAASFAPDSEIKTRTFFIIPHNPTLLTYKFIFASNYFLRSMLNSVIITVGGTLVNLFFTFTMAYALSKKHFIGRSIVLNGIIFTMLFGGGMIPTYLLVKSLGLLNSYWALWLPGAISPFNFFVVKNFFQEMPQDLEDAARIDGCTEAQVLWKIILPLSKPIIATFALFYGVGHWNSWFGALLYINDAEKWPVQLILRQIVMLSTTLASDLTQFDPNFQPPQESLKMAIIVVATLPIMLLYPWLQKYFIKGMFIGSLKE

SEQ ID NO: 142 - DNA, 966 bp, Anaerocellum thermophilum DSM 6725
atgaaaaacaatattatcaagggaccaaaaaagaagggagagaatgatttaatgacagctaccacttctatatggaaaagactgaaaaaagacaaatggctgtatatcttagctctaccgggtattttgtacttcatcatttttaggtacattccaatgtttggtatagtagttgcctttcaagattttaatccgtttttagggttctggaaaagtccgtgggtaggttttgagcattttaaaacacttttcaccgaccctgattttccgatgctgtttagaaatacactgttaatttcattttacaatatacttttctatttccctgtgccaattattttagcgctactgatcaacgaggtgcgaaatcaggtttataagagaattgtccagacgtgtgtttatgttcctcactttgtttcaatggtggtcatagcaagtattacatacgtgcttctctcaagtgaggcaggggttataaataatattctgtatagcttaacaggtaaaaaaattgaatttttgacagatccaagatggttcagacctcttataataattcagagcatatggaaagaagcaggatggggaacaataatcttcttggcagcactatcaaatgttgacccaacattatatgaggctgctattgtggatggggcaacgaggtggcagcagacgtggcatatcacaataccctcaattatgagtacggtaatcatacttttcattttgcgattgggacatcttcttgatactggctttgaacagatatttttgatgaaaaatcctattaacaggtcggtagcagaagtgtttgacacatatgtttatcaagtaggggttacccaaggagcgtacagttacagtacagcggttggtctttttaagtctgttgttggactgattttgattcaggtttctaactatctgtccaagaaatttactgaaacttcattgttctaa

SEQ ID NO: 143 - protein, 321 aa, Anaerocellum thermophilum DSM 6725
MKNNIIKGPKKKGENDLMTATTSIWKRLKKDKWLYILALPGILYFIIFRYIPMFGIVVAFQDFNPFLGFWKSPWVGFEHFKTLFTDPDFPMLFRNTLLISFYNILFYFPVPIILALLINEVRNQVYKRIVQTCVYVPHFVSMVVIASITYVLLSSEAGVINNILYSLTGKKIEFLTDPRWFRPLIIIQSIWKEAGWGTIIFLAALSNVDPTLYEAAIVDGATRWQQTWHITIPSIMSTVIILFILRLGHLLDTGFEQIFLMKNPINRSVAEVFDTYVYQVGVTQGAYSYSTAVGLFKSVVGLILIQVSNYLSKKFTETSLF

SEQ ID NO: 144 - DNA, 2535 bp, Anaerocellum thermophilum DSM 6725
atgaatgagagtataaaattgaattctctttttgatgaaatcaacaaccctccagctactgccagtattattcattggtggattttttctgatgagatgaacgagaacagaataaatgctgagcttgattatatttcaaatcttggctttaagcaagtattaattgcagtaggacacaatgtttcgcctaaatatttgacacatggttggtttgaaatggtaaaatttgcagttctccaagctaaaaaaagaggagttaaagtgtggattgccgatgaagggacatatccaagtggctttgctggcgaaacttttaataagaagtatcctcacaaaaggatgaaggctattattgttgagaaggagtttattattgagggtaatttatgtgaagttgaacctcactctggtacaattgggattttggccaaagacatgaaccagaataaatactttgcttttgaaaagcttgaatttagtagcggatttttatacttgccctatcattcgacttggcaaataaaagtaatatcttcagcttacaggacatctccaacaagatacgttcaccatccaacaggtgcaaaagatactacattttcactttgtgattatcttgactatgaagctgtcaatctattcataagtgaggtatatgaaaaatataaagcttatatgggaaatgaatttggaaagacaataattggatttttcgctgacgaacctgattattctatttctggactaccatatacggataatatatttgatatattttacaatgaacaaggatacgacgttaaaaagtacataccgtatttctttaaagagcaattagatgaaaaaataaaaagagtaaaagcagattactgggatgtatggagcaatatttttacaaatactttctttaagcagatctacaaatggtgtgaggcaaatggcctcaaatttgtagtacatctaaatcatgaagatatgatagaacaccttaccaaatctgaaggacagttcttttcgcatatgaagtatgttcatattccagcaattgatgtaatttggagacaaatctggtatgacaaaaaagcaatattccctaaatacgcttcttctgtttctcatattaaaaatattgctcagaccttttcagagagttttgcagtatatggacaaggtatatctgttgagcaaatcaaatgggtagttgattaccagtttgcaatggacataaatctatttttgacctcaatcttcaagtatctttatgaccatccgcaaaattatttctttccagaggtaattaagtatattaataccatttcatatcttctctatgtaagcaccccttgtacaaaggttctggtttactttcctacaccggatctgtgggcaggtgaaaatatgtctgcttcaaaagcaatggaaattggcaatgcacttttagagaaccagattgattttgatttttttgaccattctcttttagaatatctggaaattaaaaaccatagaatatacgctaacaatagaaaagaatacgacattgttattcttccgcctataaagtatttgccacaagatctgttcagatttttaaagcttttctcaagcaaaggagggaagattattttcttcgagaactctcctttgtttgtttataacaaaacctttacatcgtttttccactttgtagatagagaaataggtgtggttgttgaaagtatcgagcagctttcaaaaatggttgaaaaagatgtcactgttgtagacagcaaagatgttagagttcttcataaaagaatagaaggcaataatctgatttttctcttcaatgtttcaggtacttcatttttgggtaagataatattaaaattttctaagaaaaatgtatatatatgggatcatatacagaataaatttttaatggtttcaaatatcaaaagtaataaaaaaaacatacaattagaactctatatacatccatatcagactttggttttaatagcaagtgatgagtatgtagatggaattcaaaaaacaacactgcttggaagcttaccgagaacagtcttggaattaaacgataactgggaaattcattttgataaagattttgttttgttttcagatttaaaagattggcaaagcttgggctttggtgactattctggcagtgtagtttatagaaaaatattttcgttttctcatgatgactttattaaaaataaacatcttttcctcaactgccccaatgtaaagtactctgcaaaggtttggttaaataaaagatatcttggtgtaagagctttttcgccttttatgtgggatataacagaggcattgaaaattggtgagaatgaacttgtgattgaagttcaaaacacccctgcagcagctctacttggaacacaagaaaaattggaaaaattaagaaaagaggcagagaagaacttttatctttctatttctctaaaatttgacctggaaatggtccaatcaggattgttgcctccagttgctattgtttctttagaatga

SEQ ID NO: 145 - protein, 844 aa, Anaerocellum thermophilum DSM 6725
MNESIKLNSLFDEINNPPATASIIHWWIFSDEMNENRINAELDYISNLGFKQVLIAVGHNVSPKYLTHGWFEMVKFAVLQAKKRGVKVWIADEGTYPSGFAGETFNKKYPHKRMKAIIVEKEFIIEGNLCEVEPHSGTIGILAKDMNQNKYFAFEKLEFSSGFLYLPYHSTWQIKVISSAYRTSPTRYVHHPTGAKDTTFSLCDYLDYEAVNLFISEVYEKYKAYMGNEFGKTIIGFFADEPDYSISGLPYTDNIFDIFYNEQGYDVKKYIPYFFKEQLDEKIKRVKADYWDVWSNIFTNTFFKQIYKWCEANGLKFVVHLNHEDMIEHLTKSEGQFFSHMKYVHIPAIDVIWRQIWYDKKAIFPKYASSVSHIKNIAQTFSESFAVYGQGISVEQIKWVVDYQFAMDINLFLTSIFKYLYDHPQNYFFPEVIKYINTISYLLYVSTPCTKVLVYFPTPDLWAGENMSASKAMEIGNALLENQIDFDFFDHSLLEYLEIKNHRIYANNRKEYDIVILPPIKYLPQDLFRFLKLFSSKGGKIIFFENSPLFVYNKTFTSFFHFVDREIGVVVESIEQLSKMVEKDVTVVDSKDVRVLHKRIEGNNLIFLFNVSGTSFLGKIILKFSKKNVYIWDHIQNKFLMVSNIKSNKKNIQLELYIHPYQTLVLIASDEYVDGIQKTTLLGSLPRTVLELNDNWEIHFDKDFVLFSDLKDWQSLGFGDYSGSVVYRKIFSFSHDDFIKNKHLFLNCPNVKYSAKVWLNKRYLGVRAFSPFMWDITEALKIGENELVIEVQNTPAAALLGTQEKLEKLRKEAEKNFYLSISLKFDLEMVQSGLLPPVAIVSLE

SEQ ID NO: 146 - DNA, 882 bp, Anaerocellum thermophilum DSM 6725
atgctaaaaagacaggatatttatatccgtgacccttttattatgcctgtaaaagaaaaaagggtgtactatatgtttggtacaactgataaaaattgctggggaaatgaaaaagcaacaggctttgattattatgtaacctatgaccttgaaaattttgatgggccatatccagcattcaggcctcctcagaatttttgggcagacagaaatttctgggcgccagaggtgcacttttataacggcaaatattatatgtttgcaacattttgttcaccagaaggaaaaaggggaactgcgattcttgtttcagatactgtagaaggaccatatgttgaacatagtcttggacctgttacaccaaaggattggatgtgccttgatggaacactttacatagatgaagattcaaatccctggcttgtattttgccgtgaatgggttgaggtttatgatggtcagattttggcagcaagactttcaaatgatttgaaaagagttattgatgagcccgttttactctttacagcctccagcgcaccttggacagcaattataagagttaaagatggcagagaatgttttgtgacagatggtccgtttttattcagaacccaaaacggtactcttttgatgctatggtcaagttttgatagagaaagaagatattgtgttggagttgcaaaatcactgtcaggcaatattttaggtccatggcagcacaaaacagagccaattttttctaatgacggtggtcatggcatgctttttagaacctttgaaggtgaccttatgctttcaattcatactccaaattcaaggggcaatgagaggcctttgtttataaaaattgatgataaaaatttagaagacgagttttga

SEQ ID NO: 147 - protein, 293 aa, Anaerocellum thermophilum DSM 6725
MLKRQDIYIRDPFIMPVKEKRVYYMFGTTDKNCWGNEKATGFDYYVTYDLENFDGPYPAFRPPQNFWADRNFWAPEVHFYNGKYYMFATFCSPEGKRGTAILVSDTVEGPYVEHSLGPVTPKDWMCLDGTLYIDEDSNPWLVFCREWVEVYDGQILAARLSNDLKRVIDEPVLLFTASSAPWTAIIRVKDGRECFVTDGPFLFRTQNGTLLMLWSSFDRERRYCVGVAKSLSGNILGPWQHKTEPIFSNDGGHGMLFRTFEGDLMLSIHTPNSRGNERPLFIKIDDKNLEDEF

SEQ ID NO: 148 - DNA, 1788 bp, Anaerocellum thermophilum DSM 6725
atgataagaaaaaatggagtttataaaagcaagattttcaaaaagaattttgtgcagatagttattgttcccattgtgataataactattcttgggcttttctcatgcatcattatagaacaatacgttaaaaatgaaataaacaaaaatttagagacaatgctaatacaaagcaaaaacaatgtcgagcttatgctcggtgagatagactatctttatatggtatttgggataaacaaagatgtgacccttcagataaagaggattttgaactcaatgtatttttctttagaagatatctggcagattaacatggtcaaaaatgttttaaattcaatctcatattcaaagccgttcatacattccatctatgtttatttcgaaaatcctgaagggaattttatagttaccccagatggaatgactaattttcagtatttttatgacaaatggtggtttgatcagtataaagaaaataaagcattaatgtgggtagagagaagaaaaattcaaccttacaattttactggagaatcaattgatgttttgaccatctacaaaaggataaaatctgcatattctgatgtgaatgagggtgttattgttcttaatctgtattacgaccaggtaaaaaagctcttaagccttaaaagttcgctccctcagcatgcaatgtacatattagatcaaaatggaaatgttttggtatcaaatgaatcagataactctaatacctcaagtatggccctcctaaaaaaagaaacagacaactatctcacaaaaagattagagtcaaaaaaatacaacttaacctttgtttcagtaattcccaaaaattatctttacagcatccctatcagacttttcaaggtgacactggtgctacttttaatttttatagttatcgcttttgctgcctcatactacattgccaaagtaaattacaggaatattaaaaagattatagatacaataaattcagcaacagaaggaaaaccaccaaaagaaattaaaattacttcaaatgatgaatatgggtatatcatgtacaatgtaatcaagaactttattgaaaaacattatctgacaacacgccttcaagctttggagcttttagcattgcaggctcagattaaccctcattttcttttcaatactttagaacacatatatcttaaaactttagcacttacaggcaccccaaacgagattacaaaaatgatagaaaacctttcggctatactcaaatattctctgagcaatccaaaaattactatcttcctaagggaagaaattaaagctacacaggcatatattgagcttgtaaaagcaagatataaagataagtttgatgtgttttgggactatagtgaagatgtgcttgagataaaagtgatgaagcttttattccagccgctcatagaaaattcaatctatcatgggataaaaccttgcgaaaagagatgtggaataaaaatcaggataagaaaattaaaagataccagtgattggctttgtatatgggtaattgacaatggaattgggatgagcaaagaaaagttagaggaggtacaaggcaggctttcacaggattttgacttttcagatcatattgggcttttaaacaccaatgaaaggttaaagctcaactatgggggtaactttaaactcaaggtttggagcaagctgggtttggggacaattgtaaaaataattcttcctgtgaattttgaggaccgaaaggagaatgaaatagatgctaaaaagacaggatatttatatccgtga

SEQ ID NO: 149 - protein, 595 aa, Anaerocellum thermophilum DSM 6725
MIRKNGVYKSKIFKKNFVQIVIVPIVIITILGLFSCIIIEQYVKNEINKNLETMLIQSKNNVELMLGEIDYLYMVFGINKDVTLQIKRILNSMYFSLEDIWQINMVKNVLNSISYSKPFIHSIYVYFENPEGNFIVTPDGMTNFQYFYDKWWFDQYKENKALMWVERRKIQPYNFTGESIDVLTIYKRIKSAYSDVNEGVIVLNLYYDQVKKLLSLKSSLPQHAMYILDQNGNVLVSNESDNSNTSSMALLKKETDNYLTKRLESKKYNLTFVSVIPKNYLYSIPIRLFKVTLVLLLIFIVIAFAASYYIAKVNYRNIKKIIDTINSATEGKPPKEIKITSNDEYGYIMYNVIKNFIEKHYLTTRLQALELLALQAQINPHFLFNTLEHIYLKTLALTGTPNEITKMIENLSAILKYSLSNPKITIFLREEIKATQAYIELVKARYKDKFDVFWDYSEDVLEIKVMKLLFQPLIENSIYHGIKPCEKRCGIKIRIRKLKDTSDWLCIWVIDNGIGMSKEKLEEVQGRLSQDFDFSDHIGLLNTNERLKLNYGGNFKLKVWSKLGLGTIVKIILPVNFEDRKENEIDAKKTGYLYP

SEQ ID NO: 150 - DNA, 759 bp, Anaerocellum thermophilum DSM 6725
atgtcttacaagatgattatcgtggaggatgaaagtgagataagacaggggctttttacctgctttccatggaataaacttggttttgaagttgttggcctgtttgaaaatggaaagcaggctttagattatattttgcaaaataaggttgatgttgttttttgcgatattaaaatgcctgttatgagtggaattgaccttgcaagggttttgtttttaaataacatccctgtaaaagttgtttttataagtggttatcaggactttgaatttgctcaaaaagcaatgaaatacggggtaaggtactacataacaaaaccagcaacgtatgatgaagtaatagagatatttgggctaataaaaaaagagcttgacctacagtcaaaaaatccgcaaactcacagtgaagaggtaaaaaaacaagatttaatttctattgacaatcctgaaaatgtgattgaaactgtgaaagagtatataaaaagaaactataaagaggccacgcttgaggatgctgcaaagcttgtctatatgaatccttattatcttagccggcttttcaaatgcaaaactggtaaaaacttttctgattatctgatggaagtcaggatgagaaaagctctggaacttatgaaaattcctatatataaacttcatgagataggtgaaatggttggttacaagaaccctaagaactttacaagagcttttaaaaaatatttcggcaagtctccttcagagtttgttcaggagagataa

SEQ ID NO: 151 - protein, 252 aa, Anaerocellum thermophilum DSM 6725
MSYKMIIVEDESEIRQGLFTCFPWNKLGFEVVGLFENGKQALDYILQNKVDVVFCDIKMPVMSGIDLARVLFLNNIPVKVVFISGYQDFEFAQKAMKYGVRYYITKPATYDEVIEIFGLIKKELDLQSKNPQTHSEEVKKQDLISIDNPENVIETVKEYIKRNYKEATLEDAAKLVYMNPYYLSRLFKCKTGKNFSDYLMEVRMRKALELMKIPIYKLHEIGEMVGYKNPKNFTRAFKKYFGKSPSEFVQER

SEQ ID NO: 152 - DNA, 858 bp, Anaerocellum thermophilum DSM 6725
atgtactcccaaaacaaaatagtaaaaaatactatttctctttttataacttatgcatttttgattctatttggactgtttatgatttatccacttttgtgggttgtatctgcggcatttaaatcaaatgatgagatattcaaatcgctgtctttatttcctcaaaaaattgttacagactcatttataaaaggatggcaggggacagggcaatatacatttggtagattttttgcaaataaccttatacttgttattccagttgtattttttacaataatatcctctacacttgtggcatatgggtttgcaaggtttaattttccactgaaaagattattttttgtgatactaatttcaacattaatgctgccagactctgtaaaccttattccaagatatattcttttcaatgcatttggatgggtagatagttacaaaccatttataattccatcaatgtttgcatcaactccattttttgtgtttatgatgatacagtttatgagagggcttccaagagaaatagaagaagctgctataattgatggctgcaactctttccagatacttttgcgtataacgggtcctctttgtaaaacagcaatgatttcaatgggaattttccagtttatctggacttggaatgactttttaggaccccttatatatatcaacagcgttgaaaaatacacaattgcccttggacttagaatgtgtgttgacagtgcagctgcaattgcttggaaccagatcatggctatgactgttatagcaatgctaccgtgtattatcatcttctttgctgctcaaaaatacttcgttgaaggaattgcaacaagcggaataaaaggttaa

SEQ ID NO: 153 - protein, 285 aa, Anaerocellum thermophilum DSM 6725
MYSQNKIVKNTISLFITYAFLILFGLFMIYPLLWVVSAAFKSNDEIFKSLSLFPQKIVTDSFIKGWQGTGQYTFGRFFANNLILVIPVVFFTIISSTLVAYGFARFNFPLKRLFFVILISTLMLPDSVNLIPRYILFNAFGWVDSYKPFIIPSMFASTPFFVFMMIQFMRGLPREIEEAAIIDGCNSFQILLRITGPLCKTAMISMGIFQFIWTWNDFLGPLIYINSVEKYTIALGLRMCVDSAAAIAWNQIMAMTVIAMLPCIIIFFAAQKYFVEGIATSGIKG

SEQ ID NO: 154 - DNA, 897 bp, Anaerocellum thermophilum DSM 6725
atggtagttcgctatagaaaaaaagactttgttggttttttatatatactaccttggcttattggatttttgatttttaggctatatccatttattatgtctttctactactctttcagtgattatacaatgttgaagccgcctcgctatgttggattatataacttcatttatatgttcacaaaggatgaactgtttccaaaagcacttttaaacactataaagtatgttataataactgttccgcttaaaatctcgtttgcactttttgttgcaataatattgaacatgaaactgaaaggaataaatcttttcagaacagtatattatcttccttctatcttcggtgggtctgttgcaatctcgattttgtggagatttttgtttatgaaagaaggtatagtaaacaagttcttaagtctttttagaatagaaggtataaactggcttggagacccaagaatagctatgttttcagtaagccttcttgcggtgtggcagtttgggtcatctatggtactatttttagcaagacttaaagagataccatcagaactttacgaagcagcactggttgatggagcatcaagactaaaaatgtttacaaagataactcttcctatgatttcacctataatgtttttcaaccttgtgatgcagaccataaatgctttccaagaatttactggaccgtacatcatcacaggcggtggacctgtcaattccacctatcttttgagtatgctcatatatgacaatgcatttaagtattttagaatgggttatgcggcagcactttcctgggttcagtttgtgataatattaatctttactgcatttatatttaggtcttctacttattggacatattacgagtatgatgaagggaggttctaa

SEQ ID NO: 155 - protein, 298 aa, Anaerocellum thermophilum DSM 6725
MVVRYRKKDFVGFLYILPWLIGFLIFRLYPFIMSFYYSFSDYTMLKPPRYVGLYNFIYMFTKDELFPKALLNTIKYVIITVPLKISFALFVAIILNMKLKGINLFRTVYYLPSIFGGSVAISILWRFLFMKEGIVNKFLSLFRIEGINWLGDPRIAMFSVSLLAVWQFGSSMVLFLARLKEIPSELYEAALVDGASRLKMFTKITLPMISPIMFFNLVMQTINAFQEFTGPYIITGGGPVNSTYLLSMLIYDNAFKYFRMGYAAALSWVQFVIILIFTAFIFRSSTYWTYYEYDEGRF

SEQ ID NO: 156 - DNA, 1317 bp, Anaerocellum thermophilum DSM 6725
gtgataaaaagtaaaaggttaattgcaactcttgtgttagtagtttttactatgtcgatcttctttgctttttcgaccgctgggtctgagaaagctaaggcagcatcgaaaaaggttacactcaggtttatgtggtggggcggagaggcaagacacaaagccactttggcagcaattcaggcgtatatgaagaaataccctaatgtaagaattaatgcagagtatggcggtattgaaggttacatgcagaagctcattacccagcttgtgggaagaaccgctccagatataatccagattgacgttacatggattggtgagctgagcagccagggagatttctttgcagaccttaaaactttcaaagaggtcaacttaaagccatttgaagagaagtttttaaaagactggtgctattcaaacggaaaacttattggacttccaacaggtgttaatgcttcggtacttcaatataataaagagttttttaagaagtttaatatcgacgaaaatacagtttggacgtgggataacttactatcaatagctgaaaaagtacacaaaaaagataaaaatagttatttgcttaattttgatcaaatcctctgttactatgttttgacatcgtatattggtcaaaaaacaggaaaggattggattttagatgattatacattaggatttaataggaatcagttgatagaagcttttacttatttgaaaaaattatttgatgtaggagctattcaaccttttgcagaaagtgcgccatttcaaggtaagccagaacaaaatccaaaatggctaaaaggagaattagggattttatggaattggacttcaacttatgctgcaaataaagctatgattccaagtttggcgatgacattaccacccaggggcaacaacttgaaaaactatgcagtaactgtcagaccgtcgcaattgttatctgtaaataaactttcgaagaatgctaaagaagctgcaaaatttattaactggtttttaaacgataaacaagctgctctaatactcactgatgtaagaggagttcctgcaagttcaagtgccagagatgcattgttaaaggcaaataaattagatccagaaatattgagggttacaaacgaagcggtaaagtatgcagcaaaaccacagaatgcactatcacagaatcaagaaatagcaaatatagcatatgatatcattcagcagcttgcatacaaacagctaacaccgacacaggctgcagataaattgatagcattatataaacaaaaactttctgagcttaaaagaatgcagtctcgatag

SEQ ID NO: 157 - protein, 438 aa, Anaerocellum thermophilum DSM 6725
VIKSKRLIATLVLVVFTMSIFFAFSTAGSEKAKAASKKVTLRFMWWGGEARHKATLAAIQAYMKKYPNVRINAEYGGIEGYMQKLITQLVGRTAPDIIQIDVTWIGELSSQGDFFADLKTFKEVNLKPFEEKFLKDWCYSNGKLIGLPTGVNASVLQYNKEFFKKFNIDENTVWTWDNLLSIAEKVHKKDKNSYLLNFDQILCYYVLTSYIGQKTGKDWILDDYTLGFNRNQLIEAFTYLKKLFDVGAIQPFAESAPFQGKPEQNPKWLKGELGILWNWTSTYAANKAMIPSLAMTLPPRGNNLKNYAVTVRPSQLLSVNKLSKNAKEAAKFINWFLNDKQAALILTDVRGVPASSSARDALLKANKLDPEILRVTNEAVKYAAKPQNALSQNQEIANIAYDIIQQLAYKQLTPTQAADKLIALYKQKLSELKRMQSR

SEQ ID NO: 158 - DNA, 1227 bp, Anaerocellum thermophilum DSM 6725
atgaaaaaagtattcttacaatttggttctgggttaggtcccatgtctcgatcccttccaatcgcagaagggttacaaagagaaggatatatcgtaaagtattttggatttgaaaatgcaaaaccatatatgaataaaataggaattgaagagttatcagaaaattttaatatcaaggatattaaaaaaggagtgcaaactcctaattggtattgtgcagagcaattttgggaaataataggatatggtaatatggaatgggtagaaaaaaaagttgaagaattaatagaatatttaaaagatttttctcctgattttataatatcagaccttggtatattaagttgtattgctgcaagaataatggacatacctttgatagctataactcaaagttgttatcatcctaacattgcttttggaagaataagatggtgggaagaagaacaaaatttaaagtttacattaactgagaaattaaataattattttaagaaaaaaggtgtttcacaattaaattcttttgaagaaatttttactggtagtttaaccataattcccagttttcctgaatttgatccaataaataatccttcagaatttaacacatattatgttggtcccatattatgggatccattagacatggctaaagaagagtatataaaattgtttaacagagataaaaataagcctacaattttttgctatacagcaagattttacgacaatgtgggtgaaagtggaattattatttttaaaacattactttcagcattaaaaaaatttgatgctaacattattttttctacagggagtgattcggacaggaaaatagcaaaagagattttaaactcttacggaattgatgaagagaaatttagcattattgattgggttccaatgggaattgcttatggaaactctgatgttgttatccatcatggaggccatggaagttgtttaggtcaatttttgtatgaggtaccttcattaattatacctactcatactgaacgagagtataatgcaagaatttgcaccaatatgggagtttctaaatttataaaaagagaagacattgaaaaagcagatatattagctgaaattgttgagattttaactaactcaagttttaaagaaagattacacttttggcatactaaactaaatgaatataattttacaggtgtaaataaagttttagaattgattcaaaaattataa

SEQ ID NO: 159 - protein, 408 aa, Anaerocellum thermophilum DSM 6725
MKKVFLQFGSGLGPMSRSLPIAEGLQREGYIVKYFGFENAKPYMNKIGIEELSENFNIKDIKKGVQTPNWYCAEQFWEIIGYGNMEWVEKKVEELIEYLKDFSPDFIISDLGILSCIAARIMDIPLIAITQSCYHPNIAFGRIRWWEEEQNLKFTLTEKLNNYFKKKGVSQLNSFEEIFTGSLTIIPSFPEFDPINNPSEFNTYYVGPILWDPLDMAKEEYIKLFNRDKNKPTIFCYTARFYDNVGESGIIIFKTLLSALKKFDANIIFSTGSDSDRKIAKEILNSYGIDEEKFSIIDWVPMGIAYGNSDVVIHHGGHGSCLGQFLYEVPSLIIPTHTEREYNARICTNMGVSKFIKREDIEKADILAEIVEILTNSSFKERLHFWHTKLNEYNFTGVNKVLELIQKL

SEQ ID NO: 160 - DNA, 1317 bp, Anaerocellum thermophilum DSM 6725
atgaagtacttcaaagacattccagaagtaaaatatgaaggaccacagtcggacaacccatttgctttcaagtactacaatcctgacgaaatcattgacggcaagcctttgaaagaccaccttcgttttgctattgcttactggcacacattctgtgcaacaggaagcgatccgtttggacaacctacaattgttcgtccttgggataagttttcaaaccgaatggacaacgcaaaagcaagggttgaggcagcatttgaattttttgaactgttagatgtaccatttttctgcttccatgacagagatattgcacctgaaggggaaaatttaaaagagtcaaataagaatttggatgagattgtttctttaataaaagagtatttgaaaaccagcaagacaaaagtattatggggaacagcaaacctattttcacatccgcgatatgttcatggtgctgcaacatcctgcaatgccgatgtttttgcatatgcagcagcgcaagtgaaaaaggcgttagaggttacaaaagagcttggcggcgaaaactatgtgttctggggcggaagggaaggttatgagacacttctaaatacagatatgggattggaacttgataaccttgcaagatttttgcatatggcggttgagtatgcaaaggaaataggttttgacggacagtttttaatagaaccaaaaccaaaagagccaactaagcatcagtacgattttgattcggctcatgtttatggatttttgaaaaagtatgatcttgacaaatacttcaagctcaacatagaggtaaaccatgcaaccttagcaggacatgatttccaccatgagttgagatttgcgcgaataaacaacatgcttggttcaattgacgctaacatgggcgatttgcttttgggctgggatacagatcagttcccaacagatgtaagacttactacacttgctatgtatgaggttattaaagctggtggttttgacaaaggtggacttaactttgacgcaaaggtaagaagaggttcttttgagcttgaagacttggtcattggtcacattgctggcatggatgcttttgctaaaggcttcaagattgcgtataagcttgttaaagatggcgtatttgataaatttatagatgagagatacaagagctacaaagaaggaatcggtgctaagattgtaagcggtgaagcaaacttcaagatgttagaggaatatgctctgtctcttgacaagatagaaaataaatctggcaagcaagagcttcttgagatgattttgaacaaatatatgttcagcgaataa

SEQ ID NO: 161 - protein, 438 aa, Anaerocellum thermophilum DSM 6725
MKYFKDIPEVKYEGPQSDNPFAFKYYNPDEIIDGKPLKDHLRFAIAYWHTFCATGSDPFGQPTIVRPWDKFSNRMDNAKARVEAAFEFFELLDVPFFCFHDRDIAPEGENLKESNKNLDEIVSLIKEYLKTSKTKVLWGTANLFSHPRYVHGAATSCNADVFAYAAAQVKKALEVTKELGGENYVFWGGREGYETLLNTDMGLELDNLARFLHMAVEYAKEIGFDGQFLIEPKPKEPTKHQYDFDSAHVYGFLKKYDLDKYFKLNIEVNHATLAGHDFHHELRFARINNMLGSIDANMGDLLLGWDTDQFPTDVRLTTLAMYEVIKAGGFDKGGLNFDAKVRRGSFELEDLVIGHIAGMDAFAKGFKIAYKLVKDGVFDKFIDERYKSYKEGIGAKIVSGEANFKMLEEYALSLDKIENKSGKQELLEMILNKYMFSE

SEQ ID NO: 162 - DNA, 1914 bp, Anaerocellum thermophilum DSM 6725
atgagaatgctattaaaaaggtctcttgctctgctggtaagtattgtccttgtattttcgttgttcttaagtgtttttccacagcaagcaagagcacaagataccataaaaattgtaggtaactggcaggatgctggtaactggaattttgacagctctaatattgttttgagtgaaacctcaactccgggactttattatggtgagtatacttttaaaactggtggttcttatgagttcaaagcagttataaatggtagtatatggtgtacaggcgctccaaaagtagcagataataatactaacatacctcttaatgttacagatggtcagacagttaagttttggttcttcaaaaattctcaacttgtaattgacagcactcattttccaaatgggcctgatagtttagtagggcaaaattcttttaaatttgtaggagtaaataatgaatggaaccctaatgatggtaagtatcaatttataagagtcccagatgcaacatatacctatttatatgataatagttacaatgatttttcgataccatatggatttaagattataataggtggttttgggaacctatcatgggcatggaatggtgcaaaagaaggttcagttgttaaatttaaagagggtggagataatattgacctgaaagaatttaaagaatctaatgataatcttctaaaaacaaaatttttccttgatatcctaaacggctggctctttacagaaaaagatttgacaaatatccaaccagtagattttgcaaataacggctctgtaattggtggaacttcagttcatcttacatggacaccatatacaccaaatgcaaatccactttcagccaagctttactacaaaataattgatgtgagcaatgatagcgagttggttagtctcaccgattacagcacaatccacaaaatagatatcccaagagagtggataggaaagacaattaaaattattgcaaatgccaagataggtgaaataacaggacctaatgttgagtttacattaaatattgttgacttgccacaaaacttaatagttgaagcagctaactatataacatacaactctatagatgagacatttttaaatttggctcaaggtgatacagttcaaaacataacttctaacttcgcagttgttacatcatatgtttacaatgtggtgtacgagggaaatagttatccactgacatttaatattgactgggagagcagtaattcatctgtattaacaataaatggtagtacagttgtggttacaaggccaacacaaggtgaccttcaggttgagttgagagcaagagcaagatttggtgctatttcagcagatggacaaaaaatatttactcttacagtaaagaaattcctattaggagtagatggtggaatacctgttacatttaacgtaacagttccagattatactcctgataatgacaatatttacattgctggagattttaagactgataagcttccaaaatgggaccctgtaggtatcaaattgataaaggtcggagataaaaaatatagtataacaatgtatcttccaccgaatgtaacaattgaatataaatatacccgcggaagttggtcaaaagtagaaaaggatgcatttggtaatgaaatatcaaatagagttctgcagataaataatcaggctgtgacaaaaaatgatgtagttgaagcatttgcggaccttggagctgtcaagcaaggtcttccaacggtgaatttggtcatcaatgttcctcaacaaactgtaattgatacaggtgatggaggaagggtaataaaagcagaaggtggtgctatttcagttccaaatactaagaacactgttttgatagatattcggagtgcattaaatgctattatattaaagtaa

SEQ ID NO: 163 - protein, 637 aa, Anaerocellum thermophilum DSM 6725
MRMLLKRSLALLVSIVLVFSLFLSVFPQQARAQDTIKIVGNWQDAGNWNFDSSNIVLSETSTPGLYYGEYTFKTGGSYEFKAVINGSIWCTGAPKVADNNTNIPLNVTDGQTVKFWFFKNSQLVIDSTHFPNGPDSLVGQNSFKFVGVNNEWNPNDGKYQFIRVPDATYTYLYDNSYNDFSIPYGFKIIIGGFGNLSWAWNGAKEGSVVKFKEGGDNIDLKEFKESNDNLLKTKFFLDILNGWLFTEKDLTNIQPVDFANNGSVIGGTSVHLTWTPYTPNANPLSAKLYYKIIDVSNDSELVSLTDYSTIHKIDIPREWIGKTIKIIANAKIGEITGPNVEFTLNIVDLPQNLIVEAANYITYNSIDETFLNLAQGDTVQNITSNFAVVTSYVYNVVYEGNSYPLTFNIDWESSNSSVLTINGSTVVVTRPTQGDLQVELRARARFGAISADGQKIFTLTVKKFLLGVDGGIPVTFNVTVPDYTPDNDNIYIAGDFKTDKLPKWDPVGIKLIKVGDKKYSITMYLPPNVTIEYKYTRGSWSKVEKDAFGNEISNRVLQINNQAVTKNDVVEAFADLGAVKQGLPTVNLVINVPQQTVIDTGDGGRVIKAEGGAISVPNTKNTVLIDIRSALNAIILK